(19) United States
(12) Patent Application Publication
(10) Pub. No.: US 2024/0197681 A1
(43) Pub. Date: Jun. 20, 2024

(54) METHODS AND COMPOSITIONS RELATING TO PSYCHEDELICS AND SEROTONIN RECEPTOR MODULATORS

(71) Applicants: Terran Biosciences Inc., New York, NY (US); University of Maryland, Baltimore, Baltimore, MD (US)

(72) Inventors: Sam CLARK, New York, NY (US); Scott THOMPSON, Baltimore, MD (US)

(21) Appl. No.: 18/553,170

(22) PCT Filed: Apr. 1, 2022
(86) PCT No.: PCT/US22/23067

§ 371 Date: Sep. 28, 2023

Related U.S. Application Data
(60) Provisional application No. 63/308,206, filed on Feb. 9, 2022.
(60) Provisional application No. 63/294,801, filed on Dec. 29, 2021.
(60) Provisional application No. 63/274,308, filed on Nov. 1, 2021.
(60) Provisional application No. 63/169,722, filed on Apr. 1, 2021.

(51) Int. Cl. A61K 31/4045; A61K 9/20; A61K 31/137; A61K 31/44; A61K 31/522; A61K 31/675; A61K 39/00; A61K 45/06; A61P 25/24

(52) U.S. Cl. CPC A61K 31/4045; A61K 9/20; A61K 31/137; A61K 31/44; A61K 31/522; A61K 31/675; A61K 45/06; A61P 25/24; A61K 2039/545

(57) ABSTRACT

Provided herein are compositions relating to psychedelics and serotonin receptor modulators. Further provided herein are methods of treating a disease or disorder (e.g., depression or diseases or disorders related to depression) comprising administering psychedelics and serotonin receptor modulators.

### CROSS REFERENCE TO RELATED APPLICATIONS

This application claims the benefit of U.S. Provisional Patent Application No. 63/169,722, filed Apr. 1, 2021, U.S. Provisional Patent Application No. 63/274,308, filed on Nov. 1, 2021, U.S. Provisional Patent Application No. 63/294,801, filed on Dec. 29, 2021, and U.S. Provisional Patent Application No. 63/308,206, filed Feb. 9, 2022, the content of each of which is incorporated by reference herein in its entirety.

### GOVERNMENT RIGHTS STATEMENT

This invention was made with government support under grant number MH086828 awarded by the National Institutes of Health. The government has certain rights in the invention.

### BRIEF SUMMARY

Psychedelic substances have been used by humans for millennia for spiritual and medicinal purposes. Clinical research has recently begun to provide evidence supporting their use as therapeutics for numerous neuropsychiatric disorders, including obsessive-compulsive disorder, posttraumatic stress disorder, and treatment-resistant depression (TRD). However, the use of psychedelic substances such as psilocybin are not widely utilized for therapeutics because of the intense psychedelic-induced alterations in sensory perception and consciousness that they produce. The therapeutic potential of psychedelics could be improved if the psychedelic response could be diminished without impairing the therapeutic response. Provided herein are compositions and methods relating to psychedelics and serotonin receptor modulators that provide therapeutic effects while reducing the psychedelic-induced alterations in sensory perception and consciousness.

Described herein, in certain embodiments, are compositions comprising: a) a psychedelic; b) a serotonin receptor modulator; and c) an excipient, wherein the serotonin receptor modulator is released at most about 3 hours prior to the release of the psychedelic. In some embodiments, the psychedelic is an agonist for a serotonin receptor. In some embodiments, the serotonin receptor is serotonin receptor 1B, serotonin receptor 4, serotonin receptor 6, or serotonin receptor 7. In some embodiments, the psychedelic is selected from the group consisting of psilocybin, psilocin, baeocystin, norbaeocystin, lisurgide, LSD, dimethyltryptamine, carboxamindotryptamine, ibogaine, tabernanthalog, 3,4-methylenedioxy-methamphetamine (MDMA), 1-acetyl LSD, O-acetyl psilocin, mescaline (3,4,5-trimethoxy phenethylamine), proscaline (2-(3,5-dimethoxy-4-propoxyphenyl)ethanamine), metaescaline (2-(3-ethoxy-4,5-dimethoxyphenyl)ethanamine), allylescaline (4-Allyloxy-3,5-dimethyloxy phenylethylamine), methallylescaline (4-Methallyloxy-3,5 dimethoxyphenethylamine), asymbescaline (3,4-Diethoxy-5-methoxyphenethylamine), or a pharmaceutically acceptable salt, solvate, metabolite, deuterated analogue, derivative, prodrug, or combinations thereof. In some embodiments, the psychedelic is LSD or a pharmaceutically acceptable salt, solvate, metabolite, deuterated analogue, derivative, or prodrug thereof. In some embodiments, the LSD derivative is 1P-LSD, 1B-LSD, ETH-LAD, IP-ETH-LAD, AL-LAD, LSZ, LSM-775, or 1-(4-Bromofuro[2,3-f][1]benzofuran-8-yl)propan-2-amine. In some embodiments, the psychedelic is mescaline or a pharmaceutically acceptable salt, solvate, metabolite, deuterated analogue, derivative, or prodrug thereof. In some embodiments, the mescaline derivative is mescaline-NBOMe, proscaline (2-(3,5-dimethoxy-4-propoxyphenyl)ethanamine), or metaescaline (2-(3-ethoxy-4,5-dimethoxyphenyl)ethanamine). In some embodiments, the psychedelic is a phenethylamine or a tryptamine, or a pharmaceutically acceptable salt, solvate, metabolite, deuterated analogue, derivative, or prodrug thereof. In some embodiments, the phenethylamine or the tryptamine is selected from the group consisting of 25I-NBOH, N-(2-Methoxybenzyl)-2-(3,4,5-trimethoxyphenyl)ethanamine, N-(2-hydroxybenzyl)-2,5-dimethoxy-4-iodo-phenethylamine, N-(2-hydroxybenzyl)-2,5-dimethoxy-4-chloro-phenethylamine, N-(2-hydroxybenzyl)-2,5-dimethoxy-4-bromo-phenethylamine, 4-Allyloxy-3,5-dimethyloxyphenylethylamine, N-(2-fluorobenzyl)-2,5-dimethoxy-4-iodo-phenethylamine, 2,5-dimethoxy-4-tert-butylthio-phenethylamine, 2,5-dimethoxy-4-propylthio-phenethylamine, 2,5-dimethoxy-4-propylphenethylamine, 2,5-dimethoxy-4-nitrophenethylamine, 2,5-dimethoxy-4-nitroamphetamine, 2,5-dimethoxy-4-methylphenethylamine, 2,5-dimethoxy-4-methylamphetamine, 2,5-dimethoxy-4-isopropylthio-phenethylamine, 2,5-dimethoxy-4-iodophenethylamine, 2,5-dimethoxy-4-iodoamphetamine, 2,5-dimethoxy-4-fluorophenethylamine, 2,5-dimethoxy-4-ethylthio-phenethylamine, 2,5-dimethoxy-4-ethylphenethylamine, 2,5-dimethoxy-4-cyclopropylmethylthio-phenethylamine, 2,5-dimethoxy-4-chlorophenethylamine, 2,5-dimethoxy-4-chloroamphetamine, 2,5-dimethoxy-4-bromophenethylamine, 2,5-dimethoxy-4-bromoamphetamine, 2,5-dimethoxy-4-bromo-ß-ketophenethylamine, 2,5-dimethoxy-4-(2-fluoroethylthio)-phenethylamine, 2-(4-propyl-2,5-dimethoxyphenyl)-N-[(2-methoxyphenyl)methyl]ethanamine, 2-(4-methyl-2,5-dimethoxyphenyl)-N-[(2-methoxyphenyl)methyl]ethanamine, 2-(4-iodo-2,5-dimethoxyphenyl)-N-[(2-methoxyphenyl)methyl]ethanamine, 2-(4-fluoro-2,5-dimethoxyphenyl)-N-[(2-methoxyphenyl)methyl]ethanamine, 2-(4-ethyl-2,5-dimethoxyphenyl)-N-[(2-methoxyphenyl)methyl]ethanamine, 2-(4-chloro-2,5-dimethoxyphenyl)-N-[(2-methoxyphenyl)methyl]ethanamine, 2-(4-bromo-2,5-dimethoxyphenyl)-N-[(2-methoxyphenyl)methyl]ethanamine, 4-AcO-MET, 4-AcO-MALT and 4-AcO-DALT, Aeruginascin or N,N,N-trimethyl-4-phosphoryloxytryptamine, 4-Hydroxy-N,N,N-trimethyltryptamine, 5-meo-DMT, Ibogaine, [3-(2-Dimethylaminoethyl)-1H-indol-4-yl] dihydrogen phosphate, 4-hydroxytryptamine, 4-hydroxy-N,N-dimethyl-tryptamine, [3-(2-methylaminoethyl)-1H-indol-4-yl] dihydrogen phosphate, 4-hydroxy-N-methyltryptamine, [3-(aminoethyl)-1H-indol-4-yl] dihydrogen phosphate, [3-(2-trimethylaminoethyl)-1H-indol-4-yl] dihydrogen phosphate, and 4-hydroxy-N,N,N-trimethyltryptamine, 6-Allyl-N,N-diethyl-NL, N,N-Dibu-tyl-T, N,N-Diethyl-T, N,N-Diisopropyl-T, 5-Methyoxy-alpha-methyl-T, N,N-Dimethyl-T, 2, alpha-Dimethyl-T, alpha, N-Dimethyl-T, N,N-Dipropyl-T, N-Ethyl-N-isopropyl-T, alpha-Ethyl-T, 6,N,N-Triethyl-NL, 3,4-Dihydro-7-methoxy-1-methyl-C, 7-Methyoxy-1-methyl-C, N,N-Dibutyl-4-hydroxy-T, N,N-Diethyl-4-hydroxy-T, N,N-Diisopropyl-4-hydroxy-T, N,N-Dimethyl-4-hydroxy-T, N,N-Dimethyl-5-hydroxy-T, N, N-Dipropyl-4-hydroxy-T, N-Ethyl-4-hydroxy-N-methyl-T, 4-Hydroxy-N-isopropyl-N-methyl-T, 4-Hydroxy-N-methyl-N-propyl-T, 4-Hydroxy-N,N-tetram-ethylene-T Ibogaine, N,N-Diethyl-L, N-Butyl-N-methyl-T, N,N-Diisopropyl-4,5-methylenedioxy-T, N,N-Diisopropyl-5,6-methylenedioxy-T, N,N-Dimethyl-4,5-methylenedioxy-T, N,N-Dimethyl-5,6-methylenedioxy-T, N-Isopropyl-N-methyl-5,6-methylenedioxy-T, N,N-Diethyl-2-methyl-T, 2,N,N-Trimethyl-T, N-Acetyl-5-methoxy-T, N,N-Diethyl-5-methoxy-T, N,N-Diisopropyl-5-methoxy-T, 5-Methoxy-N, N-dimethyl-T, N-Isopropyl-4-methoxy-N-methyl-T, N-Iso-propyl-5-methoxy-N-methyl-T,5,6-Dimethoxy-N-isopropyl-N-methyl-T, 5-Methoxy-N-methyl-T, 5-Methoxy-N,N-tetramethylene-T, 6-Methoxy-1-methyl-1,2,3,4-tetrahydro-C, 5-Methoxy-2,N,N-trimethyl-T, N,N-Dimethyl-5-methylthio-T, N-Isopropyl-N-methyl-T, alpha-Methyl-T, N-Ethyl-T, N-Methyl-T, 6-Propyl-N L, N,N-Tetramethylene-T, Tryptamine, 7-Methoxy-1-methyl-1, 2,3,4-tetrahydro-C, alpha, N-Dimethyl-5-methoxy-T, or a pharmaceutically acceptable salt, solvate, metabolite, deuterated analogue, derivative, prodrug, or combinations thereof. In some embodiments, the psychedelic is psilocybin or a pharmaceutically acceptable salt, solvate, metabolite, deuterated analogue, derivative, or prodrug thereof. In some embodiments, the psychedelic is 1-acetyl LSD or a pharmaceutically acceptable salt, solvate, metabolite, deuterated analogue, derivative, or prodrug thereof. In some embodiments, the psychedelic is O-acetyl psilocin, or a pharmaceutically acceptable salt, solvate, metabolite, deuterated analogue, derivative, or prodrug thereof. In some embodiments, the serotonin receptor modulator is a serotonin receptor antagonist. In some embodiments, the serotonin receptor modulator is a serotonin receptor inverse agonist. In some embodiments, the serotonin receptor is serotonin receptor 2A. In some embodiments, the serotonin receptor modulator comprises glemanserin (MDL-11,939), eplivanserin (SR-46,349), ketanserin, ritanserin, altanserin, acepromazine, mianserin, mirtazapine, quetiapine, SB204741, SB206553, SB242084, LY272015, SB243213, Blonanserin, SB200646, RS102221, nefazodone, volinanserin (MDL-100,907), pimavanserin (ACO-103), nelotanserin, lorcaserin, flibanserin, roluperiodone or a pharmaceutically acceptable salt, solvate, metabolite, deuterated analogue, derivative, prodrug, or combinations thereof. In some embodiments, the serotonin receptor modulator is ketanserin or a pharmaceutically acceptable salt, solvate, metabolite, deuterated analogue, derivative, or prodrug thereof. In some embodiments, the serotonin receptor modulator is pimavanserin or a pharmaceutically acceptable salt, solvate, metabolite, deuterated analogue, derivative, or prodrug thereof. In some embodiments, the serotonin receptor modulator is eplivanserin or a pharmaceutically acceptable salt, solvate, metabolite, deuterated analogue, derivative, or prodrug thereof. In some embodiments, the serotonin receptor modulator is flibanserin or a pharmaceutically acceptable salt, solvate, metabolite, deuterated analogue, derivative, or prodrug thereof. In some embodiments, the serotonin receptor modulator is roluperiodone or a pharmaceutically acceptable salt, solvate, metabolite, deuterated analogue, derivative, or prodrug thereof. In some embodiments, the serotonin receptor modulator is released at most about 2 hours prior to the release of the psychedelic. In some embodiments, the serotonin receptor modulator is released at most about 1.5 hours prior to the release of the psychedelic. In some embodiments, the serotonin receptor modulator is released at most about 1 hour prior to the release of the psychedelic. In some embodiments, the serotonin receptor modulator is released at about 1 hour prior to the release of the psychedelic. In some embodiments, the psychedelic is provided at a dose of about 100 milligrams (mg) to about 1 gram (g). In some embodiments, the psychedelic is provided at a dose of about 100 mg to about 800 mg. In some embodiments, the psychedelic is provided at a dose of about 100 mg to about 500 mg. In some embodiments, the psychedelic is provided at a dose of about 10 mg to about 400 mg. In some embodiments, the psychedelic is provided at a dose of about 20 mg to about 200 mg. In some embodiments, the psychedelic is provided at a dose of about 10 mg to about 100 mg. In some embodiments, the psychedelic is provided at a dose of about 10 micrograms (μg) to about 400 μg. In some embodiments, the psychedelic is provided at a dose of about 20 μg to about 200 μg. In some embodiments, the psychedelic is provided at a dose of about 10 μg to about 100 μg. In some embodiments, the serotonin receptor modulator is provided at a dose of about 10 mg to about 350 mg. In some embodiments, the serotonin receptor modulator is provided at a dose of about 20 mg to about 200 mg. In some embodiments, the serotonin receptor modulator is provided at a dose of about 10 mg to about 100 mg. In some embodiments, the psychedelic is provided at a dose of about 10 mg to about 100 mg and the serotonin receptor modulator is provided at a dose of about 10 mg to about 100 mg. In some embodiments, the psychedelic is psilocybin or a pharmaceutically acceptable salt, solvate, metabolite, deuterated analogue, derivative, or prodrug thereof and the serotonin receptor modulator is ketanserin or a pharmaceutically acceptable salt, solvate, metabolite, deuterated analogue, derivative, or prodrug thereof. In some embodiments, the psychedelic is psilocybin or a pharmaceutically acceptable salt, solvate, metabolite, deuterated analogue, derivative, or prodrug thereof and provided at a dose of about 10 mg to about 100 mg and the serotonin receptor modulator is ketanserin or a pharmaceutically acceptable salt, solvate, metabolite, deuterated analogue, derivative, or prodrug thereof provided at a dose of about 10 mg to about 100 mg. In some embodiments, the composition is in a form of a single pill, single ampoule, or single unit dosage form.

Described herein, in certain embodiments, are methods of treating a disease or disorder in a subject in need thereof, the method comprising administering to the subject a composition comprising: a) a psychedelic; b) a serotonin receptor modulator; and c) an excipient, wherein the serotonin receptor modulator is released at most about 3 hours prior to the release of the psychedelic. In some embodiments, the disease or disorder is depression or a disease or disorder related to depression. In some embodiments, the depression is major depressive disorder, persistent depressive disorder, bipolar disorder, treatment resistant depression (TRD), postpartum depression, premenstrual dysphoric disorder, or seasonal affective disorder. In some embodiments, the disease or disorder related to depression is anxiety. In some embodiments, the psychedelic is an agonist for a serotonin receptor. In some embodiments, the serotonin receptor is serotonin receptor 1B, serotonin receptor 4, serotonin receptor 6, or serotonin receptor 7. In some embodiments, the psychedelic is selected from the group consisting of psilocybin, psilocin, baeocystin, norbaeocystin, lisurgide, LSD, dimethyltryptamine, carboxamindotryptamine, ibogaine, tabernanthalog, 3,4-methylenedioxy-methamphetamine (MDMA), 1-acetyl LSD, O-acetyl psilocin, mescaline (3,4,5-trimethoxy phenethylamine), proscaline (2-(3,5-dimethoxy-4-propoxyphenyl)ethanamine), metaescaline (2-(3-ethoxy-4,5-dimethoxyphenyl)ethanamine), allylescaline (4-Allyloxy-3,5-dimethyloxy phenylethylamine), methallylescaline (4-Methallyloxy-3,5 dimethoxyphenethylamine), asymbescaline (3,4-Diethoxy-5-methoxyphenethylamine), or a pharmaceutically acceptable salt, solvate, metabolite, deuterated analogue, derivative, prodrug, or combinations thereof. In some embodiments, the psychedelic is LSD or a pharmaceutically acceptable salt, solvate, metabolite, deuterated analogue, derivative, or prodrug thereof. In some embodiments, the LSD derivative is 1P-LSD, 1B-LSD, ETH-LAD, IP-ETH-LAD, AL-LAD, LSZ, LSM-775, 1-(4-Bromofuro[2,3-f][1]benzofuran-8-yl)propan-2-amine. In some embodiments, the psychedelic is mescaline or a pharmaceutically acceptable salt, solvate, metabolite, deuterated analogue, derivative, or prodrug thereof. In some embodiments, the mescaline derivative is mescaline-NBOMe, proscaline (2-(3,5-dimethoxy-4-propoxyphenyl)ethanamine), or metaescaline (2-(3-ethoxy-4,5-dimethoxyphenyl)ethanamine). In some embodiments, the psychedelic is a phenethylamine or a tryptamine, or a pharmaceutically acceptable salt, solvate, metabolite, deuterated analogue, derivative, or prodrug thereof. In some embodiments, the phenethylamine or the tryptamine is selected from the group consisting of 25I-NBOH, N-(2-Methoxybenzyl)-2-(3,4,5-trimethoxyphenyl)ethanamine, N-(2-hydroxybenzyl)-2,5-dimethoxy-4-iodo-phenethylamine, N-(2-hydroxybenzyl)-2,5-dimethoxy-4-chloro-phenethylamine, N-(2-hydroxybenzyl)-2,5-dimethoxy-4-bromo-phenethylamine, 4-Allyloxy-3,5-dimethyloxyphenylethylamine, N-(2-fluorobenzyl)-2,5-dimethoxy-4-iodo-phenethylamine, 2,5-dimethoxy-4-tert-butylthio-phenethylamine, 2,5-dimethoxy-4-propylthio-phenethylamine, 2,5-dimethoxy-4-propylphenethylamine, 2,5-dimethoxy-4-nitrophenethylamine, 2,5-dimethoxy-4-nitroamphetamine, 2,5-dimethoxy-4-methylphenethylamine, 2,5-dimethoxy-4-methylamphetamine, 2,5-dimethoxy-4-isopropylthio-phenethylamine, 2,5-dimethoxy-4-iodophenethylamine, 2,5-dimethoxy-4-iodoamphetamine, 2,5-dimethoxy-4-fluorophenethylamine, 2,5-dimethoxy-4-ethylthio-phenethylamine, 2,5-dimethoxy-4-ethylphenethylamine, 2,5-dimethoxy-4-cyclopropylmethylthio-phenethylamine, 2,5-dimethoxy-4-chlorophenethylamine, 2,5-dimethoxy-4-chloroamphetamine, 2,5-dimethoxy-4-bromophenethylamine, 2,5-dimethoxy-4-bromoamphetamine, 2,5-dimethoxy-4-bromo-ß-ketophenethylamine, 2,5-dimethoxy-4-(2-fluoroethylthio)-phenethylamine, 2-(4-propyl-2,5-dimethoxyphenyl)-N-[(2-methoxyphenyl)methyl]ethanamine, 2-(4-methyl-2,5-dimethoxyphenyl)-N-[(2-methoxyphenyl)methyl]ethanamine, 2-(4-iodo-2,5-dimethoxyphenyl)-N-[(2-methoxyphenyl)methyl]ethanamine, 2-(4-fluoro-2,5-dimethoxyphenyl)-N-[(2-methoxyphenyl)methyl]ethanamine, 2-(4-ethyl-2,5-dimethoxyphenyl)-N-[(2-methoxyphenyl)methyl]ethanamine, 2-(4-chloro-2,5-dimethoxyphenyl)-N-[(2-methoxyphenyl)methyl]ethanamine, 2-(4-bromo-2,5-dimethoxyphenyl)-N-[(2-methoxyphenyl)methyl]ethanamine, 4-AcO-MET, 4-AcO-MALT and 4-AcO-DALT, Aeruginascin or N,N,N-trimethyl-4-phosphoryloxytryptamine, 4-Hydroxy-N,N,N-trimethyltryptamine, 5-meo-DMT, Ibogaine, [3-(2-Dimethylaminoethyl)-1H-indol-4-yl] dihydrogen phosphate, 4-hydroxytryptamine, 4-hydroxy-N,N-dimethyl-tryptamine, [3-(2-methylaminoethyl)-1H-indol-4-yl] dihydrogen phosphate, 4-hydroxy-N-methyltryptamine, [3-(aminoethyl)-1H-indol-4-yl] dihydrogen phosphate, [3-(2-trimethylaminoethyl)-1H-indol-4-yl] dihydrogen phosphate, and 4-hydroxy-N,N,N-trimethyltryptamine, 6-Allyl-N,N-diethyl-NL, N,N-Dibu-tyl-T, N,N-Diethyl-T, N,N-Diisopropyl-T, 5-Methyoxy-alpha-methyl-T, N,N-Dimethyl-T, 2, alpha-Dimethyl-T, alpha, N-Dimethyl-T, N,N-Dipropyl-T, N-Ethyl-N-isopropyl-T, alpha-Ethyl-T, 6,N,N-Triethyl-NL, 3,4-Dihydro-7-methoxy-1-methyl-C, 7-Methyoxy-1-methyl-C, N,N-Dibutyl-4-hydroxy-T, N,N-Diethyl-4-hydroxy-T, N,N-Diisopropyl-4-hydroxy-T, N,N-Dimethyl-4-hydroxy-T, N,N-Dimethyl-5-hydroxy-T, N, N-Dipropyl-4-hydroxy-T, N-Ethyl-4-hydroxy-N-methyl-T, 4-Hydroxy-N-isopropyl-N-methyl-T, 4-Hydroxy-N-methyl-N-propyl-T, 4-Hydroxy-N,N-tetram-ethylene-T Ibogaine, N,N-Diethyl-L, N-Butyl-N-methyl-T, N,N-Diisopropyl-4,5-methylenedioxy-T, N,N-Diisopropyl-5,6-methylenedioxy-T, N,N-Dimethyl-4,5-methylenedioxy-T, N,N-Dimethyl-5,6-methylenedioxy-T, N-Isopropyl-N-methyl-5,6-methylenedioxy-T, N,N-Diethyl-2-methyl-T, 2,N,N-Trimethyl-T, N-Acetyl-5-methoxy-T, N,N-Diethyl-5-methoxy-T, N,N-Diisopropyl-5-methoxy-T, 5-Methoxy-N, N-dimethyl-T, N-Isopropyl-4-methoxy-N-methyl-T, N-Iso-propyl-5-methoxy-N-methyl-T,5,6-Dimethoxy-N-isopropyl-N-methyl-T, 5-Methoxy-N-methyl-T, 5-Methoxy-N,N-tetramethylene-T, 6-Methoxy-1-methyl-1,2,3,4-tetrahydro-C, 5-Methoxy-2, N, N-trimethyl-T, N, N-Dimethyl-5-methylthio-T, N-Isopropyl-N-methyl-T, alpha-Methyl-T, N-Ethyl-T, N-Methyl-T, 6-Propyl-N L, N,N-Tetramethylene-T, Tryptamine, 7-Methoxy-1-methyl-1, 2,3,4-tetrahydro-C, alpha, N-Dimethyl-5-methoxy-T, or a pharmaceutically acceptable salt, solvate, metabolite, deuterated analogue, derivative, prodrug, or combinations thereof. In some embodiments, the psychedelic is psilocybin or a pharmaceutically acceptable salt, solvate, metabolite, deuterated analogue, derivative, or prodrug thereof. In some embodiments, the psychedelic is 1-acetyl LSD or a pharmaceutically acceptable salt, solvate, metabolite, deuterated analogue, derivative, or prodrug thereof. In some embodiments, the psychedelic is O-acetyl psilocin, or a pharmaceutically acceptable salt, solvate, metabolite, deuterated analogue, derivative, or prodrug thereof. In some embodiments, the serotonin receptor modulator is a serotonin receptor antagonist. In some embodiments, the serotonin receptor modulator is a serotonin receptor inverse agonist. In some embodiments, the serotonin receptor is serotonin receptor 2A. In some embodiments, the serotonin receptor modulator comprises ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, AMDA, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, and RS-102221, or a pharmaceutically acceptable salt, solvate, metabolite, deuterated analogue, derivative, prodrug, or combinations thereof. In some embodiments, the serotonin receptor modulator is ketanserin or a pharmaceutically acceptable salt, solvate, metabolite, deuterated analogue, derivative, or prodrug thereof. In some embodiments, the serotonin receptor modulator is pimavanserin or a pharmaceutically acceptable salt, solvate, metabolite, deuterated analogue, derivative, or prodrug thereof. In some embodiments, the serotonin receptor modulator is eplivanserin or a pharmaceutically acceptable salt, solvate, metabolite, deuterated analogue, derivative, or prodrug thereof. In some embodiments, the serotonin receptor modulator is released at most about 2 hours prior to the release of the psychedelic. In some embodiments, the serotonin receptor modulator is released at most about 1.5 hours prior to the release of the psychedelic. In some embodiments, the serotonin receptor modulator is released at most about 1 hour prior to the release of the psychedelic. In some embodiments, the serotonin receptor modulator is released at about 1 hour prior to the release of the psychedelic. In some embodiments, the psychedelic is provided at a dose of about 100 milligrams (mg) to about 1 gram (g). In some embodiments, the psychedelic is provided at a dose of about 100 mg to about 800 mg. In some embodiments, the psychedelic is provided at a dose of about 100 mg to about 500 mg. In some embodiments, the psychedelic is provided at a dose of about 10 mg to about 400 mg. In some embodiments, the psychedelic is provided at a dose of about 20 mg to about 200 mg. In some embodiments, the psychedelic is provided at a dose of about 10 mg to about 100 mg. In some embodiments, the psychedelic is provided at a dose of about 10 micrograms (μg) to about 400 μg. In some embodiments, the psychedelic is provided at a dose of about 20 μg to about 200 μg. In some embodiments, the psychedelic is provided at a dose of about 10 μg to about 100 μg. In some embodiments, the serotonin receptor modulator is provided at a dose of about 10 mg to about 350 mg. In some embodiments, the serotonin receptor modulator is provided at a dose of about 20 mg to about 200 mg. In some embodiments, the serotonin receptor modulator is provided at a dose of about 10 mg to about 100 mg. In some embodiments, the psychedelic is provided at a dose of about 10 mg to about 100 mg and the serotonin receptor modulator is provided at a dose of about 10 mg to about 100 mg. In some embodiments, the composition is in a form of a single pill, single ampoule, or single unit dosage form. In some embodiments, the psychedelic is psilocybin or a pharmaceutically acceptable salt, solvate, metabolite, deuterated analogue, derivative, or prodrug thereof and the serotonin receptor modulator is ketanserin or a pharmaceutically acceptable salt, solvate, metabolite, deuterated analogue, derivative, or prodrug thereof. In some embodiments, the psychedelic is psilocybin or a pharmaceutically acceptable salt, solvate, metabolite, deuterated analogue, derivative, or prodrug thereof and provided at a dose of about 10 mg to about 100 mg and the serotonin receptor modulator is ketanserin or a pharmaceutically acceptable salt, solvate, metabolite, deuterated analogue, derivative, or prodrug thereof provided at a dose of about 10 mg to about 100 mg.

In some embodiments, the composition is in a form of a single pill, single ampoule, or single unit dosage form.

In some embodiments, the composition is in a single unit dosage form.

In another aspect, also provided herein are methods of treating a disease or disorder in a subject in need thereof, the method comprising administering to the subject a psychedelic and a serotonin receptor modulator, wherein the serotonin receptor is administered prior to the administration of the psychedelic, and wherein the serotonin receptor modulator is administered at most about 3 hours prior to the administration of the psychedelic.

In yet another aspect, also provided herein are methods of treating a disease or disorder in a subject in need thereof, the method comprising (i) administering to the subject a serotonin receptor modulator to pretreat the subject, and (ii) administering to the subject a psychedelic, and wherein the serotonin receptor modulator is administered at most about 3 hours prior to the administration of the psychedelic.

In yet another aspect, also provided herein are methods of reducing the adverse effects of a psychedelic in the treatment of a disease or disorder in a subject in need thereof, the method comprising (i) administering to the subject a serotonin receptor modulator to pretreat the subject, and (ii) administering to the subject a psychedelic, and wherein the serotonin receptor modulator is administered at most about 3 hours prior to the administration of the psychedelic.

In some embodiments, the disease or disorder is depression or a disease or disorder related to depression.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIGS. 1A-1C illustrate restoration of hedonic behavior after chronic stress by psilocybin that is unaffected by ketanserin. FIG. 1A illustrates an experimental timeline illustrating when hedonic behaviors were measured in relation to chronic multimodal stress (CMMS) and drug treatment. FIG. 1B illustrates a graph demonstrating CMMS significantly decreased sucrose preference (SP) compared to baseline across all treatment groups: vehicle-vehicle (gray; p=0.0012; n=12), ketanserin-vehicle (blue; p-0.0012; n=6), vehicle-psilocybin (yellow; p=0.0012; n=13), ketanserin-psilocybin (green; p=0.0012; n=7). Treatment with psilocybin (1 mg/kg, i.p.) significantly increased SP compared to values after CMMS, whether animals were pretreated with ketanserin (2 mg/kg; p=0.042) or a vehicle control (p=0.0012). Neither injection with vehicle (p-0.078) nor ketanserin alone (p=0.87; n=6) had a significant effect on SP following CMMS. Three-way repeated measures ANOVA revealed a significant effect of stress (F2,68=60.26, p<0.0001), and interaction of Stress ×Psilocybin (F2,68-4.50, p-0.015), but not for Stress ×Psilocybin ×Ketanserin (F2,68=0.05917, p-0.9426). FIG. 1C illustrates a graph demonstrating CMMS significantly decreased preference for female urine compared to baseline: vehicle-vehicle (p=0.013; n=6), ketanserin-vehicle (p=0.0012; n=4), vehicle-psilocybin (p=0.0012; n=5), ketanserin-psilocybin (p=0.0012; n=4). Treatment with psilocybin significantly increased preference for the scent of female urine compared to values after CMMS whether animals were pretreated with ketanserin (p=0.0024) or vehicle (p=0.0012). Following CMMS, neither injection with vehicle (p=0.43) nor ketanserin alone (p=0.072) had a significant effect on female urine preference. Stress significantly reduced female urine preference in all groups (F2,30=43.41, p<0.0001) and a three-way repeated measures ANOVA showed a significant interaction between Stress ×Psilocybin (F2,30-4.26, p<0.024), but not between Stress ×Psilocybin ×Ketanserin (F2,30=0.8677, p=0.4302). The figure bars represent the group means±SEM. The reported post-hoc comparisons were corrected with the Holm-Sidak method. *p<0.05, ** p<0.005; ns, not significant.

FIGS. 2A-2D illustrate graphs demonstrating pretreatment with ketanserin effectively blocks 5-HT2A receptor activation. FIG. 2A illustrates a graph demonstrating pretreatment with ketanserin (2 mg/kg) 60 minutes prior to psilocybin (1 mg/kg) administration prevents head-twitching during the 15 minutes immediately following drug injection in mice (ket-psil v. ket-veh, p=0.26). The difference between groups (F3,25=6.96, p=0.0015) was driven by the increase in head-twitching seen in vehicle-psilocybin animals (veh-veh v. veh-psil, p=0.0045). FIG. 2B illustrates a graph demonstrating in animals treated with psilocybin (n=14), there was no effect of head-twitching (F1, 12=0.53, p=0.48) but a significant effect of time (F2, 24=38.90, p<0.0001), revealing significant differences between stress and post-treatment SPT in both high head-twitching (p=0.0005) and low head-twitching (p=0.038) mice. Of these also used in the FUST (n=9), there was no effect of head-twitching (F1, 7=0.1040, p=0.7565) but a significant effect of time (F2,14=23.05, p<0.0001), showing a significant difference in female urine preferences between high (p=0.0007) and low (p=0.0086) head-twitching mice between post-stress and post-treatment timepoints. A two-tailed, unpaired t-test comparing AMPA:NMDA ratios between high (n=8) and low (n=6) head-twitching mice found no difference between groups (p=0.7717). FIG. 2C illustrates a graph demonstrating in a separate cohort of animals, psilocybin (10 mg/kg, n=4) acutely reduced hippocampal local field potential activity in the low frequency range with a peak effect around 15-25 minutes following psilocybin injection. This effect was inhibited by pretreatment with ketanserin (2 mg/kg, n=4). FIG. 2D illustrates a graph demonstrating pretreatment with ketanserin significantly attenuated psilocybin-induced reductions in the delta frequency band 15-25 minutes following psilocybin injection. 2-way ANOVA revealed significant interaction of Frequency×Ketanserin (F4, 30=3.66, p=0.0152) and a post hoc analysis revealed a significant difference between Vehicle-Psilocybin and Ketanserin-Psilocybin in the delta band (t=4.99, df=30, p=0.00012). The figure bars represent the group means±SEM. * p<0.05, ** p<0.01, *** p<0.001.

FIGS. 3A-3D illustrate graphs demonstrating psilocybin strengthens hippocampal TA-CAI synapses following CMMS. FIG. 3A illustrates field EPSPs (fEPSPs) from a single stimulation intensity from one hippocampal slice per group, recorded in Mg2 free ACSF (black), after wash-in of DNQX (50 uM; dark grey) and then APV (80 uM; light grey) to isolate AMPA- and NMDAR-mediated components. FIG. 3B illustrates a graph of AMPA:NMDA ratios. Mice subjected to CMMS and treated with psilocybin had higher AMPA:NMDA ratios compared to stressed mice given only vehicle (gray, n=12) or ketanserin (blue, n=7), regardless of whether they were pretreated with ketanserin (green, n=7; p=0.0002) or vehicle (yellow, n=13; p-0.0003). Two-way ANOVA showed a significant effect of psilocybin (F1,34=34.79, p<0.0001), but not ketanserin×psilocybin (F1,34=1.422, p=0.2414). FIG. 3C illustrates a graph demonstrating psilocybin increased the AMPA:FV ratio of the fEPSP (two-way ANOVA: F1,34=4.378, p=0.044). FIG. 3D illustrates a graph demonstrating treatment with psilocybin did not change the NMDA:FV ratio of the fEPSP (two-way ANOVA: F1,34=2.077, p=0.16). AMPA:NMDA, AMPA:FV and NMDA:FV for each animal is shown along with group means±SEM. * p<. 05, *** p<. 0005, ns=not significant.

FIG. 4 illustrates a graph demonstrating psilocybin had no effects in resilient animals. Resilient mice were defined as those exhibiting a high sucrose preference (>70%) following 14 days CMMS. Injection of psilocybin (1 mg/kg, ip) had no effect on sucrose preference in resilient animals (n=3, red), nor did injection of vehicle (n=7, blue). Two-way repeated measures ANOVA: F1,14=0.14, p=0.72. Sucrose preference is presented as the group means±SEM with data from individual animals superimposed.

FIGS. 5A-5B illustrate graphs demonstrating ketanserin pretreatment enhances hypolocomotive effects of psilocybin. A separate cohort of animals were pretreated with saline vehicle (n=9) or ketanserin (n=9; 2 mg/kg) 60 minutes prior to psilocybin (5 mg/kg) administration. Following injection with psilocybin, mice were immediately placed in open field arenas and video recorded for 90 minutes. FIG. 5A illustrates a graph demonstrating ketanserin-psilocybin mice travelled less at 0-30 minutes compared to controls (n=6; p=0.0019) and vehicle pretreated mice (p=0.0059), and at 30-60 minutes (p=0.0008) compared to controls. Vehicle-psilocybin mice displayed hypolocomotion compared to controls at 30-60 minutes timepoint (p=0.005). There was a significant effect of treatment (F2,21=7.56, p=0.0034), time (F2, 42=10.69, p=0.0002) and interaction of time×treatment (F4, 42=3.17, p=0.023). FIG. 5B illustrates a graph demonstrating mice treated with ketanserin-psilocybin spent less time in the center of the arena compared to control mice at 0-30 minutes (p=0.017) and 30-60 minutes (p=0.0001), while vehicle-psilocybin mice displayed less center time compared to controls at 30-60 minute timepoint (p=0.0077). There was a significant effect of treatment on time in center (F2, 21=7.085, p=0.0045) but no effect of time (F2, 42=2.58, p=0.088). Individual points represent the mean+/−SEM for treatment groups at each timepoint. ##v. control, p<0.01, ** v. veh-psil, p<0.01, ###v. control, p<. 001.

FIG. 6 illustrates a graph demonstrating synaptic strength is correlated with changes in sucrose preference. Changes in sucrose preference in individual mice between baseline and post-treatment measurements are significantly correlated with the average AMPA:NMDA ratio TA-CAI synaptic strength (y=1.725x−44.06, R2=0.18, p=0.0072).

FIG. 7 illustrates a graph demonstrating psilocybin has no effect on immobility time in the forced swim test. A cohort of unstressed C57Bl/6J mice were injected with psilocybin (n=10 males, n=10 females; 1 mg/kg) or saline (n=10 males, n=10 females). Time spent immobile during the forced swim test was measured 1, 3, and 7 days post-injection. There was no effect of psilocybin (F1, 18=2.28, p=0.15) or time (F2, 36=1.53, p=0.23) in the female cohort. In the male cohort, there was a significant effect of time (F2, 36=3.85, p=0.031) but no effect of psilocybin (F1, 18=0.36, p=0.56). Post-hoc comparisons in the male cohort revealed a significant difference in immobility time between days 3 and 7 (p=0.031), but no differences between saline and psilocybin treated animals at any timepoint. The height of the bars represents the mean+/−SEM. * p<0.05.

FIG. 8 illustrates dense Global Brain Connectivity (GBC) maps showing the difference in GBC (AGBC) for the ketanserin (Ket)+LSD-treated subjects vs placebo (Pla)+Pla contrast at the early time point (75-minute) after treatment.

FIG. 9 illustrates dense GBC maps showing the difference in GBC (AGBC) for the placebo+LSD-treated subjects vs placebo+placebo contrast at the early time point (75-minute) after treatment.

FIG. 10 illustrates dense GBC maps showing the difference in GBC (AGBC) for the ketanserin+LSD-treated subjects vs placebo+placebo contrast at the late time point (300-minute) after treatment.

FIG. 11 illustrates dense GBC maps showing the difference in GBC (AGBC) for the placebo+LSD-treated subjects vs placebo+placebo contrast at the late time point (300-minute) after treatment.

FIG. 12 illustrates dense GBC maps showing the difference in GBC (AGBC) for the ketanserin+LSD-treated subjects vs placebo+placebo contrast for the session average after treatment.

FIG. 13 illustrates dense GBC maps showing the difference in GBC (AGBC) for the placebo+LSD-treated subjects vs placebo+placebo contrast for the session average after treatment.

FIG. 14 illustrates a collection of panels showing average session results, early session time point (75-minute) results and late session time point (300-minute) results for the difference in Functional Connectivity (AFC) between the test conditions. The left of each row indicates the test conditions and the top of each column indicates the time points. Positive and negative contrasts are rendered, respectively, in red and blue hues. Nodes along the edges indicate (cortical and subcortical) parcels of the CAP-NP parcellation.

FIG. 15 illustrates related effects of Ket+LSD and Pla+LSD on parcels' GBC values. Panels show 2d-histograms across parcels, indicating how pretreatment+treatment effects for Ket+LSD and Pla+LSD (relative to the placebo+placebo condition) were related. At the early time point (left panel; 75 minutes), the bulk of the distribution scattered around 0 effects, but some parcels with strong effects (bottom right) led to a net negative correlation. At the late time point (right panel; 300 minutes), a strong positive correlation was observed. Table 1 also shows correlations between GBC changes in parcels induced by Pla+LSD and Ket+LSD (relative to Pla+Pla).

FIG. 16 illustrates related effects of Ket+LSD and Pla+LSD on parcels' Functional Connectivity (FC) values. Panels show 2d-histograms across connections between parcels, indicating how pretreatment+treatment effects for Ket+LSD and Pla+LSD (relative to the placebo+placebo condition) are related. At the early time point (left panel; 75 minutes), effects between the two conditions were weakly correlated, whereas at the late time point (right panel; 300 minutes) a strong positive correlation was observed. Table 2 also shows correlations between FC changes in parcels induced by Pla+LSD and Ket+LSD (relative to Pla+Pla).

FIG. 17 illustrates AGBC confidence intervals for the early time point. Confidence intervals were calculated assuming a t-distribution. The CAP-NP network to which a parcel belongs is indicated by labels on the row. Additionally, a confidence interval was plotted in a desaturated (light) shade of grayscale if the FDR-BH corrected p-value is ≥ 0.05. Top row indicates Pla+LSD vs Pla+Pla. Middle row indicates Ket+LSD vs Pla+Pla. Bottom row indicates Pla+LSD vs Ket+LSD.

FIG. 18 illustrates AGBC confidence intervals for the late time point. Confidence intervals were calculated assuming a t-distribution. The CAP-NP network to which a parcel belongs is indicated by labels on the row. Additionally, a confidence interval was plotted in a desaturated (light) shade of grayscale if the FDR-BH corrected p-value is ≥ 0.05. Top row indicates Pla+LSD vs Pla+Pla. Middle row indicates Ket+LSD vs Pla+Pla. Bottom row indicates Pla+LSD vs Ket+LSD.

FIG. 19 illustrates network FC contrasts. For each pair of networks, all connection contrasts (i.e. AFC values) between these two networks were averaged resulting in a network×network FC contrast matrix. Networks are defined in the CAP-NP atlas. The contrasts shown here are average contrasts for a given network pair (left column of panels: 75 minutes after treatment; right column of panels: 300 minutes after treatment).

FIG. 20 illustrates the time course of subjective drug effects for the labeled subject groups. Five Dimension Altered States of Consciousness Questionnaire short version scores were assessed at 180, 250, and 360 minutes after second drug administration for the means across scales, and the scale scores for Pla, LSD, and Ket+LSD conditions. Scores are expressed as percent of the scale maximum. Data are expressed as means±/−the standard error of the mean (SEM). Abbreviations indicate Blissful State: BS, Changed Meaning of Percepts: CMP, Disembodiment: D, Elementary Imagery: El, Spiritual Experience: SE. N−=23. The first graph indicates the mean across scales. The second and third graphs indicates that the subjective LSD effects were still intense and high above placebo at 360 minutes. The fourth graph indicates that at no time point is there a significant presentation of self-reported altered states of consciousness in the Ket+LSD test subject group compared with the other graphs. This figure is reprinted from eLife Preller 2018. (Preller et al. eLife. 2018 Oct. 25; 7:e35082. Changes in global and thalamic brain connectivity in LSD-induced altered states of consciousness are attributable to the 5-HT2A receptor, PMID: 30355445).

### DETAILED DESCRIPTION

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an”, and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the term “modified release” coating encompasses coatings that delay release, sustain release, extend release, prevent release, minimize release and/or otherwise prolong the release of a drug relative to formulations lacking such coatings which release a drug relatively quickly (i.e., “immediate release” compositions). The term “modified release” encompasses “sustained release,” “extended release,” “delayed release,” and the like. The term “modified release” is used interchangeably with “controlled release” or “delayed release”. The term “modified-release” or “delayed release” dosage composition refers broadly to a dosage form showing one or more modified-release properties, as described herein.

### Compositions

Psychedelics have the potential to be used for therapeutics for treating various neuropsychiatric disorders including depression. However, use of psychedelics for treatment are associated with negative effects such as psychedelic-induced alterations in sensory perception and consciousness. These negative effects would be greatly reduced if the psychedelic response could be diminished without impairing the therapeutic response. Described herein, in certain embodiments, are compositions comprising a psychedelic and a serotonin receptor modulator that provide therapeutic effects while reducing the effects associated with psychedelics. In some embodiments, the psychedelic and the serotonin receptor modulator are provided such that the serotonin receptor modulator is released a certain time before the psychedelic. Compositions and methods as described herein, in certain embodiments, provide an anti-depressive effect without alteration in sensory perception and consciousness or with a minimal alteration in sensory perception and consciousness.

Many potential benefits for psychedelics in treating neurological conditions have been contemplated. Numerous neurological conditions are resistant to current treatments in a significant segment of the population and present widespread socioeconomic burden and personal suffering. Several problems have persisted in attempts to implement psychedelics into potential treatments for common and oftentimes intractable conditions such as depression and treatment-resistant depression. One such problem is the triggering of non-ordinary states of consciousness including hallucinogenic experiences in subjects in a which a psychedelic is administered. Another such problem is that the psychedelic-induced alterations in neural function which lead to the non-ordinary states of consciousness were believed to be the bases for many of the potentially contemplated therapeutic benefits of such psychedelics. The solutions provided herein solve these problems by demonstrating novel compositions and methods in which therapeutic benefits for treating or alleviating symptoms of a neurological condition using a psychedelic have been discovered that can be persist or be amplified by specifically blocking a component of the psychedelic which elicits the non-ordinary states of consciousness. This alleviates the undesirable and burdensome side-effects for utilizing psychedelics as therapeutics.

Described herein, in some embodiments, are compositions comprising a psychedelic and a serotonin receptor modulator, wherein the serotonin receptor modulator is released at most about 3 hours prior to the release of the psychedelic. In some embodiments, the serotonin receptor modulator is released at most about 2 hours prior to the release of the psychedelic. In some embodiments, the serotonin receptor modulator is released at most about 1.5 hours prior to the release of the psychedelic. In some embodiments, the serotonin receptor modulator is released at most about 1 hour prior to the release of the psychedelic. In some embodiments, the serotonin receptor modulator is released at about 1 hour prior to the release of the psychedelic. In some embodiments, the serotonin receptor modulator is released at most about 5 minutes, 10 minutes, 20 minutes, 30 minutes, 40 minutes, 50 minutes, 1 hour, 1.25 hours, 1.5 hours, 2 hours, or 3 hours prior to the release of the psychedelic. In some embodiments, the serotonin receptor modulator is released at most about 3 hours, 4 hours, 5 hours, 6 hours, 7 hours, 8 hours, 9 hours, or more than 9 hours prior to the release of the psychedelic. In some embodiments, the serotonin receptor modulator is released in a range of about 5 minutes to about 3 hours, about 10 minutes to about 3 hours, about 20 minutes to about 3 hours, about 30 minutes to about 3 hours, about 40 minutes to about 3 hours, about 50 minutes to about 3 hours, about 1 hour to about 3 hours, about 5 minutes to about 2 hours, about 10 minutes to about 2 hours, about 20 minutes to about 2 hours, about 30 minutes to about 2 hours, about 40 minutes to about 2 hours, about 50 minutes to about 2 hours, about 1 hour to about 2 hours, about 5 minutes to about 1 hour, about 10 minutes to about 1 hour, about 20 minutes to about 1 hour, about 30 minutes to about 1 hour, about 40 minutes to about 1 hour, or about 50 minutes to about 1 hour prior to the release of the psychedelic.

In some embodiments, the serotonin receptor modulator is released at least about 0.5 hours prior to the release of the psychedelic. In some embodiments, the serotonin receptor modulator is released at least about 1 hour prior to the release of the psychedelic.

In some embodiments, the serotonin receptor modulator is released at most about 6 hours prior to the release of the psychedelic. In some embodiments, the serotonin receptor modulator is released at most about 5 hours prior to the release of the psychedelic. In some embodiments, the serotonin receptor modulator is released at most about 4 hours prior to the release of the psychedelic. In some embodiments, the serotonin receptor modulator is released at most about 3 hours prior to the release of the psychedelic.

In a preferred embodiment, the serotonin receptor modulator is released at about 1 hour to about 3 hours prior to the release of the psychedelic.

Described herein, in some embodiments, are compositions comprising a psychedelic and a serotonin receptor modulator, wherein the serotonin receptor modulator is used to pretreat prior to the release of the psychedelic. In some embodiments, the serotonin receptor modulator is used to pretreat at most about 2 hours prior to the release of the psychedelic. In some embodiments, the serotonin receptor modulator is used to pretreat at most about 1 hour prior to the release of the psychedelic. In some embodiments, the serotonin receptor modulator is used to pretreat at most about 1.5 hours prior to the release of the psychedelic. In some embodiments, the serotonin receptor modulator is used to pretreat at about 1 hour prior to the release of the psychedelic. In some embodiments, the serotonin receptor modulator is used to pretreat at most about 5 minutes, 10 minutes, 20 minutes, 30 minutes, 40 minutes, 50 minutes, 1 hour, 1.25 hours, 1.5 hours, 2 hours, or 3 hours prior to the release of the psychedelic. In some embodiments, the serotonin receptor modulator is used to pretreat at most about 3 hours, 4 hours, 5 hours, 6 hours, 7 hours, 8 hours, 9 hours, or more than 9 hours prior to the release of the psychedelic. In some embodiments, the serotonin receptor modulator is used to pretreat in a range of about 5 minutes to about 3 hours, about 10 minutes to about 3 hours, about 20 minutes to about 3 hours, about 30 minutes to about 3 hours, about 40 minutes to about 3 hours, about 50 minutes to about 3 hours, about 1 hour to about 3 hours, about 5 minutes to about 2 hours, about 10 minutes to about 2 hours, about 20 minutes to about 2 hours, about 30 minutes to about 2 hours, about 40 minutes to about 2 hours, about 50 minutes to about 2 hours, about 1 hour to about 2 hours, about 5 minutes to about 1 hour, about 10 minutes to about 1 hour, about 20 minutes to about 1 hour, about 30 minutes to about 1 hour, about 40 minutes to about 1 hour, or about 50 minutes to about 1 hour.

Described herein, in some embodiments, are compositions comprising a psychedelic and a serotonin receptor modulator, wherein the psychedelic modulates a serotonin receptor. In some embodiments, the psychedelic is a serotonergic psychedelic (also known as serotonergic hallucinogen). Serotonergic psychedelics are a subclass of psychedelic drugs with a method of action strongly tied to the neurotransmitter serotonin. Serotonin (often referred to as 5-HT, short for its full chemical name 5-hydroxytryptamine) is a naturally occurring neurotransmitter which is tied to positive mood, certain involuntary muscle control, and countless other functions, many of which are not yet fully understood. In some embodiments, the psychedelic is a serotonin receptor agonist. In some embodiments, the psychedelic is a partial agonist. In some embodiments, the serotonin receptor is serotonin receptor 1, serotonin receptor 2, serotonin receptor 4, serotonin receptor 5, serotonin receptor 6, or serotonin receptor 7. In some embodiments, the serotonin receptor is serotonin receptor 1A, serotonin receptor 1B, serotonin receptor 1D, serotonin receptor 1E, serotonin receptor IF, serotonin receptor 2A, serotonin receptor 2B, serotonin receptor 2C, serotonin receptor 4, serotonin receptor 5A, serotonin receptor 5B, serotonin receptor 6, or serotonin receptor 7. In some embodiments, the serotonin receptor is serotonin receptor 1B, serotonin receptor 4, serotonin receptor 6, or serotonin receptor 7.

### Psychedelics

In some embodiments, the psychedelic is selected from the group consisting of psilocybin, psilocin, baeocystin, norbaeocystin, lisurgide, lysergic acid diethylamide (LSD), 1-acetyl LSD, N,N-Dimethyltryptamine (DMT), 5-methoxy-N,N-dimethyltryptamine (5-MeO-DMT), ibogaine, 3,4-methylenedioxy-methamphetamine (MDMA), O-acetylpsilocin (4-Acetoxy-N,N-dimethyltryptamine), mescaline (3,4,5-trimethoxy-phenethylamine), 2C-B (4-Bromo-2,5-dimethoxyphenethylamine), phenethylamine (PEA), carboxamindotryptamine, proscaline (2-(3,5-dimethoxy-4-propoxyphenyl)ethanamine), metaescaline (2-(3-ethoxy-4,5-dimethoxyphenyl)ethanamine), allylescaline (4-Allyloxy-3,5-dimethyloxy phenylethylamine), methallylescaline (4-Methallyloxy-3,5-dimethoxyphenethylamine), 3,4-Methylenedioxy-A (MDA), 3,4-methylenedioxy-N-ethylamphetamine (MDE), asymbescaline (3,4-Diethoxy-5-methoxyphenethylamine), mescaline-NBOMe, IP-LSD, 1B-LSD, ETH-LAD, 1P-ETH-LAD, AL-LAD, LSZ, LSM-775, 1-(4-Bromofuro[2,3-f][1]benzofuran-8-yl)propan-2-amine, 25I-NBOH, N-(2-Methoxybenzyl)-2-(3,4,5-trimethoxyphenyl)ethanamine, N-(2-hydroxybenzyl)-2,5-dimethoxy-4-iodo-phenethylamine, N-(2-hydroxybenzyl)-2,5-dimethoxy-4-chloro-phenethylamine, N-(2-hydroxybenzyl)-2,5-dimethoxy-4-bromo-phenethylamine, 4-Allyloxy-3,5-dimethyloxyphenylethylamine, N-(2-fluorobenzyl)-2,5-dimethoxy-4-iodo-phenethylamine, 2,5-dimethoxy-4-tert-butylthio-phenethylamine, 2,5-dimethoxy-4-propylthio-phenethylamine, 2,5-dimethoxy-4-propylphenethylamine, 2,5-dimethoxy-4-nitrophenethylamine, 2,5-dimethoxy-4-nitroamphetamine, 2,5-dimethoxy-4-methylphenethylamine, 2,5-dimethoxy-4-methylamphetamine, 2,5-dimethoxy-4-isopropylthio-phenethylamine, 2,5-dimethoxy-4-iodophenethylamine, 2,5-dimethoxy-4-iodoamphetamine, 2,5-dimethoxy-4-fluorophenethylamine, 2,5-dimethoxy-4-ethylthio-phenethylamine, 2,5-dimethoxy-4-ethylphenethylamine, 2,5-dimethoxy-4-cyclopropylmethylthio-phenethylamine, 2,5-dimethoxy-4-chlorophenethylamine, 2,5-dimethoxy-4-chloroamphetamine, 2,5-dimethoxy-4-bromoamphetamine, 2,5-dimethoxy-4-bromo-ß-ketophenethylamine, 2,5-dimethoxy-4-(2-fluoroethylthio)-phenethylamine, 2-(4-propyl-2,5-dimethoxyphenyl)-N-[(2-methoxyphenyl)methyl]ethanamine, 2-(4-methyl-2,5-dimethoxyphenyl)-N-[(2-methoxyphenyl)methyl]ethanamine, 2-(4-iodo-2,5-dimethoxyphenyl)-N-[(2-methoxyphenyl)methyl]ethanamine, 2-(4-fluoro-2,5-dimethoxyphenyl)-N-[(2-methoxyphenyl)methyl]ethanamine, 2-(4-ethyl-2,5-dimethoxyphenyl)-N-[(2-methoxyphenyl)methyl]ethanamine, 2-(4-chloro-2,5-dimethoxyphenyl)-N-[(2-methoxyphenyl)methyl]ethanamine, 2-(4-bromo-2,5-dimethoxyphenyl)-N-[(2-methoxyphenyl)methyl]ethanamine, 2-Bromo-4,5-methylenedioxy-A (2-Br-4,5-MDA), 4-Bromo-3,5-dimethoxy-A (4-Br-3,5-DMA), 3,4-Dimethyl-2,5-dimethoxy-PEA (2C-G), 3,4-Trimethylene-2,5-dimethoxy-PEA (2C-G-3), 3,4-Trimethylene-2,5-dimethoxy-A (G-3), 3,4-Tetramethylene-2,5-dimethoxy-PEA (2C-G-4), 3,4-Tetramethylene-2,5-dimethoxy-A (G-4), 3,4-Norbornyl-2,5-dimethoxy-PEA (2C-G-5), 3,4-Norbornyl-2,5-dimethoxy-A (G-5), 1,4-Dimethoxynaphthyl-2-ethylamine (2C-G-N), 1,4-Dimethoxynaphthyl-2-isopropylamine (G-N), 2,5-Dimethoxy-PEA (2C-H), 4-Ethoxy-3,5-dimethoxy-A (3C-E), 4-Ethoxy-3,5-dimethoxy-PEA, 4-Benzyloxy-3,5-dimethoxy-A (3C-BZ), 4-Isopropoxy-2,5-dimethoxy-PEA (2C-O-4), 4-Methylseleno-2,5-dimethoxy-PEA (2C-SE), 4-Methylthio-2,5-dimethoxy-PEA (2C-T), 4-Isopropylthio-2,6-dimethoxy-PEA (psi-2C-T-4), 4-(2-Methoxyethylthio)-2,5-dimethoxy-PEA (2C-T-13), 4-Cyclopropylthio-2,5-dimethoxy-PEA (2C-T-15), 4-(s)-Butylthio-2,5-dimethoxy-PEA (2C-T-17), 4-Acetoxy-N-methyl-N-ethyltryptamine (4-AcO-MET), 4-Acetoxy-N-methyl-N-allyltryptamine (4-AcO-MALT), 4-Acetyloxy-N,N-diallyltryptamine (4-AcO-DALT), N,N,N-trimethyl-4-phosphoryloxytryptamine (aeruginascin), 4-Hydroxy-N,N,N-trimethyltryptamine, [3-(2-Dimethylaminoethyl)-1H-indol-4-yl] dihydrogen phosphate, 4-hydroxytryptamine, 4-hydroxy-N,N-dimethyltryptamine, [3-(2-methylaminoethyl)-1H-indol-4-yl] dihydrogen phosphate, 4-hydroxy-N-methyltryptamine, [3-(aminoethyl)-1H-indol-4-yl] dihydrogen phosphate, [3-(2-trimethylaminoethyl)-1H-indol-4-yl] dihydrogen phosphate, and 4-hydroxy-N,N,N-trimethyltryptamine, 6-Allyl-N,N-diethyl-NL, N,N-Dibutyl-T, N,N-Diethyl-T, N,N-Diisopropyl-T, alpha-methyl-T, 5-Methyoxy-alpha-methyl-T, 2,alpha-Dimethyl-T, alpha,N-Dimethyl-T, N,N-Dipropyl-T, N-Ethyl-N-isopropyl-T, alpha-Ethyl-T, 6,N,N-Triethyl-NL, 3,4-Dihydro-7-methoxy 1-methyl-C, 7-Methyoxy-1-methyl-C, N,N-Dibutyl-4-hydroxy-T, N,N-Diethyl-4-hydroxy-T, N,N-Diisopropyl-4-hydroxy-T, N,N-Dimethyl-4-hydroxy-T, N,N-Dimethyl-5-hydroxy-T, N,N-Dipropyl-4-hydroxy-T, N-Ethyl-4-hydroxy-N-methyl-T, 4-Hydroxy-N-isopropyl-N-methyl-T, 4-Hydroxy-N-methyl-N-propyl-T, 4-Hydroxy-N,N-tetramethylene-T, Ibogaine, N-Butyl-N-methyl-T, N,N-Diisopropyl-4,5-methylenedioxy-T, N,N-Diisopropyl-5,6-methylenedioxy-T, N,N-Dimethyl-4,5-methylenedioxy-T, 2,N-Dimethyl-4,5-methylenedioxy-A, N,N-Dimethyl-5,6-methylenedioxy-T, N-Isopropyl-N-methyl-5,6-methylenedioxy-T, N,N-Diethyl-2-methyl-T, 2, N, N-Trimethyl-T, N-Acetyl-5-methoxy-T, N, N-Diethyl-5-methoxy-T, N,N-Diisopropyl-5-methoxy-T, N-Isopropyl-4-methoxy-N-methyl-T, N-Isopropyl-5-methoxy-N-methyl-T,5,6-Dimethoxy-Nisopropyl-N-methyl-T, 5-Methoxy-N-methyl-T, 5-Methoxy-N,N-tetramethylene-T, 6-Methoxy-1-methyl-1,2,3,4-tetrahydro-C, 5-Methoxy-2,N, N-trimethyl-T, N, N-Dimethyl-5-methylthio-T, N-Isopropyl-N-methyl-T, alpha Methyl-T, N-Ethyl-T, N-Methyl-T, 6-Propyl-N L, N,N-Tetramethylene-T, Tryptamine, 7-Methoxy-1-methyl-1, 2,3,4-tetrahydro-C, alpha, N-Dimethyl-5-methoxy-T, alpha-Ethyl-3,4,5-trimethoxy-PEA (AEM), 4-Methylthio-2,5-dimethoxy-A (ALEPH), 4-Ethylthio-2,5-dimethoxy-A (ALEPH-2), 4-Isopropylthio-2,5-dimethoxy-A (ALEPH-4), 4-Phenylthio-2,5-dimethoxy-A (ALEPH-6), 4-Propylthio-2,5-dimethoxy-A (ALEPH-7), 2,5-Dimethoxy-alpha-ethyl-4-methyl-PEA (ARIADNE), 4-Butoxy-3,5-dimethoxy-PEA, 2,5-Dimethoxy-4,N-dimethyl-A (BEATRICE), 2,5-Bismethylthio-4-methyl-A (BIS-TOM), 4-Bromo-2,5,beta-trimethoxy-PEA (BOB), 2,5,beta-Trimethoxy-4-methyl-PEA (BOD), beta-Methoxy-3,4-methylenedioxy-PEA (BOH), 2,5-Dimethoxy-beta-hydroxy-4-methyl-PEA (BOHD), 3,4,5, beta-Tetramethoxy-PEA (BOM), 4-Cyclopropylmethoxy-3,5-dimethoxy-PEA (CPM), 4-Trideuteromethyl-3,5-dimethoxy-PEA (4-D), 3,4,5-trimethoxy-beta,beta-dideuterophenethylamine (beta-D), 4-Methyl-3,5-Dimethoxy-PEA, 2,4-Dimethoxy-A (2,4-DMA), 2,5-Dimethoxy-A (2,5-DMA), 3,4-Dimethoxy-A (3,4-DMA), 2-(2,5-Dimethoxy-4-methylphenyl)-cyclopropylamine (DMCPA), 3,4-Dimethoxy-beta-hydroxy-PEA (DME), 2,5-Dimethoxy-3,4-methylenedioxy-A (DMMDA), 2,3-Dimethoxy-4,5-methylenedioxy-A (DMMDA-2), 3,4-Dimethoxy-PEA (DMPEA), 2,5-dimethoxy-4-(n)-amylamphetamine (DOAM), 4-(2-Fluoroethyl)-2,5-dimethoxy-A (DOEF), 4-Ethyl-2,5-dimethoxy-A (DOET), 4-Methyl-2,6-dimethoxy-A (psi-DOM), 4-Propyl-2,5-dimethoxy-A (DOPR), 2,4,5-Triethoxy-A (EEE), 4-Diethoxy-5-methoxy-A (EEM), 2,5-Diethoxy-4-methoxy-A (EME), 2-Ethoxy-4,5-dimethoxy-A (EMM), N,alpha-diethyl-3,4-methylenedioxy-PEA (ETHYL-J), N-Ethyl-alpha-propyl-3,4-methylenedioxy-PEA (ETHYL-K), Benzofuran-2-methyl-5-methoxy-6-(2-aminopropane), Benzofuran-2,2-dimethyl-5-methoxy-6-(2-aminopropane), N-Hydroxy-N-methyl-3,4-methylenedioxy-A (FLEA), 3,4-Dimethyl-2,5-dimethoxy-A, 2,5-Dimethoxy-N-hydroxy-4-ethylthio-PEA (HOT-2), 2,5-Dimethoxy-N-hydroxy-4-(n)-propylthio-PEA (HOT-7), 2,5-Dimethoxy-N-hydroxy-4-(s)-butylthio-PEA (HOT-17), 2,5-Dimethoxy-N,N-dimethyl-4-iodo-A (IDNNA), 2,3,4-Trimethoxy-PEA (IM), 3,5-Dimethoxy-4-isopropoxy-PEA (IP), 5-Ethoxy-2-methoxy-4-methyl-A (IRIS), alpha-Ethyl-3,4-methylenedioxy-PEA, 3-Methoxy-4,5-methylenedioxy-PEA, 3-Methoxy-4,5-methylenedioxy-A (MMDA), 2-Methoxy-4,5-methylenedioxy-A (MMDA-2), 2-Methoxy-3,4-methylenedioxy-A (MMDA-3a), 4-Methoxy-2,3-methylenedioxy-A (MMDA-3b), 4-methoxyamphetamine, N-Allyl-3,4-methylenedioxy-A (MDAL), N-Butyl-3,4-methylenedioxy-A (MDBU), N-Benzyl-3,4-methylenedioxy-A (MDBZ), N-Cyclopropylmethyl-3,4-methylenedioxy-A (MDCPM), N,N-Dimethyl-3,4-methylenedioxy-A (MDDM), N-(2-Hydroxyethyl)-3,4-methylenedioxy-A, N-Isopropyl-3,4-methylenedioxy-A (MDIP), N-Methyl-3,4-ethylenedioxy-A (MDMC), N-Methoxy-3,4-methylenedioxy-A, N-(2-Methoxyethyl)-3,4-methylenedioxy-A, alpha, alpha,N-Trimethyl-3,4-methylenedioxy-PEA (MDMP), N-Hydroxy-3,4-methylenedioxy-A (MDOH), 3,4-Methylenedioxy-PEA, alpha, alpha-Dimethyl-3,4-methylenedioxy-PEA (MDPH), N-Propargyl-3,4-methylenedioxy-A (MDPL), N-Propyl-3,4-methylenedioxy-A (MDPR), 3,4-Dimethoxy-5-ethoxy-PEA (ME), 3-methoxy-4,5-Ethylenedioxy-A (MEDA), 2-Methoxy-4,5-diethoxy-A (MEE), 2,5-Dimethoxy-4-ethoxy-A (MEM), 3-Methoxy-4-ethoxy-PEA, 5-Bromo-2,4-dimethoxy-A, 5-Methylthio-2,4-dimethoxy-A, N-Methyl-2,5-dimethoxy-A, 4-Bromo-2,5-dimethoxy-N-methyl-A, N-Methyl-alpha-ethyl-3,4-methylenedioxy-PEA, N-Methyl-alpha-propyl-3,4-methylenedioxy-PEA, N-Methyl-4-methoxy-A, N-Methyl-2-methoxy-4,5-methylenedioxy-A, 2,4-Dimethoxy-5-ethoxy-A (MME), 3,4-Dimethoxy-5-propoxy-PEA (MP), 2,5-Dimethoxy-4-propoxy-A (MPM), 2-Methylthio-4,5-dimethoxy-A, 3,5-Dimethoxy-4-phenethyloxy-PEA (PE), 4-Propynyloxy-3,5-dimethoxy-PEA, 3,5-Diethoxy-4-methoxy-PEA, 3,4,5-Tetramethoxy-A, 4-Ethoxy-3-ethylthio-5-methoxy-PEA, 3-Ethoxy-4-ethylthio-5-methoxy-PEA, 3,4-Diethoxy-5-methylthio-PEA, 4-Thiobutoxy-3,5-dimethoxy-PEA, 4-Ethoxy-5-methoxy-3-methylthio-PEA (3-TE), 3,5-Dimethoxy-4-ethylthio-PEA (4-TE), 2-Methylthio-3,4-dimethoxy-PEA (2-TIM), 3-Methylthio-2,4-dimethoxy-PEA (3-TIM), 4-Methylthio-2,3-dimethoxy-PEA (4-TIM), 3-Methylthio-4,5-dimethoxy-PEA (3-TM), 4-Methylthio-3,5-dimethoxy-PEA (4-TM), 3,4,5-Trimethoxy-A (TMA), 2,4,5-Trimethoxy-A (TMA-2), 2,3,4-Trimethoxy-A (TMA-3), 2,3,5-Trimethoxy-A (TMA-4), 2,3,6-Trimethoxy-A (TMA-5), 2,4,6-Trimethoxy-A (TMA-6), 4,5-Dimethoxy-3-ethylthio-PEA (3-TME), 3-Ethoxy-5-methoxy-4-methylthio-PEA (4-TME), 3-Ethoxy-4-methoxy-5-methylthio-PEA (5-TME), 2-Methylthio-3,4-methylenedioxy-A, 4,5-Thiomethyleneoxy-2-methoxy-A, 2,4,5-Trimethoxy-PEA, 4-Ethyl-5-methoxy-2-methylthio-A (2-TOET), 4-Ethyl-2-methoxy-5-methylthio-A (5-TOET), 4-Ethyl-2-methoxy-5-methylthio-A (2-TOM), 2-Methoxy-4-methyl-5-methylthio-A (5-TOM), 2-Methoxy-4-methyl-5-methylsulfinyl-A (TOMSO), 4-Propylthio-3,5-dimethoxy-PEA (TP), 3,4,5-Triethoxy-PEA (TRIS), 3-Ethoxy-5-ethylthio-4-methoxy-PEA (3-TSB), 3,5-Diethoxy-4-methylthio-PEA (4-TSB), 4,5-Diethoxy-3-ethylthio-PEA (3-T-TRIS), 3,5-Diethoxy-4-ethylthio-PEA (4-T-TRIS), 2-(2-Chlorophenyl)-2-(methylamino)cyclohexanone (ketamine), 8-methoxy-3-methyl-1,2,3,4,5,6-hexahydroazepino[4,5-b]indole (tabernanthalog), 2-Bromolysergic acid diethylamide, 5-methoxy-2,3-dihydro-1H-inden-2-amine (MEAI), N-methyl-N-allyltryptamine (MALT),N-ethyl-N-propyltryptamine (EPT), 5-Methoxy-N,N-diallyltryptamine (5-MeO-DALT), 6-Methoxy-N,N-dimethyltryptamine (6-MeO-DMT), 6-fluoro-N,N,-dimethyltryptamine (6-Fluoro-DMT), N-methyl-N-propyltryptamine (MPT), N-Methyl-N-isopropyltryptamine (MiPT), N,N-Dimethyl-N-allyltryptamine (DMALT), 4-Acetoxy-N,N,N-trimethyltryptamine (4-AcO-TMT), 4-Acetoxy-N,N-dimethyl-N-ethyltryptamine (4-OAc-DMET), 4-Acetoxy-N,N-dimethyl-N-propyltryptamine (4-AcO-DMPT), N-(4-bromophenyl)adamantan-2-amine (bromantane), 3-(6-(4-fluoro-3-methoxyphenoxy)pyrimidin-4-yl)-5,5-dimethylimidazolidine-2,4-dione, N-(4-((2-fluorobenzyl)oxy)benzyl)-2-(trifluoromethyl)thiazole-4-carboxamide, 4-{trans-2-[4-(3-Fluorophenyl)pyrimidin-2-yl]cyclopropyl}benzenesulfonamide, sodium 6-methoxy-2-methyl-3-(3,4,5-trimethoxybenzoyl)benzofuran-7-yl phosphate, 1-ethyl-6-(indan-2-ylamino)-3-(morpholine-4-carbonyl)-1,8-naphthyridin-4-one, 4-((1S,3S)-3-(5-cyclopentyl-1,2,4-oxadiazol-3-yl)-2,2-dimethylcyclopropyl)benzenesulfonamide, (2S,5R)-5-(4-((1-(5-fluoro-2-(trifluoromethoxy)phenyl)-1H-tetrazol-5-yl)oxy)phenyl)pyrrolidine-2-carboxamide, N-(4-fluorophenethyl)-3-methylisoxazole-4-sulfonamide, (7-hydroxy-6-methoxy-2-methylbenzofuran-3-yl)(3,4,5-trimethoxyphenyl)methanone(7-hydroxy-6-methoxy-2-methylbenzofuran-3-yl)(3,4,5-trimethoxyphenyl)methanone, 6-((2,3-dihydro-1H-inden-2-yl)amino)-1-ethyl-3-(1-methyl-1H-imidazol-2-yl)-1,8-naphthyridin-4(1H)-one, 2-(2-(Allyloxy)-5-fluorophenyl)cyclopropyl)methanamine, 1,5-dimethyl-N-(2-(trifluoromethyl)pyridin-4-yl)-1H-indole-3-carboxamide, (2-(5-Fluoro-2-(2-fluoroethoxy)phenyl)cyclopropyl)methanamine, (2-(5-Chloro-2-(2-fluoroethoxy)phenyl)cyclopropyl)methanamine, (2-(5-Chloro-2-((2-fluoroallyl)oxy)phenyl)cyclopropyl)methanamine, 5,6-dimethoxy-2,3-dihydro-1H-inden-2-amine, (R)-1-(5-Methoxy-1H-Indol-1-YL)-N,N-Dimethylpropan-2-Amine, N—N-disisopropyltryptamine-4-glutarate, 2-methoxy-7-methyl-5,6,7,8,9,10-hexahydropyrido[3′,2′:4,5]pyrrolo[2,3-d]azepine, 1-(5-methoxy-1H-indol-1-yl)-N,N,2-trimethylpropan-2-amine, 2-(5-methoxy-1H-pyrrolo[2,3-c]pyridin-1-yl)-N,N-dimethylethanamine, (R)—N,N-diethyl-1,3,4,5-tetrahydrobenzo[cd]indol-4-amine, (S)—N,N-diethyl-1,3,4,5-tetrahydrobenzo[cd]indol-4-amin, 2-(4-allyl-2,5-dimethoxyphenyl)ethanamine, N-Ethyl-2-(5-Fluoro-1H-Indol-3-YL)-N-Methylethan-1-Amine, (R)-2-(methylamino)-2-phenylcyclohexanone, (R)-2-(D3-methylamino)-2-phenylcyclohexanone, (S)-2-(methylamino)-2-phenylcyclohexanone, (S)-2-(D3-methylamino)-2-phenylcyclohexanone, (S)-3-(2,5-dimethoxy-4-(trifluoromethyl)phenyl)piperidine, 3-methyl-methcathinone (3-MMC), 3-(2-(Bis(Methyl-D3)Amino)Ethyl-1,1,2,2-D4)-1H-Indol-4-YL(9Z,12Z)-Octadeca-9,12-Dienoate, (R)-3-((1-(Methyl-d3)Pyrolidin-2-YL)Methyl)-1H-Indol-4-OL, 5-(2-methylaminopropyl)benzofuran, 6-(2-methylaminopropyl)benzofuran, 2-chloro-N,N,-dimethyltryptamine,2-bromo-N,N,-dimethyltryptamine, 2-bromo-4-acetoxy-N,N-dimethyltryptamine, 2-chloro-4-methoxy-N,N,-dimethyltryptamine, 1-(3-(2-(dimethylamino)ethyl)-1H-indol-4-yl)-N-methylmethanesulfonamide, DMT-alpha, alpha-d2 (DMT-d2), psilocin-alpha, alpha-d2 (psilocin-d2), aeruginascin-alpha-alpha-d2 (aeruginascin-d2), razoxane, dexrazoxane, N-Allyl-3,4-methylenedioxy-amphetamine (MDAL), N-Butyl-3,4-methylenedioxyamphetamine (MDBU), N-Benzyl-3,4-methylenedioxyamphetamine (MDBZ), N-Cyclopropylmethyl-3,4-methylenedioxyamphetamine (MDCPM), N,N-Dimethyl-3,4-methylenedioxyamphetamine (MDDM), N-Ethyl-3,4-methylenedioxyamphetamine (MDE; MDEA), N-(2-Hydroxyethyl)-3,4-methylenedioxy amphetamine (MDHOET), N-Isopropyl-3,4-methylenedioxyamphetamine (MDIP), N-Methyl-3,4-ethylenedioxyamphetamine (MDMC), N-Methoxy-3,4-methylenedioxyamphetamine (MDMEO), N-(2-Methoxyethyl)-3,4-methylenedioxyamphetamine (MDMEOET), alpha, alpha,N-Trimethyl-3,4-methylenedioxyphenethylamine (MDMP), 3,4-Methylenedioxy-N-methylphentermine, N-Hydroxy-3,4-methylenedioxyamphetamine (MDOH), 3,4-Methylenedioxyphenethylamine (MDPEA), alpha,alpha-Dimethyl-3,4-methylenedioxyphenethylamine (MDPH; 3,4-methylenedioxyphentermine), N-Propargyl-3,4-methylenedioxyamphetamine (MDPL), Methylenedioxy-2-aminoindane (MDAI), 1,3-Benzodioxolyl-N-methylbutanamine (MBDB)N-methyl-1,3-benzodioxolylbutanamine (MBDB), 3,4-methylenedioxy-N-methyl-a-ethylphenylethylamine,3,4-Methylenedioxyamphetamine (MDA), Methylone (also known as “3,4-methylenedioxy-N-methylcathinone), Ethylone, (also known as 3,4-methylenedioxy-N ethylcathinone), GHB or Gamma, Hydroxybutyrate or sodium oxybate, N-Propyl-3,4 methylenedioxyamphetamine (MDPR) or a pharmaceutically acceptable salt, solvate, metabolite, deuterated analogue, derivative, prodrug, or combinations thereof. In some embodiments, the psychedelic is selected from the group consisting of psilocybin, psilocin, baeocystin, norbaeocystin, lisurgide, LSD, dimethyltryptamine, carboxamindotryptamine, ibogaine, tabernanthalog, 3,4-methylenedioxy-methamphetamine (MDMA), 1-acetyl LSD, O-acetyl psilocin, mescaline (3,4,5-trimethoxy phenethylamine), proscaline (2-(3,5-dimethoxy-4-propoxyphenyl)ethanamine), metaescaline (2-(3-ethoxy-4,5-dimethoxyphenyl)ethanamine), allylescaline (4-Allyloxy-3,5-dimethyloxy phenylethylamine), methallylescaline (4-Methallyloxy-3,5 dimethoxyphenethylamine), and asymbescaline (3,4-Diethoxy-5-methoxyphenethylamine), or a pharmaceutically acceptable salt, solvate, metabolite, deuterated analogue, derivative, prodrug, or combinations thereof. In some embodiments, the psychedelic is psilocybin or psilocin, or a pharmaceutically acceptable salt, solvate, metabolite, deuterated analogue, derivative, prodrug, or combinations thereof. In some embodiments, the psychedelic is [3-(2-dimethylaminoethyl)-1H-indol-4-yl] dihydrogen phosphate, 4-hydroxy-N,N-dimethyltryptamine, [3-(2-methylaminoethyl)-1H-indol-4-yl] dihydrogen phosphate, 4-hydroxy-N-methyltryptamine, [3-(aminoethyl)-1H-indol-4-yl] dihydrogen phosphate, 4-hydroxytryptamine, [3-(2-trimethylaminoethyl)-1H-indol-4-yl] dihydrogen phosphate, 4-hydroxy-N,N,N-trimethyltryptamine, or a pharmaceutically acceptable salt, solvate, metabolite, deuterated analogue, derivative, prodrug, or combinations thereof. In some embodiments, the psychedelic is 1-acetyl LSD (ALD-52) or a pharmaceutically acceptable salt, solvate, metabolite, deuterated analogue, derivative, prodrug, or combinations thereof. In some embodiments, the psychedelic is O-acetyl psilocin (psilacetin) or a pharmaceutically acceptable salt, solvate, metabolite, deuterated analogue, derivative, prodrug, or combinations thereof.

In some embodiments, the psychedelic is LSD or a pharmaceutically acceptable salt, solvate, metabolite, deuterated analogue, derivative, or prodrug thereof. In some embodiments, the LSD derivative is 1P-LSD, 1B-LSD, ETH-LAD, IP-ETH-LAD, AL-LAD, LSZ, LSM-775, 1-(4-Bromofuro[2,3-f][1]benzofuran-8-yl)propan-2-amine.

In some embodiments, the psychedelic is mescaline or a pharmaceutically acceptable salt, solvate, metabolite, deuterated analogue, derivative, or prodrug thereof. In some embodiments, the mescaline derivative is mescaline-NBOMe, proscaline (2-(3,5-dimethoxy-4-propoxyphenyl)ethanamine), or metaescaline (2-(3-ethoxy-4,5-dimethoxyphenyl)ethanamine).

In some embodiments, the psychedelic is a phenethylamine, a tryptamine, or a pharmaceutically acceptable salt, solvate, metabolite, deuterated analogue, derivative, or prodrug thereof selected from 25I-NBOH, N-(2-Methoxybenzyl)-2-(3,4,5-trimethoxyphenyl)ethanamine, N-(2-hydroxybenzyl)-2,5-dimethoxy-4-iodo-phenethylamine, N-(2-hydroxybenzyl)-2,5-dimethoxy-4-chloro-phenethylamine, N-(2-hydroxybenzyl)-2,5-dimethoxy-4-bromo-phenethylamine, 4-Allyloxy-3,5-dimethyloxyphenylethylamine, N-(2-fluorobenzyl)-2,5-dimethoxy-4-iodo-phenethylamine, 2,5-dimethoxy-4-tert-butylthio-phenethylamine, 2,5-dimethoxy-4-propylthio-phenethylamine, 2,5-dimethoxy-4-propylphenethylamine, 2,5-dimethoxy-4-nitrophenethylamine, 2,5-dimethoxy-4-nitroamphetamine, 2,5-dimethoxy-4-methylphenethylamine, 2,5-dimethoxy-4-methylamphetamine, 2,5-dimethoxy-4-isopropylthio-phenethylamine, 2,5-dimethoxy-4-iodophenethylamine, 2,5-dimethoxy-4-iodoamphetamine, 2,5-dimethoxy-4-fluorophenethylamine, 2,5-dimethoxy-4-ethylthio-phenethylamine, 2,5-dimethoxy-4-ethylphenethylamine, 2,5-dimethoxy-4-cyclopropylmethylthio-phenethylamine, 2,5-dimethoxy-4-chlorophenethylamine, 2,5-dimethoxy-4-chloroamphetamine, 2,5-dimethoxy-4-bromophenethylamine, 2,5-dimethoxy-4-bromoamphetamine, 2,5-dimethoxy-4-bromo-ß-ketophenethylamine, 2,5-dimethoxy-4-(2-fluoroethylthio)-phenethylamine, 2-(4-propyl-2,5-dimethoxyphenyl)-N-[(2-methoxyphenyl)methyl]ethanamine, 2-(4-methyl-2,5-dimethoxyphenyl)-N-[(2-methoxyphenyl)methyl]ethanamine, 2-(4-iodo-2,5-dimethoxyphenyl)-N-[(2-methoxyphenyl)methyl]ethanamine, 2-(4-fluoro-2,5-dimethoxyphenyl)-N-[(2-methoxyphenyl)methyl]ethanamine, 2-(4-ethyl-2,5-dimethoxyphenyl)-N-[(2-methoxyphenyl)methyl]ethanamine, 2-(4-chloro-2,5-dimethoxyphenyl)-N-[(2-methoxyphenyl)methyl]ethanamine, 2-(4-bromo-2,5-dimethoxyphenyl)-N-[(2-methoxyphenyl)methyl]ethanamine, 4-AcO-MET, 4-ACO-MALT and 4-AcO-DALT, Aeruginascin or N,N,N-trimethyl-4-phosphoryloxytryptamine, 4-Hydroxy-N,N,N-trimethyltryptamine, 5-meo-DMT, Ibogaine, [3-(2-Dimethylaminoethyl)-1H-indol-4-yl] dihydrogen phosphate, 4-hydroxytryptamine, 4-hydroxy-N,N-dimethyl-tryptamine, [3-(2-methylaminoethyl)-1H-indol-4-yl] dihydrogen phosphate, 4-hydroxy-N-methyltryptamine, [3-(aminoethyl)-1H-indol-4-yl] dihydrogen phosphate, [3-(2-trimethylaminoethyl)-1H-indol-4-yl] dihydrogen phosphate, and 4-hydroxy-N,N,N-trimethyltryptamine, 6-Allyl-N,N-diethyl-NL, N,N-Dibu-tyl-T, N,N-Diethyl-T, N,N-Diisopropyl-T, 5-Methyoxy-alpha-methyl-T, N,N-Dimethyl-T, 2, alpha-Dimethyl-T, alpha, N-Dimethyl-T, N,N-Dipropyl-T, N-Ethyl-N-isopropyl-T, alpha-Ethyl-T, 6,N,N-Triethyl-NL, 3,4-Dihydro-7-methoxy-1-methyl-C, 7-Methyoxy-1-methyl-C, N,N-Dibutyl-4-hydroxy-T, N,N-Diethyl-4-hydroxy-T, N,N-Diisopropyl-4-hydroxy-T, N,N-Dimethyl-4-hydroxy-T, N,N-Dimethyl-5-hydroxy-T, N, N-Dipropyl-4-hydroxy-T, N-Ethyl-4-hydroxy-N-methyl-T, 4-Hydroxy-N-isopropyl-N-methyl-T, 4-Hydroxy-N-methyl-N-propyl-T, 4-Hydroxy-N,N-tetram-ethylene-T Ibogaine, N,N-Diethyl-L, N-Butyl-N-methyl-T, N,N-Diisopropyl-4,5-methylenedioxy-T, N,N-Diisopropyl-5,6-methylenedioxy-T, N,N-Dimethyl-4,5-methylenedioxy-T, N,N-Dimethyl-5,6-methylenedioxy-T, N-Isopropyl-N-methyl-5,6-methylenedioxy-T, N,N-Diethyl-2-methyl-T, 2,N,N-Trimethyl-T, N-Acetyl-5-methoxy-T, N,N-Diethyl-5-methoxy-T, N,N-Diisopropyl-5-methoxy-T, 5-Methoxy-N, N-dimethyl-T, N-Isopropyl-4-methoxy-N-methyl-T, N-Iso-propyl-5-methoxy-N-methyl-T,5,6-Dimethoxy-N-isopropyl-N-methyl-T, 5-Methoxy-N-methyl-T, 5-Methoxy-N,N-tetramethylene-T, 6-Methoxy-1-methyl-1,2,3,4-tetrahydro-C, 5-Methoxy-2,N,N-trimethyl-T, N,N-Dimethyl-5-methylthio-T, N-Isopropyl-N-methyl-T, alpha-Methyl-T, N-Ethyl-T, N-Methyl-T, 6-Propyl-N L, N,N-Tetramethylene-T, Tryptamine, 7-Methoxy-1-methyl-1, 2,3,4-tetrahydro-C, alpha,N-Dimethyl-5-methoxy-T, or a pharmaceutically acceptable salt, solvate, metabolite, deuterated analogue, derivative, prodrug, or combinations thereof.

In some embodiments, the psychedelic is selected from the group consisting of:

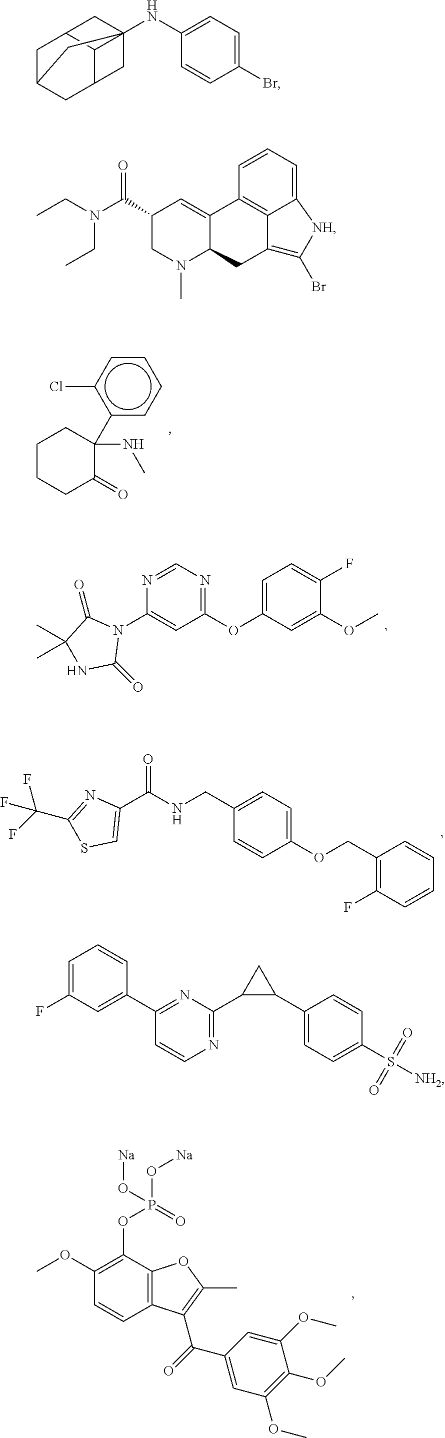

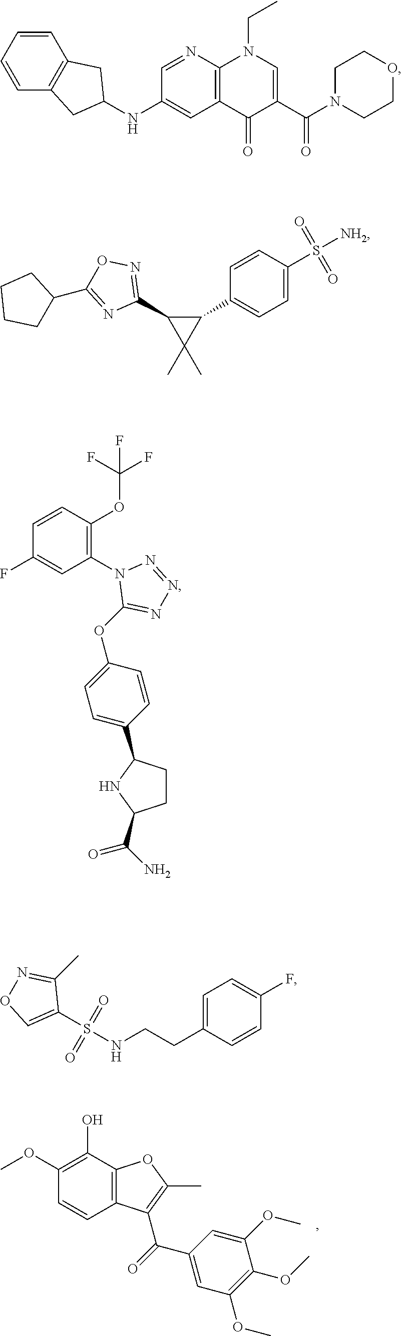

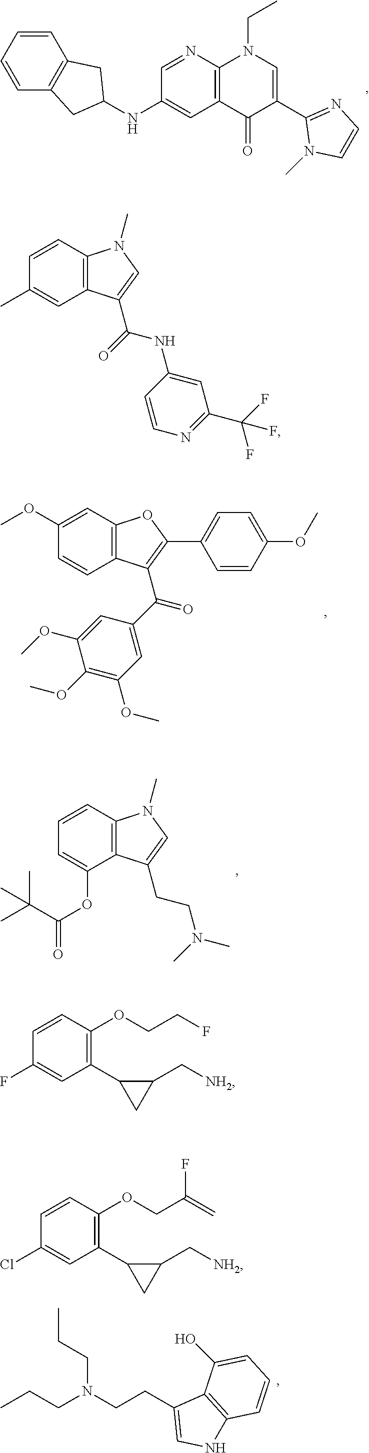

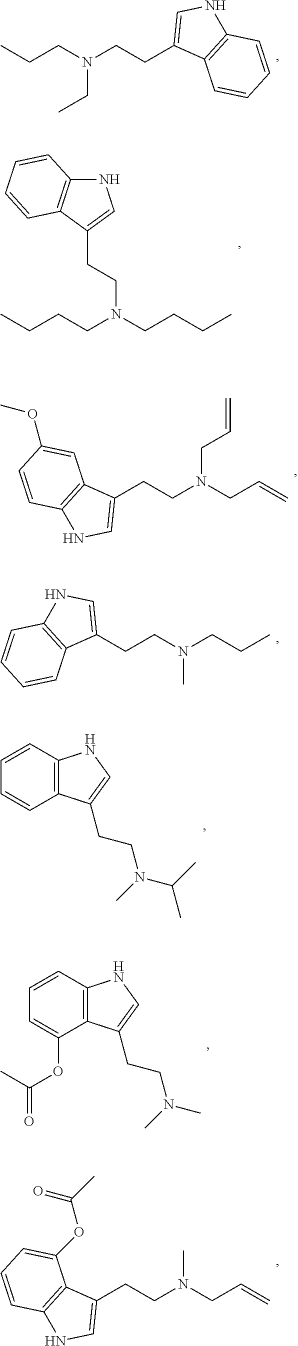

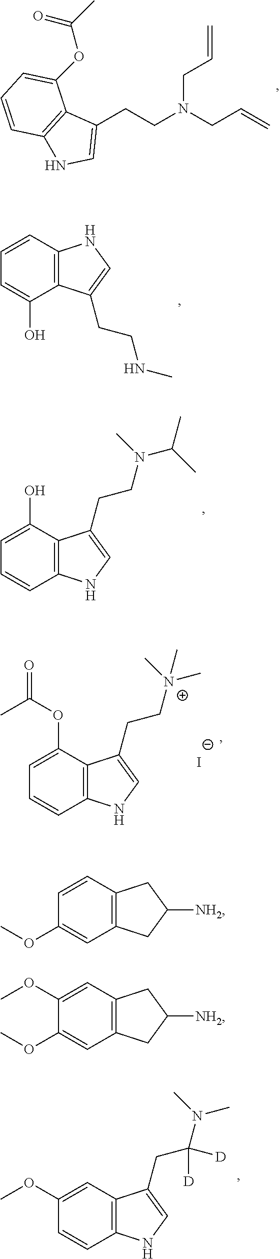

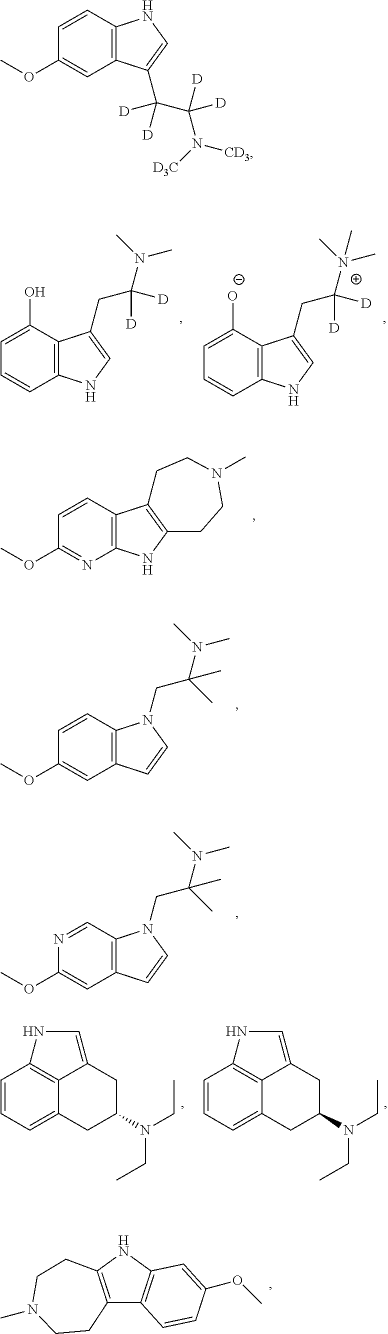

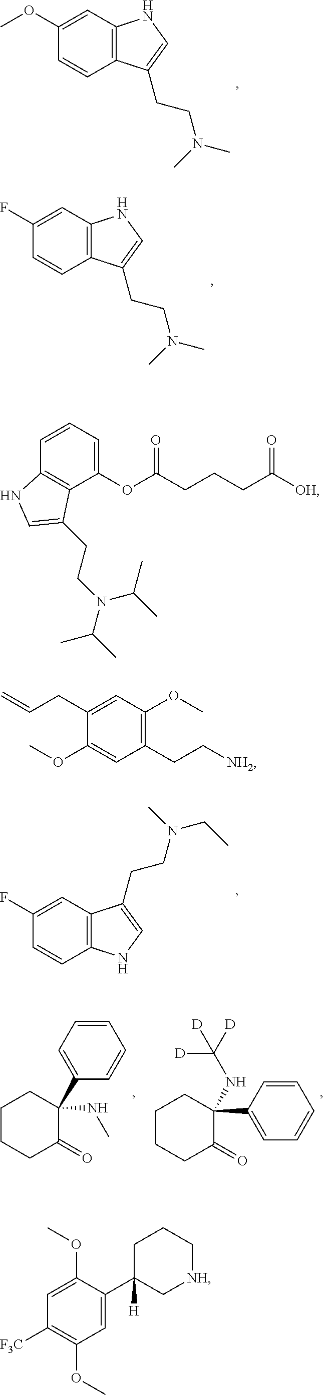

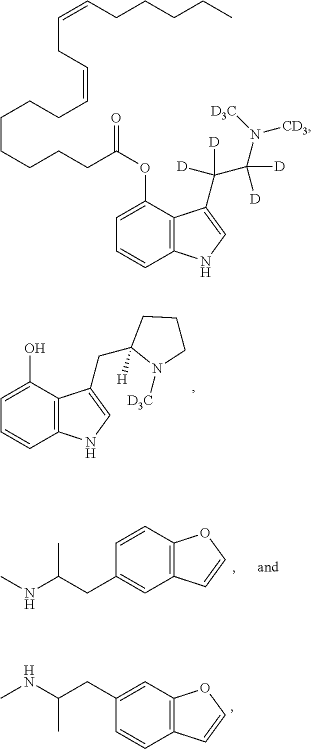 [SMILES: [2H]C([2H])([2H])N(C([2H])([2H])[2H])C([2H])([2H])C([2H])([2H])c1c[nH]c2cccc(OC(=O)CCCCCCC/C=C\C/C=C\CCCCC)c12 ; [2H]C([2H])([2H])N1CCC[C@@H]1Cc1c[nH]c2cccc(O)c12 ; CNC(C)Cc1ccc2occc2c1 ; CNC(C)Cc1ccc2ccoc2c1]

or a pharmaceutically acceptable salt, ester, solvate, metabolite, deuterated analogue, derivative, prodrug, or combinations thereof.

In some embodiments, the psilocybin or psilocin is present in the form of an extract from a mushroom and/or truffle (sclerotium). In some embodiments, the mushroom or truffle is from the genus Psilocybe, Gymnopilus, Panaeolus, Copelandia, Hypholoma, Pluteus, Inocybe, Conocybe, Panaeolina, Gerronema, Agrocybe, Galerina and/or Mycena. In some embodiments, the mushroom or truffle is P. azurescens, P. semilanceata, P. cyanescens, P. cubensis, P. subcubensis, P. tampanensis, P. mexicana, P. atlantis, and/or P. semilanceata.

Described herein, in some embodiments, are compositions comprising a psychedelic and a serotonin receptor modulator, wherein the serotonin receptor modulator is a serotonin receptor antagonist. Further described herein, in some embodiments, are compositions comprising a psychedelic and a serotonin receptor modulator, wherein the serotonin receptor modulator is a serotonin receptor inverse agonist. Further described herein, in some embodiments, are compositions comprising a psychedelic and a serotonin receptor modulator, wherein the serotonin receptor modulator is a serotonin receptor allosteric modulator. In some embodiments, the serotonin receptor is serotonin receptor 1, serotonin receptor 2, serotonin receptor 4, serotonin receptor 5, serotonin receptor 6, or serotonin receptor 7. In some embodiments, the serotonin receptor is serotonin receptor 1A, serotonin receptor 1B, serotonin receptor 1D, serotonin receptor 1E, serotonin receptor 1F, serotonin receptor 2A, serotonin receptor 2B, serotonin receptor 2C, serotonin receptor 4, serotonin receptor 5A, serotonin receptor 5B, serotonin receptor 6, or serotonin receptor 7. In some embodiments the serotonin receptor is serotonin receptor 2A.

### Serotonin Receptor Modulators

In some embodiments, the serotonin receptor modulator described herein is selected from ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, AMDA, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, and RS-102221, or a pharmaceutically acceptable salt, solvate, metabolite, deuterated analogue, derivative, prodrug, or combinations thereof.

In some embodiments, the serotonin receptor modulator described herein comprises kMDL-11,939, eplivanserin (SR-46,349), ketanserin, ritanserin, altanserin, acepromazine, mianserin, mirtazapine, quetiapine, SB204741, SB206553, SB242084, LY272015, SB243213, Blonanserin, SB200646, RS102221, nefazodone, MDL-100,907, pimavanserin, nelotanserin, lorcaserin, flibanserin, and roluperiodone, or a pharmaceutically acceptable salt, solvate, metabolite, deuterated analogue, derivative, prodrug, or combinations thereof. In some embodiments, the serotonin receptor modulator is ketanserin or a pharmaceutically acceptable salt, solvate, metabolite, deuterated analogue, derivative, or prodrug thereof. In some embodiments, the serotonin receptor modulator is pimavanserin or a pharmaceutically acceptable salt, solvate, metabolite, deuterated analogue, derivative, or prodrug thereof.

In some embodiments, the serotonin receptor modulator is ketanserin or a pharmaceutically acceptable salt thereof. In some embodiments, the serotonin receptor modulator is ketanserin.

In some embodiments, the serotonin receptor modulator is pimavanserin or a pharmaceutically acceptable salt thereof. In some embodiments, the serotonin receptor modulator is pimavanserin. In some embodiments, the serotonin receptor modulator is eplivanserin or a pharmaceutically acceptable salt, solvate, metabolite, deuterated analogue, derivative, or prodrug thereof.

In some embodiments, the serotonin receptor modulator is eplivanserin or a pharmaceutically acceptable salt thereof. In some embodiments, the serotonin receptor modulator is eplivanserin.

### Co-Administration of Psychedelic and Serotonin Receptor Modulator

In some embodiments, the psychedelic is selected from the group consisting of psilocybin, 4-Acetoxy-DMT, LSD, ALD-52, 1P-LSD, DMT, 5-MeO-DMT, 2C-B, ibogaine, MDMA, and DOM.

In some embodiments, the serotonin receptor modulator is selected from the group consisting of eplivanserin, volinanserin, ketanserin, ritanserin, pimavanserin, nelotanserin, pruvanserin, and flibanserin.

In some embodiments, the psychedelic is selected from the group consisting of psilocybin, 4-Acetoxy-DMT, LSD, ALD-52, 1P-LSD, DMT, 5-MeO-DMT, 2C-B, ibogaine, MDMA, and DOM, and the serotonin receptor modulator is eplivanserin or volinanserin.

In some embodiments, the psychedelic is selected from the group consisting of psilocybin, 4-Acetoxy-DMT, LSD, ALD-52, 1P-LSD, DMT, 5-MeO-DMT, 2C-B, ibogaine, MDMA, and DOM, and the serotonin receptor modulator is eplivanserin.

In some embodiments, the psychedelic is selected from the group consisting of psilocybin, 4-Acetoxy-DMT, LSD, ALD-52, 1P-LSD, DMT, 5-MeO-DMT, 2C-B, ibogaine, MDMA, and DOM, and the serotonin receptor modulator is volinanserin.

In some embodiments, the psychedelic is selected from the group consisting of psilocybin, 4-Acetoxy-DMT, LSD, ALD-52, 1P-LSD, DMT, 5-MeO-DMT, 2C-B, ibogaine, MDMA, and DOM, and the serotonin receptor modulator is selected from the group consisting of eplivanserin, volinanserin, ritanserin, pimavanserin, nelotanserin, pruvanserin, and flibanserin.

In some embodiments, the psychedelic is selected from the group consisting of psilocybin, 4-Acetoxy-DMT, LSD, ALD-52, 1P-LSD, DMT, 5-MeO-DMT, 2C-B, ibogaine, MDMA, and DOM, and the serotonin receptor modulator is selected from the group consisting of eplivanserin, volinanserin, ketanserin, pimavanserin, nelotanserin, pruvanserin, and flibanserin.

In some embodiments, the psychedelic is selected from the group consisting of psilocybin, 4-Acetoxy-DMT, LSD, ALD-52, 1P-LSD, DMT, 5-MeO-DMT, 2C-B, ibogaine, MDMA, and DOM, and the serotonin receptor modulator is selected from the group consisting of eplivanserin, volinanserin, pimavanserin, nelotanserin, pruvanserin, and flibanserin.

In some embodiments, the psychedelic is selected from the group consisting of psilocybin, 4-Acetoxy-DMT, LSD, ALD-52, 1P-LSD, DMT, 5-MeO-DMT, 2C-B, ibogaine, MDMA, and DOM, and the serotonin receptor modulator is selected from the group consisting of pimavanserin, nelotanserin, pruvanserin, and flibanserin.

In some embodiments, the psychedelic is selected from the group consisting of 4-Acetoxy-DMT, LSD, ALD-52, 1P-LSD, DMT, 5-MeO-DMT, 2C-B, ibogaine, MDMA, and DOM, and the serotonin receptor modulator is selected from the group consisting of eplivanserin, volinanserin, ketanserin, ritanserin, pimavanserin, nelotanserin, pruvanserin, and flibanserin.

In some embodiments, the psychedelic is selected from the group consisting of 4-Acetoxy-DMT, LSD, ALD-52, 1P-LSD, 5-MeO-DMT, 2C-B, ibogaine, MDMA, and DOM, and the serotonin receptor modulator is selected from the group consisting of eplivanserin, volinanserin, ketanserin, ritanserin, pimavanserin, nelotanserin, pruvanserin, and flibanserin.

In some embodiments, the psychedelic is selected from the group consisting of 4-Acetoxy-DMT, ALD-52, 1P-LSD, 5-MeO-DMT, 2C-B, ibogaine, MDMA, and DOM, and the serotonin receptor modulator is selected from the group consisting of eplivanserin, volinanserin, ketanserin, ritanserin, pimavanserin, nelotanserin, pruvanserin, and flibanserin.

In some embodiments, the psychedelic is 4-Acetoxy-DMT, and the serotonin receptor modulator is selected from the group consisting of eplivanserin, volinanserin, ketanserin, ritanserin, pimavanserin, nelotanserin, pruvanserin, and flibanserin.

In some embodiments, the psychedelic is ALD-52, and the serotonin receptor modulator is selected from the group consisting of eplivanserin, volinanserin, ketanserin, ritanserin, pimavanserin, nelotanserin, pruvanserin, and flibanserin.

In some embodiments, the psychedelic is IP-LSD, and the serotonin receptor modulator is selected from the group consisting of eplivanserin, volinanserin, ketanserin, ritanserin, pimavanserin, nelotanserin, pruvanserin, and flibanserin.

In some embodiments, the psychedelic is 2C-B, and the serotonin receptor modulator is selected from the group consisting of eplivanserin, volinanserin, ketanserin, ritanserin, pimavanserin, nelotanserin, pruvanserin, and flibanserin.

In some embodiments, the psychedelic is ibogaine, and the serotonin receptor modulator is selected from the group consisting of eplivanserin, volinanserin, ketanserin, ritanserin, pimavanserin, nelotanserin, pruvanserin, and flibanserin.

In some embodiments, the psychedelic is MDMA, and the serotonin receptor modulator is selected from the group consisting of eplivanserin, volinanserin, ketanserin, ritanserin, pimavanserin, nelotanserin, pruvanserin, and flibanserin.

In some embodiments, the psychedelic is DOM, and the serotonin receptor modulator is selected from the group consisting of eplivanserin, volinanserin, ketanserin, ritanserin, pimavanserin, nelotanserin, pruvanserin, and flibanserin.

In some embodiments, the psychedelic is 4-Acetoxy-DMT, and the serotonin receptor modulator is eplivanserin.

In some embodiments, the psychedelic is ALD-52, and the serotonin receptor modulator is eplivanserin.

In some embodiments, the psychedelic is 1P-LSD, and the serotonin receptor modulator is eplivanserin.

In some embodiments, the psychedelic is 2C-B, and the serotonin receptor modulator is eplivanserin.

In some embodiments, the psychedelic is ibogaine, and the serotonin receptor modulator is eplivanserin.

In some embodiments, the psychedelic is MDMA, and the serotonin receptor modulator is eplivanserin.

In some embodiments, the psychedelic is DOM, and the serotonin receptor modulator is eplivanserin.

In some embodiments, the psychedelic is 4-Acetoxy-DMT, and the serotonin receptor modulator is volinanserin.

In some embodiments, the psychedelic is ALD-52, and the serotonin receptor modulator is volinanserin.

In some embodiments, the psychedelic is 1P-LSD, and the serotonin receptor modulator is volinanserin.

In some embodiments, the psychedelic is 2C-B, and the serotonin receptor modulator is volinanserin.

In some embodiments, the psychedelic is ibogaine, and the serotonin receptor modulator is volinanserin.

In some embodiments, the psychedelic is MDMA, and the serotonin receptor modulator is volinanserin.

In some embodiments, the psychedelic is DOM, and the serotonin receptor modulator is volinanserin.

In some embodiments, the psychedelic is 4-Acetoxy-DMT, and the serotonin receptor modulator is ketanserin.

In some embodiments, the psychedelic is ALD-52, and the serotonin receptor modulator is ketanserin.

In some embodiments, the psychedelic is 1P-LSD, and the serotonin receptor modulator is ketanserin.

In some embodiments, the psychedelic is 2C-B, and the serotonin receptor modulator is ketanserin.

In some embodiments, the psychedelic is ibogaine, and the serotonin receptor modulator is ketanserin.

In some embodiments, the psychedelic is MDMA, and the serotonin receptor modulator is ketanserin.

In some embodiments, the psychedelic is DOM, and the serotonin receptor modulator is ketanserin.

In some embodiments, the psychedelic is 4-Acetoxy-DMT, and the serotonin receptor modulator is ritanserin.

In some embodiments, the psychedelic is ALD-52, and the serotonin receptor modulator is ritanserin.

In some embodiments, the psychedelic is 1P-LSD, and the serotonin receptor modulator is ritanserin.

In some embodiments, the psychedelic is 2C-B, and the serotonin receptor modulator is ritanserin.

In some embodiments, the psychedelic is ibogaine, and the serotonin receptor modulator is ritanserin.

In some embodiments, the psychedelic is MDMA, and the serotonin receptor modulator is ritanserin.

In some embodiments, the psychedelic is DOM, and the serotonin receptor modulator is ritanserin.

In some embodiments, the psychedelic is 4-Acetoxy-DMT, and the serotonin receptor modulator is pimavanserin.

In some embodiments, the psychedelic is ALD-52, and the serotonin receptor modulator is pimavanserin.

In some embodiments, the psychedelic is 1P-LSD, and the serotonin receptor modulator is pimavanserin.

In some embodiments, the psychedelic is 2C-B, and the serotonin receptor modulator is pimavanserin.

In some embodiments, the psychedelic is ibogaine, and the serotonin receptor modulator is pimavanserin.

In some embodiments, the psychedelic is MDMA, and the serotonin receptor modulator is pimavanserin.

In some embodiments, the psychedelic is DOM, and the serotonin receptor modulator is pimavanserin.

In some embodiments, the psychedelic is 4-Acetoxy-DMT, and the serotonin receptor modulator is nelotanserin.

In some embodiments, the psychedelic is ALD-52, and the serotonin receptor modulator is nelotanserin.

In some embodiments, the psychedelic is 1P-LSD, and the serotonin receptor modulator is nelotanserin.

In some embodiments, the psychedelic is 2C-B, and the serotonin receptor modulator is nelotanserin.

In some embodiments, the psychedelic is ibogaine, and the serotonin receptor modulator is selected from the group consisting of nelotanserin.

In some embodiments, the psychedelic is MDMA, and the serotonin receptor modulator is nelotanserin.

In some embodiments, the psychedelic is DOM, and the serotonin receptor modulator is nelotanserin.

In some embodiments, the psychedelic is 4-Acetoxy-DMT, and the serotonin receptor modulator is flibanserin.

In some embodiments, the psychedelic is ALD-52, and the serotonin receptor modulator is flibanserin.

In some embodiments, the psychedelic is 1P-LSD, and the serotonin receptor modulator is flibanserin.

In some embodiments, the psychedelic is 2C-B, and the serotonin receptor modulator is flibanserin.

In some embodiments, the psychedelic is ibogaine, and the serotonin receptor modulator is flibanserin.

In some embodiments, the psychedelic is MDMA, and the serotonin receptor modulator is flibanserin.

In some embodiments, the psychedelic is DOM, and the serotonin receptor modulator is flibanserin.

In some embodiments, the psychedelic is selected from the group consisting of psilocybin, 4-Acetoxy-DMT, LSD, ALD-52, 1P-LSD, DMT, and 5-MeO-DMT, 2C-B, ibogaine, MDMA, and DOM, and the serotonin receptor modulator is flibanserin.

In some embodiments, the psychedelic is selected from the group consisting of psilocybin, 4-Acetoxy-DMT, LSD, ALD-52, 1P-LSD, DMT, and 5-MeO-DMT, 2C-B, ibogaine, MDMA, and DOM, and the serotonin receptor modulator is selected from the group consisting of eplivanserin, volinanserin, ketanserin, ritanserin, pimavanserin, nelotanserin, pruvanserin, and flibanserin.

In some embodiments, the psychedelic is selected from the group consisting of psilocybin, LSD, DMT, and 5-MeO-DMT5-MeO-DMT and the serotonin receptor modulator is selected from the group consisting of eplivanserin, volinanserin, ritanserin, pimavanserin, nelotanserin, and pruvanserin.

In some embodiments, the psychedelic is selected from the group consisting of psilocybin, LSD, DMT, and 5-MeO-DMT5-MeO-DMT and the serotonin receptor modulator is selected from the group consisting of eplivanserin, volinanserin, pimavanserin, nelotanserin, and pruvanserin.

In some embodiments, the psychedelic is selected from the group consisting of psilocybin, LSD, DMT, and 5-MeO-DMT5-MeO-DMT and the serotonin receptor modulator is eplivanserin or volinanserin.

In some embodiments, the psychedelic is selected from the group consisting of psilocybin, LSD, DMT, and 5-MeO-DMT5-MeO-DMT and the serotonin receptor modulator is eplivanserin.

In some embodiments, the psychedelic is selected from the group consisting of psilocybin, LSD, DMT, and 5-MeO-DMT5-MeO-DMT and the serotonin receptor modulator is volinanserin.

In some embodiments, the psychedelic is psilocybin and the serotonin receptor modulator is eplivanserin or volinanserin. In some embodiments, the psychedelic is LSD, and the serotonin receptor modulator is eplivanserin or volinanserin. In some embodiments, the psychedelic is DMT, and the serotonin receptor modulator is eplivanserin or volinanserin. In some embodiments, the psychedelic is 5-MeO-DMT5-MeO-DMT and the serotonin receptor modulator is eplivanserin or volinanserin.

In some embodiments, the psychedelic is selected from the group consisting of psilocybin, LSD, DMT, and 5-MeO-DMT5-MeO-DMT and the serotonin receptor modulator is selected from the group consisting of pimavanserin, nelotanserin, and pruvanserin.

In some embodiments, the psychedelic is psilocybin, and the serotonin receptor modulator is selected from the group consisting of pimavanserin, nelotanserin, and pruvanserin.

In some embodiments, the psychedelic is LSD, and the serotonin receptor modulator is selected from the group consisting of pimavanserin, nelotanserin, and pruvanserin.

In some embodiments, the psychedelic is DMT, and the serotonin receptor modulator is selected from the group consisting of pimavanserin, nelotanserin, and pruvanserin.

In some embodiments, the psychedelic is 5-MeO-DMT5-MeO-DMT and the serotonin receptor modulator is selected from the group consisting of pimavanserin, nelotanserin, and pruvanserin.

In some embodiments, the psychedelic is psilocybin, and the serotonin receptor modulator is selected from the group consisting of pimavanserin, and nelotanserin.

In some embodiments, the psychedelic is LSD, and the serotonin receptor modulator is selected from the group consisting of pimavanserin, and nelotanserin.

In some embodiments, the psychedelic is DMT, and the serotonin receptor modulator is selected from the group consisting of pimavanserin, and nelotanserin.

In some embodiments, the psychedelic is 5-MeO-DMT5-MeO-DMT and the serotonin receptor modulator is selected from the group consisting of pimavanserin, and nelotanserin.

In some embodiments, the serotonin receptor modulator is eplivanserin and the psychedelic is psilocybin. In some embodiments, the serotonin receptor modulator is volinanserin and the psychedelic is psilocybin. In some embodiments, the serotonin receptor modulator is ketanserin and the psychedelic is psilocybin. In some embodiments, the serotonin receptor modulator is ritanserin and the psychedelic is psilocybin. In some embodiments, the serotonin receptor modulator is pimavanserin and the psychedelic is psilocybin. In some embodiments, the serotonin receptor modulator is nelotanserin and the psychedelic is psilocybin. In some embodiments, the serotonin receptor modulator is pruvanserin and the psychedelic is psilocybin.

In some embodiments, the serotonin receptor modulator is eplivanserin and the psychedelic is LSD. In some embodiments, the serotonin receptor modulator is volinanserin and the psychedelic is LSD. In some embodiments, the serotonin receptor modulator is ketanserin and the psychedelic is LSD. In some embodiments, the serotonin receptor modulator is ritanserin and the psychedelic is LSD. In some embodiments, the serotonin receptor modulator is pimavanserin and the psychedelic is LSD. In some embodiments, the serotonin receptor modulator is nelotanserin and the psychedelic is LSD. In some embodiments, the serotonin receptor modulator is pruvanserin and the psychedelic is LSD.

In some embodiments, the serotonin receptor modulator is eplivanserin and the psychedelic is DMT. In some embodiments, the serotonin receptor modulator is volinanserin and the psychedelic is DMT. In some embodiments, the serotonin receptor modulator is ketanserin and the psychedelic is DMT. In some embodiments, the serotonin receptor modulator is ritanserin and the psychedelic is DMT. In some embodiments, the serotonin receptor modulator is pimavanserin and the psychedelic is DMT. In some embodiments, the serotonin receptor modulator is nelotanserin and the psychedelic is DMT. In some embodiments, the serotonin receptor modulator is pruvanserin and the psychedelic is DMT.

In some embodiments, the serotonin receptor modulator is eplivanserin and the psychedelic is 5-MeO-DMT. In some embodiments, the serotonin receptor modulator is volinanserin and the psychedelic is 5-MeO-DMT. In some embodiments, the serotonin receptor modulator is ketanserin and the psychedelic is 5-MeO-DMT. In some embodiments, the serotonin receptor modulator is ritanserin and the psychedelic is 5-MeO-DMT. In some embodiments, the serotonin receptor modulator is pimavanserin and the psychedelic is 5-MeO-DMT. In some embodiments, the serotonin receptor modulator is nelotanserin and the psychedelic is 5-MeO-DMT. In some embodiments, the serotonin receptor modulator is pruvanserin and the psychedelic is 5-MeO-DMT.

In some embodiments, each of the combinations of psychedelics and serotonin receptor modulators described herein are contemplated at each of the following dosages and dose ranges described herein. In some embodiments, each of the combinations of psychedelics and serotonin receptor modulators described herein are contemplated at each of the following administration time periods described herein. In some embodiments, each of the combinations of psychedelics and serotonin receptor modulators described herein are contemplated at each of the following dosages and dose ranges and each of the following administration time periods described herein.

Described herein, in some embodiments, are compositions comprising a psychedelic and a serotonin receptor modulator, wherein the psychedelic (e.g., psilocybin, psilocin, O-acetyl psilocin, LSD, 1-acetyl LSD) is provided at varying doses. In some embodiments, the psychedelic is provided in a range about 10 milligrams (mg) to 400 mg, about 10 mg to about 300 mg, about 10 mg to about 200 mg, about 10 mg to about 100 mg, about 10 mg to about 80 mg, about 10 mg to about 70 mg, about 10 mg to about 60 mg, about 10 mg to about 50 mg, about 10 mg to about 40 mg, about 20 mg to 400 mg, about 20 mg to about 300 mg, about 20 mg to about 200 mg, about 20 mg to about 100 mg, about 20 mg to about 80 mg, about 20 mg to about 70 mg, about 20 mg to about 60 mg, about 20 mg to about 50 mg, about 20 mg to about 40 mg, about 50 mg to 400 mg, about 50 mg to about 300 mg, about 50 mg to about 200 mg, about 50 mg to about 100 mg, about 50 mg to about 80 mg, about 50 mg to about 70 mg, or about 50 mg to about 60 mg. In some embodiments, the psychedelic is provided at about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 240 mg, about 250 mg, about 260 mg, about 270 mg, about 275 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, 410 mg, about 420 mg, about 430 mg, about 440 mg, about 450 mg, about 460 mg, about 470 mg, about 480 mg, about 490 mg, or about 500 mg. In some embodiments, the psychedelic is a phenethylamine or a tryptamine, or a pharmaceutically acceptable salt, solvate, metabolite, deuterated analogue, derivative, or prodrug thereof.

In some embodiments, the psychedelic is provided in a range about 10 micrograms (μg) to 400 μg, about 10 μg to about 300 μg, about 10 μg to about 200 μg, about 10 μg to about 100 μg, about 10 μg to about 80 μg, about 10 μg to about 70 μg, about 10 μg to about 60 μg, about 10 μg to about 50 μg, about 10 μg to about 40 μg, about 20 μg to 400 μg, about 20 μg to about 300 μg, about 20 μg to about 200 μg, about 20 μg to about 100 μg, about 20 μg to about 80 μg, about 20 μg to about 70 μg, about 20 μg to about 60 μg, about 20 μg to about 50 μg, about 20 μg to about 40 μg, about 50 μg to 400 μg, about 50 μg to about 300 μg, about 50 μg to about 200 μg, about 50 μg to about 100 μg, about 50 μg to about 80 μg, about 50 μg to about 70 μg, or about 50 μg to about 60 μg. In some embodiments, the psychedelic is provided at about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 105 μg, about 110 μg, about 115 μg, about 120 μg, about 125 μg, about 130 μg, about 135 μg, about 140 μg, about 145 μg, about 150 μg, about 155 μg, about 160 μg, about 165 μg, about 170 μg, about 175 μg, about 180 μg, about 185 μg, about 190 μg, about 195 μg, about 200 μg, about 205 μg, about 210 μg, about 215 μg, about 220 μg, about 225 μg, about 230 μg, about 240 μg, about 250 μg, about 260 μg, about 270 μg, about 275 μg, about 280 μg, about 290 μg, about 300 μg, about 310 μg, about 320 μg, about 330 μg, about 340 μg, about 350 μg, about 360 μg, about 370 μg, about 380 μg, about 390 μg, about 400 μg, 410 μg, about 420 μg, about 430 μg, about 440 μg, about 450 μg, about 460 μg, about 470 μg, about 480 μg, about 490 μg, or about 500 μg. In some embodiments, the psychedelic is LSD or a pharmaceutically acceptable salt, solvate, metabolite, deuterated analogue, derivative, or prodrug thereof. In some embodiments, the LSD derivative is 1P-LSD, 1B-LSD, ETH-LAD, IP-ETH-LAD, AL-LAD, LSZ, LSM-775, 1-(4-Bromofuro[2,3-f] [1]benzofuran-8-yl)propan-2-amine.

In some embodiments, the psychedelic is provided in a range about 100 milligrams (mg) to 4 grams (g), about 100 mg to about 3 g, about 100 mg to about 2 g, about 100 mg to about 1 g, about 100 mg to about 800 mg, about 100 mg to about 700 mg, about 100 mg to about 600 mg, about 100 mg to about 500 mg, about 100 mg to about 400 mg, about 200 mg to 4 g, about 200 mg to about 3 g, about 200 mg to about 2 g, about 200 mg to about 1 g, about 200 mg to about 800 mg, about 200 mg to about 700 mg, about 200 mg to about 600 mg, about 200 mg to about 500 mg, about 200 mg to about 400 mg, about 500 mg to 4 g, about 500 mg to about 3 g, about 500 mg to about 2 g, about 500 mg to about 1 g, about 500 mg to about 800 mg, about 500 mg to about 700 mg, or about 500 mg to about 600 mg. In some embodiments, the psychedelic is provided at about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1 g, about 2 g, about 3 g, about 4 g, or about 5 g. In some embodiments, the psychedelic is mescaline or a pharmaceutically acceptable salt, solvate, metabolite, deuterated analogue, derivative, or prodrug thereof. In some embodiments, the mescaline derivative is mescaline-NBOMe, proscaline (2-(3,5-dimethoxy-4-propoxyphenyl)ethanamine), or metaescaline (2-(3-ethoxy-4,5-dimethoxyphenyl)ethanamine).

Described herein, in some embodiments, are compositions comprising a psychedelic and a serotonin receptor modulator, wherein the serotonin receptor modulator (e.g., ketanserin, pimavanserin) is provided at varying doses. In some embodiments, the serotonin receptor modulator is provided in a range about 10 mg to 400 mg, about 10 mg to about 300 mg, about 10 mg to about 200 mg, about 10 mg to about 100 mg, about 10 mg to about 80 mg, about 10 mg to about 70 mg, about 10 mg to about 60 mg, about 10 mg to about 50 mg, about 10 mg to about 40 mg, about 20 mg to 400 mg, about 20 mg to about 300 mg, about 20 mg to about 200 mg, about 20 mg to about 100 mg, about 20 mg to about 80 mg, about 20 mg to about 70 mg, about 20 mg to about 60 mg, about 20 mg to about 50 mg, about 20 mg to about 40 mg, about 50 mg to 400 mg, about 50 mg to about 300 mg, about 50 mg to about 200 mg, about 50 mg to about 100 mg, about 50 mg to about 80 mg, about 50 mg to about 70 mg, or about 50 mg to about 60 mg. In some embodiments, the serotonin receptor is provided at about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 240 mg, about 250 mg, about 260 mg, about 270 mg, about 275 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, 410 mg, about 420 mg, about 430 mg, about 440 mg, about 450 mg, about 460 mg, about 470 mg, about 480 mg, about 490 mg, or about 500 mg.

In some embodiments, the serotonin receptor modulator is selected from the group consisting of ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, AMDA, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, and RS-102221, or a pharmaceutically acceptable salt, solvate, metabolite, deuterated analogue, derivative, prodrug, or combinations thereof.

In some embodiments, the serotonin receptor modulator is selected from the group consisting of MDL-11,939, eplivanserin (SR-46,349), ketanserin, ritanserin, altanserin, acepromazine, mianserin, mirtazapine, quetiapine, SB204741, SB206553, SB242084, LY272015, SB243213, Blonanserin, SB200646, RS102221, nefazodone, MDL-100,907, pimavanserin, nelotanserin, lorcaserin, flibanserin, roluperiodone or a pharmaceutically acceptable salt, solvate, metabolite, deuterated analogue, derivative, prodrug, or combinations thereof. In some embodiments, the serotonin receptor modulator is ketanserin or a pharmaceutically acceptable salt, solvate, metabolite, deuterated analogue, derivative, or prodrug thereof.

In some embodiments, the serotonin receptor modulator is ketanserin or a pharmaceutically acceptable salt thereof. In some embodiments, the serotonin receptor modulator is ketanserin.

In some embodiments, the serotonin receptor modulator is pimavanserin or a pharmaceutically acceptable salt thereof. In some embodiments, the serotonin receptor modulator is pimavanserin.

In some embodiments, the serotonin receptor modulator is eplivanserin or a pharmaceutically acceptable salt, solvate, metabolite, deuterated analogue, derivative, or prodrug thereof.

In some embodiments, the serotonin receptor modulator is eplivanserin or a pharmaceutically acceptable salt thereof. In some embodiments, the serotonin receptor modulator is eplivanserin.

In some embodiments, the serotonin receptor modulator is eplivanserin and the psychedelic is psilocybin, wherein the eplivanserin is provided between about 1 mg to about 40 mg, or about 5 mg to about 10 mg, and the psilocybin is provided between about 10 mg to about 50 mg, or about 25 mg to about 30 mg.

In some embodiments, the serotonin receptor modulator is volinanserin and the psychedelic is psilocybin, wherein the volinanserin is provided in about 1 mg to about 60 mg, or about 5 mg to about 20 mg, and the psilocybin is provided between about 10 mg to about 50 mg, or about 25 mg to about 30 mg.

In some embodiments, the serotonin receptor modulator is ketanserin and the psychedelic is psilocybin, wherein the ketanserin is provided in about 10 mg to about 80 mg, about 30 mg to about 50 mg, or about 40 mg and the psilocybin is provided between about 10 mg to about 50 mg, or about 25 mg to about 30 mg.

In some embodiments, the serotonin receptor modulator is ritanserin and the psychedelic is psilocybin, wherein the ritanserin is provided in about 1 mg to about 40 mg, or about 2.5 mg to about 10 mg, and the psilocybin is provided between about 10 mg to about 50 mg, or about 25 mg to about 30 mg.

In some embodiments, the serotonin receptor modulator is pimavanserin and the psychedelic is psilocybin, wherein the pimavanserin is provided in about 1 mg to about 60 mg, or about 17 mg to about 34 mg, and the psilocybin is provided between about 10 mg to about 50 mg, or about 25 mg to about 30 mg.

In some embodiments, the serotonin receptor modulator is nelotanserin and the psychedelic is psilocybin, wherein the nelotanserin is provided in about 1 mg to about 80 mg, or about 40 mg to about 80 mg, and the psilocybin is provided between about 10 mg to about 50 mg, or about 25 mg to about 30 mg.

In some embodiments, the serotonin receptor modulator is pruvanserin and the psychedelic is psilocybin, wherein the pruvanserin is provided in about 1 mg to about 40 mg, or about 3 mg to about 10 mg, and the psilocybin is present between about 10 mg to about 50 mg, or about 25 mg to about 30 mg.

In some embodiments, the serotonin receptor modulator is flibanserin and the psychedelic is psilocybin, wherein the flibanserin is present in about 10 mg to about 200 mg, or about 80 mg to about 120 mg, or about 100 mg and the psilocybin is present between about 10 mg to about 50 mg, or about 25 mg to about 30 mg.

In some embodiments, the serotonin receptor modulator is eplivanserin and the psychedelic is o-acetyl-psilocin (4-Acetoxy-DMT), wherein the eplivanserin is present in about 1 mg to about 40 mg, or about 5 mg to about 10 mg, and the 4-Acetoxy-DMT is present between about 5 mg to about 50 mg, or about 10 mg to about 25 mg.

In some embodiments, the serotonin receptor modulator is volinanserin and the psychedelic is 4-Acetoxy-DMT, wherein the volinanserin is present in about 1 mg to about 60 mg, or about 5 mg to about 20 mg, and the 4-Acetoxy-DMT is present between about 5 mg to about 50 mg, or about 10 mg to about 25 mg.

In some embodiments, the serotonin receptor modulator is ketanserin and the psychedelic is 4-Acetoxy-DMT, wherein the ketanserin is present in about 10 mg to about 80 mg, about 30 mg to about 50 mg, or about 40 mg and the 4-Acetoxy-DMT is present between about 5 mg to about 50 mg, or about 10 mg to about 25 mg.

In some embodiments, the serotonin receptor modulator is ritanserin and the psychedelic is 4-Acetoxy-DMT, wherein the ritanserin is present in about 1 mg to about 40 mg, or about 2.5 mg to about 10 mg, and the 4-Acetoxy-DMT is present between about 5 mg to about 50 mg, or about 10 mg to about 25 mg.

In some embodiments, the serotonin receptor modulator is pimavanserin and the psychedelic is 4-Acetoxy-DMT, wherein the pimavanserin is present in about 1 mg to about 60 mg, or about 17 mg to about 34 mg, and the 4-Acetoxy-DMT is present between about 5 mg to about 50 mg, or about 10 mg to about 25 mg.

In some embodiments, the serotonin receptor modulator is nelotanserin and the psychedelic is 4-Acetoxy-DMT, wherein the nelotanserin is present in about 1 mg to about 80 mg, or about 40 mg to about 80 mg, and the 4-Acetoxy-DMT is present between about 5 mg to about 50 mg, or about 10 mg to about 25 mg.

In some embodiments, the serotonin receptor modulator is pruvanserin and the psychedelic is 4-Acetoxy-DMT, wherein the pruvanserin is present in about 1 mg to about 40 mg, or about 3 mg to about 10 mg, and the 4-Acetoxy-DMT is present between about 5 mg to about 50 mg, or about 10 mg to about 25 mg.

In some embodiments, the serotonin receptor modulator is flibanserin and the psychedelic is 4-Acetoxy-DMT, wherein the flibanserin is present in about 10 mg to about 200 mg, or about 80 mg to about 120 mg, or about 100 mg and the 4-Acetoxy-DMT is present between about 5 mg to about 50 mg, or about 10 mg to about 25 mg

In some embodiments, the serotonin receptor modulator is eplivanserin and the psychedelic is LSD, wherein the eplivanserin is present in about 1 mg to about 40 mg, or about 5 mg to about 10 mg, and the LSD is present between about 1 microgram to about 400 micrograms, or about 50 micrograms to about 200 micrograms.

In some embodiments, the serotonin receptor modulator is volinanserin and the psychedelic is LSD, wherein the volinanserin is present in about 1 mg to about 60 mg, or about 5 mg to about 20 mg, and the LSD is present between about 1 microgram to about 400 micrograms, or about 50 micrograms to about 200 micrograms.

In some embodiments, the serotonin receptor modulator is ketanserin and the psychedelic is LSD, wherein the ketanserin is present in about 10 mg to about 80 mg, about 30 mg to about 50 mg, or about 40 mg and the LSD is present between about 1 microgram to about 400 micrograms, or about 50 micrograms to about 200 micrograms.

In some embodiments, the serotonin receptor modulator is ritanserin and the psychedelic is LSD, wherein the ritanserin is present in about 1 mg to about 40 mg, or about 2.5 mg to about 10 mg, and the LSD is present between about 1 microgram to about 400 micrograms, or about 50 micrograms to about 200 micrograms.

In some embodiments, the serotonin receptor modulator is pimavanserin and the psychedelic is LSD, wherein the pimavanserin is present in about 1 mg to about 60 mg, or about 17 mg to about 34 mg, and the LSD is present between about 1 microgram to about 400 micrograms, or about 50 micrograms to about 200 micrograms.

In some embodiments, the serotonin receptor modulator is nelotanserin and the psychedelic is LSD, wherein the nelotanserin is present in about 1 mg to about 80 mg, or about 40 mg to about 80 mg, and the LSD is present between about 1 microgram to about 400 micrograms, or about 50 micrograms to about 200 micrograms.

In some embodiments, the serotonin receptor modulator is pruvanserin and the psychedelic is LSD, wherein the pruvanserin is present in about 1 mg to about 40 mg, or about 3 mg to about 10 mg, and the LSD is present between about 1 microgram to about 400 micrograms, or about 50 micrograms to about 200 micrograms.

In some embodiments, the serotonin receptor modulator is flibanserin and the psychedelic is LSD, wherein the flibanserin is present in about 10 mg to about 200 mg, or about 80 mg to about 120 mg, or about 100 mg and the LSD is present between about 1 microgram to about 400 micrograms, or about 50 micrograms to about 200 micrograms.

In some embodiments, the serotonin receptor modulator is eplivanserin and the psychedelic is ALD-52 (i.e. 1-acetyl-LSD), wherein the eplivanserin is present in about 1 mg to about 40 mg, or about 5 mg to about 10 mg, and the ALD-52 is present between about 1 microgram to about 400 micrograms, or about 50 micrograms to about 200 micrograms.

In some embodiments, the serotonin receptor modulator is volinanserin and the psychedelic is ALD-52 (i.e. 1-acetyl-LSD), wherein the volinanserin is present in about 1 mg to about 60 mg, or about 5 mg to about 20 mg, and the ALD-52 is present between about 1 microgram to about 400 micrograms, or about 50 micrograms to about 200 micrograms.

In some embodiments, the serotonin receptor modulator is ketanserin and the psychedelic is ALD-52, wherein the ketanserin is present in about 10 mg to about 80 mg, about 30 mg to about 50 mg, or about 40 mg and the ALD-52 is present between about 1 microgram to about 400 micrograms, or about 50 micrograms to about 200 micrograms.

In some embodiments, the serotonin receptor modulator is ritanserin and the psychedelic is ALD-52, wherein the ritanserin is present in about 1 mg to about 40 mg, or about 2.5 mg to about 10 mg, and the ALD-52 is present between about 1 microgram to about 400 micrograms, or about 50 micrograms to about 200 micrograms.

In some embodiments, the serotonin receptor modulator is pimavanserin and the psychedelic is ALD-52, wherein the pimavanserin is present in about 1 mg to about 60 mg, or about 17 mg to about 34 mg, and the ALD-52 is present between about 1 microgram to about 400 micrograms, or about 50 micrograms to about 200 micrograms.

In some embodiments, the serotonin receptor modulator is nelotanserin and the psychedelic is ALD-52, wherein the nelotanserin is present in about 1 mg to about 80 mg, or about 40 mg to about 80 mg, and the ALD-52 is present between about 1 microgram to about 400 micrograms, or about 50 micrograms to about 200 micrograms.

In some embodiments, the serotonin receptor modulator is pruvanserin and the psychedelic is ALD-52, wherein the pruvanserin is present in about 1 mg to about 40 mg, or about 3 mg to about 10 mg, and the ALD-52 is present between about 1 microgram to about 400 micrograms, or about 50 micrograms to about 200 micrograms.

In some embodiments, the serotonin receptor modulator is flibanserin and the psychedelic is ALD-52, wherein the flibanserin is present in about 10 mg to about 200 mg, or about 80 mg to about 120 mg, or about 100 mg and the ALD-52 is present between about 1 microgram to about 400 micrograms, or about 50 micrograms to about 200 micrograms.

In some embodiments, the serotonin receptor modulator is eplivanserin and the psychedelic is 1P-LSD (i.e. 1-propionyl-LSD), wherein the eplivanserin is present in about 1 mg to about 40 mg, or about 5 mg to about 10 mg, and the IP-LSD is present between about 1 microgram to about 400 micrograms, or about 50 micrograms to about 200 micrograms.

In some embodiments, the serotonin receptor modulator is volinanserin and the psychedelic is 1P-LSD, wherein the volinanserin is present in about 1 mg to about 60 mg, or about 5 mg to about 20 mg, and the IP-LSD is present between about 1 microgram to about 400 micrograms, or about 50 micrograms to about 200 micrograms.

In some embodiments, the serotonin receptor modulator is ketanserin and the psychedelic is 1P-LSD, wherein the ketanserin is present in about 10 mg to about 80 mg, about 30 mg to about 50 mg, or about 40 mg and the IP-LSD is present between about 1 microgram to about 400 micrograms, or about 50 micrograms to about 200 micrograms.

In some embodiments, the serotonin receptor modulator is ritanserin and the psychedelic is IP-LSD, wherein the ritanserin is present in about 1 mg to about 40 mg, or about 2.5 mg to about 10 mg, and the IP-LSD is present between about 1 microgram to about 400 micrograms, or about 50 micrograms to about 200 micrograms.

In some embodiments, the serotonin receptor modulator is pimavanserin and the psychedelic is 1P-LSD, wherein the pimavanserin is present in about 1 mg to about 60 mg, or about 17 mg to about 34 mg, and the IP-LSD is present between about 1 microgram to about 400 micrograms, or about 50 micrograms to about 200 micrograms.

In some embodiments, the serotonin receptor modulator is nelotanserin and the psychedelic is 1P-LSD, wherein the nelotanserin is present in about 1 mg to about 80 mg, or about 40 mg to about 80 mg, and the IP-LSD is present between about 1 microgram to about 400 micrograms, or about 50 micrograms to about 200 micrograms.

In some embodiments, the serotonin receptor modulator is pruvanserin and the psychedelic is 1P-LSD, wherein the pruvanserin is present in about 1 mg to about 40 mg, or about 3 mg to about 10 mg, and the IP-LSD is present between about 1 microgram to about 400 micrograms, or about 50 micrograms to about 200 micrograms.

In some embodiments, the serotonin receptor modulator is flibanserin and the psychedelic is 1P-LSD, wherein the flibanserin is present in about 10 mg to about 200 mg, or about 80 mg to about 120 mg, or about 100 mg and the 1P-LSD is present between about 1 microgram to about 400 micrograms, or about 50 micrograms to about 200 micrograms.

In some embodiments, the serotonin receptor modulator is eplivanserin and the psychedelic is DMT, wherein the eplivanserin is present in about 1 mg to about 40 mg, or about 5 mg to about 10 mg, and the DMT is present between about 1 mg and 60 mg.

In some embodiments, the serotonin receptor modulator is volinanserin and the psychedelic is DMT, wherein the volinanserin is present in about 1 mg to about 60 mg, or about 5 mg to about 20 mg, and the DMT is present between about 1 mg and 60 mg.

In some embodiments, the serotonin receptor modulator is ketanserin and the psychedelic is DMT, wherein the ketanserin is present in about 10 mg to about 80 mg, about 30 mg to about 50 mg, or about 40 mg and the DMT is present between about 1 mg and 60 mg.

In some embodiments, the serotonin receptor modulator is ritanserin and the psychedelic is DMT, wherein the ritanserin is present in about 1 mg to about 40 mg, or about 2.5 mg to about 10 mg, and the DMT is present between about 1 mg and 60 mg.

In some embodiments, the serotonin receptor modulator is pimavanserin and the psychedelic is DMT, wherein the pimavanserin is present in about 1 mg to about 60 mg, or about 17 mg to about 34 mg, and the DMT is present between about 1 mg and 60 mg.

In some embodiments, the serotonin receptor modulator is nelotanserin and the psychedelic is DMT, wherein the nelotanserin is present in about 1 mg to about 80 mg, or about 40 mg to about 80 mg, and the DMT is present between about 1 mg and 60 mg.

In some embodiments, the serotonin receptor modulator is pruvanserin and the psychedelic is DMT, wherein the pruvanserin is present in about 1 mg to about 40 mg, or about 3 mg to about 10 mg, and the DMT is present between about 1 mg and 60 mg.

In some embodiments, the serotonin receptor modulator is flibanserin and the psychedelic is DMT, wherein the flibanserin is present in about 10 mg to about 200 mg, or about 80 mg to about 120 mg, or about 100 mg and the DMT is present between about 1 mg and 60 mg. In some embodiments, the serotonin receptor modulator is eplivanserin and the psychedelic is 5-MeO-DMT, wherein the eplivanserin is present in about 1 mg to about 40 mg, or about 5 mg to about 10 mg, and the 5-MeO-DMT is present between about 1 mg and 30 mg.

In some embodiments, the serotonin receptor modulator is volinanserin and the psychedelic is 5-MeO-DMT, wherein the volinanserin is present in about 1 mg to about 60 mg, or about 5 mg to about 20 mg, and the 5-MeO-DMT is present between about 1 mg and 30 mg.

In some embodiments, the serotonin receptor modulator is ketanserin and the psychedelic is 5-MeO-DMT, wherein the ketanserin is present in about 10 mg to about 80 mg, about 30 mg to about 50 mg, or about 40 mg and the 5-MeO-DMT is present between about 1 mg and 30 mg.

In some embodiments, the serotonin receptor modulator is ritanserin and the psychedelic is 5-MeO-DMT, wherein the ritanserin is present in about 1 mg to about 40 mg, or about 2.5 mg to about 10 mg, and the 5-MeO-DMT is present between about 1 mg and 30 mg.

In some embodiments, the serotonin receptor modulator is pimavanserin and the psychedelic is 5-MeO-DMT, wherein the pimavanserin is present in about 1 mg to about 60 mg, or about 17 mg to about 34 mg, and the 5-MeO-DMT is present between about 1 mg and 30 mg.

In some embodiments, the serotonin receptor modulator is nelotanserin and the psychedelic is 5-MeO-DMT, wherein the nelotanserin is present in about 1 mg to about 80 mg, or about 40 mg to about 80 mg, and the 5-MeO-DMT is present between about 1 mg and 30 mg.

In some embodiments, the serotonin receptor modulator is pruvanserin and the psychedelic is 5-MeO-DMT, wherein the pruvanserin is present in about 1 mg to about 40 mg, or about 3 mg to about 10 mg, and the 5-MeO-DMT is present between about 1 mg and 30 mg.

In some embodiments, the serotonin receptor modulator is flibanserin and the psychedelic is 5-MeO-DMT, wherein the flibanserin is present in about 10 mg to about 200 mg, or about 80 mg to about 120 mg, or about 100 mg and the 5-MeO-DMT is present between about 1 mg and 30 mg.

In some embodiments, the serotonin receptor modulator is eplivanserin and the psychedelic is 2C-B, wherein the eplivanserin is present in about 1 mg to about 40 mg, or about 5 mg to about 10 mg, and the 2C-B is present between about 1 mg and 40 mg.

In some embodiments, the serotonin receptor modulator is volinanserin and the psychedelic is 2C-B, wherein the volinanserin is present in about 1 mg to about 60 mg, or about 5 mg to about 20 mg, and the 2C-B is present between about 1 mg and 40 mg.

In some embodiments, the serotonin receptor modulator is ketanserin and the psychedelic is 2C-B, wherein the ketanserin is present in about 10 mg to about 80 mg, about 30 mg to about 50 mg, or about 40 mg and the 2C-B is present between about 1 mg and 40 mg.

In some embodiments, the serotonin receptor modulator is ritanserin and the psychedelic is 2C-B, wherein the ritanserin is present in about 1 mg to about 40 mg, or about 2.5 mg to about 10 mg, and the 2C-B is present between about 1 mg and 40 mg.

In some embodiments, the serotonin receptor modulator is pimavanserin and the psychedelic is 2C-B, wherein the pimavanserin is present in about 1 mg to about 60 mg, or about 17 mg to about 34 mg, and the 2C-B is present between about 1 mg and 40 mg.

In some embodiments, the serotonin receptor modulator is nelotanserin and the psychedelic is 2C-B, wherein the nelotanserin is present in about 1 mg to about 80 mg, or about 40 mg to about 80 mg, and the 2C-B is present between about 1 mg and 40 mg.

In some embodiments, the serotonin receptor modulator is pruvanserin and the psychedelic is 2C-B, wherein the pruvanserin is present in about 1 mg to about 40 mg, or about 3 mg to about 10 mg, and the 2C-B is present between about 1 mg and 40 mg.

In some embodiments, the serotonin receptor modulator is flibanserin and the psychedelic is 2C-B, wherein the flibanserin is present in about 10 mg to about 200 mg, or about 80 mg to about 120 mg, or about 100 mg and the 2C-B is present between about 1 mg and 40 mg.

In some embodiments, the serotonin receptor modulator is eplivanserin and the psychedelic is mescaline, wherein the eplivanserin is present in about 1 mg to about 40 mg, or about 5 mg to about 10 mg, and the mescaline is present between about 50 mg and about 800 mg, or between about 200 mg and 500 mg.

In some embodiments, the serotonin receptor modulator is volinanserin and the psychedelic is mescaline, wherein the volinanserin is present in about 1 mg to about 60 mg, or about 5 mg to about 20 mg, and the mescaline is present between about 50 mg and about 800 mg, or between about 200 mg and 500 mg.

In some embodiments, the serotonin receptor modulator is ketanserin and the psychedelic is mescaline, wherein the ketanserin is present in about 10 mg to about 80 mg, about 30 mg to about 50 mg, or about 40 mg and the mescaline is present between about 50 mg and about 800 mg, or between about 200 mg and 500 mg.

In some embodiments, the serotonin receptor modulator is ritanserin and the psychedelic is mescaline, wherein the ritanserin is present in about 1 mg to about 40 mg, or about 2.5 mg to about 10 mg, and the mescaline is present between about 50 mg and about 800 mg, or between about 200 mg and 500 mg.

In some embodiments, the serotonin receptor modulator is pimavanserin and the psychedelic is mescaline, wherein the pimavanserin is present in about 1 mg to about 60 mg, or about 17 mg to about 34 mg, and the mescaline is present between about 50 mg and about 800 mg, or between about 200 mg and 500 mg.

In some embodiments, the serotonin receptor modulator is nelotanserin and the psychedelic is mescaline, wherein the nelotanserin is present in about 1 mg to about 80 mg, or about 40 mg to about 80 mg, and the mescaline is present between about 50 mg and about 800 mg, or between about 200 mg and 500 mg.

In some embodiments, the serotonin receptor modulator is pruvanserin and the psychedelic is mescaline, wherein the pruvanserin is present in about 1 mg to about 40 mg, or about 3 mg to about 10 mg, and the mescaline is present between about 50 mg and about 800 mg, or between about 200 mg and 500 mg.

In some embodiments, the serotonin receptor modulator is flibanserin and the psychedelic is mescaline, wherein the flibanserin is present in about 10 mg to about 200 mg, or about 80 mg to about 120 mg, or about 100 mg and the mescaline is present between about 50 mg and about 800 mg, or between about 200 mg and 500 mg.

In some embodiments, the serotonin receptor modulator is eplivanserin and the psychedelic is 12-methoxyibogamine (ibogaine), wherein the eplivanserin is present in about 1 mg to about 40 mg, or about 5 mg to about 10 mg, and the ibogaine is present between about 50 mg and about 1,000 mg, or between about 500 mg and 800 mg.

In some embodiments, the serotonin receptor modulator is volinanserin and the psychedelic is ibogaine, wherein the volinanserin is present in about 1 mg to about 60 mg, or about 5 mg to about 20 mg, and the ibogaine is present between about 50 mg and about 1,000 mg, or between about 500 mg and 800 mg.

In some embodiments, the serotonin receptor modulator is ketanserin and the psychedelic is ibogaine, wherein the ketanserin is present in about 10 mg to about 80 mg, about 30 mg to about 50 mg, or about 40 mg and the ibogaine is present between about 50 mg and about 1,000 mg, or between about 500 mg and 800 mg.

In some embodiments, the serotonin receptor modulator is ritanserin and the psychedelic is ibogaine, wherein the ritanserin is present in about 1 mg to about 40 mg, or about 2.5 mg to about 10 mg, and the ibogaine is present between about 50 mg and about 1,000 mg, or between about 500 mg and 800 mg.

In some embodiments, the serotonin receptor modulator is pimavanserin and the psychedelic is ibogaine, wherein the pimavanserin is present in about 1 mg to about 60 mg, or about 17 mg to about 34 mg, and the ibogaine is present between about 50 mg and about 1,000 mg, or between about 500 mg and 800 mg.

In some embodiments, the serotonin receptor modulator is nelotanserin and the psychedelic is ibogaine, wherein the nelotanserin is present in about 1 mg to about 80 mg, or about 40 mg to about 80 mg, and the ibogaine is present between about 50 mg and about 1,000 mg, or between about 500 mg and 800 mg.

In some embodiments, the serotonin receptor modulator is pruvanserin and the psychedelic is ibogaine, wherein the pruvanserin is present in about 1 mg to about 40 mg, or about 3 mg to about 10 mg, and the ibogaine is present between about 50 mg and about 1,000 mg, or between about 500 mg and 800 mg.

In some embodiments, the serotonin receptor modulator is flibanserin and the psychedelic is ibogaine, wherein the flibanserin is present in about 10 mg to about 200 mg, or about 80 mg to about 120 mg, or about 100 mg and the ibogaine is present between about 50 mg and about 1,000 mg, or between about 500 mg and 800 mg.

In some embodiments, the serotonin receptor modulator is eplivanserin and the psychedelic is MDMA, wherein the eplivanserin is present in about 1 mg to about 40 mg, or about 5 mg to about 10 mg, and the MDMA is present between about 50 mg and about 200 mg, or between about 80 mg and 120 mg.

In some embodiments, the serotonin receptor modulator is volinanserin and the psychedelic is MDMA, wherein the volinanserin is present in about 1 mg to about 60 mg, or about 5 mg to about 20 mg, and the MDMA is present between about 50 mg and about 200 mg, or between about 80 mg and 120 mg.

In some embodiments, the serotonin receptor modulator is ketanserin and the psychedelic is MDMA, wherein the ketanserin is present in about 10 mg to about 80 mg, about 30 mg to about 50 mg, or about 40 mg and the MDMA is present between about 50 mg and about 200 mg, or between about 80 mg and 120 mg.

In some embodiments, the serotonin receptor modulator is ritanserin and the psychedelic is MDMA, wherein the ritanserin is present in about 1 mg to about 40 mg, or about 2.5 mg to about 10 mg, and the MDMA is present between about 50 mg and about 200 mg, or between about 80 mg and 120 mg.

In some embodiments, the serotonin receptor modulator is pimavanserin and the psychedelic is MDMA, wherein the pimavanserin is present in about 1 mg to about 60 mg, or about 17 mg to about 34 mg, and the MDMA is present between about 50 mg and about 200 mg, or between about 80 mg and 120 mg.

In some embodiments, the serotonin receptor modulator is nelotanserin and the psychedelic is MDMA, wherein the nelotanserin is present in about 1 mg to about 80 mg, or about 40 mg to about 80 mg, and the MDMA is present between about 50 mg and about 200 mg, or between about 80 mg and 120 mg.

In some embodiments, the serotonin receptor modulator is pruvanserin and the psychedelic is MDMA, wherein the pruvanserin is present in about 1 mg to about 40 mg, or about 3 mg to about 10 mg, and the MDMA is present between about 50 mg and about 200 mg, or between about 80 mg and 120 mg.

In some embodiments, the serotonin receptor modulator is flibanserin and the psychedelic is MDMA, wherein the flibanserin is present in about 10 mg to about 200 mg, or about 80 mg to about 120 mg, or about 100 mg and the MDMA is present between about 50 mg and about 200 mg, or between about 80 mg and 120 mg.

In some embodiments, the serotonin receptor modulator is eplivanserin and the psychedelic is DOM, wherein the eplivanserin is present in about 1 mg to about 40 mg, or about 5 mg to about 10 mg, and the DOM is present between about 0.5 mg and about 15 mg, or about 5 mg.

In some embodiments, the serotonin receptor modulator is volinanserin and the psychedelic is DOM, wherein the volinanserin is present in about 1 mg to about 60 mg, or about 5 mg to about 20 mg, and the DOM is present between about 0.5 mg and about 15 mg, or about 5 mg.

In some embodiments, the serotonin receptor modulator is ketanserin and the psychedelic is DOM, wherein the ketanserin is present in about 10 mg to about 80 mg, about 30 mg to about 50 mg, or about 40 mg and the DOM is present between about 0.5 mg and about 15 mg, or about 5 mg.

In some embodiments, the serotonin receptor modulator is ritanserin and the psychedelic is DOM, wherein the ritanserin is present in about 1 mg to about 40 mg, or about 2.5 mg to about 10 mg, and the DOM is present between about 0.5 mg and about 15 mg, or about 5 mg.

In some embodiments, the serotonin receptor modulator is pimavanserin and the psychedelic is DOM, wherein the pimavanserin is present in about 1 mg to about 60 mg, or about 17 mg to about 34 mg, and the DOM is present between about 0.5 mg and about 15 mg, or about 5 mg.

In some embodiments, the serotonin receptor modulator is nelotanserin and the psychedelic is DOM, wherein the nelotanserin is present in about 1 mg to about 80 mg, or about 40 mg to about 80 mg, and the DOM is present between about 0.5 mg and about 15 mg, or about 5 mg.

In some embodiments, the serotonin receptor modulator is pruvanserin and the psychedelic is DOM, wherein the pruvanserin is present in about 1 mg to about 40 mg, or about 3 mg to about 10 mg, and the DOM is present between about 0.5 mg and about 15 mg, or about 5 mg.

In some embodiments, the serotonin receptor modulator is flibanserin and the psychedelic is DOM, wherein the flibanserin is present in about 10 mg to about 200 mg, or about 80 mg to about 120 mg, or about 100 mg and the DOM is present between about 0.5 mg and about 15 mg, or about 5 mg.

Described herein, in some embodiments, are compositions comprising a psychedelic and a serotonin receptor modulator, wherein the psychedelic and the serotonin receptor modulator are provided in a single form or single unit dosage form. In some embodiments, the single form is in the form of a pill, ampoule, vial, or tablet.

### Methods of Use

In another aspect, provided herein are methods of treating a disease or disorder in a subject in need thereof, the method comprising administering to the subject a composition comprising: a psychedelic; a serotonin receptor modulator; and an excipient, wherein the serotonin receptor modulator is released at most about 3 hours prior to the release of the psychedelic. In some embodiments, the disease or disorder is depression or a disease or disorder related to depression. In some embodiments, the depression is major depressive disorder, persistent depressive disorder, bipolar disorder, treatment resistant depression (TRD), postpartum depression, premenstrual dysphoric disorder, or seasonal affective disorder. In some embodiments, the disease or disorder related to depression is anxiety. In some embodiments, methods of treating depression or a disease or disorder related to depression comprise treating the symptoms associated with the depression or the disease or disorder related to depression.

Described herein are methods of treating depression or a disease or disorder related to depression in a subject in need thereof, the method comprising administering to the subject a composition comprising: a psychedelic; a serotonin receptor modulator; and an excipient, wherein the serotonin receptor modulator is released at most about 3 hours prior to the release of the psychedelic. In some embodiments, the depression is major depressive disorder, persistent depressive disorder, bipolar disorder, treatment resistant depression (TRD), postpartum depression, premenstrual dysphoric disorder, or seasonal affective disorder. In some embodiments, the disease or disorder related to depression is anxiety. In some embodiments, methods of treating depression or a disease or disorder related to depression comprise treating the symptoms associated with the depression or the disease or disorder related to depression.

Methods as described herein, in some embodiments, comprise administering composition comprising a psychedelic and a serotonin receptor modulator, wherein the serotonin receptor modulator attenuates the activation of the serotonin receptor. In some embodiments, the serotonin receptor modulator attenuates the activation of the serotonin receptor that results in psychedelic-induced perceptual alterations but does not attenuate the activation of the serotonin receptor resulting in neural changes. In some embodiments, the serotonin receptor modulator attenuates the activation of the serotonin receptor by the psychedelic. In some embodiments, the serotonin receptor modulator attenuates the activation of the serotonin receptor by 5-95%, 10-90%, 20-80%, 30-70%, 40-60%, 50-95%, 65-85%, or 75-95%. In some embodiments, the serotonin receptor modulator attenuates the activation of the serotonin receptor by at least about or about 5%, 10%, 15%, 20%, 25%, 30%, 35%, 40%, 45%, 50%, 55%, 60%, 65%, 70%, 75%, 80%, 85%, 90%, 95%, or greater than 95%. In some embodiments, the serotonin receptor modulator attenuates the activation of the serotonin receptor by at least about or about 5%. In some embodiments, the serotonin receptor modulator attenuates the activation of the serotonin receptor by at least about or about 10%. In some embodiments, the serotonin receptor modulator attenuates the activation of the serotonin receptor by at least about or about 50%. In some embodiments, the serotonin receptor modulator substantially blocks or blocks the activation of the serotonin receptor. In some embodiments, the serotonin receptor modulator substantially blocks or blocks the activation of the serotonin receptor by the psychedelic.

In some embodiments, methods as described herein comprise administering composition comprising a psychedelic and a serotonin receptor modulator, wherein the serotonin receptor modulator attenuates the activation of the serotonin receptor prior to administration of the psychedelic. In some embodiments, the serotonin receptor modulator attenuates the activation of the serotonin receptor by 5-95%, 10-90%, 20-80%, 30-70%, 40-60%, 50-95%, 65-85%, or 75-95% prior to administration of the psychedelic. In some embodiments, the serotonin receptor modulator attenuates the activation of the serotonin receptor by at least about or about 5%, 10%, 15%, 20%, 25%, 30%, 35%, 40%, 45%, 50%, 55%, 60%, 65%, 70%, 75%, 80%, 85%, 90%, 95%, or greater than 95% prior to administration of the psychedelic. In some embodiments, the serotonin receptor modulator attenuates the activation of the serotonin receptor by at least about or about 5% prior to administration of the psychedelic. In some embodiments, the serotonin receptor modulator attenuates the activation of the serotonin receptor by at least about or about 10% prior to administration of the psychedelic. In some embodiments, the serotonin receptor modulator attenuates the activation of the serotonin receptor by at least about or about 50% prior to administration of the psychedelic. In some embodiments, the serotonin receptor modulator substantially blocks or blocks the activation of the serotonin receptor prior to administration of the psychedelic.

Methods as described herein, in some embodiments, comprise administering composition comprising a psychedelic and a serotonin receptor modulator, wherein the serotonin receptor modulator attenuates the psychedelic-induced perceptual alterations. In some embodiments, psychedelic-induced perceptual alterations comprise changes in consciousness. In some embodiments, the serotonin receptor modulator attenuates the psychedelic-induced perceptual alterations by 5-95%, 10-90%, 20-80%, 30-70%, 40-60%, 50-95%, 65-85%, or 75-95%. In some embodiments, the serotonin receptor modulator attenuates the psychedelic-induced perceptual alterations by at least about or about 5%, 10%, 15%, 20%, 25%, 30%, 35%, 40%, 45%, 50%, 55%, 60%, 65%, 70%, 75%, 80%, 85%, 90%, 95%, or greater than 95%. In some embodiments, the serotonin receptor modulator attenuates the psychedelic-induced perceptual alterations by at least about or about 5%. In some embodiments, the serotonin receptor modulator attenuates the psychedelic-induced perceptual alterations by at least about or about 10%. In some embodiments, the serotonin receptor modulator attenuates the psychedelic-induced perceptual alterations by at least about or about 50%. In some embodiments, the serotonin receptor modulator substantially blocks or blocks the psychedelic-induced perceptual alterations.

Methods as described herein, in some embodiments, comprise administering composition comprising a psychedelic and a serotonin receptor modulator, wherein the serotonin receptor modulator enhances the anti-depressive effects of the psychedelic. In some embodiments, the serotonin receptor modulator improves the anti-depressive effects of the psychedelic by 5-95%, 10-90%, 20-80%, 30-70%, 40-60%, 50-95%, 65-85%, or 75-95%. In some embodiments, the serotonin receptor modulator improves the anti-depressive effects of the psychedelic by at least about or about 5%, 10%, 15%, 20%, 25%, 30%, 35%, 40%, 45%, 50%, 55%, 60%, 65%, 70%, 75%, 80%, 85%, 90%, 95%, or greater than 95%. In some embodiments, the serotonin receptor modulator improves the anti-depressive effects of the psychedelic by at least about or about 5%. In some embodiments, the serotonin receptor modulator improves the anti-depressive effects of the psychedelic by at least about or about 10%. In some embodiments, the serotonin receptor modulator improves the anti-depressive effects of the psychedelic by at least about or about 50%.

In some embodiments, the serotonin receptor modulator attenuates the activation of the serotonin receptor when the serotonin receptor modulator is administered or released from the composition provided herein to pretreat prior to the release of the psychedelic for a certain time. In some embodiments, the serotonin receptor modulator attenuates the activation of the serotonin receptor when the serotonin receptor modulator is administered or released from the composition provided herein to pretreat at an early time point (e.g., at most about 1.5 hours). In some embodiments, the serotonin receptor modulator attenuates the activation of the serotonin receptor when the serotonin receptor modulator is administered or released from the composition provided herein to pretreat at most about 2 hours prior to the release of the psychedelic. In some embodiments, the serotonin receptor modulator attenuates the activation of the serotonin receptor when the serotonin receptor modulator is administered or released from the composition provided herein to pretreat at most about 1 hour prior to the release of the psychedelic. In some embodiments, the serotonin receptor modulator attenuates the activation of the serotonin receptor when the serotonin receptor modulator is administered or released from the composition provided herein to pretreat at most about 1.5 hours prior to the release of the psychedelic. In some embodiments, the serotonin receptor modulator attenuates the activation of the serotonin receptor when the serotonin receptor modulator is administered or released from the composition provided herein to pretreat at about 1 hour prior to the release of the psychedelic. In some embodiments, the serotonin receptor modulator attenuates the activation of the serotonin receptor when the serotonin receptor modulator is used to pretreat at about or most about 5 minutes, 10 minutes, 20 minutes, 30 minutes, 40 minutes, 50 minutes, 1 hour, 1.25 hours, 1.5 hours, 2 hours, or 3 hours prior to the release of the psychedelic. In some embodiments, the serotonin receptor modulator attenuates the activation of the serotonin receptor when the serotonin receptor modulator is used to pretreat at most about 3 hours, 4 hours, 5 hours, 6 hours, 7 hours, 8 hours, 9 hours, or more than 9 hours prior to the release of the psychedelic. In some embodiments, the serotonin receptor modulator attenuates the activation of the serotonin receptor when the serotonin receptor modulator is used to pretreat in a range of about 5 minutes to about 3 hours, about 10 minutes to about 3 hours, about 20 minutes to about 3 hours, about 30 minutes to about 3 hours, about 40 minutes to about 3 hours, about 50 minutes to about 3 hours, about 1 hour to about 3 hours, about 5 minutes to about 2 hours, about 10 minutes to about 2 hours, about 20 minutes to about 2 hours, about 30 minutes to about 2 hours, about 40 minutes to about 2 hours, about 50 minutes to about 2 hours, about 1 hour to about 2 hours, about 5 minutes to about 1 hour, about 10 minutes to about 1 hour, about 20 minutes to about 1 hour, about 30 minutes to about 1 hour, about 40 minutes to about 1 hour, or about 50 minutes to about 1 hour prior to the release of the psychedelic.

In another aspect, also provided herein are methods of treating a disease or disorder in a subject in need thereof, the method comprising administering to the subject a psychedelic and a serotonin receptor modulator, wherein the serotonin receptor is administered prior to the administration of the psychedelic, and wherein the serotonin receptor modulator is administered at most about 3 hours prior to the administration of the psychedelic. In some embodiments, the disease or disorder is depression or a disease or disorder related to depression.

In another aspect, also provided herein are methods of treating a disease or disorder in a subject in need thereof, the method comprising administering to the subject a psychedelic and a serotonin receptor modulator, wherein the serotonin receptor is administered to the subject prior to the administration of the psychedelic to pretreat the subject, and wherein the serotonin receptor modulator is administered at most about 3 hours prior to the administration of the psychedelic. In some embodiments, the disease or disorder is depression or a disease or disorder related to depression.

In another aspect, also provided herein are methods of treating a disease or disorder in a subject in need thereof, the methods comprising (i) administering to the subject a serotonin receptor modulator to pretreat the subject, and (ii) administering to the subject a psychedelic, and wherein the serotonin receptor modulator is administered at most about 3 hours prior to the administration of the psychedelic. In some embodiments, the disease or disorder is depression or a disease or disorder related to depression.

In yet another aspect, also provided herein are methods of treating a disease or disorder in a subject in need thereof, the methods comprising administering to the subject a psychedelic, wherein the subject has been pretreated by a serotonin receptor modulator, and wherein the serotonin receptor modulator is administered at most about 3 hours prior to the administration of the psychedelic. In some embodiments, the disease or disorder is depression or a disease or disorder related to depression.

In yet another aspect, also provided herein are methods of reducing the adverse effects (e.g., hallucination) of a psychedelic in the treatment of a disease or disorder in a subject in need thereof, the methods comprising (i) administering to the subject a serotonin receptor modulator to pretreat the subject, and (ii) administering to the subject a psychedelic, and wherein the serotonin receptor modulator is administered at most about 3 hours prior to the administration of the psychedelic. In some embodiments, the disease or disorder is depression or a disease or disorder related to depression.

In yet another aspect, also provided herein are methods of reducing the adverse effects (e.g., hallucination) of a psychedelic in the treatment of a disease or disorder in a subject in need thereof, the methods comprising administering to the subject a psychedelic and a serotonin receptor modulator, wherein the serotonin receptor is administered prior to the administration of the psychedelic, and wherein the serotonin receptor modulator is administered at most about 3 hours prior to the administration of the psychedelic. In some embodiments, the disease or disorder is depression or a disease or disorder related to depression.

Also provided herein are methods of treating a disease or disorder in a subject in need thereof, the methods comprising administering to the subject a psychedelic, wherein the subject has been pretreated by a serotonin receptor modulator, wherein the serotonin receptor modulator is administered at most about 3 hours prior to the administration of the psychedelic, and wherein the serotonin receptor modulator suppresses the adverse effects (e.g., hallucination). In some embodiments, the disease or disorder is depression or a disease or disorder related to depression.

In certain embodiments of the methods provided herein, the co-administration of the psychedelic and the serotonin receptor modulator are therapeutically effective in treating a disease or disorder. In some embodiments, the disease or disorder is depression or a disease or disorder related to depression.

In some embodiments of the methods provided herein, the serotonin receptor modulator is administered at most about 3 hours prior to the administration of the psychedelic.

In some embodiments, the serotonin receptor modulator is administered at most about 2.5 hours prior to the administration of the psychedelic. In some embodiments, the serotonin receptor modulator is administered at most about 2 hours prior to the administration of the psychedelic. In some embodiments, the serotonin receptor modulator is administered at most about 1.5 hours prior to the administration of the psychedelic. In some embodiments, the serotonin receptor modulator is administered at most about 1 hour prior to the administration of the psychedelic. In some embodiments, the serotonin receptor modulator is administered at about 1 hour prior to the release of the psychedelic. In some embodiments, the serotonin receptor modulator is administered at least about 0.5 hours prior to the administration of the psychedelic. In some embodiments, the serotonin receptor modulator is administered at least about 1 hour prior to the release of the psychedelic. In some embodiments, the serotonin receptor modulator is administered at most about 5 minutes, 10 minutes, 20 minutes, 30 minutes, 40 minutes, 50 minutes, 1 hour, 1.25 hours, 1.5 hours, 2 hours, or 3 hours prior to the administration of the psychedelic. In some embodiments, the serotonin receptor modulator is administered at most about 3 hours, 4 hours, 5 hours, 6 hours, 7 hours, 8 hours, 9 hours, or more than 9 hours prior to the administration of the psychedelic. In some embodiments, the serotonin receptor modulator is administered in a window of about 5 minutes to about 3 hours, about 10 minutes to about 3 hours, about 20 minutes to about 3 hours, about 30 minutes to about 3 hours, about 40 minutes to about 3 hours, about 50 minutes to about 3 hours, about 1 hour to about 3 hours, about 70 minutes to about 3 hours, about 80 minutes to about 3 hours, about 90 minutes to about 3 hours, about 100 minutes to about 3 hours, about 110 minutes to about 3 hours, about 2 hours to about 3 hours, about 5 minutes to about 2 hours, about 10 minutes to about 2 hours, about 20 minutes to about 2 hours, about 30 minutes to about 2 hours, about 40 minutes to about 2 hours, about 50 minutes to about 2 hours, about 1 hour to about 2 hours, about 70 minutes to about 2 hours, about 80 minutes to about 2 hours, about 90 minutes to about 2 hours, about 100 minutes to about 2 hours, about 110 minutes to about 2 hours, about 5 minutes to about 1 hour, about 10 minutes to about 1 hour, about 20 minutes to about 1 hour, about 30 minutes to about 1 hour, about 40 minutes to about 1 hour, or about 50 minutes to about 1 hour prior to the administration of the psychedelic.

In some embodiments, the serotonin receptor modulator is administered at most about 6 hours prior to the administration of the psychedelic. In some embodiments, the serotonin receptor modulator is administered at most about 5 hours prior to the administration of the psychedelic. In some embodiments, the serotonin receptor modulator is administered at most about 4 hours prior to the administration of the psychedelic. In some embodiments, the serotonin receptor modulator is administered at most about 3 hours prior to the administration of the psychedelic.

In some embodiments, the serotonin receptor modulator is administered at least about 5 minutes, 10 minutes, 20 minutes, 30 minutes, 40 minutes, 50 minutes, 60 minutes, 70 minutes, 80 minutes, 90 minutes, 100 minutes, 110 minutes, 2 hours, 2.5 hours or 3 hours prior to the administration of the psychedelic. In some embodiments, the serotonin receptor modulator is administered at least about 3 hours, 4 hours, 5 hours, 6 hours, 7 hours, 8 hours, 9 hours, or more than 9 hours prior to the administration of the psychedelic.

In a preferred embodiment, the serotonin receptor modulator is administered at about 1 hour to about 3 hours prior to the administration of the psychedelic.

In some embodiments, the serotonin receptor modulator is eplivanserin and the psychedelic is psilocybin, wherein the eplivanserin is administered to pretreat at least 15 minutes prior to the administration of psilocybin. In some embodiments, the serotonin receptor modulator is eplivanserin and the psychedelic is psilocybin, wherein the eplivanserin is administered to pretreat at least 30 minutes prior to the psilocybin. In some embodiments, the serotonin receptor modulator is eplivanserin and the psychedelic is psilocybin, wherein the eplivanserin is administered to pretreat at least 60 minutes prior to the psilocybin. In some embodiments, the serotonin receptor modulator is eplivanserin and the psychedelic is psilocybin, wherein the eplivanserin is administered to pretreat at least 90 minutes prior to the psilocybin. In some embodiments, the serotonin receptor modulator is eplivanserin and the psychedelic is psilocybin, wherein the eplivanserin is administered to pretreat at least 120 minutes prior to the psilocybin. In some embodiments, the serotonin receptor modulator is eplivanserin and the psychedelic is psilocybin, wherein the eplivanserin is administered to pretreat at least 150 minutes prior to the psilocybin. In some embodiments, the serotonin receptor modulator is eplivanserin and the psychedelic is psilocybin, wherein the eplivanserin is administered to pretreat at least 180 minutes prior to the psilocybin. In some embodiments, the serotonin receptor modulator is eplivanserin and the psychedelic is psilocybin, wherein the eplivanserin is administered to pretreat at least 210 minutes prior to the psilocybin. In some embodiments, the serotonin receptor modulator is eplivanserin and the psychedelic is psilocybin, wherein the eplivanserin is administered to pretreat at least 240 minutes prior to the psilocybin. In some embodiments, the serotonin receptor modulator is eplivanserin and the psychedelic is psilocybin, wherein the eplivanserin is administered to pretreat at least 270 minutes prior to the psilocybin. In some embodiments, the serotonin receptor modulator is eplivanserin and the psychedelic is psilocybin, wherein the eplivanserin is administered to pretreat at least 300 minutes prior to the psilocybin. In some embodiments, the serotonin receptor modulator is eplivanserin and the psychedelic is psilocybin, wherein the eplivanserin is administered to pretreat at least 330 minutes prior to the psilocybin. In some embodiments, the serotonin receptor modulator is eplivanserin and the psychedelic is psilocybin, wherein the eplivanserin is administered to pretreat at least 360 minutes prior to the psilocybin.

In some preferred embodiments, the serotonin receptor modulator is eplivanserin and the psychedelic is psilocybin, wherein eplivanserin is administered to pretreat between about 60 minutes and about 180 minutes prior to the administration of psilocybin.

In some embodiments, the serotonin receptor modulator is volinanserin and the psychedelic is psilocybin, wherein the volinanserin is administered to pretreat at least 15 minutes prior to the administration of psilocybin. In some embodiments, the serotonin receptor modulator is volinanserin and the psychedelic is psilocybin, wherein the volinanserin is administered to pretreat at least 30 minutes prior to the psilocybin. In some embodiments, the serotonin receptor modulator is volinanserin and the psychedelic is psilocybin, wherein the volinanserin is administered to pretreat at least 60 minutes prior to the psilocybin. In some embodiments, the serotonin receptor modulator is volinanserin and the psychedelic is psilocybin, wherein the volinanserin is administered to pretreat at least 90 minutes prior to the psilocybin. In some embodiments, the serotonin receptor modulator is volinanserin and the psychedelic is psilocybin, wherein the volinanserin is administered to pretreat at least 120 minutes prior to the psilocybin. In some embodiments, the serotonin receptor modulator is volinanserin and the psychedelic is psilocybin, wherein the volinanserin is administered to pretreat at least 150 minutes prior to the psilocybin. In some embodiments, the serotonin receptor modulator is volinanserin and the psychedelic is psilocybin, wherein the volinanserin is administered to pretreat between about 15 minutes and about 150 minutes prior to the psilocybin. In some embodiments, the serotonin receptor modulator is volinanserin and the psychedelic is psilocybin, wherein the volinanserin is administered to pretreat at least 180 minutes prior to the psilocybin. In some embodiments, the serotonin receptor modulator is volinanserin and the psychedelic is psilocybin, wherein the volinanserin is administered to pretreat at least 210 minutes prior to the psilocybin. In some embodiments, the serotonin receptor modulator is volinanserin and the psychedelic is psilocybin, wherein the volinanserin is administered to pretreat at least 240 minutes prior to the psilocybin. In some embodiments, the serotonin receptor modulator is volinanserin and the psychedelic is psilocybin, wherein the volinanserin is administered to pretreat at least 270 minutes prior to the psilocybin. In some embodiments, the serotonin receptor modulator is volinanserin and the psychedelic is psilocybin, wherein the volinanserin is administered to pretreat at least 300 minutes prior to the psilocybin. In some embodiments, the serotonin receptor modulator is volinanserin and the psychedelic is psilocybin, wherein the volinanserin is administered to pretreat at least 330 minutes prior to the psilocybin. In some embodiments, the serotonin receptor modulator is volinanserin and the psychedelic is psilocybin, wherein the volinanserin is administered to pretreat at least 360 minutes prior to the psilocybin. In some preferred embodiments, the serotonin receptor modulator is volinanserin and the psychedelic is psilocybin, wherein volinanserin is administered to pretreat between about 60 minutes and about 180 minutes prior to the administration of psilocybin.

In some embodiments, the serotonin receptor modulator is ketanserin and the psychedelic is psilocybin, wherein the ketanserin is administered to pretreat at least 15 minutes prior to the administration of psilocybin. In some embodiments, the serotonin receptor modulator is ketanserin and the psychedelic is psilocybin, wherein the ketanserin is administered to pretreat at least 30 minutes prior to the psilocybin. In some embodiments, the serotonin receptor modulator is ketanserin and the psychedelic is psilocybin, wherein the ketanserin is administered to pretreat at least 60 minutes prior to the psilocybin. In some embodiments, the serotonin receptor modulator is ketanserin and the psychedelic is psilocybin, wherein the ketanserin is administered to pretreat at least 90 minutes prior to the psilocybin. In some embodiments, the serotonin receptor modulator is ketanserin and the psychedelic is psilocybin, wherein the ketanserin is administered to pretreat at least 120 minutes prior to the psilocybin. In some embodiments, the serotonin receptor modulator is ketanserin and the psychedelic is psilocybin, wherein the ketanserin is administered to pretreat at least 150 minutes prior to the psilocybin. In some embodiments, the serotonin receptor modulator is ketanserin and the psychedelic is psilocybin, wherein the ketanserin is administered to pretreat between about 15 minutes and about 150 minutes prior to the psilocybin. In some embodiments, the serotonin receptor modulator is ketanserin and the psychedelic is psilocybin, wherein the ketanserin is administered to pretreat at least 180 minutes prior to the psilocybin. In some embodiments, the serotonin receptor modulator is ketanserin and the psychedelic is psilocybin, wherein the ketanserin is administered to pretreat at least 210 minutes prior to the psilocybin. In some embodiments, the serotonin receptor modulator is ketanserin and the psychedelic is psilocybin, wherein the ketanserin is administered to pretreat at least 240 minutes prior to the psilocybin. In some embodiments, the serotonin receptor modulator is ketanserin and the psychedelic is psilocybin, wherein the ketanserin is administered to pretreat at least 270 minutes prior to the psilocybin. In some embodiments, the serotonin receptor modulator is ketanserin and the psychedelic is psilocybin, wherein the ketanserin is administered to pretreat at least 300 minutes prior to the psilocybin. In some embodiments, the serotonin receptor modulator is ketanserin and the psychedelic is psilocybin, wherein the ketanserin is administered to pretreat at least 330 minutes prior to the psilocybin. In some embodiments, the serotonin receptor modulator is ketanserin and the psychedelic is psilocybin, wherein the ketanserin is administered to pretreat at least 360 minutes prior to the psilocybin. In some preferred embodiments, the serotonin receptor modulator is ketanserin and the psychedelic is psilocybin, wherein ketanserin is administered to pretreat between about 60 minutes and about 180 minutes prior to the administration of psilocybin.

In some embodiments, the serotonin receptor modulator is ritanserin and the psychedelic is psilocybin, wherein the ritanserin is administered to pretreat at least 15 minutes prior to the administration of psilocybin. In some embodiments, the serotonin receptor modulator is ritanserin and the psychedelic is psilocybin, wherein the ritanserin is administered to pretreat at least 30 minutes prior to the psilocybin. In some embodiments, the serotonin receptor modulator is ritanserin and the psychedelic is psilocybin, wherein the ritanserin is administered to pretreat at least 60 minutes prior to the psilocybin. In some embodiments, the serotonin receptor modulator is ritanserin and the psychedelic is psilocybin, wherein the ritanserin is administered to pretreat at least 90 minutes prior to the psilocybin. In some embodiments, the serotonin receptor modulator is ritanserin and the psychedelic is psilocybin, wherein the ritanserin is administered to pretreat at least 120 minutes prior to the psilocybin. In some embodiments, the serotonin receptor modulator is ritanserin and the psychedelic is psilocybin, wherein the ritanserin is administered to pretreat at least 150 minutes prior to the psilocybin. In some embodiments, the serotonin receptor modulator is ritanserin and the psychedelic is psilocybin, wherein the ritanserin is administered to pretreat between about 15 minutes and about 150 minutes prior to the psilocybin. In some embodiments, the serotonin receptor modulator is ritanserin and the psychedelic is psilocybin, wherein the ritanserin is administered to pretreat at least 180 minutes prior to the psilocybin. In some embodiments, the serotonin receptor modulator is ritanserin and the psychedelic is psilocybin, wherein the ritanserin is administered to pretreat at least 210 minutes prior to the psilocybin. In some embodiments, the serotonin receptor modulator is ritanserin and the psychedelic is psilocybin, wherein the ritanserin is administered to pretreat at least 240 minutes prior to the psilocybin. In some embodiments, the serotonin receptor modulator is ritanserin and the psychedelic is psilocybin, wherein the ritanserin is administered to pretreat at least 270 minutes prior to the psilocybin. In some embodiments, the serotonin receptor modulator is ritanserin and the psychedelic is psilocybin, wherein the ritanserin is administered to pretreat at least 300 minutes prior to the psilocybin. In some embodiments, the serotonin receptor modulator is ritanserin and the psychedelic is psilocybin, wherein the ritanserin is administered to pretreat at least 330 minutes prior to the psilocybin. In some embodiments, the serotonin receptor modulator is ritanserin and the psychedelic is psilocybin, wherein the ritanserin is administered to pretreat at least 360 minutes prior to the psilocybin. In some preferred embodiments, the serotonin receptor modulator is ritanserin and the psychedelic is psilocybin, wherein ritanserin is administered to pretreat between about 60 minutes and about 180 minutes prior to the administration of psilocybin.

In some embodiments, the serotonin receptor modulator is pimavanserin and the psychedelic is psilocybin, wherein the pimavanserin is administered to pretreat at least 15 minutes prior to the administration of psilocybin. In some embodiments, the serotonin receptor modulator is pimavanserin and the psychedelic is psilocybin, wherein the pimavanserin is administered to pretreat at least 30 minutes prior to the psilocybin. In some embodiments, the serotonin receptor modulator is pimavanserin and the psychedelic is psilocybin, wherein the pimavanserin is administered to pretreat at least 60 minutes prior to the psilocybin. In some embodiments, the serotonin receptor modulator is pimavanserin and the psychedelic is psilocybin, wherein the pimavanserin is administered to pretreat at least 90 minutes prior to the psilocybin. In some embodiments, the serotonin receptor modulator is pimavanserin and the psychedelic is psilocybin, wherein the pimavanserin is administered to pretreat at least 120 minutes prior to the psilocybin. In some embodiments, the serotonin receptor modulator is pimavanserin and the psychedelic is psilocybin, wherein the pimavanserin is administered to pretreat at least 150 minutes prior to the psilocybin. In some embodiments, the serotonin receptor modulator is pimavanserin and the psychedelic is psilocybin, wherein the pimavanserin is administered to pretreat between about 15 minutes and about 150 minutes prior to the psilocybin. In some embodiments, the serotonin receptor modulator is pimavanserin and the psychedelic is psilocybin, wherein the pimavanserin is administered to pretreat at least 180 minutes prior to the psilocybin. In some embodiments, the serotonin receptor modulator is pimavanserin and the psychedelic is psilocybin, wherein the pimavanserin is administered to pretreat at least 210 minutes prior to the psilocybin. In some embodiments, the serotonin receptor modulator is pimavanserin and the psychedelic is psilocybin, wherein the pimavanserin is administered to pretreat at least 240 minutes prior to the psilocybin. In some embodiments, the serotonin receptor modulator is pimavanserin and the psychedelic is psilocybin, wherein the pimavanserin is administered to pretreat at least 270 minutes prior to the psilocybin. In some embodiments, the serotonin receptor modulator is pimavanserin and the psychedelic is psilocybin, wherein the pimavanserin is administered to pretreat at least 300 minutes prior to the psilocybin. In some embodiments, the serotonin receptor modulator is pimavanserin and the psychedelic is psilocybin, wherein the pimavanserin is administered to pretreat at least 330 minutes prior to the psilocybin. In some embodiments, the serotonin receptor modulator is pimavanserin and the psychedelic is psilocybin, wherein the pimavanserin is administered to pretreat at least 360 minutes prior to the psilocybin. In some preferred embodiments, the serotonin receptor modulator is pimavanserin and the psychedelic is psilocybin, wherein pimavanserin is administered to pretreat between about 60 minutes and about 180 minutes prior to the administration of psilocybin.

In some embodiments, the serotonin receptor modulator is nelotanserin and the psychedelic is psilocybin, wherein the nelotanserin is administered to pretreat at least 15 minutes prior to the administration of psilocybin. In some embodiments, the serotonin receptor modulator is nelotanserin and the psychedelic is psilocybin, wherein the nelotanserin is administered to pretreat at least 30 minutes prior to the psilocybin. In some embodiments, the serotonin receptor modulator is nelotanserin and the psychedelic is psilocybin, wherein the nelotanserin is administered to pretreat at least 60 minutes prior to the psilocybin. In some embodiments, the serotonin receptor modulator is nelotanserin and the psychedelic is psilocybin, wherein the nelotanserin is administered to pretreat at least 90 minutes prior to the psilocybin. In some embodiments, the serotonin receptor modulator is nelotanserin and the psychedelic is psilocybin, wherein the nelotanserin is administered to pretreat at least 120 minutes prior to the psilocybin. In some embodiments, the serotonin receptor modulator is nelotanserin and the psychedelic is psilocybin, wherein the nelotanserin is administered to pretreat at least 150 minutes prior to the psilocybin. In some embodiments, the serotonin receptor modulator is nelotanserin and the psychedelic is psilocybin, wherein the nelotanserin is administered to pretreat between about 15 minutes and about 150 minutes prior to the psilocybin. In some embodiments, the serotonin receptor modulator is nelotanserin and the psychedelic is psilocybin, wherein the nelotanserin is administered to pretreat at least 180 minutes prior to the psilocybin. In some embodiments, the serotonin receptor modulator is nelotanserin and the psychedelic is psilocybin, wherein the nelotanserin is administered to pretreat at least 210 minutes prior to the psilocybin. In some embodiments, the serotonin receptor modulator is nelotanserin and the psychedelic is psilocybin, wherein the nelotanserin is administered to pretreat at least 240 minutes prior to the psilocybin. In some embodiments, the serotonin receptor modulator is nelotanserin and the psychedelic is psilocybin, wherein the nelotanserin is administered to pretreat at least 270 minutes prior to the psilocybin. In some embodiments, the serotonin receptor modulator is nelotanserin and the psychedelic is psilocybin, wherein the nelotanserin is administered to pretreat at least 300 minutes prior to the psilocybin. In some embodiments, the serotonin receptor modulator is nelotanserin and the psychedelic is psilocybin, wherein the nelotanserin is administered to pretreat at least 330 minutes prior to the psilocybin. In some embodiments, the serotonin receptor modulator is nelotanserin and the psychedelic is psilocybin, wherein the nelotanserin is administered to pretreat at least 360 minutes prior to the psilocybin.

In some preferred embodiments, the serotonin receptor modulator is nelotanserin and the psychedelic is psilocybin, wherein nelotanserin is administered to pretreat between about 60 minutes and about 180 minutes prior to the administration of psilocybin.

In some embodiments, the serotonin receptor modulator is pruvanserin and the psychedelic is psilocybin, wherein the pruvanserin is administered to pretreat at least 15 minutes prior to the administration of psilocybin. In some embodiments, the serotonin receptor modulator is pruvanserin and the psychedelic is psilocybin, wherein the pruvanserin is administered to pretreat at least 30 minutes prior to the psilocybin. In some embodiments, the serotonin receptor modulator is pruvanserin and the psychedelic is psilocybin, wherein the pruvanserin is administered to pretreat at least 60 minutes prior to the psilocybin. In some embodiments, the serotonin receptor modulator is pruvanserin and the psychedelic is psilocybin, wherein the pruvanserin is administered to pretreat at least 90 minutes prior to the psilocybin. In some embodiments, the serotonin receptor modulator is pruvanserin and the psychedelic is psilocybin, wherein the pruvanserin is administered to pretreat at least 120 minutes prior to the psilocybin. In some embodiments, the serotonin receptor modulator is pruvanserin and the psychedelic is psilocybin, wherein the pruvanserin is administered to pretreat at least 150 minutes prior to the psilocybin. In some embodiments, the serotonin receptor modulator is pruvanserin and the psychedelic is psilocybin, wherein the pruvanserin is administered to pretreat between about 15 minutes and about 150 minutes prior to the psilocybin. In some embodiments, the serotonin receptor modulator is pruvanserin and the psychedelic is psilocybin, wherein the pruvanserin is administered to pretreat at least 180 minutes prior to the psilocybin. In some embodiments, the serotonin receptor modulator is pruvanserin and the psychedelic is psilocybin, wherein the pruvanserin is administered to pretreat at least 210 minutes prior to the psilocybin. In some embodiments, the serotonin receptor modulator is pruvanserin and the psychedelic is psilocybin, wherein the pruvanserin is administered to pretreat at least 240 minutes prior to the psilocybin. In some embodiments, the serotonin receptor modulator is pruvanserin and the psychedelic is psilocybin, wherein the pruvanserin is administered to pretreat at least 270 minutes prior to the psilocybin. In some embodiments, the serotonin receptor modulator is pruvanserin and the psychedelic is psilocybin, wherein the pruvanserin is administered to pretreat at least 300 minutes prior to the psilocybin. In some embodiments, the serotonin receptor modulator is pruvanserin and the psychedelic is psilocybin, wherein the pruvanserin is administered to pretreat at least 330 minutes prior to the psilocybin. In some embodiments, the serotonin receptor modulator is pruvanserin and the psychedelic is psilocybin, wherein the pruvanserin is administered to pretreat at least 360 minutes prior to the psilocybin. In some preferred embodiments, the serotonin receptor modulator is pruvanserin and the psychedelic is psilocybin, wherein pruvanserin is administered to pretreat between about 60 minutes and about 180 minutes prior to the administration of psilocybin.

In some embodiments, the serotonin receptor modulator is eplivanserin and the psychedelic is 4-Acetoxy-DMT, wherein the eplivanserin is administered to pretreat at least 15 minutes prior to the administration of 4-Acetoxy-DMT. In some embodiments, the serotonin receptor modulator is eplivanserin and the psychedelic is 4-Acetoxy-DMT, wherein the eplivanserin is administered to pretreat at least 30 minutes prior to the 4-Acetoxy-DMT. In some embodiments, the serotonin receptor modulator is eplivanserin and the psychedelic is 4-Acetoxy-DMT, wherein the eplivanserin is administered to pretreat at least 60 minutes prior to the 4-Acetoxy-DMT. In some embodiments, the serotonin receptor modulator is eplivanserin and the psychedelic is 4-Acetoxy-DMT, wherein the eplivanserin is administered to pretreat at least 90 minutes prior to the 4-Acetoxy-DMT. In some embodiments, the serotonin receptor modulator is eplivanserin and the psychedelic is 4-Acetoxy-DMT, wherein the eplivanserin is administered to pretreat at least 120 minutes prior to the 4-Acetoxy-DMT. In some embodiments, the serotonin receptor modulator is eplivanserin and the psychedelic is 4-Acetoxy-DMT, wherein the eplivanserin is administered to pretreat at least 150 minutes prior to the 4-Acetoxy-DMT. In some embodiments, the serotonin receptor modulator is eplivanserin and the psychedelic is 4-Acetoxy-DMT, wherein the eplivanserin is administered to pretreat at least 180 minutes prior to the 4-Acetoxy-DMT. In some embodiments, the serotonin receptor modulator is eplivanserin and the psychedelic is 4-Acetoxy-DMT, wherein the eplivanserin is administered to pretreat at least 210 minutes prior to the 4-Acetoxy-DMT. In some embodiments, the serotonin receptor modulator is eplivanserin and the psychedelic is 4-Acetoxy-DMT, wherein the eplivanserin is administered to pretreat at least 240 minutes prior to the 4-Acetoxy-DMT. In some embodiments, the serotonin receptor modulator is eplivanserin and the psychedelic is 4-Acetoxy-DMT, wherein the eplivanserin is administered to pretreat at least 270 minutes prior to the 4-Acetoxy-DMT. In some embodiments, the serotonin receptor modulator is eplivanserin and the psychedelic is 4-Acetoxy-DMT, wherein the eplivanserin is administered to pretreat at least 300 minutes prior to the 4-Acetoxy-DMT. In some embodiments, the serotonin receptor modulator is eplivanserin and the psychedelic is 4-Acetoxy-DMT, wherein the eplivanserin is administered to pretreat at least 330 minutes prior to the 4-Acetoxy-DMT. In some embodiments, the serotonin receptor modulator is eplivanserin and the psychedelic is 4-Acetoxy-DMT, wherein the eplivanserin is administered to pretreat at least 360 minutes prior to the 4-Acetoxy-DMT.

In some preferred embodiments, the serotonin receptor modulator is eplivanserin and the psychedelic is 4-Acetoxy-DMT, wherein eplivanserin is administered to pretreat between about 60 minutes and about 180 minutes prior to the administration of 4-Acetoxy-DMT.

In some embodiments, the serotonin receptor modulator is volinanserin and the psychedelic is 4-Acetoxy-DMT, wherein the volinanserin is administered to pretreat at least 15 minutes prior to the administration of 4-Acetoxy-DMT. In some embodiments, the serotonin receptor modulator is volinanserin and the psychedelic is 4-Acetoxy-DMT, wherein the volinanserin is administered to pretreat at least 30 minutes prior to the 4-Acetoxy-DMT. In some embodiments, the serotonin receptor modulator is volinanserin and the psychedelic is 4-Acetoxy-DMT, wherein the volinanserin is administered to pretreat at least 60 minutes prior to the 4-Acetoxy-DMT. In some embodiments, the serotonin receptor modulator is volinanserin and the psychedelic is 4-Acetoxy-DMT, wherein the volinanserin is administered to pretreat at least 90 minutes prior to the 4-Acetoxy-DMT. In some embodiments, the serotonin receptor modulator is volinanserin and the psychedelic is 4-Acetoxy-DMT, wherein the volinanserin is administered to pretreat at least 120 minutes prior to the 4-Acetoxy-DMT. In some embodiments, the serotonin receptor modulator is volinanserin and the psychedelic is 4-Acetoxy-DMT, wherein the volinanserin is administered to pretreat at least 150 minutes prior to the 4-Acetoxy-DMT. In some embodiments, the serotonin receptor modulator is volinanserin and the psychedelic is 4-Acetoxy-DMT, wherein the volinanserin is administered to pretreat between about 15 minutes and about 150 minutes prior to the 4-Acetoxy-DMT. In some embodiments, the serotonin receptor modulator is volinanserin and the psychedelic is 4-Acetoxy-DMT, wherein the volinanserin is administered to pretreat at least 180 minutes prior to the 4-Acetoxy-DMT. In some embodiments, the serotonin receptor modulator is volinanserin and the psychedelic is 4-Acetoxy-DMT, wherein the volinanserin is administered to pretreat at least 210 minutes prior to the 4-Acetoxy-DMT. In some embodiments, the serotonin receptor modulator is volinanserin and the psychedelic is 4-Acetoxy-DMT, wherein the volinanserin is administered to pretreat at least 240 minutes prior to the 4-Acetoxy-DMT. In some embodiments, the serotonin receptor modulator is volinanserin and the psychedelic is 4-Acetoxy-DMT, wherein the volinanserin is administered to pretreat at least 270 minutes prior to the 4-Acetoxy-DMT. In some embodiments, the serotonin receptor modulator is volinanserin and the psychedelic is 4-Acetoxy-DMT, wherein the volinanserin is administered to pretreat at least 300 minutes prior to the 4-Acetoxy-DMT. In some embodiments, the serotonin receptor modulator is volinanserin and the psychedelic is 4-Acetoxy-DMT, wherein the volinanserin is administered to pretreat at least 330 minutes prior to the 4-Acetoxy-DMT. In some embodiments, the serotonin receptor modulator is volinanserin and the psychedelic is 4-Acetoxy-DMT, wherein the volinanserin is administered to pretreat at least 360 minutes prior to the 4-Acetoxy-DMT. In some preferred embodiments, the serotonin receptor modulator is volinanserin and the psychedelic is 4-Acetoxy-DMT, wherein volinanserin is administered to pretreat between about 60 minutes and about 180 minutes prior to the administration of 4-Acetoxy-DMT.

In some embodiments, the serotonin receptor modulator is ketanserin and the psychedelic is 4-Acetoxy-DMT, wherein the ketanserin is administered to pretreat at least 15 minutes prior to the administration of 4-Acetoxy-DMT. In some embodiments, the serotonin receptor modulator is ketanserin and the psychedelic is 4-Acetoxy-DMT, wherein the ketanserin is administered to pretreat at least 30 minutes prior to the 4-Acetoxy-DMT. In some embodiments, the serotonin receptor modulator is ketanserin and the psychedelic is 4-Acetoxy-DMT, wherein the ketanserin is administered to pretreat at least 60 minutes prior to the 4-Acetoxy-DMT. In some embodiments, the serotonin receptor modulator is ketanserin and the psychedelic is 4-Acetoxy-DMT, wherein the ketanserin is administered to pretreat at least 90 minutes prior to the 4-Acetoxy-DMT. In some embodiments, the serotonin receptor modulator is ketanserin and the psychedelic is 4-Acetoxy-DMT, wherein the ketanserin is administered to pretreat at least 120 minutes prior to the 4-Acetoxy-DMT. In some embodiments, the serotonin receptor modulator is ketanserin and the psychedelic is 4-Acetoxy-DMT, wherein the ketanserin is administered to pretreat at least 150 minutes prior to the 4-Acetoxy-DMT. In some embodiments, the serotonin receptor modulator is ketanserin and the psychedelic is 4-Acetoxy-DMT, wherein the ketanserin is administered to pretreat between about 15 minutes and about 150 minutes prior to the 4-Acetoxy-DMT. In some embodiments, the serotonin receptor modulator is ketanserin and the psychedelic is 4-Acetoxy-DMT, wherein the ketanserin is administered to pretreat at least 180 minutes prior to the 4-Acetoxy-DMT. In some embodiments, the serotonin receptor modulator is ketanserin and the psychedelic is 4-Acetoxy-DMT, wherein the ketanserin is administered to pretreat at least 210 minutes prior to the 4-Acetoxy-DMT. In some embodiments, the serotonin receptor modulator is ketanserin and the psychedelic is 4-Acetoxy-DMT, wherein the ketanserin is administered to pretreat at least 240 minutes prior to the 4-Acetoxy-DMT. In some embodiments, the serotonin receptor modulator is ketanserin and the psychedelic is 4-Acetoxy-DMT, wherein the ketanserin is administered to pretreat at least 270 minutes prior to the 4-Acetoxy-DMT. In some embodiments, the serotonin receptor modulator is ketanserin and the psychedelic is 4-Acetoxy-DMT, wherein the ketanserin is administered to pretreat at least 300 minutes prior to the 4-Acetoxy-DMT. In some embodiments, the serotonin receptor modulator is ketanserin and the psychedelic is 4-Acetoxy-DMT, wherein the ketanserin is administered to pretreat at least 330 minutes prior to the 4-Acetoxy-DMT. In some embodiments, the serotonin receptor modulator is ketanserin and the psychedelic is 4-Acetoxy-DMT, wherein the ketanserin is administered to pretreat at least 360 minutes prior to the 4-Acetoxy-DMT. In some preferred embodiments, the serotonin receptor modulator is ketanserin and the psychedelic is 4-Acetoxy-DMT, wherein ketanserin is administered to pretreat between about 60 minutes and about 180 minutes prior to the administration of 4-Acetoxy-DMT.

In some embodiments, the serotonin receptor modulator is ritanserin and the psychedelic is 4-Acetoxy-DMT, wherein the ritanserin is administered to pretreat at least 15 minutes prior to the administration of 4-Acetoxy-DMT. In some embodiments, the serotonin receptor modulator is ritanserin and the psychedelic is 4-Acetoxy-DMT, wherein the ritanserin is administered to pretreat at least 30 minutes prior to the 4-Acetoxy-DMT. In some embodiments, the serotonin receptor modulator is ritanserin and the psychedelic is 4-Acetoxy-DMT, wherein the ritanserin is administered to pretreat at least 60 minutes prior to the 4-Acetoxy-DMT. In some embodiments, the serotonin receptor modulator is ritanserin and the psychedelic is 4-Acetoxy-DMT, wherein the ritanserin is administered to pretreat at least 90 minutes prior to the 4-Acetoxy-DMT. In some embodiments, the serotonin receptor modulator is ritanserin and the psychedelic is 4-Acetoxy-DMT, wherein the ritanserin is administered to pretreat at least 120 minutes prior to the 4-Acetoxy-DMT. In some embodiments, the serotonin receptor modulator is ritanserin and the psychedelic is 4-Acetoxy-DMT, wherein the ritanserin is administered to pretreat at least 150 minutes prior to the 4-Acetoxy-DMT. In some embodiments, the serotonin receptor modulator is ritanserin and the psychedelic is 4-Acetoxy-DMT, wherein the ritanserin is administered to pretreat between about 15 minutes and about 150 minutes prior to the 4-Acetoxy-DMT. In some embodiments, the serotonin receptor modulator is ritanserin and the psychedelic is 4-Acetoxy-DMT, wherein the ritanserin is administered to pretreat at least 180 minutes prior to the 4-Acetoxy-DMT. In some embodiments, the serotonin receptor modulator is ritanserin and the psychedelic is 4-Acetoxy-DMT, wherein the ritanserin is administered to pretreat at least 210 minutes prior to the 4-Acetoxy-DMT. In some embodiments, the serotonin receptor modulator is ritanserin and the psychedelic is 4-Acetoxy-DMT, wherein the ritanserin is administered to pretreat at least 240 minutes prior to the 4-Acetoxy-DMT. In some embodiments, the serotonin receptor modulator is ritanserin and the psychedelic is 4-Acetoxy-DMT, wherein the ritanserin is administered to pretreat at least 270 minutes prior to the 4-Acetoxy-DMT. In some embodiments, the serotonin receptor modulator is ritanserin and the psychedelic is 4-Acetoxy-DMT, wherein the ritanserin is administered to pretreat at least 300 minutes prior to the 4-Acetoxy-DMT. In some embodiments, the serotonin receptor modulator is ritanserin and the psychedelic is 4-Acetoxy-DMT, wherein the ritanserin is administered to pretreat at least 330 minutes prior to the 4-Acetoxy-DMT. In some embodiments, the serotonin receptor modulator is ritanserin and the psychedelic is 4-Acetoxy-DMT, wherein the ritanserin is administered to pretreat at least 360 minutes prior to the 4-Acetoxy-DMT. In some preferred embodiments, the serotonin receptor modulator is ritanserin and the psychedelic is 4-Acetoxy-DMT, wherein ritanserin is administered to pretreat between about 60 minutes and about 180 minutes prior to the administration of 4-Acetoxy-DMT.

In some embodiments, the serotonin receptor modulator is pimavanserin and the psychedelic is 4-Acetoxy-DMT, wherein the pimavanserin is administered to pretreat at least 15 minutes prior to the administration of 4-Acetoxy-DMT. In some embodiments, the serotonin receptor modulator is pimavanserin and the psychedelic is 4-Acetoxy-DMT, wherein the pimavanserin is administered to pretreat at least 30 minutes prior to the 4-Acetoxy-DMT. In some embodiments, the serotonin receptor modulator is pimavanserin and the psychedelic is 4-Acetoxy-DMT, wherein the pimavanserin is administered to pretreat at least 60 minutes prior to the 4-Acetoxy-DMT. In some embodiments, the serotonin receptor modulator is pimavanserin and the psychedelic is 4-Acetoxy-DMT, wherein the pimavanserin is administered to pretreat at least 90 minutes prior to the 4-Acetoxy-DMT. In some embodiments, the serotonin receptor modulator is pimavanserin and the psychedelic is 4-Acetoxy-DMT, wherein the pimavanserin is administered to pretreat at least 120 minutes prior to the 4-Acetoxy-DMT. In some embodiments, the serotonin receptor modulator is pimavanserin and the psychedelic is 4-Acetoxy-DMT, wherein the pimavanserin is administered to pretreat at least 150 minutes prior to the 4-Acetoxy-DMT. In some embodiments, the serotonin receptor modulator is pimavanserin and the psychedelic is 4-Acetoxy-DMT, wherein the pimavanserin is administered to pretreat between about 15 minutes and about 150 minutes prior to the 4-Acetoxy-DMT. In some embodiments, the serotonin receptor modulator is pimavanserin and the psychedelic is 4-Acetoxy-DMT, wherein the pimavanserin is administered to pretreat at least 180 minutes prior to the 4-Acetoxy-DMT. In some embodiments, the serotonin receptor modulator is pimavanserin and the psychedelic is 4-Acetoxy-DMT, wherein the pimavanserin is administered to pretreat at least 210 minutes prior to the 4-Acetoxy-DMT. In some embodiments, the serotonin receptor modulator is pimavanserin and the psychedelic is 4-Acetoxy-DMT, wherein the pimavanserin is administered to pretreat at least 240 minutes prior to the 4-Acetoxy-DMT. In some embodiments, the serotonin receptor modulator is pimavanserin and the psychedelic is 4-Acetoxy-DMT, wherein the pimavanserin is administered to pretreat at least 270 minutes prior to the 4-Acetoxy-DMT. In some embodiments, the serotonin receptor modulator is pimavanserin and the psychedelic is 4-Acetoxy-DMT, wherein the pimavanserin is administered to pretreat at least 300 minutes prior to the 4-Acetoxy-DMT. In some embodiments, the serotonin receptor modulator is pimavanserin and the psychedelic is 4-Acetoxy-DMT, wherein the pimavanserin is administered to pretreat at least 330 minutes prior to the 4-Acetoxy-DMT. In some embodiments, the serotonin receptor modulator is pimavanserin and the psychedelic is 4-Acetoxy-DMT, wherein the pimavanserin is administered to pretreat at least 360 minutes prior to the 4-Acetoxy-DMT. In some preferred embodiments, the serotonin receptor modulator is pimavanserin and the psychedelic is 4-Acetoxy-DMT, wherein pimavanserin is administered to pretreat between about 60 minutes and about 180 minutes prior to the administration of 4-Acetoxy-DMT.

In some embodiments, the serotonin receptor modulator is nelotanserin and the psychedelic is 4-Acetoxy-DMT, wherein the nelotanserin is administered to pretreat at least 15 minutes prior to the administration of 4-Acetoxy-DMT. In some embodiments, the serotonin receptor modulator is nelotanserin and the psychedelic is 4-Acetoxy-DMT, wherein the nelotanserin is administered to pretreat at least 30 minutes prior to the 4-Acetoxy-DMT. In some embodiments, the serotonin receptor modulator is nelotanserin and the psychedelic is 4-Acetoxy-DMT, wherein the nelotanserin is administered to pretreat at least 60 minutes prior to the 4-Acetoxy-DMT. In some embodiments, the serotonin receptor modulator is nelotanserin and the psychedelic is 4-Acetoxy-DMT, wherein the nelotanserin is administered to pretreat at least 90 minutes prior to the 4-Acetoxy-DMT. In some embodiments, the serotonin receptor modulator is nelotanserin and the psychedelic is 4-Acetoxy-DMT, wherein the nelotanserin is administered to pretreat at least 120 minutes prior to the 4-Acetoxy-DMT. In some embodiments, the serotonin receptor modulator is nelotanserin and the psychedelic is 4-Acetoxy-DMT, wherein the nelotanserin is administered to pretreat at least 150 minutes prior to the 4-Acetoxy-DMT. In some embodiments, the serotonin receptor modulator is nelotanserin and the psychedelic is 4-Acetoxy-DMT, wherein the nelotanserin is administered to pretreat between about 15 minutes and about 150 minutes prior to the 4-Acetoxy-DMT. In some embodiments, the serotonin receptor modulator is nelotanserin and the psychedelic is 4-Acetoxy-DMT, wherein the nelotanserin is administered to pretreat at least 180 minutes prior to the 4-Acetoxy-DMT. In some embodiments, the serotonin receptor modulator is nelotanserin and the psychedelic is 4-Acetoxy-DMT, wherein the nelotanserin is administered to pretreat at least 210 minutes prior to the 4-Acetoxy-DMT. In some embodiments, the serotonin receptor modulator is nelotanserin and the psychedelic is 4-Acetoxy-DMT, wherein the nelotanserin is administered to pretreat at least 240 minutes prior to the 4-Acetoxy-DMT. In some embodiments, the serotonin receptor modulator is nelotanserin and the psychedelic is 4-Acetoxy-DMT, wherein the nelotanserin is administered to pretreat at least 270 minutes prior to the 4-Acetoxy-DMT. In some embodiments, the serotonin receptor modulator is nelotanserin and the psychedelic is 4-Acetoxy-DMT, wherein the nelotanserin is administered to pretreat at least 300 minutes prior to the 4-Acetoxy-DMT. In some embodiments, the serotonin receptor modulator is nelotanserin and the psychedelic is 4-Acetoxy-DMT, wherein the nelotanserin is administered to pretreat at least 330 minutes prior to the 4-Acetoxy-DMT. In some embodiments, the serotonin receptor modulator is nelotanserin and the psychedelic is 4-Acetoxy-DMT, wherein the nelotanserin is administered to pretreat at least 360 minutes prior to the 4-Acetoxy-DMT. In some preferred embodiments, the serotonin receptor modulator is nelotanserin and the psychedelic is 4-Acetoxy-DMT, wherein nelotanserin is administered to pretreat between about 60 minutes and about 180 minutes prior to the administration of 4-Acetoxy-DMT.

In some embodiments, the serotonin receptor modulator is pruvanserin and the psychedelic is 4-Acetoxy-DMT, wherein the pruvanserin is administered to pretreat at least 15 minutes prior to the administration of 4-Acetoxy-DMT. In some embodiments, the serotonin receptor modulator is pruvanserin and the psychedelic is 4-Acetoxy-DMT, wherein the pruvanserin is administered to pretreat at least 30 minutes prior to the 4-Acetoxy-DMT. In some embodiments, the serotonin receptor modulator is pruvanserin and the psychedelic is 4-Acetoxy-DMT, wherein the pruvanserin is administered to pretreat at least 60 minutes prior to the 4-Acetoxy-DMT. In some embodiments, the serotonin receptor modulator is pruvanserin and the psychedelic is 4-Acetoxy-DMT, wherein the pruvanserin is administered to pretreat at least 90 minutes prior to the 4-Acetoxy-DMT. In some embodiments, the serotonin receptor modulator is pruvanserin and the psychedelic is 4-Acetoxy-DMT, wherein the pruvanserin is administered to pretreat at least 120 minutes prior to the 4-Acetoxy-DMT. In some embodiments, the serotonin receptor modulator is pruvanserin and the psychedelic is 4-Acetoxy-DMT, wherein the pruvanserin is administered to pretreat at least 150 minutes prior to the 4-Acetoxy-DMT. In some embodiments, the serotonin receptor modulator is pruvanserin and the psychedelic is 4-Acetoxy-DMT, wherein the pruvanserin is administered to pretreat between about 15 minutes and about 150 minutes prior to the 4-Acetoxy-DMT. In some embodiments, the serotonin receptor modulator is pruvanserin and the psychedelic is 4-Acetoxy-DMT, wherein the pruvanserin is administered to pretreat at least 180 minutes prior to the 4-Acetoxy-DMT. In some embodiments, the serotonin receptor modulator is pruvanserin and the psychedelic is 4-Acetoxy-DMT, wherein the pruvanserin is administered to pretreat at least 210 minutes prior to the 4-Acetoxy-DMT. In some embodiments, the serotonin receptor modulator is pruvanserin and the psychedelic is 4-Acetoxy-DMT, wherein the pruvanserin is administered to pretreat at least 240 minutes prior to the 4-Acetoxy-DMT. In some embodiments, the serotonin receptor modulator is pruvanserin and the psychedelic is 4-Acetoxy-DMT, wherein the pruvanserin is administered to pretreat at least 270 minutes prior to the 4-Acetoxy-DMT. In some embodiments, the serotonin receptor modulator is pruvanserin and the psychedelic is 4-Acetoxy-DMT, wherein the pruvanserin is administered to pretreat at least 300 minutes prior to the 4-Acetoxy-DMT. In some embodiments, the serotonin receptor modulator is pruvanserin and the psychedelic is 4-Acetoxy-DMT, wherein the pruvanserin is administered to pretreat at least 330 minutes prior to the 4-Acetoxy-DMT. In some embodiments, the serotonin receptor modulator is pruvanserin and the psychedelic is 4-Acetoxy-DMT, wherein the pruvanserin is administered to pretreat at least 360 minutes prior to the 4-Acetoxy-DMT. In some preferred embodiments, the serotonin receptor modulator is pruvanserin and the psychedelic is 4-Acetoxy-DMT, wherein pruvanserin is administered to pretreat between about 60 minutes and about 180 minutes prior to the administration of 4-Acetoxy-DMT.

In some embodiments, the serotonin receptor modulator is eplivanserin and the psychedelic is LSD, wherein the eplivanserin is administered to pretreat at least 15 minutes prior to the administration of LSD. In some embodiments, the serotonin receptor modulator is eplivanserin and the psychedelic is LSD, wherein the eplivanserin is administered to pretreat at least 30 minutes prior to the LSD. In some embodiments, the serotonin receptor modulator is eplivanserin and the psychedelic is LSD, wherein the eplivanserin is administered to pretreat at least 60 minutes prior to the LSD. In some embodiments, the serotonin receptor modulator is eplivanserin and the psychedelic is LSD, wherein the eplivanserin is administered to pretreat at least 90 minutes prior to the LSD. In some embodiments, the serotonin receptor modulator is eplivanserin and the psychedelic is LSD, wherein the eplivanserin is administered to pretreat at least 120 minutes prior to the LSD. In some embodiments, the serotonin receptor modulator is eplivanserin and the psychedelic is LSD, wherein the eplivanserin is administered to pretreat at least 150 minutes prior to the LSD. In some embodiments, the serotonin receptor modulator is eplivanserin and the psychedelic is LSD, wherein the eplivanserin is administered to pretreat at least 180 minutes prior to the LSD. In some embodiments, the serotonin receptor modulator is eplivanserin and the psychedelic is LSD, wherein the eplivanserin is administered to pretreat at least 210 minutes prior to the LSD. In some embodiments, the serotonin receptor modulator is eplivanserin and the psychedelic is LSD, wherein the eplivanserin is administered to pretreat at least 240 minutes prior to the LSD. In some embodiments, the serotonin receptor modulator is eplivanserin and the psychedelic is LSD, wherein the eplivanserin is administered to pretreat at least 270 minutes prior to the LSD. In some embodiments, the serotonin receptor modulator is eplivanserin and the psychedelic is LSD, wherein the eplivanserin is administered to pretreat at least 300 minutes prior to the LSD. In some embodiments, the serotonin receptor modulator is eplivanserin and the psychedelic is LSD, wherein the eplivanserin is administered to pretreat at least 330 minutes prior to the LSD. In some embodiments, the serotonin receptor modulator is eplivanserin and the psychedelic is LSD, wherein the eplivanserin is administered to pretreat at least 360 minutes prior to the LSD.

In some preferred embodiments, the serotonin receptor modulator is eplivanserin and the psychedelic is LSD, wherein eplivanserin is administered to pretreat between about 60 minutes and about 180 minutes prior to the administration of LSD.

In some embodiments, the serotonin receptor modulator is volinanserin and the psychedelic is LSD, wherein the volinanserin is administered to pretreat at least 15 minutes prior to the administration of LSD. In some embodiments, the serotonin receptor modulator is volinanserin and the psychedelic is LSD, wherein the volinanserin is administered to pretreat at least 30 minutes prior to the LSD. In some embodiments, the serotonin receptor modulator is volinanserin and the psychedelic is LSD, wherein the volinanserin is administered to pretreat at least 60 minutes prior to the LSD. In some embodiments, the serotonin receptor modulator is volinanserin and the psychedelic is LSD, wherein the volinanserin is administered to pretreat at least 90 minutes prior to the LSD. In some embodiments, the serotonin receptor modulator is volinanserin and the psychedelic is LSD, wherein the volinanserin is administered to pretreat at least 120 minutes prior to the LSD. In some embodiments, the serotonin receptor modulator is volinanserin and the psychedelic is LSD, wherein the volinanserin is administered to pretreat at least 150 minutes prior to the LSD. In some embodiments, the serotonin receptor modulator is volinanserin and the psychedelic is LSD, wherein the volinanserin is administered to pretreat between about 15 minutes and about 150 minutes prior to the LSD. In some embodiments, the serotonin receptor modulator is volinanserin and the psychedelic is LSD, wherein the volinanserin is administered to pretreat at least 180 minutes prior to the LSD. In some embodiments, the serotonin receptor modulator is volinanserin and the psychedelic is LSD, wherein the volinanserin is administered to pretreat at least 210 minutes prior to the LSD. In some embodiments, the serotonin receptor modulator is volinanserin and the psychedelic is LSD, wherein the volinanserin is administered to pretreat at least 240 minutes prior to the LSD. In some embodiments, the serotonin receptor modulator is volinanserin and the psychedelic is LSD, wherein the volinanserin is administered to pretreat at least 270 minutes prior to the LSD. In some embodiments, the serotonin receptor modulator is volinanserin and the psychedelic is LSD, wherein the volinanserin is administered to pretreat at least 300 minutes prior to the LSD. In some embodiments, the serotonin receptor modulator is volinanserin and the psychedelic is LSD, wherein the volinanserin is administered to pretreat at least 330 minutes prior to the LSD. In some embodiments, the serotonin receptor modulator is volinanserin and the psychedelic is LSD, wherein the volinanserin is administered to pretreat at least 360 minutes prior to the LSD.

In some preferred embodiments, the serotonin receptor modulator is volinanserin and the psychedelic is LSD, wherein volinanserin is administered to pretreat between about 60 minutes and about 180 minutes prior to the administration of LSD.

In some embodiments, the serotonin receptor modulator is ketanserin and the psychedelic is LSD, wherein the ketanserin is administered to pretreat at least 15 minutes prior to the administration of LSD. In some embodiments, the serotonin receptor modulator is ketanserin and the psychedelic is LSD, wherein the ketanserin is administered to pretreat at least 30 minutes prior to the LSD. In some embodiments, the serotonin receptor modulator is ketanserin and the psychedelic is LSD, wherein the ketanserin is administered to pretreat at least 60 minutes prior to the LSD. In some embodiments, the serotonin receptor modulator is ketanserin and the psychedelic is LSD, wherein the ketanserin is administered to pretreat at least 90 minutes prior to the LSD. In some embodiments, the serotonin receptor modulator is ketanserin and the psychedelic is LSD, wherein the ketanserin is administered to pretreat at least 120 minutes prior to the LSD. In some embodiments, the serotonin receptor modulator is ketanserin and the psychedelic is LSD, wherein the ketanserin is administered to pretreat at least 150 minutes prior to the LSD. In some embodiments, the serotonin receptor modulator is ketanserin and the psychedelic is LSD, wherein the ketanserin is administered to pretreat between about 15 minutes and about 150 minutes prior to the LSD. In some embodiments, the serotonin receptor modulator is ketanserin and the psychedelic is LSD, wherein the ketanserin is administered to pretreat at least 180 minutes prior to the LSD. In some embodiments, the serotonin receptor modulator is ketanserin and the psychedelic is LSD, wherein the ketanserin is administered to pretreat at least 210 minutes prior to the LSD. In some embodiments, the serotonin receptor modulator is ketanserin and the psychedelic is LSD, wherein the ketanserin is administered to pretreat at least 240 minutes prior to the LSD. In some embodiments, the serotonin receptor modulator is ketanserin and the psychedelic is LSD, wherein the ketanserin is administered to pretreat at least 270 minutes prior to the LSD. In some embodiments, the serotonin receptor modulator is ketanserin and the psychedelic is LSD, wherein the ketanserin is administered to pretreat at least 300 minutes prior to the LSD. In some embodiments, the serotonin receptor modulator is ketanserin and the psychedelic is LSD, wherein the ketanserin is administered to pretreat at least 330 minutes prior to the LSD. In some embodiments, the serotonin receptor modulator is ketanserin and the psychedelic is LSD, wherein the ketanserin is administered to pretreat at least 360 minutes prior to the LSD. In some preferred embodiments, the serotonin receptor modulator is ketanserin and the psychedelic is LSD, wherein ketanserin is administered to pretreat between about 60 minutes and about 180 minutes prior to the administration of LSD.

In some embodiments, the serotonin receptor modulator is ritanserin and the psychedelic is LSD, wherein the ritanserin is administered to pretreat at least 15 minutes prior to the administration of LSD. In some embodiments, the serotonin receptor modulator is ritanserin and the psychedelic is LSD, wherein the ritanserin is administered to pretreat at least 30 minutes prior to the LSD. In some embodiments, the serotonin receptor modulator is ritanserin and the psychedelic is LSD, wherein the ritanserin is administered to pretreat at least 60 minutes prior to the LSD. In some embodiments, the serotonin receptor modulator is ritanserin and the psychedelic is LSD, wherein the ritanserin is administered to pretreat at least 90 minutes prior to the LSD. In some embodiments, the serotonin receptor modulator is ritanserin and the psychedelic is LSD, wherein the ritanserin is administered to pretreat at least 120 minutes prior to the LSD. In some embodiments, the serotonin receptor modulator is ritanserin and the psychedelic is LSD, wherein the ritanserin is administered to pretreat at least 150 minutes prior to the LSD. In some embodiments, the serotonin receptor modulator is ritanserin and the psychedelic is LSD, wherein the ritanserin is administered to pretreat between about 15 minutes and about 150 minutes prior to the LSD. In some embodiments, the serotonin receptor modulator is ritanserin and the psychedelic is LSD, wherein the ritanserin is administered to pretreat at least 180 minutes prior to the LSD. In some embodiments, the serotonin receptor modulator is ritanserin and the psychedelic is LSD, wherein the ritanserin is administered to pretreat at least 210 minutes prior to the LSD. In some embodiments, the serotonin receptor modulator is ritanserin and the psychedelic is LSD, wherein the ritanserin is administered to pretreat at least 240 minutes prior to the LSD. In some embodiments, the serotonin receptor modulator is ritanserin and the psychedelic is LSD, wherein the ritanserin is administered to pretreat at least 270 minutes prior to the LSD. In some embodiments, the serotonin receptor modulator is ritanserin and the psychedelic is LSD, wherein the ritanserin is administered to pretreat at least 300 minutes prior to the LSD. In some embodiments, the serotonin receptor modulator is ritanserin and the psychedelic is LSD, wherein the ritanserin is administered to pretreat at least 330 minutes prior to the LSD. In some embodiments, the serotonin receptor modulator is ritanserin and the psychedelic is LSD, wherein the ritanserin is administered to pretreat at least 360 minutes prior to the LSD. In some preferred embodiments, the serotonin receptor modulator is ritanserin and the psychedelic is LSD, wherein ritanserin is administered to pretreat between about 60 minutes and about 180 minutes prior to the administration of LSD.

In some embodiments, the serotonin receptor modulator is pimavanserin and the psychedelic is LSD, wherein the pimavanserin is administered to pretreat at least 15 minutes prior to the administration of LSD. In some embodiments, the serotonin receptor modulator is pimavanserin and the psychedelic is LSD, wherein the pimavanserin is administered to pretreat at least 30 minutes prior to the LSD. In some embodiments, the serotonin receptor modulator is pimavanserin and the psychedelic is LSD, wherein the pimavanserin is administered to pretreat at least 60 minutes prior to the LSD. In some embodiments, the serotonin receptor modulator is pimavanserin and the psychedelic is LSD, wherein the pimavanserin is administered to pretreat at least 90 minutes prior to the LSD. In some embodiments, the serotonin receptor modulator is pimavanserin and the psychedelic is LSD, wherein the pimavanserin is administered to pretreat at least 120 minutes prior to the LSD. In some embodiments, the serotonin receptor modulator is pimavanserin and the psychedelic is LSD, wherein the pimavanserin is administered to pretreat at least 150 minutes prior to the LSD. In some embodiments, the serotonin receptor modulator is pimavanserin and the psychedelic is LSD, wherein the pimavanserin is administered to pretreat between about 15 minutes and about 150 minutes prior to the LSD. In some embodiments, the serotonin receptor modulator is pimavanserin and the psychedelic is LSD, wherein the pimavanserin is administered to pretreat at least 180 minutes prior to the LSD. In some embodiments, the serotonin receptor modulator is pimavanserin and the psychedelic is LSD, wherein the pimavanserin is administered to pretreat at least 210 minutes prior to the LSD. In some embodiments, the serotonin receptor modulator is pimavanserin and the psychedelic is LSD, wherein the pimavanserin is administered to pretreat at least 240 minutes prior to the LSD. In some embodiments, the serotonin receptor modulator is pimavanserin and the psychedelic is LSD, wherein the pimavanserin is administered to pretreat at least 270 minutes prior to the LSD. In some embodiments, the serotonin receptor modulator is pimavanserin and the psychedelic is LSD, wherein the pimavanserin is administered to pretreat at least 300 minutes prior to the LSD. In some embodiments, the serotonin receptor modulator is pimavanserin and the psychedelic is LSD, wherein the pimavanserin is administered to pretreat at least 330 minutes prior to the LSD. In some embodiments, the serotonin receptor modulator is pimavanserin and the psychedelic is LSD, wherein the pimavanserin is administered to pretreat at least 360 minutes prior to the LSD. In some preferred embodiments, the serotonin receptor modulator is pimavanserin and the psychedelic is LSD, wherein pimavanserin is administered to pretreat between about 60 minutes and about 180 minutes prior to the administration of LSD.

In some embodiments, the serotonin receptor modulator is nelotanserin and the psychedelic is LSD, wherein the nelotanserin is administered to pretreat at least 15 minutes prior to the administration of LSD. In some embodiments, the serotonin receptor modulator is nelotanserin and the psychedelic is LSD, wherein the nelotanserin is administered to pretreat at least 30 minutes prior to the LSD. In some embodiments, the serotonin receptor modulator is nelotanserin and the psychedelic is LSD, wherein the nelotanserin is administered to pretreat at least 60 minutes prior to the LSD. In some embodiments, the serotonin receptor modulator is nelotanserin and the psychedelic is LSD, wherein the nelotanserin is administered to pretreat at least 90 minutes prior to the LSD. In some embodiments, the serotonin receptor modulator is nelotanserin and the psychedelic is LSD, wherein the nelotanserin is administered to pretreat at least 120 minutes prior to the LSD. In some embodiments, the serotonin receptor modulator is nelotanserin and the psychedelic is LSD, wherein the nelotanserin is administered to pretreat at least 150 minutes prior to the LSD. In some embodiments, the serotonin receptor modulator is nelotanserin and the psychedelic is LSD, wherein the nelotanserin is administered to pretreat between about 15 minutes and about 150 minutes prior to the LSD. In some embodiments, the serotonin receptor modulator is nelotanserin and the psychedelic is LSD, wherein the nelotanserin is administered to pretreat at least 180 minutes prior to the LSD. In some embodiments, the serotonin receptor modulator is nelotanserin and the psychedelic is LSD, wherein the nelotanserin is administered to pretreat at least 210 minutes prior to the LSD. In some embodiments, the serotonin receptor modulator is nelotanserin and the psychedelic is LSD, wherein the nelotanserin is administered to pretreat at least 240 minutes prior to the LSD. In some embodiments, the serotonin receptor modulator is nelotanserin and the psychedelic is LSD, wherein the nelotanserin is administered to pretreat at least 270 minutes prior to the LSD. In some embodiments, the serotonin receptor modulator is nelotanserin and the psychedelic is LSD, wherein the nelotanserin is administered to pretreat at least 300 minutes prior to the LSD. In some embodiments, the serotonin receptor modulator is nelotanserin and the psychedelic is LSD, wherein the nelotanserin is administered to pretreat at least 330 minutes prior to the LSD. In some embodiments, the serotonin receptor modulator is nelotanserin and the psychedelic is LSD, wherein the nelotanserin is administered to pretreat at least 360 minutes prior to the LSD. In some preferred embodiments, the serotonin receptor modulator is nelotanserin and the psychedelic is LSD, wherein nelotanserin is administered to pretreat between about 60 minutes and about 180 minutes prior to the administration of LSD.

In some embodiments, the serotonin receptor modulator is pruvanserin and the psychedelic is LSD, wherein the pruvanserin is administered to pretreat at least 15 minutes prior to the administration of LSD. In some embodiments, the serotonin receptor modulator is pruvanserin and the psychedelic is LSD, wherein the pruvanserin is administered to pretreat at least 30 minutes prior to the LSD. In some embodiments, the serotonin receptor modulator is pruvanserin and the psychedelic is LSD, wherein the pruvanserin is administered to pretreat at least 60 minutes prior to the LSD. In some embodiments, the serotonin receptor modulator is pruvanserin and the psychedelic is LSD, wherein the pruvanserin is administered to pretreat at least 90 minutes prior to the LSD. In some embodiments, the serotonin receptor modulator is pruvanserin and the psychedelic is LSD, wherein the pruvanserin is administered to pretreat at least 120 minutes prior to the LSD. In some embodiments, the serotonin receptor modulator is pruvanserin and the psychedelic is LSD, wherein the pruvanserin is administered to pretreat at least 150 minutes prior to the LSD. In some embodiments, the serotonin receptor modulator is pruvanserin and the psychedelic is LSD, wherein the pruvanserin is administered to pretreat between about 15 minutes and about 150 minutes prior to the LSD. In some embodiments, the serotonin receptor modulator is pruvanserin and the psychedelic is LSD, wherein the pruvanserin is administered to pretreat at least 180 minutes prior to the LSD. In some embodiments, the serotonin receptor modulator is pruvanserin and the psychedelic is LSD, wherein the pruvanserin is administered to pretreat at least 210 minutes prior to the LSD. In some embodiments, the serotonin receptor modulator is pruvanserin and the psychedelic is LSD, wherein the pruvanserin is administered to pretreat at least 240 minutes prior to the LSD. In some embodiments, the serotonin receptor modulator is pruvanserin and the psychedelic is LSD, wherein the pruvanserin is administered to pretreat at least 270 minutes prior to the LSD. In some embodiments, the serotonin receptor modulator is pruvanserin and the psychedelic is LSD, wherein the pruvanserin is administered to pretreat at least 300 minutes prior to the LSD. In some embodiments, the serotonin receptor modulator is pruvanserin and the psychedelic is LSD, wherein the pruvanserin is administered to pretreat at least 330 minutes prior to the LSD. In some embodiments, the serotonin receptor modulator is pruvanserin and the psychedelic is LSD, wherein the pruvanserin is administered to pretreat at least 360 minutes prior to the LSD. In some preferred embodiments, the serotonin receptor modulator is pruvanserin and the psychedelic is LSD, wherein pruvanserin is administered to pretreat between about 60 minutes and about 180 minutes prior to the administration of LSD.

In some embodiments, the serotonin receptor modulator is eplivanserin and the psychedelic is ALD-52, wherein the eplivanserin is administered to pretreat at least 15 minutes prior to the administration of ALD-52. In some embodiments, the serotonin receptor modulator is eplivanserin and the psychedelic is ALD-52, wherein the eplivanserin is administered to pretreat at least 30 minutes prior to the ALD-52. In some embodiments, the serotonin receptor modulator is eplivanserin and the psychedelic is ALD-52, wherein the eplivanserin is administered to pretreat at least 60 minutes prior to the ALD-52. In some embodiments, the serotonin receptor modulator is eplivanserin and the psychedelic is ALD-52, wherein the eplivanserin is administered to pretreat at least 90 minutes prior to the ALD-52. In some embodiments, the serotonin receptor modulator is eplivanserin and the psychedelic is ALD-52, wherein the eplivanserin is administered to pretreat at least 120 minutes prior to the ALD-52. In some embodiments, the serotonin receptor modulator is eplivanserin and the psychedelic is ALD-52, wherein the eplivanserin is administered to pretreat at least 150 minutes prior to the ALD-52. In some embodiments, the serotonin receptor modulator is eplivanserin and the psychedelic is ALD-52, wherein the eplivanserin is administered to pretreat at least 180 minutes prior to the ALD-52. In some embodiments, the serotonin receptor modulator is eplivanserin and the psychedelic is ALD-52, wherein the eplivanserin is administered to pretreat at least 210 minutes prior to the ALD-52. In some embodiments, the serotonin receptor modulator is eplivanserin and the psychedelic is ALD-52, wherein the eplivanserin is administered to pretreat at least 240 minutes prior to the ALD-52. In some embodiments, the serotonin receptor modulator is eplivanserin and the psychedelic is ALD-52, wherein the eplivanserin is administered to pretreat at least 270 minutes prior to the ALD-52. In some embodiments, the serotonin receptor modulator is eplivanserin and the psychedelic is ALD-52, wherein the eplivanserin is administered to pretreat at least 300 minutes prior to the ALD-52. In some embodiments, the serotonin receptor modulator is eplivanserin and the psychedelic is ALD-52, wherein the eplivanserin is administered to pretreat at least 330 minutes prior to the ALD-52. In some embodiments, the serotonin receptor modulator is eplivanserin and the psychedelic is ALD-52, wherein the eplivanserin is administered to pretreat at least 360 minutes prior to the ALD-52.

In some preferred embodiments, the serotonin receptor modulator is eplivanserin and the psychedelic is ALD-52, wherein eplivanserin is administered to pretreat between about 60 minutes and about 180 minutes prior to the administration of ALD-52.

In some embodiments, the serotonin receptor modulator is volinanserin and the psychedelic is ALD-52, wherein the volinanserin is administered to pretreat at least 15 minutes prior to the ALD-52. In some embodiments, the serotonin receptor modulator is volinanserin and the psychedelic is ALD-52, wherein the volinanserin is administered to pretreat at least 30 minutes prior to the ALD-52. In some embodiments, the serotonin receptor modulator is volinanserin and the psychedelic is ALD-52, wherein the volinanserin is administered to pretreat at least 60 minutes prior to the ALD-52. In some embodiments, the serotonin receptor modulator is volinanserin and the psychedelic is ALD-52, wherein the volinanserin is administered to pretreat at least 90 minutes prior to the ALD-52. In some embodiments, the serotonin receptor modulator is volinanserin and the psychedelic is ALD-52, wherein the volinanserin is administered to pretreat at least 120 minutes prior to the ALD-52. In some embodiments, the serotonin receptor modulator is volinanserin and the psychedelic is ALD-52, wherein the volinanserin is administered to pretreat at least 150 minutes prior to the ALD-52. In some embodiments, the serotonin receptor modulator is volinanserin and the psychedelic is ALD-52, wherein the volinanserin is administered to pretreat between about 15 minutes and about 150 minutes prior to the ALD-52. In some embodiments, the serotonin receptor modulator is volinanserin and the psychedelic is ALD-52, wherein the volinanserin is administered to pretreat at least 180 minutes prior to the ALD-52. In some embodiments, the serotonin receptor modulator is volinanserin and the psychedelic is ALD-52, wherein the volinanserin is administered to pretreat at least 210 minutes prior to the ALD-52. In some embodiments, the serotonin receptor modulator is volinanserin and the psychedelic is ALD-52, wherein the volinanserin is administered to pretreat at least 240 minutes prior to the ALD-52. In some embodiments, the serotonin receptor modulator is volinanserin and the psychedelic is ALD-52, wherein the volinanserin is administered to pretreat at least 270 minutes prior to the ALD-52. In some embodiments, the serotonin receptor modulator is volinanserin and the psychedelic is ALD-52, wherein the volinanserin is administered to pretreat at least 300 minutes prior to the ALD-52. In some embodiments, the serotonin receptor modulator is volinanserin and the psychedelic is ALD-52, wherein the volinanserin is administered to pretreat at least 330 minutes prior to the ALD-52. In some embodiments, the serotonin receptor modulator is volinanserin and the psychedelic is ALD-52, wherein the volinanserin is administered to pretreat at least 360 minutes prior to the ALD-52. In some preferred embodiments, the serotonin receptor modulator is volinanserin and the psychedelic is ALD-52, wherein volinanserin is administered to pretreat between about 60 minutes and about 180 minutes prior to the administration of ALD-52.

In some embodiments, the serotonin receptor modulator is ketanserin and the psychedelic is ALD-52, wherein the ketanserin is administered to pretreat at least 15 minutes prior to the administration of ALD-52. In some embodiments, the serotonin receptor modulator is ketanserin and the psychedelic is ALD-52, wherein the ketanserin is administered to pretreat at least 30 minutes prior to the ALD-52. In some embodiments, the serotonin receptor modulator is ketanserin and the psychedelic is ALD-52, wherein the ketanserin is administered to pretreat at least 60 minutes prior to the ALD-52. In some embodiments, the serotonin receptor modulator is ketanserin and the psychedelic is ALD-52, wherein the ketanserin is administered to pretreat at least 90 minutes prior to the ALD-52. In some embodiments, the serotonin receptor modulator is ketanserin and the psychedelic is ALD-52, wherein the ketanserin is administered to pretreat at least 120 minutes prior to the ALD-52. In some embodiments, the serotonin receptor modulator is ketanserin and the psychedelic is ALD-52, wherein the ketanserin is administered to pretreat at least 150 minutes prior to the ALD-52. In some embodiments, the serotonin receptor modulator is ketanserin and the psychedelic is ALD-52, wherein the ketanserin is administered to pretreat between about 15 minutes and about 150 minutes prior to the ALD-52. In some embodiments, the serotonin receptor modulator is ketanserin and the psychedelic is ALD-52, wherein the ketanserin is administered to pretreat at least 180 minutes prior to the ALD-52. In some embodiments, the serotonin receptor modulator is ketanserin and the psychedelic is ALD-52, wherein the ketanserin is administered to pretreat at least 210 minutes prior to the ALD-52. In some embodiments, the serotonin receptor modulator is ketanserin and the psychedelic is ALD-52, wherein the ketanserin is administered to pretreat at least 240 minutes prior to the ALD-52. In some embodiments, the serotonin receptor modulator is ketanserin and the psychedelic is ALD-52, wherein the ketanserin is administered to pretreat at least 270 minutes prior to the ALD-52. In some embodiments, the serotonin receptor modulator is ketanserin and the psychedelic is ALD-52, wherein the ketanserin is administered to pretreat at least 300 minutes prior to the ALD-52. In some embodiments, the serotonin receptor modulator is ketanserin and the psychedelic is ALD-52, wherein the ketanserin is administered to pretreat at least 330 minutes prior to the ALD-52. In some embodiments, the serotonin receptor modulator is ketanserin and the psychedelic is ALD-52, wherein the ketanserin is administered to pretreat at least 360 minutes prior to the ALD-52. In some preferred embodiments, the serotonin receptor modulator is ketanserin and the psychedelic is ALD-52, wherein ketanserin is administered to pretreat between about 60 minutes and about 180 minutes prior to the administration of ALD-52.

In some embodiments, the serotonin receptor modulator is ritanserin and the psychedelic is ALD-52, wherein the ritanserin is administered to pretreat at least 15 minutes prior to the ALD-52. In some embodiments, the serotonin receptor modulator is ritanserin and the psychedelic is ALD-52, wherein the ritanserin is administered to pretreat at least 30 minutes prior to the ALD-52. In some embodiments, the serotonin receptor modulator is ritanserin and the psychedelic is ALD-52, wherein the ritanserin is administered to pretreat at least 60 minutes prior to the ALD-52. In some embodiments, the serotonin receptor modulator is ritanserin and the psychedelic is ALD-52, wherein the ritanserin is administered to pretreat at least 90 minutes prior to the ALD-52. In some embodiments, the serotonin receptor modulator is ritanserin and the psychedelic is ALD-52, wherein the ritanserin is administered to pretreat at least 120 minutes prior to the ALD-52. In some embodiments, the serotonin receptor modulator is ritanserin and the psychedelic is ALD-52, wherein the ritanserin is administered to pretreat at least 150 minutes prior to the ALD-52. In some embodiments, the serotonin receptor modulator is ritanserin and the psychedelic is ALD-52, wherein the ritanserin is administered to pretreat between about 15 minutes and about 150 minutes prior to the ALD-52. In some embodiments, the serotonin receptor modulator is ritanserin and the psychedelic is ALD-52, wherein the ritanserin is administered to pretreat at least 180 minutes prior to the ALD-52. In some embodiments, the serotonin receptor modulator is ritanserin and the psychedelic is ALD-52, wherein the ritanserin is administered to pretreat at least 210 minutes prior to the ALD-52. In some embodiments, the serotonin receptor modulator is ritanserin and the psychedelic is ALD-52, wherein the ritanserin is administered to pretreat at least 240 minutes prior to the ALD-52. In some embodiments, the serotonin receptor modulator is ritanserin and the psychedelic is ALD-52, wherein the ritanserin is administered to pretreat at least 270 minutes prior to the ALD-52. In some embodiments, the serotonin receptor modulator is ritanserin and the psychedelic is ALD-52, wherein the ritanserin is administered to pretreat at least 300 minutes prior to the ALD-52. In some embodiments, the serotonin receptor modulator is ritanserin and the psychedelic is ALD-52, wherein the ritanserin is administered to pretreat at least 330 minutes prior to the ALD-52. In some embodiments, the serotonin receptor modulator is ritanserin and the psychedelic is ALD-52, wherein the ritanserin is administered to pretreat at least 360 minutes prior to the ALD-52. In some preferred embodiments, the serotonin receptor modulator is ritanserin and the psychedelic is ALD-52, wherein ritanserin is administered to pretreat between about 60 minutes and about 180 minutes prior to the administration of ALD-52.

In some embodiments, the serotonin receptor modulator is pimavanserin and the psychedelic is ALD-52, wherein the pimavanserin is administered to pretreat at least 15 minutes prior to the ALD-52. In some embodiments, the serotonin receptor modulator is pimavanserin and the psychedelic is ALD-52, wherein the pimavanserin is administered to pretreat at least 30 minutes prior to the ALD-52. In some embodiments, the serotonin receptor modulator is pimavanserin and the psychedelic is ALD-52, wherein the pimavanserin is administered to pretreat at least 60 minutes prior to the ALD-52. In some embodiments, the serotonin receptor modulator is pimavanserin and the psychedelic is ALD-52, wherein the pimavanserin is administered to pretreat at least 90 minutes prior to the ALD-52. In some embodiments, the serotonin receptor modulator is pimavanserin and the psychedelic is ALD-52, wherein the pimavanserin is administered to pretreat at least 120 minutes prior to the ALD-52. In some embodiments, the serotonin receptor modulator is pimavanserin and the psychedelic is ALD-52, wherein the pimavanserin is administered to pretreat at least 150 minutes prior to the ALD-52. In some embodiments, the serotonin receptor modulator is pimavanserin and the psychedelic is ALD-52, wherein the pimavanserin is administered to pretreat between about 15 minutes and about 150 minutes prior to the ALD-52. In some embodiments, the serotonin receptor modulator is pimavanserin and the psychedelic is ALD-52, wherein the pimavanserin is administered to pretreat at least 180 minutes prior to the ALD-52. In some embodiments, the serotonin receptor modulator is pimavanserin and the psychedelic is ALD-52, wherein the pimavanserin is administered to pretreat at least 210 minutes prior to the ALD-52. In some embodiments, the serotonin receptor modulator is pimavanserin and the psychedelic is ALD-52, wherein the pimavanserin is administered to pretreat at least 240 minutes prior to the ALD-52. In some embodiments, the serotonin receptor modulator is pimavanserin and the psychedelic is ALD-52, wherein the pimavanserin is administered to pretreat at least 270 minutes prior to the ALD-52. In some embodiments, the serotonin receptor modulator is pimavanserin and the psychedelic is ALD-52, wherein the pimavanserin is administered to pretreat at least 300 minutes prior to the ALD-52. In some embodiments, the serotonin receptor modulator is pimavanserin and the psychedelic is ALD-52, wherein the pimavanserin is administered to pretreat at least 330 minutes prior to the ALD-52. In some embodiments, the serotonin receptor modulator is pimavanserin and the psychedelic is ALD-52, wherein the pimavanserin is administered to pretreat at least 360 minutes prior to the ALD-52. In some preferred embodiments, the serotonin receptor modulator is pimavanserin and the psychedelic is ALD-52, wherein pimavanserin is administered to pretreat between about 60 minutes and about 180 minutes prior to the administration of ALD-52.

In some embodiments, the serotonin receptor modulator is nelotanserin and the psychedelic is ALD-52, wherein the nelotanserin is administered to pretreat at least 15 minutes prior to the ALD-52. In some embodiments, the serotonin receptor modulator is nelotanserin and the psychedelic is ALD-52, wherein the nelotanserin is administered to pretreat at least 30 minutes prior to the ALD-52. In some embodiments, the serotonin receptor modulator is nelotanserin and the psychedelic is ALD-52, wherein the nelotanserin is administered to pretreat at least 60 minutes prior to the ALD-52. In some embodiments, the serotonin receptor modulator is nelotanserin and the psychedelic is ALD-52, wherein the nelotanserin is administered to pretreat at least 90 minutes prior to the ALD-52. In some embodiments, the serotonin receptor modulator is nelotanserin and the psychedelic is ALD-52, wherein the nelotanserin is administered to pretreat at least 120 minutes prior to the ALD-52. In some embodiments, the serotonin receptor modulator is nelotanserin and the psychedelic is ALD-52, wherein the nelotanserin is administered to pretreat at least 150 minutes prior to the ALD-52. In some embodiments, the serotonin receptor modulator is nelotanserin and the psychedelic is ALD-52, wherein the nelotanserin is administered to pretreat between about 15 minutes and about 150 minutes prior to the ALD-52. In some embodiments, the serotonin receptor modulator is nelotanserin and the psychedelic is ALD-52, wherein the nelotanserin is administered to pretreat at least 180 minutes prior to the ALD-52. In some embodiments, the serotonin receptor modulator is nelotanserin and the psychedelic is ALD-52, wherein the nelotanserin is administered to pretreat at least 210 minutes prior to the ALD-52. In some embodiments, the serotonin receptor modulator is nelotanserin and the psychedelic is ALD-52, wherein the nelotanserin is administered to pretreat at least 240 minutes prior to the ALD-52. In some embodiments, the serotonin receptor modulator is nelotanserin and the psychedelic is ALD-52, wherein the nelotanserin is administered to pretreat at least 270 minutes prior to the ALD-52. In some embodiments, the serotonin receptor modulator is nelotanserin and the psychedelic is ALD-52, wherein the nelotanserin is administered to pretreat at least 300 minutes prior to the ALD-52. In some embodiments, the serotonin receptor modulator is nelotanserin and the psychedelic is ALD-52, wherein the nelotanserin is administered to pretreat at least 330 minutes prior to the ALD-52. In some embodiments, the serotonin receptor modulator is nelotanserin and the psychedelic is ALD-52, wherein the nelotanserin is administered to pretreat at least 360 minutes prior to the ALD-52. In some preferred embodiments, the serotonin receptor modulator is nelotanserin and the psychedelic is ALD-52, wherein nelotanserin is administered to pretreat between about 60 minutes and about 180 minutes prior to the administration of ALD-52.

In some embodiments, the serotonin receptor modulator is pruvanserin and the psychedelic is ALD-52, wherein the pruvanserin is administered to pretreat at least 15 minutes prior to the ALD-52. In some embodiments, the serotonin receptor modulator is pruvanserin and the psychedelic is ALD-52, wherein the pruvanserin is administered to pretreat at least 30 minutes prior to the ALD-52. In some embodiments, the serotonin receptor modulator is pruvanserin and the psychedelic is ALD-52, wherein the pruvanserin is administered to pretreat at least 60 minutes prior to the ALD-52. In some embodiments, the serotonin receptor modulator is pruvanserin and the psychedelic is ALD-52, wherein the pruvanserin is administered to pretreat at least 90 minutes prior to the ALD-52. In some embodiments, the serotonin receptor modulator is pruvanserin and the psychedelic is ALD-52, wherein the pruvanserin is administered to pretreat at least 120 minutes prior to the ALD-52. In some embodiments, the serotonin receptor modulator is pruvanserin and the psychedelic is ALD-52, wherein the pruvanserin is administered to pretreat at least 150 minutes prior to the ALD-52. In some embodiments, the serotonin receptor modulator is pruvanserin and the psychedelic is ALD-52, wherein the pruvanserin is administered to pretreat between about 15 minutes and about 150 minutes prior to the ALD-52. In some embodiments, the serotonin receptor modulator is pruvanserin and the psychedelic is ALD-52, wherein the pruvanserin is administered to pretreat at least 180 minutes prior to the ALD-52. In some embodiments, the serotonin receptor modulator is pruvanserin and the psychedelic is ALD-52, wherein the pruvanserin is administered to pretreat at least 210 minutes prior to the ALD-52. In some embodiments, the serotonin receptor modulator is pruvanserin and the psychedelic is ALD-52, wherein the pruvanserin is administered to pretreat at least 240 minutes prior to the ALD-52. In some embodiments, the serotonin receptor modulator is pruvanserin and the psychedelic is ALD-52, wherein the pruvanserin is administered to pretreat at least 270 minutes prior to the ALD-52. In some embodiments, the serotonin receptor modulator is pruvanserin and the psychedelic is ALD-52, wherein the pruvanserin is administered to pretreat at least 300 minutes prior to the ALD-52. In some embodiments, the serotonin receptor modulator is pruvanserin and the psychedelic is ALD-52, wherein the pruvanserin is administered to pretreat at least 330 minutes prior to the ALD-52. In some embodiments, the serotonin receptor modulator is pruvanserin and the psychedelic is ALD-52, wherein the pruvanserin is administered to pretreat at least 360 minutes prior to the ALD-52. In some preferred embodiments, the serotonin receptor modulator is pruvanserin and the psychedelic is ALD-52, wherein pruvanserin is administered to pretreat between about 60 minutes and about 180 minutes prior to the administration of ALD-52.

In some embodiments, the serotonin receptor modulator is eplivanserin and the psychedelic is 1P-LSD, wherein the eplivanserin is administered to pretreat at least 15 minutes prior to the administration of 1P-LSD. In some embodiments, the serotonin receptor modulator is eplivanserin and the psychedelic is IP-LSD, wherein the eplivanserin is administered to pretreat at least 30 minutes prior to the 1P-LSD. In some embodiments, the serotonin receptor modulator is eplivanserin and the psychedelic is 1P-LSD, wherein the eplivanserin is administered to pretreat at least 60 minutes prior to the 1P-LSD. In some embodiments, the serotonin receptor modulator is eplivanserin and the psychedelic is IP-LSD, wherein the eplivanserin is administered to pretreat at least 90 minutes prior to the IP-LSD. In some embodiments, the serotonin receptor modulator is eplivanserin and the psychedelic is IP-LSD, wherein the eplivanserin is administered to pretreat at least 120 minutes prior to the IP-LSD. In some embodiments, the serotonin receptor modulator is eplivanserin and the psychedelic is 1P-LSD, wherein the eplivanserin is administered to pretreat at least 150 minutes prior to the IP-LSD. In some embodiments, the serotonin receptor modulator is eplivanserin and the psychedelic is 1P-LSD, wherein the eplivanserin is administered to pretreat at least 180 minutes prior to the 1P-LSD. In some embodiments, the serotonin receptor modulator is eplivanserin and the psychedelic is 1P-LSD, wherein the eplivanserin is administered to pretreat at least 210 minutes prior to the IP-LSD. In some embodiments, the serotonin receptor modulator is eplivanserin and the psychedelic is IP-LSD, wherein the eplivanserin is administered to pretreat at least 240 minutes prior to the IP-LSD. In some embodiments, the serotonin receptor modulator is eplivanserin and the psychedelic is IP-LSD, wherein the eplivanserin is administered to pretreat at least 270 minutes prior to the IP-LSD. In some embodiments, the serotonin receptor modulator is eplivanserin and the psychedelic is 1P-LSD, wherein the eplivanserin is administered to pretreat at least 300 minutes prior to the IP-LSD. In some embodiments, the serotonin receptor modulator is eplivanserin and the psychedelic is IP-LSD, wherein the eplivanserin is administered to pretreat at least 330 minutes prior to the 1P-LSD. In some embodiments, the serotonin receptor modulator is eplivanserin and the psychedelic is IP-LSD, wherein the eplivanserin is administered to pretreat at least 360 minutes prior to the IP-LSD.

In some preferred embodiments, the serotonin receptor modulator is eplivanserin and the psychedelic is 1P-LSD, wherein eplivanserin is administered to pretreat between about 60 minutes and about 180 minutes prior to the administration of IP-LSD.

In some embodiments, the serotonin receptor modulator is volinanserin and the psychedelic is 1P-LSD, wherein the volinanserin is administered to pretreat at least 15 minutes prior to the IP-LSD. In some embodiments, the serotonin receptor modulator is volinanserin and the psychedelic is IP-LSD, wherein the volinanserin is administered to pretreat at least 30 minutes prior to the IP-LSD. In some embodiments, the serotonin receptor modulator is volinanserin and the psychedelic is 1P-LSD, wherein the volinanserin is administered to pretreat at least 60 minutes prior to the IP-LSD. In some embodiments, the serotonin receptor modulator is volinanserin and the psychedelic is 1P-LSD, wherein the volinanserin is administered to pretreat at least 90 minutes prior to the IP-LSD. In some embodiments, the serotonin receptor modulator is volinanserin and the psychedelic is IP-LSD, wherein the volinanserin is administered to pretreat at least 120 minutes prior to the IP-LSD. In some embodiments, the serotonin receptor modulator is volinanserin and the psychedelic is 1P-LSD, wherein the volinanserin is administered to pretreat at least 150 minutes prior to the IP-LSD. In some embodiments, the serotonin receptor modulator is volinanserin and the psychedelic is IP-LSD, wherein the volinanserin is administered to pretreat between about 15 minutes and about 150 minutes prior to the IP-LSD. In some embodiments, the serotonin receptor modulator is volinanserin and the psychedelic is 1P-LSD, wherein the volinanserin is administered to pretreat at least 180 minutes prior to the 1P-LSD. In some embodiments, the serotonin receptor modulator is volinanserin and the psychedelic is IP-LSD, wherein the volinanserin is administered to pretreat at least 210 minutes prior to the IP-LSD. In some embodiments, the serotonin receptor modulator is volinanserin and the psychedelic is IP-LSD, wherein the volinanserin is administered to pretreat at least 240 minutes prior to the IP-LSD. In some embodiments, the serotonin receptor modulator is volinanserin and the psychedelic is IP-LSD, wherein the volinanserin is administered to pretreat at least 270 minutes prior to the IP-LSD. In some embodiments, the serotonin receptor modulator is volinanserin and the psychedelic is 1P-LSD, wherein the volinanserin is administered to pretreat at least 300 minutes prior to the IP-LSD. In some embodiments, the serotonin receptor modulator is volinanserin and the psychedelic is 1P-LSD, wherein the volinanserin is administered to pretreat at least 330 minutes prior to the IP-LSD. In some embodiments, the serotonin receptor modulator is volinanserin and the psychedelic is 1P-LSD, wherein the volinanserin is administered to pretreat at least 360 minutes prior to the IP-LSD. In some preferred embodiments, the serotonin receptor modulator is volinanserin and the psychedelic is IP-LSD, wherein volinanserin is administered to pretreat between about 60 minutes and about 180 minutes prior to the administration of 1P-LSD.

In some embodiments, the serotonin receptor modulator is ketanserin and the psychedelic is 1P-LSD, wherein the ketanserin is administered to pretreat at least 15 minutes prior to the administration of IP-LSD. In some embodiments, the serotonin receptor modulator is ketanserin and the psychedelic is 1P-LSD, wherein the ketanserin is administered to pretreat at least 30 minutes prior to the 1P-LSD. In some embodiments, the serotonin receptor modulator is ketanserin and the psychedelic is 1P-LSD, wherein the ketanserin is administered to pretreat at least 60 minutes prior to the 1P-LSD. In some embodiments, the serotonin receptor modulator is ketanserin and the psychedelic is 1P-LSD, wherein the ketanserin is administered to pretreat at least 90 minutes prior to the IP-LSD. In some embodiments, the serotonin receptor modulator is ketanserin and the psychedelic is 1P-LSD, wherein the ketanserin is administered to pretreat at least 120 minutes prior to the 1P-LSD. In some embodiments, the serotonin receptor modulator is ketanserin and the psychedelic is 1P-LSD, wherein the ketanserin is administered to pretreat at least 150 minutes prior to the 1P-LSD. In some embodiments, the serotonin receptor modulator is ketanserin and the psychedelic is 1P-LSD, wherein the ketanserin is administered to pretreat between about 15 minutes and about 150 minutes prior to the 1P-LSD. In some embodiments, the serotonin receptor modulator is ketanserin and the psychedelic is IP-LSD, wherein the ketanserin is administered to pretreat at least 180 minutes prior to the IP-LSD. In some embodiments, the serotonin receptor modulator is ketanserin and the psychedelic is 1P-LSD, wherein the ketanserin is administered to pretreat at least 210 minutes prior to the IP-LSD. In some embodiments, the serotonin receptor modulator is ketanserin and the psychedelic is IP-LSD, wherein the ketanserin is administered to pretreat at least 240 minutes prior to the IP-LSD. In some embodiments, the serotonin receptor modulator is ketanserin and the psychedelic is 1P-LSD, wherein the ketanserin is administered to pretreat at least 270 minutes prior to the IP-LSD. In some embodiments, the serotonin receptor modulator is ketanserin and the psychedelic is IP-LSD, wherein the ketanserin is administered to pretreat at least 300 minutes prior to the IP-LSD. In some embodiments, the serotonin receptor modulator is ketanserin and the psychedelic is 1P-LSD, wherein the ketanserin is administered to pretreat at least 330 minutes prior to the 1P-LSD. In some embodiments, the serotonin receptor modulator is ketanserin and the psychedelic is 1P-LSD, wherein the ketanserin is administered to pretreat at least 360 minutes prior to the IP-LSD. In some preferred embodiments, the serotonin receptor modulator is ketanserin and the psychedelic is 1P-LSD, wherein ketanserin is administered to pretreat between about 60 minutes and about 180 minutes prior to the administration of 1P-LSD.

In some embodiments, the serotonin receptor modulator is ritanserin and the psychedelic is 1P-LSD, wherein the ritanserin is administered to pretreat at least 15 minutes prior to the 1P-LSD. In some embodiments, the serotonin receptor modulator is ritanserin and the psychedelic is 1P-LSD, wherein the ritanserin is administered to pretreat at least 30 minutes prior to the 1P-LSD. In some embodiments, the serotonin receptor modulator is ritanserin and the psychedelic is 1P-LSD, wherein the ritanserin is administered to pretreat at least 60 minutes prior to the IP-LSD. In some embodiments, the serotonin receptor modulator is ritanserin and the psychedelic is 1P-LSD, wherein the ritanserin is administered to pretreat at least 90 minutes prior to the 1P-LSD. In some embodiments, the serotonin receptor modulator is ritanserin and the psychedelic is 1P-LSD, wherein the ritanserin is administered to pretreat at least 120 minutes prior to the 1P-LSD. In some embodiments, the serotonin receptor modulator is ritanserin and the psychedelic is 1P-LSD, wherein the ritanserin is administered to pretreat at least 150 minutes prior to the 1P-LSD. In some embodiments, the serotonin receptor modulator is ritanserin and the psychedelic is 1P-LSD, wherein the ritanserin is administered to pretreat between about 15 minutes and about 150 minutes prior to the IP-LSD. In some embodiments, the serotonin receptor modulator is ritanserin and the psychedelic is 1P-LSD, wherein the ritanserin is administered to pretreat at least 180 minutes prior to the 1P-LSD. In some embodiments, the serotonin receptor modulator is ritanserin and the psychedelic is 1P-LSD, wherein the ritanserin is administered to pretreat at least 210 minutes prior to the IP-LSD. In some embodiments, the serotonin receptor modulator is ritanserin and the psychedelic is 1P-LSD, wherein the ritanserin is administered to pretreat at least 240 minutes prior to the IP-LSD. In some embodiments, the serotonin receptor modulator is ritanserin and the psychedelic is 1P-LSD, wherein the ritanserin is administered to pretreat at least 270 minutes prior to the IP-LSD. In some embodiments, the serotonin receptor modulator is ritanserin and the psychedelic is 1P-LSD, wherein the ritanserin is administered to pretreat at least 300 minutes prior to the IP-LSD. In some embodiments, the serotonin receptor modulator is ritanserin and the psychedelic is 1P-LSD, wherein the ritanserin is administered to pretreat at least 330 minutes prior to the IP-LSD. In some embodiments, the serotonin receptor modulator is ritanserin and the psychedelic is IP-LSD, wherein the ritanserin is administered to pretreat at least 360 minutes prior to the IP-LSD. In some preferred embodiments, the serotonin receptor modulator is ritanserin and the psychedelic is 1P-LSD, wherein ritanserin is administered to pretreat between about 60 minutes and about 180 minutes prior to the administration of 1P-LSD.

In some embodiments, the serotonin receptor modulator is pimavanserin and the psychedelic is 1P-LSD, wherein the pimavanserin is administered to pretreat at least 15 minutes prior to the IP-LSD. In some embodiments, the serotonin receptor modulator is pimavanserin and the psychedelic is 1P-LSD, wherein the pimavanserin is administered to pretreat at least 30 minutes prior to the IP-LSD. In some embodiments, the serotonin receptor modulator is pimavanserin and the psychedelic is 1P-LSD, wherein the pimavanserin is administered to pretreat at least 60 minutes prior to the IP-LSD. In some embodiments, the serotonin receptor modulator is pimavanserin and the psychedelic is 1P-LSD, wherein the pimavanserin is administered to pretreat at least 90 minutes prior to the IP-LSD. In some embodiments, the serotonin receptor modulator is pimavanserin and the psychedelic is IP-LSD, wherein the pimavanserin is administered to pretreat at least 120 minutes prior to the 1P-LSD. In some embodiments, the serotonin receptor modulator is pimavanserin and the psychedelic is 1P-LSD, wherein the pimavanserin is administered to pretreat at least 150 minutes prior to the IP-LSD. In some embodiments, the serotonin receptor modulator is pimavanserin and the psychedelic is 1P-LSD, wherein the pimavanserin is administered to pretreat between about 15 minutes and about 150 minutes prior to the IP-LSD. In some embodiments, the serotonin receptor modulator is pimavanserin and the psychedelic is 1P-LSD, wherein the pimavanserin is administered to pretreat at least 180 minutes prior to the IP-LSD. In some embodiments, the serotonin receptor modulator is pimavanserin and the psychedelic is 1P-LSD, wherein the pimavanserin is administered to pretreat at least 210 minutes prior to the IP-LSD. In some embodiments, the serotonin receptor modulator is pimavanserin and the psychedelic is IP-LSD, wherein the pimavanserin is administered to pretreat at least 240 minutes prior to the IP-LSD. In some embodiments, the serotonin receptor modulator is pimavanserin and the psychedelic is IP-LSD, wherein the pimavanserin is administered to pretreat at least 270 minutes prior to the IP-LSD. In some embodiments, the serotonin receptor modulator is pimavanserin and the psychedelic is 1P-LSD, wherein the pimavanserin is administered to pretreat at least 300 minutes prior to the IP-LSD. In some embodiments, the serotonin receptor modulator is pimavanserin and the psychedelic is 1P-LSD, wherein the pimavanserin is administered to pretreat at least 330 minutes prior to the 1P-LSD. In some embodiments, the serotonin receptor modulator is pimavanserin and the psychedelic is 1P-LSD, wherein the pimavanserin is administered to pretreat at least 360 minutes prior to the IP-LSD. In some preferred embodiments, the serotonin receptor modulator is pimavanserin and the psychedelic is IP-LSD, wherein pimavanserin is administered to pretreat between about 60 minutes and about 180 minutes prior to the administration of 1P-LSD.

In some embodiments, the serotonin receptor modulator is nelotanserin and the psychedelic is 1P-LSD, wherein the nelotanserin is administered to pretreat at least 15 minutes prior to the 1P-LSD. In some embodiments, the serotonin receptor modulator is nelotanserin and the psychedelic is 1P-LSD, wherein the nelotanserin is administered to pretreat at least 30 minutes prior to the 1P-LSD. In some embodiments, the serotonin receptor modulator is nelotanserin and the psychedelic is 1P-LSD, wherein the nelotanserin is administered to pretreat at least 60 minutes prior to the IP-LSD. In some embodiments, the serotonin receptor modulator is nelotanserin and the psychedelic is 1P-LSD, wherein the nelotanserin is administered to pretreat at least 90 minutes prior to the 1P-LSD. In some embodiments, the serotonin receptor modulator is nelotanserin and the psychedelic is 1P-LSD, wherein the nelotanserin is administered to pretreat at least 120 minutes prior to the IP-LSD. In some embodiments, the serotonin receptor modulator is nelotanserin and the psychedelic is 1P-LSD, wherein the nelotanserin is administered to pretreat at least 150 minutes prior to the IP-LSD. In some embodiments, the serotonin receptor modulator is nelotanserin and the psychedelic is IP-LSD, wherein the nelotanserin is administered to pretreat between about 15 minutes and about 150 minutes prior to the IP-LSD. In some embodiments, the serotonin receptor modulator is nelotanserin and the psychedelic is 1P-LSD, wherein the nelotanserin is administered to pretreat at least 180 minutes prior to the 1P-LSD. In some embodiments, the serotonin receptor modulator is nelotanserin and the psychedelic is 1P-LSD, wherein the nelotanserin is administered to pretreat at least 210 minutes prior to the IP-LSD. In some embodiments, the serotonin receptor modulator is nelotanserin and the psychedelic is IP-LSD, wherein the nelotanserin is administered to pretreat at least 240 minutes prior to the IP-LSD. In some embodiments, the serotonin receptor modulator is nelotanserin and the psychedelic is IP-LSD, wherein the nelotanserin is administered to pretreat at least 270 minutes prior to the IP-LSD. In some embodiments, the serotonin receptor modulator is nelotanserin and the psychedelic is 1P-LSD, wherein the nelotanserin is administered to pretreat at least 300 minutes prior to the 1P-LSD. In some embodiments, the serotonin receptor modulator is nelotanserin and the psychedelic is 1P-LSD, wherein the nelotanserin is administered to pretreat at least 330 minutes prior to the IP-LSD. In some embodiments, the serotonin receptor modulator is nelotanserin and the psychedelic is IP-LSD, wherein the nelotanserin is administered to pretreat at least 360 minutes prior to the IP-LSD. In some preferred embodiments, the serotonin receptor modulator is nelotanserin and the psychedelic is IP-LSD, wherein nelotanserin is administered to pretreat between about 60 minutes and about 180 minutes prior to the administration of IP-LSD.

In some embodiments, the serotonin receptor modulator is pruvanserin and the psychedelic is 1P-LSD, wherein the pruvanserin is administered to pretreat at least 15 minutes prior to the IP-LSD. In some embodiments, the serotonin receptor modulator is pruvanserin and the psychedelic is 1P-LSD, wherein the pruvanserin is administered to pretreat at least 30 minutes prior to the IP-LSD. In some embodiments, the serotonin receptor modulator is pruvanserin and the psychedelic is IP-LSD, wherein the pruvanserin is administered to pretreat at least 60 minutes prior to the IP-LSD. In some embodiments, the serotonin receptor modulator is pruvanserin and the psychedelic is IP-LSD, wherein the pruvanserin is administered to pretreat at least 90 minutes prior to the IP-LSD. In some embodiments, the serotonin receptor modulator is pruvanserin and the psychedelic is 1P-LSD, wherein the pruvanserin is administered to pretreat at least 120 minutes prior to the IP-LSD. In some embodiments, the serotonin receptor modulator is pruvanserin and the psychedelic is IP-LSD, wherein the pruvanserin is administered to pretreat at least 150 minutes prior to the IP-LSD. In some embodiments, the serotonin receptor modulator is pruvanserin and the psychedelic is 1P-LSD, wherein the pruvanserin is administered to pretreat between about 15 minutes and about 150 minutes prior to the 1P-LSD. In some embodiments, the serotonin receptor modulator is pruvanserin and the psychedelic is IP-LSD, wherein the pruvanserin is administered to pretreat at least 180 minutes prior to the 1P-LSD. In some embodiments, the serotonin receptor modulator is pruvanserin and the psychedelic is IP-LSD, wherein the pruvanserin is administered to pretreat at least 210 minutes prior to the IP-LSD. In some embodiments, the serotonin receptor modulator is pruvanserin and the psychedelic is 1P-LSD, wherein the pruvanserin is administered to pretreat at least 240 minutes prior to the 1P-LSD. In some embodiments, the serotonin receptor modulator is pruvanserin and the psychedelic is 1P-LSD, wherein the pruvanserin is administered to pretreat at least 270 minutes prior to the IP-LSD. In some embodiments, the serotonin receptor modulator is pruvanserin and the psychedelic is 1P-LSD, wherein the pruvanserin is administered to pretreat at least 300 minutes prior to the IP-LSD. In some embodiments, the serotonin receptor modulator is pruvanserin and the psychedelic is 1P-LSD, wherein the pruvanserin is administered to pretreat at least 330 minutes prior to the 1P-LSD. In some embodiments, the serotonin receptor modulator is pruvanserin and the psychedelic is 1P-LSD, wherein the pruvanserin is administered to pretreat at least 360 minutes prior to the IP-LSD. In some preferred embodiments, the serotonin receptor modulator is pruvanserin and the psychedelic is IP-LSD, wherein pruvanserin is administered to pretreat between about 60 minutes and about 180 minutes prior to the administration of 1P-LSD.

In some embodiments, the serotonin receptor modulator is eplivanserin and the psychedelic is DMT, wherein the eplivanserin is administered to pretreat at least 15 minutes prior to the administration of DMT. In some embodiments, the serotonin receptor modulator is eplivanserin and the psychedelic is DMT, wherein the eplivanserin is administered to pretreat at least 30 minutes prior to the DMT. In some embodiments, the serotonin receptor modulator is eplivanserin and the psychedelic is DMT, wherein the eplivanserin is administered to pretreat at least 60 minutes prior to the DMT. In some embodiments, the serotonin receptor modulator is eplivanserin and the psychedelic is DMT, wherein the eplivanserin is administered to pretreat at least 90 minutes prior to the DMT. In some embodiments, the serotonin receptor modulator is eplivanserin and the psychedelic is DMT, wherein the eplivanserin is administered to pretreat at least 120 minutes prior to the DMT. In some embodiments, the serotonin receptor modulator is eplivanserin and the psychedelic is DMT, wherein the eplivanserin is administered to pretreat at least 150 minutes prior to the DMT. In some embodiments, the serotonin receptor modulator is eplivanserin and the psychedelic is DMT, wherein the eplivanserin is administered to pretreat at least 180 minutes prior to the DMT. In some embodiments, the serotonin receptor modulator is eplivanserin and the psychedelic is DMT, wherein the eplivanserin is administered to pretreat at least 210 minutes prior to the DMT. In some embodiments, the serotonin receptor modulator is eplivanserin and the psychedelic is DMT, wherein the eplivanserin is administered to pretreat at least 240 minutes prior to the DMT. In some embodiments, the serotonin receptor modulator is eplivanserin and the psychedelic is DMT, wherein the eplivanserin is administered to pretreat at least 270 minutes prior to the DMT. In some embodiments, the serotonin receptor modulator is eplivanserin and the psychedelic is DMT, wherein the eplivanserin is administered to pretreat at least 300 minutes prior to the DMT. In some embodiments, the serotonin receptor modulator is eplivanserin and the psychedelic is DMT, wherein the eplivanserin is administered to pretreat at least 330 minutes prior to the DMT. In some embodiments, the serotonin receptor modulator is eplivanserin and the psychedelic is DMT, wherein the eplivanserin is administered to pretreat at least 360 minutes prior to the DMT.

In some preferred embodiments, the serotonin receptor modulator is eplivanserin and the psychedelic is DMT, wherein eplivanserin is administered to pretreat between about 60 minutes and about 180 minutes prior to the administration of DMT.

In some embodiments, the serotonin receptor modulator is volinanserin and the psychedelic is DMT, wherein the volinanserin is administered to pretreat at least 15 minutes prior to the administration of DMT. In some embodiments, the serotonin receptor modulator is volinanserin and the psychedelic is DMT, wherein the volinanserin is administered to pretreat at least 30 minutes prior to the DMT. In some embodiments, the serotonin receptor modulator is volinanserin and the psychedelic is DMT, wherein the volinanserin is administered to pretreat at least 60 minutes prior to the DMT. In some embodiments, the serotonin receptor modulator is volinanserin and the psychedelic is DMT, wherein the volinanserin is administered to pretreat at least 90 minutes prior to the DMT. In some embodiments, the serotonin receptor modulator is volinanserin and the psychedelic is DMT, wherein the volinanserin is administered to pretreat at least 120 minutes prior to the DMT. In some embodiments, the serotonin receptor modulator is volinanserin and the psychedelic is DMT, wherein the volinanserin is administered to pretreat at least 150 minutes prior to the DMT. In some embodiments, the serotonin receptor modulator is volinanserin and the psychedelic is DMT, wherein the volinanserin is administered to pretreat between about 15 minutes and about 150 minutes prior to the DMT. In some embodiments, the serotonin receptor modulator is volinanserin and the psychedelic is DMT, wherein the volinanserin is administered to pretreat at least 180 minutes prior to the DMT. In some embodiments, the serotonin receptor modulator is volinanserin and the psychedelic is DMT, wherein the volinanserin is administered to pretreat at least 210 minutes prior to the DMT. In some embodiments, the serotonin receptor modulator is volinanserin and the psychedelic is DMT, wherein the volinanserin is administered to pretreat at least 240 minutes prior to the DMT. In some embodiments, the serotonin receptor modulator is volinanserin and the psychedelic is DMT, wherein the volinanserin is administered to pretreat at least 270 minutes prior to the DMT. In some embodiments, the serotonin receptor modulator is volinanserin and the psychedelic is DMT, wherein the volinanserin is administered to pretreat at least 300 minutes prior to the DMT. In some embodiments, the serotonin receptor modulator is volinanserin and the psychedelic is DMT, wherein the volinanserin is administered to pretreat at least 330 minutes prior to the DMT. In some embodiments, the serotonin receptor modulator is volinanserin and the psychedelic is DMT, wherein the volinanserin is administered to pretreat at least 360 minutes prior to the DMT. In some preferred embodiments, the serotonin receptor modulator is volinanserin and the psychedelic is DMT, wherein volinanserin is administered to pretreat between about 60 minutes and about 180 minutes prior to the administration of DMT.

In some embodiments, the serotonin receptor modulator is ketanserin and the psychedelic is DMT, wherein the ketanserin is administered to pretreat at least 15 minutes prior to the administration of DMT. In some embodiments, the serotonin receptor modulator is ketanserin and the psychedelic is DMT, wherein the ketanserin is administered to pretreat at least 30 minutes prior to the DMT. In some embodiments, the serotonin receptor modulator is ketanserin and the psychedelic is DMT, wherein the ketanserin is administered to pretreat at least 60 minutes prior to the DMT. In some embodiments, the serotonin receptor modulator is ketanserin and the psychedelic is DMT, wherein the ketanserin is administered to pretreat at least 90 minutes prior to the DMT. In some embodiments, the serotonin receptor modulator is ketanserin and the psychedelic is DMT, wherein the ketanserin is administered to pretreat at least 120 minutes prior to the DMT. In some embodiments, the serotonin receptor modulator is ketanserin and the psychedelic is DMT, wherein the ketanserin is administered to pretreat at least 150 minutes prior to the DMT. In some embodiments, the serotonin receptor modulator is ketanserin and the psychedelic is DMT, wherein the ketanserin is administered to pretreat between about 15 minutes and about 150 minutes prior to the DMT. In some embodiments, the serotonin receptor modulator is ketanserin and the psychedelic is DMT, wherein the ketanserin is administered to pretreat at least 180 minutes prior to the DMT. In some embodiments, the serotonin receptor modulator is ketanserin and the psychedelic is DMT, wherein the ketanserin is administered to pretreat at least 210 minutes prior to the DMT. In some embodiments, the serotonin receptor modulator is ketanserin and the psychedelic is DMT, wherein the ketanserin is administered to pretreat at least 240 minutes prior to the DMT. In some embodiments, the serotonin receptor modulator is ketanserin and the psychedelic is DMT, wherein the ketanserin is administered to pretreat at least 270 minutes prior to the DMT. In some embodiments, the serotonin receptor modulator is ketanserin and the psychedelic is DMT, wherein the ketanserin is administered to pretreat at least 300 minutes prior to the DMT. In some embodiments, the serotonin receptor modulator is ketanserin and the psychedelic is DMT, wherein the ketanserin is administered to pretreat at least 330 minutes prior to the DMT. In some embodiments, the serotonin receptor modulator is ketanserin and the psychedelic is DMT, wherein the ketanserin is administered to pretreat at least 360 minutes prior to the DMT. In some preferred embodiments, the serotonin receptor modulator is ketanserin and the psychedelic is DMT, wherein ketanserin is administered to pretreat between about 60 minutes and about 180 minutes prior to the administration of DMT.

In some embodiments, the serotonin receptor modulator is ritanserin and the psychedelic is DMT, wherein the ritanserin is administered to pretreat at least 15 minutes prior to the DMT. In some embodiments, the serotonin receptor modulator is ritanserin and the psychedelic is DMT, wherein the ritanserin is administered to pretreat at least 30 minutes prior to the DMT. In some embodiments, the serotonin receptor modulator is ritanserin and the psychedelic is DMT, wherein the ritanserin is administered to pretreat at least 60 minutes prior to the DMT. In some embodiments, the serotonin receptor modulator is ritanserin and the psychedelic is DMT, wherein the ritanserin is administered to pretreat at least 90 minutes prior to the DMT. In some embodiments, the serotonin receptor modulator is ritanserin and the psychedelic is DMT, wherein the ritanserin is administered to pretreat at least 120 minutes prior to the DMT. In some embodiments, the serotonin receptor modulator is ritanserin and the psychedelic is DMT, wherein the ritanserin is administered to pretreat at least 150 minutes prior to the DMT. In some embodiments, the serotonin receptor modulator is ritanserin and the psychedelic is DMT, wherein the ritanserin is administered to pretreat between about 15 minutes and about 150 minutes prior to the DMT. In some embodiments, the serotonin receptor modulator is ritanserin and the psychedelic is DMT, wherein the ritanserin is administered to pretreat at least 180 minutes prior to the DMT. In some embodiments, the serotonin receptor modulator is ritanserin and the psychedelic is DMT, wherein the ritanserin is administered to pretreat at least 210 minutes prior to the DMT. In some embodiments, the serotonin receptor modulator is ritanserin and the psychedelic is DMT, wherein the ritanserin is administered to pretreat at least 240 minutes prior to the DMT. In some embodiments, the serotonin receptor modulator is ritanserin and the psychedelic is DMT, wherein the ritanserin is administered to pretreat at least 270 minutes prior to the DMT. In some embodiments, the serotonin receptor modulator is ritanserin and the psychedelic is DMT, wherein the ritanserin is administered to pretreat at least 300 minutes prior to the DMT. In some embodiments, the serotonin receptor modulator is ritanserin and the psychedelic is DMT, wherein the ritanserin is administered to pretreat at least 330 minutes prior to the DMT. In some embodiments, the serotonin receptor modulator is ritanserin and the psychedelic is DMT, wherein the ritanserin is administered to pretreat at least 360 minutes prior to the DMT. In some preferred embodiments, the serotonin receptor modulator is ritanserin and the psychedelic is DMT, wherein ritanserin is administered to pretreat between about 60 minutes and about 180 minutes prior to the administration of DMT.

In some embodiments, the serotonin receptor modulator is pimavanserin and the psychedelic is DMT, wherein the pimavanserin is administered to pretreat at least 15 minutes prior to the DMT. In some embodiments, the serotonin receptor modulator is pimavanserin and the psychedelic is DMT, wherein the pimavanserin is administered to pretreat at least 30 minutes prior to the DMT. In some embodiments, the serotonin receptor modulator is pimavanserin and the psychedelic is DMT, wherein the pimavanserin is administered to pretreat at least 60 minutes prior to the DMT. In some embodiments, the serotonin receptor modulator is pimavanserin and the psychedelic is DMT, wherein the pimavanserin is administered to pretreat at least 90 minutes prior to the DMT. In some embodiments, the serotonin receptor modulator is pimavanserin and the psychedelic is DMT, wherein the pimavanserin is administered to pretreat at least 120 minutes prior to the DMT. In some embodiments, the serotonin receptor modulator is pimavanserin and the psychedelic is DMT, wherein the pimavanserin is administered to pretreat at least 150 minutes prior to the DMT. In some embodiments, the serotonin receptor modulator is pimavanserin and the psychedelic is DMT, wherein the pimavanserin is administered to pretreat between about 15 minutes and about 150 minutes prior to the DMT. In some embodiments, the serotonin receptor modulator is pimavanserin and the psychedelic is DMT, wherein the pimavanserin is administered to pretreat at least 180 minutes prior to the DMT. In some embodiments, the serotonin receptor modulator is pimavanserin and the psychedelic is DMT, wherein the pimavanserin is administered to pretreat at least 210 minutes prior to the DMT. In some embodiments, the serotonin receptor modulator is pimavanserin and the psychedelic is DMT, wherein the pimavanserin is administered to pretreat at least 240 minutes prior to the DMT. In some embodiments, the serotonin receptor modulator is pimavanserin and the psychedelic is DMT, wherein the pimavanserin is administered to pretreat at least 270 minutes prior to the DMT. In some embodiments, the serotonin receptor modulator is pimavanserin and the psychedelic is DMT, wherein the pimavanserin is administered to pretreat at least 300 minutes prior to the DMT. In some embodiments, the serotonin receptor modulator is pimavanserin and the psychedelic is DMT, wherein the pimavanserin is administered to pretreat at least 330 minutes prior to the DMT. In some embodiments, the serotonin receptor modulator is pimavanserin and the psychedelic is DMT, wherein the pimavanserin is administered to pretreat at least 360 minutes prior to the DMT. In some preferred embodiments, the serotonin receptor modulator is pimavanserin and the psychedelic is DMT, wherein pimavanserin is administered to pretreat between about 60 minutes and about 180 minutes prior to the administration of DMT.

In some embodiments, the serotonin receptor modulator is nelotanserin and the psychedelic is DMT, wherein the nelotanserin is administered to pretreat at least 15 minutes prior to the DMT. In some embodiments, the serotonin receptor modulator is nelotanserin and the psychedelic is DMT, wherein the nelotanserin is administered to pretreat at least 30 minutes prior to the DMT. In some embodiments, the serotonin receptor modulator is nelotanserin and the psychedelic is DMT, wherein the nelotanserin is administered to pretreat at least 60 minutes prior to the DMT. In some embodiments, the serotonin receptor modulator is nelotanserin and the psychedelic is DMT, wherein the nelotanserin is administered to pretreat at least 90 minutes prior to the DMT. In some embodiments, the serotonin receptor modulator is nelotanserin and the psychedelic is DMT, wherein the nelotanserin is administered to pretreat at least 120 minutes prior to the DMT. In some embodiments, the serotonin receptor modulator is nelotanserin and the psychedelic is DMT, wherein the nelotanserin is administered to pretreat at least 150 minutes prior to the DMT. In some embodiments, the serotonin receptor modulator is nelotanserin and the psychedelic is DMT, wherein the nelotanserin is administered to pretreat between about 15 minutes and about 150 minutes prior to the DMT. In some embodiments, the serotonin receptor modulator is nelotanserin and the psychedelic is DMT, wherein the nelotanserin is administered to pretreat at least 180 minutes prior to the DMT. In some embodiments, the serotonin receptor modulator is nelotanserin and the psychedelic is DMT, wherein the nelotanserin is administered to pretreat at least 210 minutes prior to the DMT. In some embodiments, the serotonin receptor modulator is nelotanserin and the psychedelic is DMT, wherein the nelotanserin is administered to pretreat at least 240 minutes prior to the DMT. In some embodiments, the serotonin receptor modulator is nelotanserin and the psychedelic is DMT, wherein the nelotanserin is administered to pretreat at least 270 minutes prior to the DMT. In some embodiments, the serotonin receptor modulator is nelotanserin and the psychedelic is DMT, wherein the nelotanserin is administered to pretreat at least 300 minutes prior to the DMT. In some embodiments, the serotonin receptor modulator is nelotanserin and the psychedelic is DMT, wherein the nelotanserin is administered to pretreat at least 330 minutes prior to the DMT. In some embodiments, the serotonin receptor modulator is nelotanserin and the psychedelic is DMT, wherein the nelotanserin is administered to pretreat at least 360 minutes prior to the DMT. In some preferred embodiments, the serotonin receptor modulator is nelotanserin and the psychedelic is DMT, wherein nelotanserin is administered to pretreat between about 60 minutes and about 180 minutes prior to the administration of DMT.

In some embodiments, the serotonin receptor modulator is pruvanserin and the psychedelic is DMT, wherein the pruvanserin is administered to pretreat at least 15 minutes prior to the DMT. In some embodiments, the serotonin receptor modulator is pruvanserin and the psychedelic is DMT, wherein the pruvanserin is administered to pretreat at least 30 minutes prior to the DMT. In some embodiments, the serotonin receptor modulator is pruvanserin and the psychedelic is DMT, wherein the pruvanserin is administered to pretreat at least 60 minutes prior to the DMT. In some embodiments, the serotonin receptor modulator is pruvanserin and the psychedelic is DMT, wherein the pruvanserin is administered to pretreat at least 90 minutes prior to the DMT. In some embodiments, the serotonin receptor modulator is pruvanserin and the psychedelic is DMT, wherein the pruvanserin is administered to pretreat at least 120 minutes prior to the DMT. In some embodiments, the serotonin receptor modulator is pruvanserin and the psychedelic is DMT, wherein the pruvanserin is administered to pretreat at least 150 minutes prior to the DMT. In some embodiments, the serotonin receptor modulator is pruvanserin and the psychedelic is DMT, wherein the pruvanserin is administered to pretreat between about 15 minutes and about 150 minutes prior to the DMT. In some embodiments, the serotonin receptor modulator is pruvanserin and the psychedelic is DMT, wherein the pruvanserin is administered to pretreat at least 180 minutes prior to the DMT. In some embodiments, the serotonin receptor modulator is pruvanserin and the psychedelic is DMT, wherein the pruvanserin is administered to pretreat at least 210 minutes prior to the DMT. In some embodiments, the serotonin receptor modulator is pruvanserin and the psychedelic is DMT, wherein the pruvanserin is administered to pretreat at least 240 minutes prior to the DMT. In some embodiments, the serotonin receptor modulator is pruvanserin and the psychedelic is DMT, wherein the pruvanserin is administered to pretreat at least 270 minutes prior to the DMT. In some embodiments, the serotonin receptor modulator is pruvanserin and the psychedelic is DMT, wherein the pruvanserin is administered to pretreat at least 300 minutes prior to the DMT. In some embodiments, the serotonin receptor modulator is pruvanserin and the psychedelic is DMT, wherein the pruvanserin is administered to pretreat at least 330 minutes prior to the DMT. In some embodiments, the serotonin receptor modulator is pruvanserin and the psychedelic is DMT, wherein the pruvanserin is administered to pretreat at least 360 minutes prior to the DMT. In some preferred embodiments, the serotonin receptor modulator is pruvanserin and the psychedelic is DMT, wherein pruvanserin is administered to pretreat between about 60 minutes and about 180 minutes prior to the administration of DMT.

In some embodiments, the serotonin receptor modulator is eplivanserin and the psychedelic is 5-MeO-DMT, wherein the eplivanserin is administered to pretreat at least 15 minutes prior to the administration of 5-MeO-DMT. In some embodiments, the serotonin receptor modulator is eplivanserin and the psychedelic is 5-MeO-DMT, wherein the eplivanserin is administered to pretreat at least 30 minutes prior to the 5-MeO-DMT. In some embodiments, the serotonin receptor modulator is eplivanserin and the psychedelic is 5-MeO-DMT, wherein the eplivanserin is administered to pretreat at least 60 minutes prior to the 5-MeO-DMT. In some embodiments, the serotonin receptor modulator is eplivanserin and the psychedelic is 5-MeO-DMT, wherein the eplivanserin is administered to pretreat at least 90 minutes prior to the 5-MeO-DMT. In some embodiments, the serotonin receptor modulator is eplivanserin and the psychedelic is 5-MeO-DMT, wherein the eplivanserin is administered to pretreat at least 120 minutes prior to the 5-MeO-DMT. In some embodiments, the serotonin receptor modulator is eplivanserin and the psychedelic is 5-MeO-DMT, wherein the eplivanserin is administered to pretreat at least 150 minutes prior to the 5-MeO-DMT. In some embodiments, the serotonin receptor modulator is eplivanserin and the psychedelic is 5-MeO-DMT, wherein the eplivanserin is administered to pretreat at least 180 minutes prior to the 5-MeO-DMT. In some embodiments, the serotonin receptor modulator is eplivanserin and the psychedelic is 5-MeO-DMT, wherein the eplivanserin is administered to pretreat at least 210 minutes prior to the 5-MeO-DMT. In some embodiments, the serotonin receptor modulator is eplivanserin and the psychedelic is 5-MeO-DMT, wherein the eplivanserin is administered to pretreat at least 240 minutes prior to the 5-MeO-DMT. In some embodiments, the serotonin receptor modulator is eplivanserin and the psychedelic is 5-MeO-DMT, wherein the eplivanserin is administered to pretreat at least 270 minutes prior to the 5-MeO-DMT. In some embodiments, the serotonin receptor modulator is eplivanserin and the psychedelic is 5-MeO-DMT, wherein the eplivanserin is administered to pretreat at least 300 minutes prior to the 5-MeO-DMT. In some embodiments, the serotonin receptor modulator is eplivanserin and the psychedelic is 5-MeO-DMT, wherein the eplivanserin is administered to pretreat at least 330 minutes prior to the 5-MeO-DMT. In some embodiments, the serotonin receptor modulator is eplivanserin and the psychedelic is 5-MeO-DMT, wherein the eplivanserin is administered to pretreat at least 360 minutes prior to the 5-MeO-DMT.

In some preferred embodiments, the serotonin receptor modulator is eplivanserin and the psychedelic is 5-MeO-DMT, wherein eplivanserin is administered to pretreat between about 60 minutes and about 180 minutes prior to the administration of 5-MeO-DMT.

In some embodiments, the serotonin receptor modulator is volinanserin and the psychedelic is 5-MeO-DMT, wherein the volinanserin is administered to pretreat at least 15 minutes prior to the administration of 5-MeO-DMT. In some embodiments, the serotonin receptor modulator is volinanserin and the psychedelic is 5-MeO-DMT, wherein the volinanserin is administered to pretreat at least 30 minutes prior to the 5-MeO-DMT. In some embodiments, the serotonin receptor modulator is volinanserin and the psychedelic is 5-MeO-DMT, wherein the volinanserin is administered to pretreat at least 60 minutes prior to the 5-MeO-DMT. In some embodiments, the serotonin receptor modulator is volinanserin and the psychedelic is 5-MeO-DMT, wherein the volinanserin is administered to pretreat at least 90 minutes prior to the 5-MeO-DMT. In some embodiments, the serotonin receptor modulator is volinanserin and the psychedelic is 5-MeO-DMT, wherein the volinanserin is administered to pretreat at least 120 minutes prior to the 5-MeO-DMT. In some embodiments, the serotonin receptor modulator is volinanserin and the psychedelic is 5-MeO-DMT, wherein the volinanserin is administered to pretreat at least 150 minutes prior to the 5-MeO-DMT. In some embodiments, the serotonin receptor modulator is volinanserin and the psychedelic is 5-MeO-DMT, wherein the volinanserin is administered to pretreat between about 15 minutes and about 150 minutes prior to the 5-MeO-DMT. In some embodiments, the serotonin receptor modulator is volinanserin and the psychedelic is 5-MeO-DMT, wherein the volinanserin is administered to pretreat at least 180 minutes prior to the 5-MeO-DMT. In some embodiments, the serotonin receptor modulator is volinanserin and the psychedelic is 5-MeO-DMT, wherein the volinanserin is administered to pretreat at least 210 minutes prior to the 5-MeO-DMT. In some embodiments, the serotonin receptor modulator is volinanserin and the psychedelic is 5-MeO-DMT, wherein the volinanserin is administered to pretreat at least 240 minutes prior to the 5-MeO-DMT. In some embodiments, the serotonin receptor modulator is volinanserin and the psychedelic is 5-MeO-DMT, wherein the volinanserin is administered to pretreat at least 270 minutes prior to the 5-MeO-DMT. In some embodiments, the serotonin receptor modulator is volinanserin and the psychedelic is 5-MeO-DMT, wherein the volinanserin is administered to pretreat at least 300 minutes prior to the 5-MeO-DMT. In some embodiments, the serotonin receptor modulator is volinanserin and the psychedelic is 5-MeO-DMT, wherein the volinanserin is administered to pretreat at least 330 minutes prior to the 5-MeO-DMT. In some embodiments, the serotonin receptor modulator is volinanserin and the psychedelic is 5-MeO-DMT, wherein the volinanserin is administered to pretreat at least 360 minutes prior to the 5-MeO-DMT. In some preferred embodiments, the serotonin receptor modulator is volinanserin and the psychedelic is 5-MeO-DMT, wherein volinanserin is administered to pretreat between about 60 minutes and about 180 minutes prior to the administration of 5-MeO-DMT.

In some embodiments, the serotonin receptor modulator is ketanserin and the psychedelic is 5-MeO-DMT, wherein the ketanserin is administered to pretreat at least 15 minutes prior to the administration of 5-MeO-DMT. In some embodiments, the serotonin receptor modulator is ketanserin and the psychedelic is 5-MeO-DMT, wherein the ketanserin is administered to pretreat at least 30 minutes prior to the 5-MeO-DMT. In some embodiments, the serotonin receptor modulator is ketanserin and the psychedelic is 5-MeO-DMT, wherein the ketanserin is administered to pretreat at least 60 minutes prior to the 5-MeO-DMT. In some embodiments, the serotonin receptor modulator is ketanserin and the psychedelic is 5-MeO-DMT, wherein the ketanserin is administered to pretreat at least 90 minutes prior to the 5-MeO-DMT. In some embodiments, the serotonin receptor modulator is ketanserin and the psychedelic is 5-MeO-DMT, wherein the ketanserin is administered to pretreat at least 120 minutes prior to the 5-MeO-DMT. In some embodiments, the serotonin receptor modulator is ketanserin and the psychedelic is 5-MeO-DMT, wherein the ketanserin is administered to pretreat at least 150 minutes prior to the 5-MeO-DMT. In some embodiments, the serotonin receptor modulator is ketanserin and the psychedelic is 5-MeO-DMT, wherein the ketanserin is administered to pretreat between about 15 minutes and about 150 minutes prior to the 5-MeO-DMT. In some embodiments, the serotonin receptor modulator is ketanserin and the psychedelic is 5-MeO-DMT, wherein the ketanserin is administered to pretreat at least 180 minutes prior to the 5-MeO-DMT. In some embodiments, the serotonin receptor modulator is ketanserin and the psychedelic is 5-MeO-DMT, wherein the ketanserin is administered to pretreat at least 210 minutes prior to the 5-MeO-DMT. In some embodiments, the serotonin receptor modulator is ketanserin and the psychedelic is 5-MeO-DMT, wherein the ketanserin is administered to pretreat at least 240 minutes prior to the 5-MeO-DMT. In some embodiments, the serotonin receptor modulator is ketanserin and the psychedelic is 5-MeO-DMT, wherein the ketanserin is administered to pretreat at least 270 minutes prior to the 5-MeO-DMT. In some embodiments, the serotonin receptor modulator is ketanserin and the psychedelic is 5-MeO-DMT, wherein the ketanserin is administered to pretreat at least 300 minutes prior to the 5-MeO-DMT. In some embodiments, the serotonin receptor modulator is ketanserin and the psychedelic is 5-MeO-DMT, wherein the ketanserin is administered to pretreat at least 330 minutes prior to the 5-MeO-DMT. In some embodiments, the serotonin receptor modulator is ketanserin and the psychedelic is 5-MeO-DMT, wherein the ketanserin is administered to pretreat at least 360 minutes prior to the 5-MeO-DMT. In some preferred embodiments, the serotonin receptor modulator is ketanserin and the psychedelic is 5-MeO-DMT, wherein ketanserin is administered to pretreat between about 60 minutes and about 180 minutes prior to the administration of 5-MeO-DMT.

In some embodiments, the serotonin receptor modulator is ritanserin and the psychedelic is 5-MeO-DMT, wherein the ritanserin is administered to pretreat at least 15 minutes prior to the 5-MeO-DMT. In some embodiments, the serotonin receptor modulator is ritanserin and the psychedelic is 5-MeO-DMT, wherein the ritanserin is administered to pretreat at least 30 minutes prior to the 5-MeO-DMT. In some embodiments, the serotonin receptor modulator is ritanserin and the psychedelic is 5-MeO-DMT, wherein the ritanserin is administered to pretreat at least 60 minutes prior to the 5-MeO-DMT. In some embodiments, the serotonin receptor modulator is ritanserin and the psychedelic is 5-MeO-DMT, wherein the ritanserin is administered to pretreat at least 90 minutes prior to the 5-MeO-DMT. In some embodiments, the serotonin receptor modulator is ritanserin and the psychedelic is 5-MeO-DMT, wherein the ritanserin is administered to pretreat at least 120 minutes prior to the 5-MeO-DMT. In some embodiments, the serotonin receptor modulator is ritanserin and the psychedelic is 5-MeO-DMT, wherein the ritanserin is administered to pretreat at least 150 minutes prior to the 5-MeO-DMT. In some embodiments, the serotonin receptor modulator is ritanserin and the psychedelic is 5-MeO-DMT, wherein the ritanserin is administered to pretreat between about 15 minutes and about 150 minutes prior to the 5-MeO-DMT. In some embodiments, the serotonin receptor modulator is ritanserin and the psychedelic is 5-MeO-DMT, wherein the ritanserin is administered to pretreat at least 180 minutes prior to the 5-MeO-DMT. In some embodiments, the serotonin receptor modulator is ritanserin and the psychedelic is 5-MeO-DMT, wherein the ritanserin is administered to pretreat at least 210 minutes prior to the 5-MeO-DMT. In some embodiments, the serotonin receptor modulator is ritanserin and the psychedelic is 5-MeO-DMT, wherein the ritanserin is administered to pretreat at least 240 minutes prior to the 5-MeO-DMT. In some embodiments, the serotonin receptor modulator is ritanserin and the psychedelic is 5-MeO-DMT, wherein the ritanserin is administered to pretreat at least 270 minutes prior to the 5-MeO-DMT. In some embodiments, the serotonin receptor modulator is ritanserin and the psychedelic is 5-MeO-DMT, wherein the ritanserin is administered to pretreat at least 300 minutes prior to the 5-MeO-DMT. In some embodiments, the serotonin receptor modulator is ritanserin and the psychedelic is 5-MeO-DMT, wherein the ritanserin is administered to pretreat at least 330 minutes prior to the 5-MeO-DMT. In some embodiments, the serotonin receptor modulator is ritanserin and the psychedelic is 5-MeO-DMT, wherein the ritanserin is administered to pretreat at least 360 minutes prior to the 5-MeO-DMT. In some preferred embodiments, the serotonin receptor modulator is ritanserin and the psychedelic is 5-MeO-DMT, wherein ritanserin is administered to pretreat between about 60 minutes and about 180 minutes prior to the administration of 5-MeO-DMT.

In some embodiments, the serotonin receptor modulator is pimavanserin and the psychedelic is 5-MeO-DMT, wherein the pimavanserin is administered to pretreat at least 15 minutes prior to the 5-MeO-DMT. In some embodiments, the serotonin receptor modulator is pimavanserin and the psychedelic is 5-MeO-DMT, wherein the pimavanserin is administered to pretreat at least 30 minutes prior to the 5-MeO-DMT. In some embodiments, the serotonin receptor modulator is pimavanserin and the psychedelic is 5-MeO-DMT, wherein the pimavanserin is administered to pretreat at least 60 minutes prior to the 5-MeO-DMT. In some embodiments, the serotonin receptor modulator is pimavanserin and the psychedelic is 5-MeO-DMT, wherein the pimavanserin is administered to pretreat at least 90 minutes prior to the 5-MeO-DMT. In some embodiments, the serotonin receptor modulator is pimavanserin and the psychedelic is 5-MeO-DMT, wherein the pimavanserin is administered to pretreat at least 120 minutes prior to the 5-MeO-DMT. In some embodiments, the serotonin receptor modulator is pimavanserin and the psychedelic is 5-MeO-DMT, wherein the pimavanserin is administered to pretreat at least 150 minutes prior to the 5-MeO-DMT. In some embodiments, the serotonin receptor modulator is pimavanserin and the psychedelic is 5-MeO-DMT, wherein the pimavanserin is administered to pretreat between about 15 minutes and about 150 minutes prior to the 5-MeO-DMT. In some embodiments, the serotonin receptor modulator is pimavanserin and the psychedelic is 5-MeO-DMT, wherein the pimavanserin is administered to pretreat at least 180 minutes prior to the 5-MeO-DMT. In some embodiments, the serotonin receptor modulator is pimavanserin and the psychedelic is 5-MeO-DMT, wherein the pimavanserin is administered to pretreat at least 210 minutes prior to the 5-MeO-DMT. In some embodiments, the serotonin receptor modulator is pimavanserin and the psychedelic is 5-MeO-DMT, wherein the pimavanserin is administered to pretreat at least 240 minutes prior to the 5-MeO-DMT. In some embodiments, the serotonin receptor modulator is pimavanserin and the psychedelic is 5-MeO-DMT, wherein the pimavanserin is administered to pretreat at least 270 minutes prior to the 5-MeO-DMT. In some embodiments, the serotonin receptor modulator is pimavanserin and the psychedelic is 5-MeO-DMT, wherein the pimavanserin is administered to pretreat at least 300 minutes prior to the 5-MeO-DMT. In some embodiments, the serotonin receptor modulator is pimavanserin and the psychedelic is 5-MeO-DMT, wherein the pimavanserin is administered to pretreat at least 330 minutes prior to the 5-MeO-DMT. In some embodiments, the serotonin receptor modulator is pimavanserin and the psychedelic is 5-MeO-DMT, wherein the pimavanserin is administered to pretreat at least 360 minutes prior to the 5-MeO-DMT. In some preferred embodiments, the serotonin receptor modulator is pimavanserin and the psychedelic is 5-MeO-DMT, wherein pimavanserin is administered to pretreat between about 60 minutes and about 180 minutes prior to the administration of 5-MeO-DMT.

In some embodiments, the serotonin receptor modulator is nelotanserin and the psychedelic is 5-MeO-DMT, wherein the nelotanserin is administered to pretreat at least 15 minutes prior to the 5-MeO-DMT. In some embodiments, the serotonin receptor modulator is nelotanserin and the psychedelic is 5-MeO-DMT, wherein the nelotanserin is administered to pretreat at least 30 minutes prior to the 5-MeO-DMT. In some embodiments, the serotonin receptor modulator is nelotanserin and the psychedelic is 5-MeO-DMT, wherein the nelotanserin is administered to pretreat at least 60 minutes prior to the 5-MeO-DMT. In some embodiments, the serotonin receptor modulator is nelotanserin and the psychedelic is 5-MeO-DMT, wherein the nelotanserin is administered to pretreat at least 90 minutes prior to the 5-MeO-DMT. In some embodiments, the serotonin receptor modulator is nelotanserin and the psychedelic is 5-MeO-DMT, wherein the nelotanserin is administered to pretreat at least 120 minutes prior to the 5-MeO-DMT. In some embodiments, the serotonin receptor modulator is nelotanserin and the psychedelic is 5-MeO-DMT, wherein the nelotanserin is administered to pretreat at least 150 minutes prior to the 5-MeO-DMT. In some embodiments, the serotonin receptor modulator is nelotanserin and the psychedelic is 5-MeO-DMT, wherein the nelotanserin is administered to pretreat between about 15 minutes and about 150 minutes prior to the 5-MeO-DMT. In some embodiments, the serotonin receptor modulator is nelotanserin and the psychedelic is 5-MeO-DMT, wherein the nelotanserin is administered to pretreat at least 180 minutes prior to the 5-MeO-DMT. In some embodiments, the serotonin receptor modulator is nelotanserin and the psychedelic is 5-MeO-DMT, wherein the nelotanserin is administered to pretreat at least 210 minutes prior to the 5-MeO-DMT. In some embodiments, the serotonin receptor modulator is nelotanserin and the psychedelic is 5-MeO-DMT, wherein the nelotanserin is administered to pretreat at least 240 minutes prior to the 5-MeO-DMT. In some embodiments, the serotonin receptor modulator is nelotanserin and the psychedelic is 5-MeO-DMT, wherein the nelotanserin is administered to pretreat at least 270 minutes prior to the 5-MeO-DMT. In some embodiments, the serotonin receptor modulator is nelotanserin and the psychedelic is 5-MeO-DMT, wherein the nelotanserin is administered to pretreat at least 300 minutes prior to the 5-MeO-DMT. In some embodiments, the serotonin receptor modulator is nelotanserin and the psychedelic is 5-MeO-DMT, wherein the nelotanserin is administered to pretreat at least 330 minutes prior to the 5-MeO-DMT. In some embodiments, the serotonin receptor modulator is nelotanserin and the psychedelic is 5-MeO-DMT, wherein the nelotanserin is administered to pretreat at least 360 minutes prior to the 5-MeO-DMT. In some preferred embodiments, the serotonin receptor modulator is nelotanserin and the psychedelic is 5-MeO-DMT, wherein nelotanserin is administered to pretreat between about 60 minutes and about 180 minutes prior to the administration of 5-MeO-DMT.

In some embodiments, the serotonin receptor modulator is pruvanserin and the psychedelic is 5-MeO-DMT, wherein the pruvanserin is administered to pretreat at least 15 minutes prior to the 5-MeO-DMT. In some embodiments, the serotonin receptor modulator is pruvanserin and the psychedelic is 5-MeO-DMT, wherein the pruvanserin is administered to pretreat at least 30 minutes prior to the 5-MeO-DMT. In some embodiments, the serotonin receptor modulator is pruvanserin and the psychedelic is 5-MeO-DMT, wherein the pruvanserin is administered to pretreat at least 60 minutes prior to the 5-MeO-DMT. In some embodiments, the serotonin receptor modulator is pruvanserin and the psychedelic is 5-MeO-DMT, wherein the pruvanserin is administered to pretreat at least 90 minutes prior to the 5-MeO-DMT. In some embodiments, the serotonin receptor modulator is pruvanserin and the psychedelic is 5-MeO-DMT, wherein the pruvanserin is administered to pretreat at least 120 minutes prior to the 5-MeO-DMT. In some embodiments, the serotonin receptor modulator is pruvanserin and the psychedelic is 5-MeO-DMT, wherein the pruvanserin is administered to pretreat at least 150 minutes prior to the 5-MeO-DMT. In some embodiments, the serotonin receptor modulator is pruvanserin and the psychedelic is 5-MeO-DMT, wherein the pruvanserin is administered to pretreat between about 15 minutes and about 150 minutes prior to the 5-MeO-DMT. In some embodiments, the serotonin receptor modulator is pruvanserin and the psychedelic is 5-MeO-DMT, wherein the pruvanserin is administered to pretreat at least 180 minutes prior to the 5-MeO-DMT. In some embodiments, the serotonin receptor modulator is pruvanserin and the psychedelic is 5-MeO-DMT, wherein the pruvanserin is administered to pretreat at least 210 minutes prior to the 5-MeO-DMT. In some embodiments, the serotonin receptor modulator is pruvanserin and the psychedelic is 5-MeO-DMT, wherein the pruvanserin is administered to pretreat at least 240 minutes prior to the 5-MeO-DMT. In some embodiments, the serotonin receptor modulator is pruvanserin and the psychedelic is 5-MeO-DMT, wherein the pruvanserin is administered to pretreat at least 270 minutes prior to the 5-MeO-DMT. In some embodiments, the serotonin receptor modulator is pruvanserin and the psychedelic is 5-MeO-DMT, wherein the pruvanserin is administered to pretreat at least 300 minutes prior to the 5-MeO-DMT. In some embodiments, the serotonin receptor modulator is pruvanserin and the psychedelic is 5-MeO-DMT, wherein the pruvanserin is administered to pretreat at least 330 minutes prior to the 5-MeO-DMT. In some embodiments, the serotonin receptor modulator is pruvanserin and the psychedelic is 5-MeO-DMT, wherein the pruvanserin is administered to pretreat at least 360 minutes prior to the 5-MeO-DMT. In some preferred embodiments, the serotonin receptor modulator is pruvanserin and the psychedelic is 5-MeO-DMT, wherein pruvanserin is administered to pretreat between about 60 minutes and about 180 minutes prior to the administration of 5-MeO-DMT.

In some embodiments, the serotonin receptor modulator is eplivanserin and the psychedelic is 2C-B, wherein the eplivanserin is administered to pretreat at least 15 minutes prior to the administration of 2C-B. In some embodiments, the serotonin receptor modulator is eplivanserin and the psychedelic is 2C-B, wherein the eplivanserin is administered to pretreat at least 30 minutes prior to the 2C-B. In some embodiments, the serotonin receptor modulator is eplivanserin and the psychedelic is 2C-B, wherein the eplivanserin is administered to pretreat at least 60 minutes prior to the 2C-B. In some embodiments, the serotonin receptor modulator is eplivanserin and the psychedelic is 2C-B, wherein the eplivanserin is administered to pretreat at least 90 minutes prior to the 2C-B. In some embodiments, the serotonin receptor modulator is eplivanserin and the psychedelic is 2C-B, wherein the eplivanserin is administered to pretreat at least 120 minutes prior to the 2C-B. In some embodiments, the serotonin receptor modulator is eplivanserin and the psychedelic is 2C-B, wherein the eplivanserin is administered to pretreat at least 150 minutes prior to the 2C-B. In some embodiments, the serotonin receptor modulator is eplivanserin and the psychedelic is 2C-B, wherein the eplivanserin is administered to pretreat at least 180 minutes prior to the 2C-B. In some embodiments, the serotonin receptor modulator is eplivanserin and the psychedelic is 2C-B, wherein the eplivanserin is administered to pretreat at least 210 minutes prior to the 2C-B. In some embodiments, the serotonin receptor modulator is eplivanserin and the psychedelic is 2C-B, wherein the eplivanserin is administered to pretreat at least 240 minutes prior to the 2C-B. In some embodiments, the serotonin receptor modulator is eplivanserin and the psychedelic is 2C-B, wherein the eplivanserin is administered to pretreat at least 270 minutes prior to the 2C-B. In some embodiments, the serotonin receptor modulator is eplivanserin and the psychedelic is 2C-B, wherein the eplivanserin is administered to pretreat at least 300 minutes prior to the 2C-B. In some embodiments, the serotonin receptor modulator is eplivanserin and the psychedelic is 2C-B, wherein the eplivanserin is administered to pretreat at least 330 minutes prior to the 2C-B. In some embodiments, the serotonin receptor modulator is eplivanserin and the psychedelic is 2C-B, wherein the eplivanserin is administered to pretreat at least 360 minutes prior to the 2C-B.

In some preferred embodiments, the serotonin receptor modulator is eplivanserin and the psychedelic is 2C-B, wherein eplivanserin is administered to pretreat between about 60 minutes and about 180 minutes prior to the administration of 2C-B.

In some embodiments, the serotonin receptor modulator is volinanserin and the psychedelic is 2C-B, wherein the volinanserin is administered to pretreat at least 15 minutes prior to the administration of 2C-B. In some embodiments, the serotonin receptor modulator is volinanserin and the psychedelic is 2C-B, wherein the volinanserin is administered to pretreat at least 30 minutes prior to the 2C-B. In some embodiments, the serotonin receptor modulator is volinanserin and the psychedelic is 2C-B, wherein the volinanserin is administered to pretreat at least 60 minutes prior to the 2C-B. In some embodiments, the serotonin receptor modulator is volinanserin and the psychedelic is 2C-B, wherein the volinanserin is administered to pretreat at least 90 minutes prior to the 2C-B. In some embodiments, the serotonin receptor modulator is volinanserin and the psychedelic is 2C-B, wherein the volinanserin is administered to pretreat at least 120 minutes prior to the 2C-B. In some embodiments, the serotonin receptor modulator is volinanserin and the psychedelic is 2C-B, wherein the volinanserin is administered to pretreat at least 150 minutes prior to the 2C-B. In some embodiments, the serotonin receptor modulator is volinanserin and the psychedelic is 2C-B, wherein the volinanserin is administered to pretreat between about 15 minutes and about 150 minutes prior to the 2C-B. In some embodiments, the serotonin receptor modulator is volinanserin and the psychedelic is 2C-B, wherein the volinanserin is administered to pretreat at least 180 minutes prior to the 2C-B. In some embodiments, the serotonin receptor modulator is volinanserin and the psychedelic is 2C-B, wherein the volinanserin is administered to pretreat at least 210 minutes prior to the 2C-B. In some embodiments, the serotonin receptor modulator is volinanserin and the psychedelic is 2C-B, wherein the volinanserin is administered to pretreat at least 240 minutes prior to the 2C-B. In some embodiments, the serotonin receptor modulator is volinanserin and the psychedelic is 2C-B, wherein the volinanserin is administered to pretreat at least 270 minutes prior to the 2C-B. In some embodiments, the serotonin receptor modulator is volinanserin and the psychedelic is 2C-B, wherein the volinanserin is administered to pretreat at least 300 minutes prior to the 2C-B. In some embodiments, the serotonin receptor modulator is volinanserin and the psychedelic is 2C-B, wherein the volinanserin is administered to pretreat at least 330 minutes prior to the 2C-B. In some embodiments, the serotonin receptor modulator is volinanserin and the psychedelic is 2C-B, wherein the volinanserin is administered to pretreat at least 360 minutes prior to the 2C-B. In some preferred embodiments, the serotonin receptor modulator is volinanserin and the psychedelic is 2C-B, wherein volinanserin is administered to pretreat between about 60 minutes and about 180 minutes prior to the administration of 2C-B.

In some embodiments, the serotonin receptor modulator is ketanserin and the psychedelic is 2C-B, wherein the ketanserin is administered to pretreat at least 15 minutes prior to the administration of 2C-B. In some embodiments, the serotonin receptor modulator is ketanserin and the psychedelic is 2C-B, wherein the ketanserin is administered to pretreat at least 30 minutes prior to the 2C-B. In some embodiments, the serotonin receptor modulator is ketanserin and the psychedelic is 2C-B, wherein the ketanserin is administered to pretreat at least 60 minutes prior to the 2C-B. In some embodiments, the serotonin receptor modulator is ketanserin and the psychedelic is 2C-B, wherein the ketanserin is administered to pretreat at least 90 minutes prior to the 2C-B. In some embodiments, the serotonin receptor modulator is ketanserin and the psychedelic is 2C-B, wherein the ketanserin is administered to pretreat at least 120 minutes prior to the 2C-B. In some embodiments, the serotonin receptor modulator is ketanserin and the psychedelic is 2C-B, wherein the ketanserin is administered to pretreat at least 150 minutes prior to the 2C-B. In some embodiments, the serotonin receptor modulator is ketanserin and the psychedelic is 2C-B, wherein the ketanserin is administered to pretreat between about 15 minutes and about 150 minutes prior to the 2C-B. In some embodiments, the serotonin receptor modulator is ketanserin and the psychedelic is 2C-B, wherein the ketanserin is administered to pretreat at least 180 minutes prior to the 2C-B. In some embodiments, the serotonin receptor modulator is ketanserin and the psychedelic is 2C-B, wherein the ketanserin is administered to pretreat at least 210 minutes prior to the 2C-B. In some embodiments, the serotonin receptor modulator is ketanserin and the psychedelic is 2C-B, wherein the ketanserin is administered to pretreat at least 240 minutes prior to the 2C-B. In some embodiments, the serotonin receptor modulator is ketanserin and the psychedelic is 2C-B, wherein the ketanserin is administered to pretreat at least 270 minutes prior to the 2C-B. In some embodiments, the serotonin receptor modulator is ketanserin and the psychedelic is 2C-B, wherein the ketanserin is administered to pretreat at least 300 minutes prior to the 2C-B. In some embodiments, the serotonin receptor modulator is ketanserin and the psychedelic is 2C-B, wherein the ketanserin is administered to pretreat at least 330 minutes prior to the 2C-B. In some embodiments, the serotonin receptor modulator is ketanserin and the psychedelic is 2C-B, wherein the ketanserin is administered to pretreat at least 360 minutes prior to the 2C-B. In some preferred embodiments, the serotonin receptor modulator is ketanserin and the psychedelic is 2C-B, wherein ketanserin is administered to pretreat between about 60 minutes and about 180 minutes prior to the administration of 2C-B.

In some embodiments, the serotonin receptor modulator is ritanserin and the psychedelic is 2C-B, wherein the ritanserin is administered to pretreat at least 15 minutes prior to the 2C-B. In some embodiments, the serotonin receptor modulator is ritanserin and the psychedelic is 2C-B, wherein the ritanserin is administered to pretreat at least 30 minutes prior to the 2C-B. In some embodiments, the serotonin receptor modulator is ritanserin and the psychedelic is 2C-B, wherein the ritanserin is administered to pretreat at least 60 minutes prior to the 2C-B. In some embodiments, the serotonin receptor modulator is ritanserin and the psychedelic is 2C-B, wherein the ritanserin is administered to pretreat at least 90 minutes prior to the 2C-B. In some embodiments, the serotonin receptor modulator is ritanserin and the psychedelic is 2C-B, wherein the ritanserin is administered to pretreat at least 120 minutes prior to the 2C-B. In some embodiments, the serotonin receptor modulator is ritanserin and the psychedelic is 2C-B, wherein the ritanserin is administered to pretreat at least 150 minutes prior to the 2C-B. In some embodiments, the serotonin receptor modulator is ritanserin and the psychedelic is 2C-B, wherein the ritanserin is administered to pretreat between about 15 minutes and about 150 minutes prior to the 2C-B. In some embodiments, the serotonin receptor modulator is ritanserin and the psychedelic is 2C-B, wherein the ritanserin is administered to pretreat at least 180 minutes prior to the 2C-B. In some embodiments, the serotonin receptor modulator is ritanserin and the psychedelic is 2C-B, wherein the ritanserin is administered to pretreat at least 210 minutes prior to the 2C-B. In some embodiments, the serotonin receptor modulator is ritanserin and the psychedelic is 2C-B, wherein the ritanserin is administered to pretreat at least 240 minutes prior to the 2C-B. In some embodiments, the serotonin receptor modulator is ritanserin and the psychedelic is 2C-B, wherein the ritanserin is administered to pretreat at least 270 minutes prior to the 2C-B. In some embodiments, the serotonin receptor modulator is ritanserin and the psychedelic is 2C-B, wherein the ritanserin is administered to pretreat at least 300 minutes prior to the 2C-B. In some embodiments, the serotonin receptor modulator is ritanserin and the psychedelic is 2C-B, wherein the ritanserin is administered to pretreat at least 330 minutes prior to the 2C-B. In some embodiments, the serotonin receptor modulator is ritanserin and the psychedelic is 2C-B, wherein the ritanserin is administered to pretreat at least 360 minutes prior to the 2C-B. In some preferred embodiments, the serotonin receptor modulator is ritanserin and the psychedelic is 2C-B, wherein ritanserin is administered to pretreat between about 60 minutes and about 180 minutes prior to the administration of 2C-B.

In some embodiments, the serotonin receptor modulator is pimavanserin and the psychedelic is 2C-B, wherein the pimavanserin is administered to pretreat at least 15 minutes prior to the 2C-B. In some embodiments, the serotonin receptor modulator is pimavanserin and the psychedelic is 2C-B, wherein the pimavanserin is administered to pretreat at least 30 minutes prior to the 2C-B. In some embodiments, the serotonin receptor modulator is pimavanserin and the psychedelic is 2C-B, wherein the pimavanserin is administered to pretreat at least 60 minutes prior to the 2C-B. In some embodiments, the serotonin receptor modulator is pimavanserin and the psychedelic is 2C-B, wherein the pimavanserin is administered to pretreat at least 90 minutes prior to the 2C-B. In some embodiments, the serotonin receptor modulator is pimavanserin and the psychedelic is 2C-B, wherein the pimavanserin is administered to pretreat at least 120 minutes prior to the 2C-B. In some embodiments, the serotonin receptor modulator is pimavanserin and the psychedelic is 2C-B, wherein the pimavanserin is administered to pretreat at least 150 minutes prior to the 2C-B. In some embodiments, the serotonin receptor modulator is pimavanserin and the psychedelic is 2C-B, wherein the pimavanserin is administered to pretreat between about 15 minutes and about 150 minutes prior to the 2C-B. In some embodiments, the serotonin receptor modulator is pimavanserin and the psychedelic is 2C-B, wherein the pimavanserin is administered to pretreat at least 180 minutes prior to the 2C-B. In some embodiments, the serotonin receptor modulator is pimavanserin and the psychedelic is 2C-B, wherein the pimavanserin is administered to pretreat at least 210 minutes prior to the 2C-B. In some embodiments, the serotonin receptor modulator is pimavanserin and the psychedelic is 2C-B, wherein the pimavanserin is administered to pretreat at least 240 minutes prior to the 2C-B. In some embodiments, the serotonin receptor modulator is pimavanserin and the psychedelic is 2C-B, wherein the pimavanserin is administered to pretreat at least 270 minutes prior to the 2C-B. In some embodiments, the serotonin receptor modulator is pimavanserin and the psychedelic is 2C-B, wherein the pimavanserin is administered to pretreat at least 300 minutes prior to the 2C-B. In some embodiments, the serotonin receptor modulator is pimavanserin and the psychedelic is 2C-B, wherein the pimavanserin is administered to pretreat at least 330 minutes prior to the 2C-B. In some embodiments, the serotonin receptor modulator is pimavanserin and the psychedelic is 2C-B, wherein the pimavanserin is administered to pretreat at least 360 minutes prior to the 2C-B. In some preferred embodiments, the serotonin receptor modulator is pimavanserin and the psychedelic is 2C-B, wherein pimavanserin is administered to pretreat between about 60 minutes and about 180 minutes prior to the administration of 2C-B.

In some embodiments, the serotonin receptor modulator is nelotanserin and the psychedelic is 2C-B, wherein the nelotanserin is administered to pretreat at least 15 minutes prior to the 2C-B. In some embodiments, the serotonin receptor modulator is nelotanserin and the psychedelic is 2C-B, wherein the nelotanserin is administered to pretreat at least 30 minutes prior to the 2C-B. In some embodiments, the serotonin receptor modulator is nelotanserin and the psychedelic is 2C-B, wherein the nelotanserin is administered to pretreat at least 60 minutes prior to the 2C-B. In some embodiments, the serotonin receptor modulator is nelotanserin and the psychedelic is 2C-B, wherein the nelotanserin is administered to pretreat at least 90 minutes prior to the 2C-B. In some embodiments, the serotonin receptor modulator is nelotanserin and the psychedelic is 2C-B, wherein the nelotanserin is administered to pretreat at least 120 minutes prior to the 2C-B. In some embodiments, the serotonin receptor modulator is nelotanserin and the psychedelic is 2C-B, wherein the nelotanserin is administered to pretreat at least 150 minutes prior to the 2C-B. In some embodiments, the serotonin receptor modulator is nelotanserin and the psychedelic is 2C-B, wherein the nelotanserin is administered to pretreat between about 15 minutes and about 150 minutes prior to the 2C-B. In some embodiments, the serotonin receptor modulator is nelotanserin and the psychedelic is 2C-B, wherein the nelotanserin is administered to pretreat at least 180 minutes prior to the 2C-B. In some embodiments, the serotonin receptor modulator is nelotanserin and the psychedelic is 2C-B, wherein the nelotanserin is administered to pretreat at least 210 minutes prior to the 2C-B. In some embodiments, the serotonin receptor modulator is nelotanserin and the psychedelic is 2C-B, wherein the nelotanserin is administered to pretreat at least 240 minutes prior to the 2C-B. In some embodiments, the serotonin receptor modulator is nelotanserin and the psychedelic is 2C-B, wherein the nelotanserin is administered to pretreat at least 270 minutes prior to the 2C-B. In some embodiments, the serotonin receptor modulator is nelotanserin and the psychedelic is 2C-B, wherein the nelotanserin is administered to pretreat at least 300 minutes prior to the 2C-B. In some embodiments, the serotonin receptor modulator is nelotanserin and the psychedelic is 2C-B, wherein the nelotanserin is administered to pretreat at least 330 minutes prior to the 2C-B. In some embodiments, the serotonin receptor modulator is nelotanserin and the psychedelic is 2C-B, wherein the nelotanserin is administered to pretreat at least 360 minutes prior to the 2C-B. In some preferred embodiments, the serotonin receptor modulator is nelotanserin and the psychedelic is 2C-B, wherein nelotanserin is administered to pretreat between about 60 minutes and about 180 minutes prior to the administration of 2C-B.

In some embodiments, the serotonin receptor modulator is pruvanserin and the psychedelic is 2C-B, wherein the pruvanserin is administered to pretreat at least 15 minutes prior to the 2C-B. In some embodiments, the serotonin receptor modulator is pruvanserin and the psychedelic is 2C-B, wherein the pruvanserin is administered to pretreat at least 30 minutes prior to the 2C-B. In some embodiments, the serotonin receptor modulator is pruvanserin and the psychedelic is 2C-B, wherein the pruvanserin is administered to pretreat at least 60 minutes prior to the 2C-B. In some embodiments, the serotonin receptor modulator is pruvanserin and the psychedelic is 2C-B, wherein the pruvanserin is administered to pretreat at least 90 minutes prior to the 2C-B. In some embodiments, the serotonin receptor modulator is pruvanserin and the psychedelic is 2C-B, wherein the pruvanserin is administered to pretreat at least 120 minutes prior to the 2C-B. In some embodiments, the serotonin receptor modulator is pruvanserin and the psychedelic is 2C-B, wherein the pruvanserin is administered to pretreat at least 150 minutes prior to the 2C-B. In some embodiments, the serotonin receptor modulator is pruvanserin and the psychedelic is 2C-B, wherein the pruvanserin is administered to pretreat between about 15 minutes and about 150 minutes prior to the 2C-B. In some embodiments, the serotonin receptor modulator is pruvanserin and the psychedelic is 2C-B, wherein the pruvanserin is administered to pretreat at least 180 minutes prior to the 2C-B. In some embodiments, the serotonin receptor modulator is pruvanserin and the psychedelic is 2C-B, wherein the pruvanserin is administered to pretreat at least 210 minutes prior to the 2C-B. In some embodiments, the serotonin receptor modulator is pruvanserin and the psychedelic is 2C-B, wherein the pruvanserin is administered to pretreat at least 240 minutes prior to the 2C-B. In some embodiments, the serotonin receptor modulator is pruvanserin and the psychedelic is 2C-B, wherein the pruvanserin is administered to pretreat at least 270 minutes prior to the 2C-B. In some embodiments, the serotonin receptor modulator is pruvanserin and the psychedelic is 2C-B, wherein the pruvanserin is administered to pretreat at least 300 minutes prior to the 2C-B. In some embodiments, the serotonin receptor modulator is pruvanserin and the psychedelic is 2C-B, wherein the pruvanserin is administered to pretreat at least 330 minutes prior to the 2C-B. In some embodiments, the serotonin receptor modulator is pruvanserin and the psychedelic is 2C-B, wherein the pruvanserin is administered to pretreat at least 360 minutes prior to the 2C-B. In some preferred embodiments, the serotonin receptor modulator is pruvanserin and the psychedelic is 2C-B, wherein pruvanserin is administered to pretreat between about 60 minutes and about 180 minutes prior to the administration of 2C-B.

In some embodiments, the serotonin receptor modulator is eplivanserin and the psychedelic is ibogaine, wherein the eplivanserin is administered to pretreat at least 15 minutes prior to the administration of ibogaine. In some embodiments, the serotonin receptor modulator is eplivanserin and the psychedelic is ibogaine, wherein the eplivanserin is administered to pretreat at least 30 minutes prior to the ibogaine. In some embodiments, the serotonin receptor modulator is eplivanserin and the psychedelic is ibogaine, wherein the eplivanserin is administered to pretreat at least 60 minutes prior to the ibogaine. In some embodiments, the serotonin receptor modulator is eplivanserin and the psychedelic is ibogaine, wherein the eplivanserin is administered to pretreat at least 90 minutes prior to the ibogaine. In some embodiments, the serotonin receptor modulator is eplivanserin and the psychedelic is ibogaine, wherein the eplivanserin is administered to pretreat at least 120 minutes prior to the ibogaine. In some embodiments, the serotonin receptor modulator is eplivanserin and the psychedelic is ibogaine, wherein the eplivanserin is administered to pretreat at least 150 minutes prior to the ibogaine. In some embodiments, the serotonin receptor modulator is eplivanserin and the psychedelic is ibogaine, wherein the eplivanserin is administered to pretreat at least 180 minutes prior to the ibogaine. In some embodiments, the serotonin receptor modulator is eplivanserin and the psychedelic is ibogaine, wherein the eplivanserin is administered to pretreat at least 210 minutes prior to the ibogaine. In some embodiments, the serotonin receptor modulator is eplivanserin and the psychedelic is ibogaine, wherein the eplivanserin is administered to pretreat at least 240 minutes prior to the ibogaine. In some embodiments, the serotonin receptor modulator is eplivanserin and the psychedelic is ibogaine, wherein the eplivanserin is administered to pretreat at least 270 minutes prior to the ibogaine. In some embodiments, the serotonin receptor modulator is eplivanserin and the psychedelic is ibogaine, wherein the eplivanserin is administered to pretreat at least 300 minutes prior to the ibogaine. In some embodiments, the serotonin receptor modulator is eplivanserin and the psychedelic is ibogaine, wherein the eplivanserin is administered to pretreat at least 330 minutes prior to the ibogaine. In some embodiments, the serotonin receptor modulator is eplivanserin and the psychedelic is ibogaine, wherein the eplivanserin is administered to pretreat at least 360 minutes prior to the ibogaine.

In some preferred embodiments, the serotonin receptor modulator is eplivanserin and the psychedelic is ibogaine, wherein eplivanserin is administered to pretreat between about 60 minutes and about 180 minutes prior to the administration of ibogaine.

In some embodiments, the serotonin receptor modulator is volinanserin and the psychedelic is ibogaine, wherein the volinanserin is administered to pretreat at least 15 minutes prior to the administration of ibogaine. In some embodiments, the serotonin receptor modulator is volinanserin and the psychedelic is ibogaine, wherein the volinanserin is administered to pretreat at least 30 minutes prior to the ibogaine. In some embodiments, the serotonin receptor modulator is volinanserin and the psychedelic is ibogaine, wherein the volinanserin is administered to pretreat at least 60 minutes prior to the ibogaine. In some embodiments, the serotonin receptor modulator is volinanserin and the psychedelic is ibogaine, wherein the volinanserin is administered to pretreat at least 90 minutes prior to the ibogaine. In some embodiments, the serotonin receptor modulator is volinanserin and the psychedelic is ibogaine, wherein the volinanserin is administered to pretreat at least 120 minutes prior to the ibogaine. In some embodiments, the serotonin receptor modulator is volinanserin and the psychedelic is ibogaine, wherein the volinanserin is administered to pretreat at least 150 minutes prior to the ibogaine. In some embodiments, the serotonin receptor modulator is volinanserin and the psychedelic is ibogaine, wherein the volinanserin is administered to pretreat between about 15 minutes and about 150 minutes prior to the ibogaine. In some embodiments, the serotonin receptor modulator is volinanserin and the psychedelic is ibogaine, wherein the volinanserin is administered to pretreat at least 180 minutes prior to the ibogaine. In some embodiments, the serotonin receptor modulator is volinanserin and the psychedelic is ibogaine, wherein the volinanserin is administered to pretreat at least 210 minutes prior to the ibogaine. In some embodiments, the serotonin receptor modulator is volinanserin and the psychedelic is ibogaine, wherein the volinanserin is administered to pretreat at least 240 minutes prior to the ibogaine. In some embodiments, the serotonin receptor modulator is volinanserin and the psychedelic is ibogaine, wherein the volinanserin is administered to pretreat at least 270 minutes prior to the ibogaine. In some embodiments, the serotonin receptor modulator is volinanserin and the psychedelic is ibogaine, wherein the volinanserin is administered to pretreat at least 300 minutes prior to the ibogaine. In some embodiments, the serotonin receptor modulator is volinanserin and the psychedelic is ibogaine, wherein the volinanserin is administered to pretreat at least 330 minutes prior to the ibogaine. In some embodiments, the serotonin receptor modulator is volinanserin and the psychedelic is ibogaine, wherein the volinanserin is administered to pretreat at least 360 minutes prior to the ibogaine. In some preferred embodiments, the serotonin receptor modulator is volinanserin and the psychedelic is ibogaine, wherein volinanserin is administered to pretreat between about 60 minutes and about 180 minutes prior to the administration of ibogaine.

In some embodiments, the serotonin receptor modulator is ketanserin and the psychedelic is ibogaine, wherein the ketanserin is administered to pretreat at least 15 minutes prior to the administration of ibogaine. In some embodiments, the serotonin receptor modulator is ketanserin and the psychedelic is ibogaine, wherein the ketanserin is administered to pretreat at least 30 minutes prior to the ibogaine. In some embodiments, the serotonin receptor modulator is ketanserin and the psychedelic is ibogaine, wherein the ketanserin is administered to pretreat at least 60 minutes prior to the ibogaine. In some embodiments, the serotonin receptor modulator is ketanserin and the psychedelic is ibogaine, wherein the ketanserin is administered to pretreat at least 90 minutes prior to the ibogaine. In some embodiments, the serotonin receptor modulator is ketanserin and the psychedelic is ibogaine, wherein the ketanserin is administered to pretreat at least 120 minutes prior to the ibogaine. In some embodiments, the serotonin receptor modulator is ketanserin and the psychedelic is ibogaine, wherein the ketanserin is administered to pretreat at least 150 minutes prior to the ibogaine. In some embodiments, the serotonin receptor modulator is ketanserin and the psychedelic is ibogaine, wherein the ketanserin is administered to pretreat between about 15 minutes and about 150 minutes prior to the ibogaine. In some embodiments, the serotonin receptor modulator is ketanserin and the psychedelic is ibogaine, wherein the ketanserin is administered to pretreat at least 180 minutes prior to the ibogaine. In some embodiments, the serotonin receptor modulator is ketanserin and the psychedelic is ibogaine, wherein the ketanserin is administered to pretreat at least 210 minutes prior to the ibogaine. In some embodiments, the serotonin receptor modulator is ketanserin and the psychedelic is ibogaine, wherein the ketanserin is administered to pretreat at least 240 minutes prior to the ibogaine. In some embodiments, the serotonin receptor modulator is ketanserin and the psychedelic is ibogaine, wherein the ketanserin is administered to pretreat at least 270 minutes prior to the ibogaine. In some embodiments, the serotonin receptor modulator is ketanserin and the psychedelic is ibogaine, wherein the ketanserin is administered to pretreat at least 300 minutes prior to the ibogaine. In some embodiments, the serotonin receptor modulator is ketanserin and the psychedelic is ibogaine, wherein the ketanserin is administered to pretreat at least 330 minutes prior to the ibogaine. In some embodiments, the serotonin receptor modulator is ketanserin and the psychedelic is ibogaine, wherein the ketanserin is administered to pretreat at least 360 minutes prior to the ibogaine. In some preferred embodiments, the serotonin receptor modulator is ketanserin and the psychedelic is ibogaine, wherein ketanserin is administered to pretreat between about 60 minutes and about 180 minutes prior to the administration of ibogaine.

In some embodiments, the serotonin receptor modulator is ritanserin and the psychedelic is ibogaine, wherein the ritanserin is administered to pretreat at least 15 minutes prior to the ibogaine. In some embodiments, the serotonin receptor modulator is ritanserin and the psychedelic is ibogaine, wherein the ritanserin is administered to pretreat at least 30 minutes prior to the ibogaine. In some embodiments, the serotonin receptor modulator is ritanserin and the psychedelic is ibogaine, wherein the ritanserin is administered to pretreat at least 60 minutes prior to the ibogaine. In some embodiments, the serotonin receptor modulator is ritanserin and the psychedelic is ibogaine, wherein the ritanserin is administered to pretreat at least 90 minutes prior to the ibogaine. In some embodiments, the serotonin receptor modulator is ritanserin and the psychedelic is ibogaine, wherein the ritanserin is administered to pretreat at least 120 minutes prior to the ibogaine. In some embodiments, the serotonin receptor modulator is ritanserin and the psychedelic is ibogaine, wherein the ritanserin is administered to pretreat at least 150 minutes prior to the ibogaine. In some embodiments, the serotonin receptor modulator is ritanserin and the psychedelic is ibogaine, wherein the ritanserin is administered to pretreat between about 15 minutes and about 150 minutes prior to the ibogaine. In some embodiments, the serotonin receptor modulator is ritanserin and the psychedelic is ibogaine, wherein the ritanserin is administered to pretreat at least 180 minutes prior to the ibogaine. In some embodiments, the serotonin receptor modulator is ritanserin and the psychedelic is ibogaine, wherein the ritanserin is administered to pretreat at least 210 minutes prior to the ibogaine. In some embodiments, the serotonin receptor modulator is ritanserin and the psychedelic is ibogaine, wherein the ritanserin is administered to pretreat at least 240 minutes prior to the ibogaine. In some embodiments, the serotonin receptor modulator is ritanserin and the psychedelic is ibogaine, wherein the ritanserin is administered to pretreat at least 270 minutes prior to the ibogaine. In some embodiments, the serotonin receptor modulator is ritanserin and the psychedelic is ibogaine, wherein the ritanserin is administered to pretreat at least 300 minutes prior to the ibogaine. In some embodiments, the serotonin receptor modulator is ritanserin and the psychedelic is ibogaine, wherein the ritanserin is administered to pretreat at least 330 minutes prior to the ibogaine. In some embodiments, the serotonin receptor modulator is ritanserin and the psychedelic is ibogaine, wherein the ritanserin is administered to pretreat at least 360 minutes prior to the ibogaine. In some preferred embodiments, the serotonin receptor modulator is ritanserin and the psychedelic is ibogaine, wherein ritanserin is administered to pretreat between about 60 minutes and about 180 minutes prior to the administration of ibogaine.

In some embodiments, the serotonin receptor modulator is pimavanserin and the psychedelic is ibogaine, wherein the pimavanserin is administered to pretreat at least 15 minutes prior to the ibogaine. In some embodiments, the serotonin receptor modulator is pimavanserin and the psychedelic is ibogaine, wherein the pimavanserin is administered to pretreat at least 30 minutes prior to the ibogaine. In some embodiments, the serotonin receptor modulator is pimavanserin and the psychedelic is ibogaine, wherein the pimavanserin is administered to pretreat at least 60 minutes prior to the ibogaine. In some embodiments, the serotonin receptor modulator is pimavanserin and the psychedelic is ibogaine, wherein the pimavanserin is administered to pretreat at least 90 minutes prior to the ibogaine. In some embodiments, the serotonin receptor modulator is pimavanserin and the psychedelic is ibogaine, wherein the pimavanserin is administered to pretreat at least 120 minutes prior to the ibogaine. In some embodiments, the serotonin receptor modulator is pimavanserin and the psychedelic is ibogaine, wherein the pimavanserin is administered to pretreat at least 150 minutes prior to the ibogaine. In some embodiments, the serotonin receptor modulator is pimavanserin and the psychedelic is ibogaine, wherein the pimavanserin is administered to pretreat between about 15 minutes and about 150 minutes prior to the ibogaine. In some embodiments, the serotonin receptor modulator is pimavanserin and the psychedelic is ibogaine, wherein the pimavanserin is administered to pretreat at least 180 minutes prior to the ibogaine. In some embodiments, the serotonin receptor modulator is pimavanserin and the psychedelic is ibogaine, wherein the pimavanserin is administered to pretreat at least 210 minutes prior to the ibogaine. In some embodiments, the serotonin receptor modulator is pimavanserin and the psychedelic is ibogaine, wherein the pimavanserin is administered to pretreat at least 240 minutes prior to the ibogaine. In some embodiments, the serotonin receptor modulator is pimavanserin and the psychedelic is ibogaine, wherein the pimavanserin is administered to pretreat at least 270 minutes prior to the ibogaine. In some embodiments, the serotonin receptor modulator is pimavanserin and the psychedelic is ibogaine, wherein the pimavanserin is administered to pretreat at least 300 minutes prior to the ibogaine. In some embodiments, the serotonin receptor modulator is pimavanserin and the psychedelic is ibogaine, wherein the pimavanserin is administered to pretreat at least 330 minutes prior to the ibogaine. In some embodiments, the serotonin receptor modulator is pimavanserin and the psychedelic is ibogaine, wherein the pimavanserin is administered to pretreat at least 360 minutes prior to the ibogaine. In some preferred embodiments, the serotonin receptor modulator is pimavanserin and the psychedelic is ibogaine, wherein pimavanserin is administered to pretreat between about 60 minutes and about 180 minutes prior to the administration of ibogaine.

In some embodiments, the serotonin receptor modulator is nelotanserin and the psychedelic is ibogaine, wherein the nelotanserin is administered to pretreat at least 15 minutes prior to the ibogaine. In some embodiments, the serotonin receptor modulator is nelotanserin and the psychedelic is ibogaine, wherein the nelotanserin is administered to pretreat at least 30 minutes prior to the ibogaine. In some embodiments, the serotonin receptor modulator is nelotanserin and the psychedelic is ibogaine, wherein the nelotanserin is administered to pretreat at least 60 minutes prior to the ibogaine. In some embodiments, the serotonin receptor modulator is nelotanserin and the psychedelic is ibogaine, wherein the nelotanserin is administered to pretreat at least 90 minutes prior to the ibogaine. In some embodiments, the serotonin receptor modulator is nelotanserin and the psychedelic is ibogaine, wherein the nelotanserin is administered to pretreat at least 120 minutes prior to the ibogaine. In some embodiments, the serotonin receptor modulator is nelotanserin and the psychedelic is ibogaine, wherein the nelotanserin is administered to pretreat at least 150 minutes prior to the ibogaine. In some embodiments, the serotonin receptor modulator is nelotanserin and the psychedelic is ibogaine, wherein the nelotanserin is administered to pretreat between about 15 minutes and about 150 minutes prior to the ibogaine. In some embodiments, the serotonin receptor modulator is nelotanserin and the psychedelic is ibogaine, wherein the nelotanserin is administered to pretreat at least 180 minutes prior to the ibogaine. In some embodiments, the serotonin receptor modulator is nelotanserin and the psychedelic is ibogaine, wherein the nelotanserin is administered to pretreat at least 210 minutes prior to the ibogaine. In some embodiments, the serotonin receptor modulator is nelotanserin and the psychedelic is ibogaine, wherein the nelotanserin is administered to pretreat at least 240 minutes prior to the ibogaine. In some embodiments, the serotonin receptor modulator is nelotanserin and the psychedelic is ibogaine, wherein the nelotanserin is administered to pretreat at least 270 minutes prior to the ibogaine. In some embodiments, the serotonin receptor modulator is nelotanserin and the psychedelic is ibogaine, wherein the nelotanserin is administered to pretreat at least 300 minutes prior to the ibogaine. In some embodiments, the serotonin receptor modulator is nelotanserin and the psychedelic is ibogaine, wherein the nelotanserin is administered to pretreat at least 330 minutes prior to the ibogaine. In some embodiments, the serotonin receptor modulator is nelotanserin and the psychedelic is ibogaine, wherein the nelotanserin is administered to pretreat at least 360 minutes prior to the ibogaine. In some preferred embodiments, the serotonin receptor modulator is nelotanserin and the psychedelic is ibogaine, wherein nelotanserin is administered to pretreat between about 60 minutes and about 180 minutes prior to the administration of ibogaine.

In some embodiments, the serotonin receptor modulator is pruvanserin and the psychedelic is ibogaine, wherein the pruvanserin is administered to pretreat at least 15 minutes prior to the ibogaine. In some embodiments, the serotonin receptor modulator is pruvanserin and the psychedelic is ibogaine, wherein the pruvanserin is administered to pretreat at least 30 minutes prior to the ibogaine. In some embodiments, the serotonin receptor modulator is pruvanserin and the psychedelic is ibogaine, wherein the pruvanserin is administered to pretreat at least 60 minutes prior to the ibogaine. In some embodiments, the serotonin receptor modulator is pruvanserin and the psychedelic is ibogaine, wherein the pruvanserin is administered to pretreat at least 90 minutes prior to the ibogaine. In some embodiments, the serotonin receptor modulator is pruvanserin and the psychedelic is ibogaine, wherein the pruvanserin is administered to pretreat at least 120 minutes prior to the ibogaine. In some embodiments, the serotonin receptor modulator is pruvanserin and the psychedelic is ibogaine, wherein the pruvanserin is administered to pretreat at least 150 minutes prior to the ibogaine. In some embodiments, the serotonin receptor modulator is pruvanserin and the psychedelic is ibogaine, wherein the pruvanserin is administered to pretreat between about 15 minutes and about 150 minutes prior to the ibogaine. In some embodiments, the serotonin receptor modulator is pruvanserin and the psychedelic is ibogaine, wherein the pruvanserin is administered to pretreat at least 180 minutes prior to the ibogaine. In some embodiments, the serotonin receptor modulator is pruvanserin and the psychedelic is ibogaine, wherein the pruvanserin is administered to pretreat at least 210 minutes prior to the ibogaine. In some embodiments, the serotonin receptor modulator is pruvanserin and the psychedelic is ibogaine, wherein the pruvanserin is administered to pretreat at least 240 minutes prior to the ibogaine. In some embodiments, the serotonin receptor modulator is pruvanserin and the psychedelic is ibogaine, wherein the pruvanserin is administered to pretreat at least 270 minutes prior to the ibogaine. In some embodiments, the serotonin receptor modulator is pruvanserin and the psychedelic is ibogaine, wherein the pruvanserin is administered to pretreat at least 300 minutes prior to the ibogaine. In some embodiments, the serotonin receptor modulator is pruvanserin and the psychedelic is ibogaine, wherein the pruvanserin is administered to pretreat at least 330 minutes prior to the ibogaine. In some embodiments, the serotonin receptor modulator is pruvanserin and the psychedelic is ibogaine, wherein the pruvanserin is administered to pretreat at least 360 minutes prior to the ibogaine. In some preferred embodiments, the serotonin receptor modulator is pruvanserin and the psychedelic is ibogaine, wherein pruvanserin is administered to pretreat between about 60 minutes and about 180 minutes prior to the administration of ibogaine.

In some embodiments, the serotonin receptor modulator is eplivanserin and the psychedelic is MDMA, wherein the eplivanserin is administered to pretreat at least 15 minutes prior to the administration of MDMA. In some embodiments, the serotonin receptor modulator is eplivanserin and the psychedelic is MDMA, wherein the eplivanserin is administered to pretreat at least 30 minutes prior to the MDMA. In some embodiments, the serotonin receptor modulator is eplivanserin and the psychedelic is MDMA, wherein the eplivanserin is administered to pretreat at least 60 minutes prior to the MDMA. In some embodiments, the serotonin receptor modulator is eplivanserin and the psychedelic is MDMA, wherein the eplivanserin is administered to pretreat at least 90 minutes prior to the MDMA. In some embodiments, the serotonin receptor modulator is eplivanserin and the psychedelic is MDMA, wherein the eplivanserin is administered to pretreat at least 120 minutes prior to the MDMA. In some embodiments, the serotonin receptor modulator is eplivanserin and the psychedelic is MDMA, wherein the eplivanserin is administered to pretreat at least 150 minutes prior to the MDMA. In some embodiments, the serotonin receptor modulator is eplivanserin and the psychedelic is MDMA, wherein the eplivanserin is administered to pretreat at least 180 minutes prior to the MDMA. In some embodiments, the serotonin receptor modulator is eplivanserin and the psychedelic is MDMA, wherein the eplivanserin is administered to pretreat at least 210 minutes prior to the MDMA. In some embodiments, the serotonin receptor modulator is eplivanserin and the psychedelic is MDMA, wherein the eplivanserin is administered to pretreat at least 240 minutes prior to the MDMA. In some embodiments, the serotonin receptor modulator is eplivanserin and the psychedelic is MDMA, wherein the eplivanserin is administered to pretreat at least 270 minutes prior to the MDMA. In some embodiments, the serotonin receptor modulator is eplivanserin and the psychedelic is MDMA, wherein the eplivanserin is administered to pretreat at least 300 minutes prior to the MDMA. In some embodiments, the serotonin receptor modulator is eplivanserin and the psychedelic is MDMA, wherein the eplivanserin is administered to pretreat at least 330 minutes prior to the MDMA. In some embodiments, the serotonin receptor modulator is eplivanserin and the psychedelic is MDMA, wherein the eplivanserin is administered to pretreat at least 360 minutes prior to the MDMA.

In some preferred embodiments, the serotonin receptor modulator is eplivanserin and the psychedelic is MDMA, wherein eplivanserin is administered to pretreat between about 60 minutes and about 180 minutes prior to the administration of MDMA.

In some embodiments, the serotonin receptor modulator is volinanserin and the psychedelic is MDMA, wherein the volinanserin is administered to pretreat at least 15 minutes prior to the administration of MDMA. In some embodiments, the serotonin receptor modulator is volinanserin and the psychedelic is MDMA, wherein the volinanserin is administered to pretreat at least 30 minutes prior to the MDMA. In some embodiments, the serotonin receptor modulator is volinanserin and the psychedelic is MDMA, wherein the volinanserin is administered to pretreat at least 60 minutes prior to the MDMA. In some embodiments, the serotonin receptor modulator is volinanserin and the psychedelic is MDMA, wherein the volinanserin is administered to pretreat at least 90 minutes prior to the MDMA. In some embodiments, the serotonin receptor modulator is volinanserin and the psychedelic is MDMA, wherein the volinanserin is administered to pretreat at least 120 minutes prior to the MDMA. In some embodiments, the serotonin receptor modulator is volinanserin and the psychedelic is MDMA, wherein the volinanserin is administered to pretreat at least 150 minutes prior to the MDMA. In some embodiments, the serotonin receptor modulator is volinanserin and the psychedelic is MDMA, wherein the volinanserin is administered to pretreat between about 15 minutes and about 150 minutes prior to the MDMA. In some embodiments, the serotonin receptor modulator is volinanserin and the psychedelic is MDMA, wherein the volinanserin is administered to pretreat at least 180 minutes prior to the MDMA. In some embodiments, the serotonin receptor modulator is volinanserin and the psychedelic is MDMA, wherein the volinanserin is administered to pretreat at least 210 minutes prior to the MDMA. In some embodiments, the serotonin receptor modulator is volinanserin and the psychedelic is MDMA, wherein the volinanserin is administered to pretreat at least 240 minutes prior to the MDMA. In some embodiments, the serotonin receptor modulator is volinanserin and the psychedelic is MDMA, wherein the volinanserin is administered to pretreat at least 270 minutes prior to the MDMA. In some embodiments, the serotonin receptor modulator is volinanserin and the psychedelic is MDMA, wherein the volinanserin is administered to pretreat at least 300 minutes prior to the MDMA. In some embodiments, the serotonin receptor modulator is volinanserin and the psychedelic is MDMA, wherein the volinanserin is administered to pretreat at least 330 minutes prior to the MDMA. In some embodiments, the serotonin receptor modulator is volinanserin and the psychedelic is MDMA, wherein the volinanserin is administered to pretreat at least 360 minutes prior to the MDMA. In some preferred embodiments, the serotonin receptor modulator is volinanserin and the psychedelic is MDMA, wherein volinanserin is administered to pretreat between about 60 minutes and about 180 minutes prior to the administration of MDMA.

In some embodiments, the serotonin receptor modulator is ketanserin and the psychedelic is MDMA, wherein the ketanserin is administered to pretreat at least 15 minutes prior to the administration of MDMA. In some embodiments, the serotonin receptor modulator is ketanserin and the psychedelic is MDMA, wherein the ketanserin is administered to pretreat at least 30 minutes prior to the MDMA. In some embodiments, the serotonin receptor modulator is ketanserin and the psychedelic is MDMA, wherein the ketanserin is administered to pretreat at least 60 minutes prior to the MDMA. In some embodiments, the serotonin receptor modulator is ketanserin and the psychedelic is MDMA, wherein the ketanserin is administered to pretreat at least 90 minutes prior to the MDMA. In some embodiments, the serotonin receptor modulator is ketanserin and the psychedelic is MDMA, wherein the ketanserin is administered to pretreat at least 120 minutes prior to the MDMA. In some embodiments, the serotonin receptor modulator is ketanserin and the psychedelic is MDMA, wherein the ketanserin is administered to pretreat at least 150 minutes prior to the MDMA. In some embodiments, the serotonin receptor modulator is ketanserin and the psychedelic is MDMA, wherein the ketanserin is administered to pretreat between about 15 minutes and about 150 minutes prior to the MDMA. In some embodiments, the serotonin receptor modulator is ketanserin and the psychedelic is MDMA, wherein the ketanserin is administered to pretreat at least 180 minutes prior to the MDMA. In some embodiments, the serotonin receptor modulator is ketanserin and the psychedelic is MDMA, wherein the ketanserin is administered to pretreat at least 210 minutes prior to the MDMA. In some embodiments, the serotonin receptor modulator is ketanserin and the psychedelic is MDMA, wherein the ketanserin is administered to pretreat at least 240 minutes prior to the MDMA. In some embodiments, the serotonin receptor modulator is ketanserin and the psychedelic is MDMA, wherein the ketanserin is administered to pretreat at least 270 minutes prior to the MDMA. In some embodiments, the serotonin receptor modulator is ketanserin and the psychedelic is MDMA, wherein the ketanserin is administered to pretreat at least 300 minutes prior to the MDMA. In some embodiments, the serotonin receptor modulator is ketanserin and the psychedelic is MDMA, wherein the ketanserin is administered to pretreat at least 330 minutes prior to the MDMA. In some embodiments, the serotonin receptor modulator is ketanserin and the psychedelic is MDMA, wherein the ketanserin is administered to pretreat at least 360 minutes prior to the MDMA. In some preferred embodiments, the serotonin receptor modulator is ketanserin and the psychedelic is MDMA, wherein ketanserin is administered to pretreat between about 60 minutes and about 180 minutes prior to the administration of MDMA.

In some embodiments, the serotonin receptor modulator is ritanserin and the psychedelic is MDMA, wherein the ritanserin is administered to pretreat at least 15 minutes prior to the MDMA. In some embodiments, the serotonin receptor modulator is ritanserin and the psychedelic is MDMA, wherein the ritanserin is administered to pretreat at least 30 minutes prior to the MDMA. In some embodiments, the serotonin receptor modulator is ritanserin and the psychedelic is MDMA, wherein the ritanserin is administered to pretreat at least 60 minutes prior to the MDMA. In some embodiments, the serotonin receptor modulator is ritanserin and the psychedelic is MDMA, wherein the ritanserin is administered to pretreat at least 90 minutes prior to the MDMA. In some embodiments, the serotonin receptor modulator is ritanserin and the psychedelic is MDMA, wherein the ritanserin is administered to pretreat at least 120 minutes prior to the MDMA. In some embodiments, the serotonin receptor modulator is ritanserin and the psychedelic is MDMA, wherein the ritanserin is administered to pretreat at least 150 minutes prior to the MDMA. In some embodiments, the serotonin receptor modulator is ritanserin and the psychedelic is MDMA, wherein the ritanserin is administered to pretreat between about 15 minutes and about 150 minutes prior to the MDMA. In some embodiments, the serotonin receptor modulator is ritanserin and the psychedelic is MDMA, wherein the ritanserin is administered to pretreat at least 180 minutes prior to the MDMA. In some embodiments, the serotonin receptor modulator is ritanserin and the psychedelic is MDMA, wherein the ritanserin is administered to pretreat at least 210 minutes prior to the MDMA. In some embodiments, the serotonin receptor modulator is ritanserin and the psychedelic is MDMA, wherein the ritanserin is administered to pretreat at least 240 minutes prior to the MDMA. In some embodiments, the serotonin receptor modulator is ritanserin and the psychedelic is MDMA, wherein the ritanserin is administered to pretreat at least 270 minutes prior to the MDMA. In some embodiments, the serotonin receptor modulator is ritanserin and the psychedelic is MDMA, wherein the ritanserin is administered to pretreat at least 300 minutes prior to the MDMA. In some embodiments, the serotonin receptor modulator is ritanserin and the psychedelic is MDMA, wherein the ritanserin is administered to pretreat at least 330 minutes prior to the MDMA. In some embodiments, the serotonin receptor modulator is ritanserin and the psychedelic is MDMA, wherein the ritanserin is administered to pretreat at least 360 minutes prior to the MDMA. In some preferred embodiments, the serotonin receptor modulator is ritanserin and the psychedelic is MDMA, wherein ritanserin is administered to pretreat between about 60 minutes and about 180 minutes prior to the administration of MDMA.

In some embodiments, the serotonin receptor modulator is pimavanserin and the psychedelic is MDMA, wherein the pimavanserin is administered to pretreat at least 15 minutes prior to the MDMA. In some embodiments, the serotonin receptor modulator is pimavanserin and the psychedelic is MDMA, wherein the pimavanserin is administered to pretreat at least 30 minutes prior to the MDMA. In some embodiments, the serotonin receptor modulator is pimavanserin and the psychedelic is MDMA, wherein the pimavanserin is administered to pretreat at least 60 minutes prior to the MDMA. In some embodiments, the serotonin receptor modulator is pimavanserin and the psychedelic is MDMA, wherein the pimavanserin is administered to pretreat at least 90 minutes prior to the MDMA. In some embodiments, the serotonin receptor modulator is pimavanserin and the psychedelic is MDMA, wherein the pimavanserin is administered to pretreat at least 120 minutes prior to the MDMA. In some embodiments, the serotonin receptor modulator is pimavanserin and the psychedelic is MDMA, wherein the pimavanserin is administered to pretreat at least 150 minutes prior to the MDMA. In some embodiments, the serotonin receptor modulator is pimavanserin and the psychedelic is MDMA, wherein the pimavanserin is administered to pretreat between about 15 minutes and about 150 minutes prior to the MDMA. In some embodiments, the serotonin receptor modulator is pimavanserin and the psychedelic is MDMA, wherein the pimavanserin is administered to pretreat at least 180 minutes prior to the MDMA. In some embodiments, the serotonin receptor modulator is pimavanserin and the psychedelic is MDMA, wherein the pimavanserin is administered to pretreat at least 210 minutes prior to the MDMA. In some embodiments, the serotonin receptor modulator is pimavanserin and the psychedelic is MDMA, wherein the pimavanserin is administered to pretreat at least 240 minutes prior to the MDMA. In some embodiments, the serotonin receptor modulator is pimavanserin and the psychedelic is MDMA, wherein the pimavanserin is administered to pretreat at least 270 minutes prior to the MDMA. In some embodiments, the serotonin receptor modulator is pimavanserin and the psychedelic is MDMA, wherein the pimavanserin is administered to pretreat at least 300 minutes prior to the MDMA. In some embodiments, the serotonin receptor modulator is pimavanserin and the psychedelic is MDMA, wherein the pimavanserin is administered to pretreat at least 330 minutes prior to the MDMA. In some embodiments, the serotonin receptor modulator is pimavanserin and the psychedelic is MDMA, wherein the pimavanserin is administered to pretreat at least 360 minutes prior to the MDMA. In some preferred embodiments, the serotonin receptor modulator is pimavanserin and the psychedelic is MDMA, wherein pimavanserin is administered to pretreat between about 60 minutes and about 180 minutes prior to the administration of MDMA.

In some embodiments, the serotonin receptor modulator is nelotanserin and the psychedelic is MDMA, wherein the nelotanserin is administered to pretreat at least 15 minutes prior to the MDMA. In some embodiments, the serotonin receptor modulator is nelotanserin and the psychedelic is MDMA, wherein the nelotanserin is administered to pretreat at least 30 minutes prior to the MDMA. In some embodiments, the serotonin receptor modulator is nelotanserin and the psychedelic is MDMA, wherein the nelotanserin is administered to pretreat at least 60 minutes prior to the MDMA. In some embodiments, the serotonin receptor modulator is nelotanserin and the psychedelic is MDMA, wherein the nelotanserin is administered to pretreat at least 90 minutes prior to the MDMA. In some embodiments, the serotonin receptor modulator is nelotanserin and the psychedelic is MDMA, wherein the nelotanserin is administered to pretreat at least 120 minutes prior to the MDMA. In some embodiments, the serotonin receptor modulator is nelotanserin and the psychedelic is MDMA, wherein the nelotanserin is administered to pretreat at least 150 minutes prior to the MDMA. In some embodiments, the serotonin receptor modulator is nelotanserin and the psychedelic is MDMA, wherein the nelotanserin is administered to pretreat between about 15 minutes and about 150 minutes prior to the MDMA. In some embodiments, the serotonin receptor modulator is nelotanserin and the psychedelic is MDMA, wherein the nelotanserin is administered to pretreat at least 180 minutes prior to the MDMA. In some embodiments, the serotonin receptor modulator is nelotanserin and the psychedelic is MDMA, wherein the nelotanserin is administered to pretreat at least 210 minutes prior to the MDMA. In some embodiments, the serotonin receptor modulator is nelotanserin and the psychedelic is MDMA, wherein the nelotanserin is administered to pretreat at least 240 minutes prior to the MDMA. In some embodiments, the serotonin receptor modulator is nelotanserin and the psychedelic is MDMA, wherein the nelotanserin is administered to pretreat at least 270 minutes prior to the MDMA. In some embodiments, the serotonin receptor modulator is nelotanserin and the psychedelic is MDMA, wherein the nelotanserin is administered to pretreat at least 300 minutes prior to the MDMA. In some embodiments, the serotonin receptor modulator is nelotanserin and the psychedelic is MDMA, wherein the nelotanserin is administered to pretreat at least 330 minutes prior to the MDMA. In some embodiments, the serotonin receptor modulator is nelotanserin and the psychedelic is MDMA, wherein the nelotanserin is administered to pretreat at least 360 minutes prior to the MDMA. In some preferred embodiments, the serotonin receptor modulator is nelotanserin and the psychedelic is MDMA, wherein nelotanserin is administered to pretreat between about 60 minutes and about 180 minutes prior to the administration of MDMA.

In some embodiments, the serotonin receptor modulator is pruvanserin and the psychedelic is MDMA, wherein the pruvanserin is administered to pretreat at least 15 minutes prior to the MDMA. In some embodiments, the serotonin receptor modulator is pruvanserin and the psychedelic is MDMA, wherein the pruvanserin is administered to pretreat at least 30 minutes prior to the MDMA. In some embodiments, the serotonin receptor modulator is pruvanserin and the psychedelic is MDMA, wherein the pruvanserin is administered to pretreat at least 60 minutes prior to the MDMA. In some embodiments, the serotonin receptor modulator is pruvanserin and the psychedelic is MDMA, wherein the pruvanserin is administered to pretreat at least 90 minutes prior to the MDMA. In some embodiments, the serotonin receptor modulator is pruvanserin and the psychedelic is MDMA, wherein the pruvanserin is administered to pretreat at least 120 minutes prior to the MDMA. In some embodiments, the serotonin receptor modulator is pruvanserin and the psychedelic is MDMA, wherein the pruvanserin is administered to pretreat at least 150 minutes prior to the MDMA. In some embodiments, the serotonin receptor modulator is pruvanserin and the psychedelic is MDMA, wherein the pruvanserin is administered to pretreat between about 15 minutes and about 150 minutes prior to the MDMA. In some embodiments, the serotonin receptor modulator is pruvanserin and the psychedelic is MDMA, wherein the pruvanserin is administered to pretreat at least 180 minutes prior to the MDMA. In some embodiments, the serotonin receptor modulator is pruvanserin and the psychedelic is MDMA, wherein the pruvanserin is administered to pretreat at least 210 minutes prior to the MDMA. In some embodiments, the serotonin receptor modulator is pruvanserin and the psychedelic is MDMA, wherein the pruvanserin is administered to pretreat at least 240 minutes prior to the MDMA. In some embodiments, the serotonin receptor modulator is pruvanserin and the psychedelic is MDMA, wherein the pruvanserin is administered to pretreat at least 270 minutes prior to the MDMA. In some embodiments, the serotonin receptor modulator is pruvanserin and the psychedelic is MDMA, wherein the pruvanserin is administered to pretreat at least 300 minutes prior to the MDMA. In some embodiments, the serotonin receptor modulator is pruvanserin and the psychedelic is MDMA, wherein the pruvanserin is administered to pretreat at least 330 minutes prior to the MDMA. In some embodiments, the serotonin receptor modulator is pruvanserin and the psychedelic is MDMA, wherein the pruvanserin is administered to pretreat at least 360 minutes prior to the MDMA. In some preferred embodiments, the serotonin receptor modulator is pruvanserin and the psychedelic is MDMA, wherein pruvanserin is administered to pretreat between about 60 minutes and about 180 minutes prior to the administration of MDMA.

In some embodiments, the serotonin receptor modulator is eplivanserin and the psychedelic is DOM, wherein the eplivanserin is administered to pretreat at least 15 minutes prior to the administration of DOM. In some embodiments, the serotonin receptor modulator is eplivanserin and the psychedelic is DOM, wherein the eplivanserin is administered to pretreat at least 30 minutes prior to the DOM. In some embodiments, the serotonin receptor modulator is eplivanserin and the psychedelic is DOM, wherein the eplivanserin is administered to pretreat at least 60 minutes prior to the DOM. In some embodiments, the serotonin receptor modulator is eplivanserin and the psychedelic is DOM, wherein the eplivanserin is administered to pretreat at least 90 minutes prior to the DOM. In some embodiments, the serotonin receptor modulator is eplivanserin and the psychedelic is DOM, wherein the eplivanserin is administered to pretreat at least 120 minutes prior to the DOM. In some embodiments, the serotonin receptor modulator is eplivanserin and the psychedelic is DOM, wherein the eplivanserin is administered to pretreat at least 150 minutes prior to the DOM. In some embodiments, the serotonin receptor modulator is eplivanserin and the psychedelic is DOM, wherein the eplivanserin is administered to pretreat at least 180 minutes prior to the DOM. In some embodiments, the serotonin receptor modulator is eplivanserin and the psychedelic is DOM, wherein the eplivanserin is administered to pretreat at least 210 minutes prior to the DOM. In some embodiments, the serotonin receptor modulator is eplivanserin and the psychedelic is DOM, wherein the eplivanserin is administered to pretreat at least 240 minutes prior to the DOM. In some embodiments, the serotonin receptor modulator is eplivanserin and the psychedelic is DOM, wherein the eplivanserin is administered to pretreat at least 270 minutes prior to the DOM. In some embodiments, the serotonin receptor modulator is eplivanserin and the psychedelic is DOM, wherein the eplivanserin is administered to pretreat at least 300 minutes prior to the DOM. In some embodiments, the serotonin receptor modulator is eplivanserin and the psychedelic is DOM, wherein the eplivanserin is administered to pretreat at least 330 minutes prior to the DOM. In some embodiments, the serotonin receptor modulator is eplivanserin and the psychedelic is DOM, wherein the eplivanserin is administered to pretreat at least 360 minutes prior to the DOM.

In some preferred embodiments, the serotonin receptor modulator is eplivanserin and the psychedelic is DOM, wherein eplivanserin is administered to pretreat between about 60 minutes and about 180 minutes prior to the administration of DOM.

In some embodiments, the serotonin receptor modulator is volinanserin and the psychedelic is DOM, wherein the volinanserin is administered to pretreat at least 15 minutes prior to the administration of DOM. In some embodiments, the serotonin receptor modulator is volinanserin and the psychedelic is DOM, wherein the volinanserin is administered to pretreat at least 30 minutes prior to the DOM. In some embodiments, the serotonin receptor modulator is volinanserin and the psychedelic is DOM, wherein the volinanserin is administered to pretreat at least 60 minutes prior to the DOM. In some embodiments, the serotonin receptor modulator is volinanserin and the psychedelic is DOM, wherein the volinanserin is administered to pretreat at least 90 minutes prior to the DOM. In some embodiments, the serotonin receptor modulator is volinanserin and the psychedelic is DOM, wherein the volinanserin is administered to pretreat at least 120 minutes prior to the DOM. In some embodiments, the serotonin receptor modulator is volinanserin and the psychedelic is DOM, wherein the volinanserin is administered to pretreat at least 150 minutes prior to the DOM. In some embodiments, the serotonin receptor modulator is volinanserin and the psychedelic is DOM, wherein the volinanserin is administered to pretreat between about 15 minutes and about 150 minutes prior to the DOM. In some embodiments, the serotonin receptor modulator is volinanserin and the psychedelic is DOM, wherein the volinanserin is administered to pretreat at least 180 minutes prior to the DOM. In some embodiments, the serotonin receptor modulator is volinanserin and the psychedelic is DOM, wherein the volinanserin is administered to pretreat at least 210 minutes prior to the DOM. In some embodiments, the serotonin receptor modulator is volinanserin and the psychedelic is DOM, wherein the volinanserin is administered to pretreat at least 240 minutes prior to the DOM. In some embodiments, the serotonin receptor modulator is volinanserin and the psychedelic is DOM, wherein the volinanserin is administered to pretreat at least 270 minutes prior to the DOM. In some embodiments, the serotonin receptor modulator is volinanserin and the psychedelic is DOM, wherein the volinanserin is administered to pretreat at least 300 minutes prior to the DOM. In some embodiments, the serotonin receptor modulator is volinanserin and the psychedelic is DOM, wherein the volinanserin is administered to pretreat at least 330 minutes prior to the DOM. In some embodiments, the serotonin receptor modulator is volinanserin and the psychedelic is DOM, wherein the volinanserin is administered to pretreat at least 360 minutes prior to the DOM. In some preferred embodiments, the serotonin receptor modulator is volinanserin and the psychedelic is DOM, wherein volinanserin is administered to pretreat between about 60 minutes and about 180 minutes prior to the administration of DOM.

In some embodiments, the serotonin receptor modulator is ketanserin and the psychedelic is DOM, wherein the ketanserin is administered to pretreat at least 15 minutes prior to the administration of DOM. In some embodiments, the serotonin receptor modulator is ketanserin and the psychedelic is DOM, wherein the ketanserin is administered to pretreat at least 30 minutes prior to the DOM. In some embodiments, the serotonin receptor modulator is ketanserin and the psychedelic is DOM, wherein the ketanserin is administered to pretreat at least 60 minutes prior to the DOM. In some embodiments, the serotonin receptor modulator is ketanserin and the psychedelic is DOM, wherein the ketanserin is administered to pretreat at least 90 minutes prior to the DOM. In some embodiments, the serotonin receptor modulator is ketanserin and the psychedelic is DOM, wherein the ketanserin is administered to pretreat at least 120 minutes prior to the DOM. In some embodiments, the serotonin receptor modulator is ketanserin and the psychedelic is DOM, wherein the ketanserin is administered to pretreat at least 150 minutes prior to the DOM. In some embodiments, the serotonin receptor modulator is ketanserin and the psychedelic is DOM, wherein the ketanserin is administered to pretreat between about 15 minutes and about 150 minutes prior to the DOM. In some embodiments, the serotonin receptor modulator is ketanserin and the psychedelic is DOM, wherein the ketanserin is administered to pretreat at least 180 minutes prior to the DOM. In some embodiments, the serotonin receptor modulator is ketanserin and the psychedelic is DOM, wherein the ketanserin is administered to pretreat at least 210 minutes prior to the DOM. In some embodiments, the serotonin receptor modulator is ketanserin and the psychedelic is DOM, wherein the ketanserin is administered to pretreat at least 240 minutes prior to the DOM. In some embodiments, the serotonin receptor modulator is ketanserin and the psychedelic is DOM, wherein the ketanserin is administered to pretreat at least 270 minutes prior to the DOM. In some embodiments, the serotonin receptor modulator is ketanserin and the psychedelic is DOM, wherein the ketanserin is administered to pretreat at least 300 minutes prior to the DOM. In some embodiments, the serotonin receptor modulator is ketanserin and the psychedelic is DOM, wherein the ketanserin is administered to pretreat at least 330 minutes prior to the DOM. In some embodiments, the serotonin receptor modulator is ketanserin and the psychedelic is DOM, wherein the ketanserin is administered to pretreat at least 360 minutes prior to the DOM. In some preferred embodiments, the serotonin receptor modulator is ketanserin and the psychedelic is DOM, wherein ketanserin is administered to pretreat between about 60 minutes and about 180 minutes prior to the administration of DOM.

In some embodiments, the serotonin receptor modulator is ritanserin and the psychedelic is DOM, wherein the ritanserin is administered to pretreat at least 15 minutes prior to the DOM. In some embodiments, the serotonin receptor modulator is ritanserin and the psychedelic is DOM, wherein the ritanserin is administered to pretreat at least 30 minutes prior to the DOM. In some embodiments, the serotonin receptor modulator is ritanserin and the psychedelic is DOM, wherein the ritanserin is administered to pretreat at least 60 minutes prior to the DOM. In some embodiments, the serotonin receptor modulator is ritanserin and the psychedelic is DOM, wherein the ritanserin is administered to pretreat at least 90 minutes prior to the DOM. In some embodiments, the serotonin receptor modulator is ritanserin and the psychedelic is DOM, wherein the ritanserin is administered to pretreat at least 120 minutes prior to the DOM. In some embodiments, the serotonin receptor modulator is ritanserin and the psychedelic is DOM, wherein the ritanserin is administered to pretreat at least 150 minutes prior to the DOM. In some embodiments, the serotonin receptor modulator is ritanserin and the psychedelic is DOM, wherein the ritanserin is administered to pretreat between about 15 minutes and about 150 minutes prior to the DOM. In some embodiments, the serotonin receptor modulator is ritanserin and the psychedelic is DOM, wherein the ritanserin is administered to pretreat at least 180 minutes prior to the DOM. In some embodiments, the serotonin receptor modulator is ritanserin and the psychedelic is DOM, wherein the ritanserin is administered to pretreat at least 210 minutes prior to the DOM. In some embodiments, the serotonin receptor modulator is ritanserin and the psychedelic is DOM, wherein the ritanserin is administered to pretreat at least 240 minutes prior to the DOM. In some embodiments, the serotonin receptor modulator is ritanserin and the psychedelic is DOM, wherein the ritanserin is administered to pretreat at least 270 minutes prior to the DOM. In some embodiments, the serotonin receptor modulator is ritanserin and the psychedelic is DOM, wherein the ritanserin is administered to pretreat at least 300 minutes prior to the DOM. In some embodiments, the serotonin receptor modulator is ritanserin and the psychedelic is DOM, wherein the ritanserin is administered to pretreat at least 330 minutes prior to the DOM. In some embodiments, the serotonin receptor modulator is ritanserin and the psychedelic is DOM, wherein the ritanserin is administered to pretreat at least 360 minutes prior to the DOM. In some preferred embodiments, the serotonin receptor modulator is ritanserin and the psychedelic is DOM, wherein ritanserin is administered to pretreat between about 60 minutes and about 180 minutes prior to the administration of DOM.

In some embodiments, the serotonin receptor modulator is pimavanserin and the psychedelic is DOM, wherein the pimavanserin is administered to pretreat at least 15 minutes prior to the DOM. In some embodiments, the serotonin receptor modulator is pimavanserin and the psychedelic is DOM, wherein the pimavanserin is administered to pretreat at least 30 minutes prior to the DOM. In some embodiments, the serotonin receptor modulator is pimavanserin and the psychedelic is DOM, wherein the pimavanserin is administered to pretreat at least 60 minutes prior to the DOM. In some embodiments, the serotonin receptor modulator is pimavanserin and the psychedelic is DOM, wherein the pimavanserin is administered to pretreat at least 90 minutes prior to the DOM. In some embodiments, the serotonin receptor modulator is pimavanserin and the psychedelic is DOM, wherein the pimavanserin is administered to pretreat at least 120 minutes prior to the DOM. In some embodiments, the serotonin receptor modulator is pimavanserin and the psychedelic is DOM, wherein the pimavanserin is administered to pretreat at least 150 minutes prior to the DOM. In some embodiments, the serotonin receptor modulator is pimavanserin and the psychedelic is DOM, wherein the pimavanserin is administered to pretreat between about 15 minutes and about 150 minutes prior to the DOM. In some embodiments, the serotonin receptor modulator is pimavanserin and the psychedelic is DOM, wherein the pimavanserin is administered to pretreat at least 180 minutes prior to the DOM. In some embodiments, the serotonin receptor modulator is pimavanserin and the psychedelic is DOM, wherein the pimavanserin is administered to pretreat at least 210 minutes prior to the DOM. In some embodiments, the serotonin receptor modulator is pimavanserin and the psychedelic is DOM, wherein the pimavanserin is administered to pretreat at least 240 minutes prior to the DOM. In some embodiments, the serotonin receptor modulator is pimavanserin and the psychedelic is DOM, wherein the pimavanserin is administered to pretreat at least 270 minutes prior to the DOM. In some embodiments, the serotonin receptor modulator is pimavanserin and the psychedelic is DOM, wherein the pimavanserin is administered to pretreat at least 300 minutes prior to the DOM. In some embodiments, the serotonin receptor modulator is pimavanserin and the psychedelic is DOM, wherein the pimavanserin is administered to pretreat at least 330 minutes prior to the DOM. In some embodiments, the serotonin receptor modulator is pimavanserin and the psychedelic is DOM, wherein the pimavanserin is administered to pretreat at least 360 minutes prior to the DOM. In some preferred embodiments, the serotonin receptor modulator is pimavanserin and the psychedelic is DOM, wherein pimavanserin is administered to pretreat between about 60 minutes and about 180 minutes prior to the administration of DOM.

In some embodiments, the serotonin receptor modulator is nelotanserin and the psychedelic is DOM, wherein the nelotanserin is administered to pretreat at least 15 minutes prior to the DOM. In some embodiments, the serotonin receptor modulator is nelotanserin and the psychedelic is DOM, wherein the nelotanserin is administered to pretreat at least 30 minutes prior to the DOM. In some embodiments, the serotonin receptor modulator is nelotanserin and the psychedelic is DOM, wherein the nelotanserin is administered to pretreat at least 60 minutes prior to the DOM. In some embodiments, the serotonin receptor modulator is nelotanserin and the psychedelic is DOM, wherein the nelotanserin is administered to pretreat at least 90 minutes prior to the DOM. In some embodiments, the serotonin receptor modulator is nelotanserin and the psychedelic is DOM, wherein the nelotanserin is administered to pretreat at least 120 minutes prior to the DOM. In some embodiments, the serotonin receptor modulator is nelotanserin and the psychedelic is DOM, wherein the nelotanserin is administered to pretreat at least 150 minutes prior to the DOM. In some embodiments, the serotonin receptor modulator is nelotanserin and the psychedelic is DOM, wherein the nelotanserin is administered to pretreat between about 15 minutes and about 150 minutes prior to the DOM. In some embodiments, the serotonin receptor modulator is nelotanserin and the psychedelic is DOM, wherein the nelotanserin is administered to pretreat at least 180 minutes prior to the DOM. In some embodiments, the serotonin receptor modulator is nelotanserin and the psychedelic is DOM, wherein the nelotanserin is administered to pretreat at least 210 minutes prior to the DOM. In some embodiments, the serotonin receptor modulator is nelotanserin and the psychedelic is DOM, wherein the nelotanserin is administered to pretreat at least 240 minutes prior to the DOM. In some embodiments, the serotonin receptor modulator is nelotanserin and the psychedelic is DOM, wherein the nelotanserin is administered to pretreat at least 270 minutes prior to the DOM. In some embodiments, the serotonin receptor modulator is nelotanserin and the psychedelic is DOM, wherein the nelotanserin is administered to pretreat at least 300 minutes prior to the DOM. In some embodiments, the serotonin receptor modulator is nelotanserin and the psychedelic is DOM, wherein the nelotanserin is administered to pretreat at least 330 minutes prior to the DOM. In some embodiments, the serotonin receptor modulator is nelotanserin and the psychedelic is DOM, wherein the nelotanserin is administered to pretreat at least 360 minutes prior to the DOM. In some preferred embodiments, the serotonin receptor modulator is nelotanserin and the psychedelic is DOM, wherein nelotanserin is administered to pretreat between about 60 minutes and about 180 minutes prior to the administration of DOM.

In some embodiments, the serotonin receptor modulator is pruvanserin and the psychedelic is DOM, wherein the pruvanserin is administered to pretreat at least 15 minutes prior to the DOM. In some embodiments, the serotonin receptor modulator is pruvanserin and the psychedelic is DOM, wherein the pruvanserin is administered to pretreat at least 30 minutes prior to the DOM. In some embodiments, the serotonin receptor modulator is pruvanserin and the psychedelic is DOM, wherein the pruvanserin is administered to pretreat at least 60 minutes prior to the DOM. In some embodiments, the serotonin receptor modulator is pruvanserin and the psychedelic is DOM, wherein the pruvanserin is administered to pretreat at least 90 minutes prior to the DOM. In some embodiments, the serotonin receptor modulator is pruvanserin and the psychedelic is DOM, wherein the pruvanserin is administered to pretreat at least 120 minutes prior to the DOM. In some embodiments, the serotonin receptor modulator is pruvanserin and the psychedelic is DOM, wherein the pruvanserin is administered to pretreat at least 150 minutes prior to the DOM. In some embodiments, the serotonin receptor modulator is pruvanserin and the psychedelic is DOM, wherein the pruvanserin is administered to pretreat between about 15 minutes and about 150 minutes prior to the DOM. In some embodiments, the serotonin receptor modulator is pruvanserin and the psychedelic is DOM, wherein the pruvanserin is administered to pretreat at least 180 minutes prior to the DOM. In some embodiments, the serotonin receptor modulator is pruvanserin and the psychedelic is DOM, wherein the pruvanserin is administered to pretreat at least 210 minutes prior to the DOM. In some embodiments, the serotonin receptor modulator is pruvanserin and the psychedelic is DOM, wherein the pruvanserin is administered to pretreat at least 240 minutes prior to the DOM. In some embodiments, the serotonin receptor modulator is pruvanserin and the psychedelic is DOM, wherein the pruvanserin is administered to pretreat at least 270 minutes prior to the DOM. In some embodiments, the serotonin receptor modulator is pruvanserin and the psychedelic is DOM, wherein the pruvanserin is administered to pretreat at least 300 minutes prior to the DOM. In some embodiments, the serotonin receptor modulator is pruvanserin and the psychedelic is DOM, wherein the pruvanserin is administered to pretreat at least 330 minutes prior to the DOM. In some embodiments, the serotonin receptor modulator is pruvanserin and the psychedelic is DOM, wherein the pruvanserin is administered to pretreat at least 360 minutes prior to the DOM. In some preferred embodiments, the serotonin receptor modulator is pruvanserin and the psychedelic is DOM, wherein pruvanserin is administered to pretreat between about 60 minutes and about 180 minutes prior to the administration of DOM.

In some embodiments, the serotonin receptor modulator is eplivanserin and the psychedelic is mescaline, wherein eplivanserin is administered to pretreat at least 15 minutes prior to the administration of mescaline. In some embodiments, the serotonin receptor modulator is eplivanserin and the psychedelic is mescaline, wherein the eplivanserin is administered to pretreat at least 30 minutes prior to the mescaline. In some embodiments, the serotonin receptor modulator is eplivanserin and the psychedelic is mescaline, wherein the eplivanserin is administered to pretreat at least 60 minutes prior to the mescaline. In some embodiments, the serotonin receptor modulator is eplivanserin and the psychedelic is mescaline, wherein the eplivanserin is administered to pretreat at least 90 minutes prior to the mescaline. In some embodiments, the serotonin receptor modulator is eplivanserin and the psychedelic is mescaline, wherein the eplivanserin is administered to pretreat at least 120 minutes prior to the mescaline. In some embodiments, the serotonin receptor modulator is eplivanserin and the psychedelic is mescaline, wherein the eplivanserin is administered to pretreat at least 150 minutes prior to the mescaline. In some embodiments, the serotonin receptor modulator is eplivanserin and the psychedelic is mescaline, wherein the eplivanserin is administered to pretreat between about 15 minutes and about 150 minutes prior to the mescaline. In some embodiments, the serotonin receptor modulator is eplivanserin and the psychedelic is mescaline, wherein the eplivanserin is administered to pretreat at least 180 minutes prior to the mescaline. In some embodiments, the serotonin receptor modulator is eplivanserin and the psychedelic is mescaline, wherein the eplivanserin is administered to pretreat at least 210 minutes prior to the mescaline. In some embodiments, the serotonin receptor modulator is eplivanserin and the psychedelic is mescaline, wherein the eplivanserin is administered to pretreat at least 240 minutes prior to the mescaline. In some embodiments, the serotonin receptor modulator is eplivanserin and the psychedelic is mescaline, wherein the eplivanserin is administered to pretreat at least 270 minutes prior to the mescaline. In some embodiments, the serotonin receptor modulator is eplivanserin and the psychedelic is mescaline, wherein the eplivanserin is administered to pretreat at least 300 minutes prior to the mescaline. In some embodiments, the serotonin receptor modulator is eplivanserin and the psychedelic is mescaline, wherein the eplivanserin is administered to pretreat at least 330 minutes prior to the mescaline. In some embodiments, the serotonin receptor modulator is eplivanserin and the psychedelic is mescaline, wherein the eplivanserin is administered to pretreat at least 360 minutes prior to the mescaline. In some preferred embodiments, the serotonin receptor modulator is eplivanserin and the psychedelic is mescaline, wherein eplivanserin is administered to pretreat between about 60 minutes and about 180 minutes prior to the administration of mescaline.

In some embodiments, the serotonin receptor modulator is volinanserin and the psychedelic is mescaline, wherein volinanserin is administered to pretreat at least 15 minutes prior to the administration of mescaline. In some embodiments, the serotonin receptor modulator is volinanserin and the psychedelic is mescaline, wherein the volinanserin is administered to pretreat at least 30 minutes prior to the mescaline. In some embodiments, the serotonin receptor modulator is volinanserin and the psychedelic is mescaline, wherein the volinanserin is administered to pretreat at least 60 minutes prior to the mescaline. In some embodiments, the serotonin receptor modulator is volinanserin and the psychedelic is mescaline, wherein the volinanserin is administered to pretreat at least 90 minutes prior to the mescaline. In some embodiments, the serotonin receptor modulator is volinanserin and the psychedelic is mescaline, wherein the volinanserin is administered to pretreat at least 120 minutes prior to the mescaline. In some embodiments, the serotonin receptor modulator is volinanserin and the psychedelic is mescaline, wherein the volinanserin is administered to pretreat at least 150 minutes prior to the mescaline. In some embodiments, the serotonin receptor modulator is volinanserin and the psychedelic is mescaline, wherein the volinanserin is administered to pretreat between about 15 minutes and about 150 minutes prior to the mescaline. In some embodiments, the serotonin receptor modulator is volinanserin and the psychedelic is mescaline, wherein the volinanserin is administered to pretreat at least 180 minutes prior to the mescaline. In some embodiments, the serotonin receptor modulator is volinanserin and the psychedelic is mescaline, wherein the volinanserin is administered to pretreat at least 210 minutes prior to the mescaline. In some embodiments, the serotonin receptor modulator is volinanserin and the psychedelic is mescaline, wherein the volinanserin is administered to pretreat at least 240 minutes prior to the mescaline. In some embodiments, the serotonin receptor modulator is volinanserin and the psychedelic is mescaline, wherein the volinanserin is administered to pretreat at least 270 minutes prior to the mescaline. In some embodiments, the serotonin receptor modulator is volinanserin and the psychedelic is mescaline, wherein the volinanserin is administered to pretreat at least 300 minutes prior to the mescaline. In some embodiments, the serotonin receptor modulator is volinanserin and the psychedelic is mescaline, wherein the volinanserin is administered to pretreat at least 330 minutes prior to the mescaline. In some embodiments, the serotonin receptor modulator is volinanserin and the psychedelic is mescaline, wherein the volinanserin is administered to pretreat at least 360 minutes prior to the mescaline. In some preferred embodiments, the serotonin receptor modulator is volinanserin and the psychedelic is mescaline, wherein volinanserin is administered to pretreat between about 60 minutes and about 180 minutes prior to the administration of mescaline.

In some embodiments, the serotonin receptor modulator is ketanserin and the psychedelic is mescaline, wherein ketanserin is administered to pretreat at least 15 minutes prior to the administration of mescaline. In some embodiments, the serotonin receptor modulator is ketanserin and the psychedelic is mescaline, wherein the ketanserin is administered to pretreat at least 30 minutes prior to the mescaline. In some embodiments, the serotonin receptor modulator is ketanserin and the psychedelic is mescaline, wherein the ketanserin is administered to pretreat at least 60 minutes prior to the mescaline. In some embodiments, the serotonin receptor modulator is ketanserin and the psychedelic is mescaline, wherein the ketanserin is administered to pretreat at least 90 minutes prior to the mescaline. In some embodiments, the serotonin receptor modulator is ketanserin and the psychedelic is mescaline, wherein the ketanserin is administered to pretreat at least 120 minutes prior to the mescaline. In some embodiments, the serotonin receptor modulator is ketanserin and the psychedelic is mescaline, wherein the ketanserin is administered to pretreat at least 150 minutes prior to the mescaline. In some embodiments, the serotonin receptor modulator is ketanserin and the psychedelic is mescaline, wherein the ketanserin is administered to pretreat between about 15 minutes and about 150 minutes prior to the mescaline. In some embodiments, the serotonin receptor modulator is ketanserin and the psychedelic is mescaline, wherein the ketanserin is administered to pretreat at least 180 minutes prior to the mescaline. In some embodiments, the serotonin receptor modulator is ketanserin and the psychedelic is mescaline, wherein the ketanserin is administered to pretreat at least 210 minutes prior to the mescaline. In some embodiments, the serotonin receptor modulator is ketanserin and the psychedelic is mescaline, wherein the ketanserin is administered to pretreat at least 240 minutes prior to the mescaline. In some embodiments, the serotonin receptor modulator is ketanserin and the psychedelic is mescaline, wherein the ketanserin is administered to pretreat at least 270 minutes prior to the mescaline. In some embodiments, the serotonin receptor modulator is ketanserin and the psychedelic is mescaline, wherein the ketanserin is administered to pretreat at least 300 minutes prior to the mescaline. In some embodiments, the serotonin receptor modulator is ketanserin and the psychedelic is mescaline, wherein the ketanserin is administered to pretreat at least 330 minutes prior to the mescaline. In some embodiments, the serotonin receptor modulator is ketanserin and the psychedelic is mescaline, wherein the ketanserin is administered to pretreat at least 360 minutes prior to the mescaline. In some preferred embodiments, the serotonin receptor modulator is ketanserin and the psychedelic is mescaline, wherein ketanserin is administered to pretreat between about 60 minutes and about 180 minutes prior to the administration of mescaline.

In some embodiments, the serotonin receptor modulator is ritanserin and the psychedelic is mescaline, wherein ritanserin is administered to pretreat at least 15 minutes prior to the administration of mescaline. In some embodiments, the serotonin receptor modulator is ritanserin and the psychedelic is mescaline, wherein the ritanserin is administered to pretreat at least 30 minutes prior to the mescaline. In some embodiments, the serotonin receptor modulator is ritanserin and the psychedelic is mescaline, wherein the ritanserin is administered to pretreat at least 60 minutes prior to the mescaline. In some embodiments, the serotonin receptor modulator is ritanserin and the psychedelic is mescaline, wherein the ritanserin is administered to pretreat at least 90 minutes prior to the mescaline. In some embodiments, the serotonin receptor modulator is ritanserin and the psychedelic is mescaline, wherein the ritanserin is administered to pretreat at least 120 minutes prior to the mescaline. In some embodiments, the serotonin receptor modulator is ritanserin and the psychedelic is mescaline, wherein the ritanserin is administered to pretreat at least 150 minutes prior to the mescaline. In some embodiments, the serotonin receptor modulator is ritanserin and the psychedelic is mescaline, wherein the ritanserin is administered to pretreat between about 15 minutes and about 150 minutes prior to the mescaline. In some embodiments, the serotonin receptor modulator is ritanserin and the psychedelic is mescaline, wherein the ritanserin is administered to pretreat at least 180 minutes prior to the mescaline. In some embodiments, the serotonin receptor modulator is ritanserin and the psychedelic is mescaline, wherein the ritanserin is administered to pretreat at least 210 minutes prior to the mescaline. In some embodiments, the serotonin receptor modulator is ritanserin and the psychedelic is mescaline, wherein the ritanserin is administered to pretreat at least 240 minutes prior to the mescaline. In some embodiments, the serotonin receptor modulator is ritanserin and the psychedelic is mescaline, wherein the ritanserin is administered to pretreat at least 270 minutes prior to the mescaline. In some embodiments, the serotonin receptor modulator is ritanserin and the psychedelic is mescaline, wherein the ritanserin is administered to pretreat at least 300 minutes prior to the mescaline. In some embodiments, the serotonin receptor modulator is ritanserin and the psychedelic is mescaline, wherein the ritanserin is administered to pretreat at least 330 minutes prior to the mescaline. In some embodiments, the serotonin receptor modulator is ritanserin and the psychedelic is mescaline, wherein the ritanserin is administered to pretreat at least 360 minutes prior to the mescaline. In some preferred embodiments, the serotonin receptor modulator is ritanserin and the psychedelic is mescaline, wherein ritanserin is administered to pretreat between about 60 minutes and about 180 minutes prior to the administration of mescaline.

In some embodiments, the serotonin receptor modulator is pimavanserin and the psychedelic is mescaline, wherein pimavanserin is administered to pretreat at least 15 minutes prior to the administration of mescaline. In some embodiments, the serotonin receptor modulator is pimavanserin and the psychedelic is mescaline, wherein the pimavanserin is administered to pretreat at least 30 minutes prior to the mescaline. In some embodiments, the serotonin receptor modulator is pimavanserin and the psychedelic is mescaline, wherein the pimavanserin is administered to pretreat at least 60 minutes prior to the mescaline. In some embodiments, the serotonin receptor modulator is pimavanserin and the psychedelic is mescaline, wherein the pimavanserin is administered to pretreat at least 90 minutes prior to the mescaline. In some embodiments, the serotonin receptor modulator is pimavanserin and the psychedelic is mescaline, wherein the pimavanserin is administered to pretreat at least 120 minutes prior to the mescaline. In some embodiments, the serotonin receptor modulator is pimavanserin and the psychedelic is mescaline, wherein the pimavanserin is administered to pretreat at least 150 minutes prior to the mescaline. In some embodiments, the serotonin receptor modulator is pimavanserin and the psychedelic is mescaline, wherein the pimavanserin is administered to pretreat between about 15 minutes and about 150 minutes prior to the mescaline. In some embodiments, the serotonin receptor modulator is pimavanserin and the psychedelic is mescaline, wherein the pimavanserin is administered to pretreat at least 180 minutes prior to the mescaline. In some embodiments, the serotonin receptor modulator is pimavanserin and the psychedelic is mescaline, wherein the pimavanserin is administered to pretreat at least 210 minutes prior to the mescaline. In some embodiments, the serotonin receptor modulator is pimavanserin and the psychedelic is mescaline, wherein the pimavanserin is administered to pretreat at least 240 minutes prior to the mescaline. In some embodiments, the serotonin receptor modulator is pimavanserin and the psychedelic is mescaline, wherein the pimavanserin is administered to pretreat at least 270 minutes prior to the mescaline. In some embodiments, the serotonin receptor modulator is pimavanserin and the psychedelic is mescaline, wherein the pimavanserin is administered to pretreat at least 300 minutes prior to the mescaline. In some embodiments, the serotonin receptor modulator is pimavanserin and the psychedelic is mescaline, wherein the pimavanserin is administered to pretreat at least 330 minutes prior to the mescaline. In some embodiments, the serotonin receptor modulator is pimavanserin and the psychedelic is mescaline, wherein the pimavanserin is administered to pretreat at least 360 minutes prior to the mescaline. In some preferred embodiments, the serotonin receptor modulator is pimavanserin and the psychedelic is mescaline, wherein pimavanserin is administered to pretreat between about 60 minutes and about 180 minutes prior to the administration of mescaline.

In some embodiments, the serotonin receptor modulator is nelotanserin and the psychedelic is mescaline, wherein nelotanserin is administered to pretreat at least 15 minutes prior to the administration of mescaline. In some embodiments, the serotonin receptor modulator is nelotanserin and the psychedelic is mescaline, wherein the nelotanserin is administered to pretreat at least 30 minutes prior to the mescaline. In some embodiments, the serotonin receptor modulator is nelotanserin and the psychedelic is mescaline, wherein the nelotanserin is administered to pretreat at least 60 minutes prior to the mescaline. In some embodiments, the serotonin receptor modulator is nelotanserin and the psychedelic is mescaline, wherein the nelotanserin is administered to pretreat at least 90 minutes prior to the mescaline. In some embodiments, the serotonin receptor modulator is nelotanserin and the psychedelic is mescaline, wherein the nelotanserin is administered to pretreat at least 120 minutes prior to the mescaline. In some embodiments, the serotonin receptor modulator is nelotanserin and the psychedelic is mescaline, wherein the nelotanserin is administered to pretreat at least 150 minutes prior to the mescaline. In some embodiments, the serotonin receptor modulator is nelotanserin and the psychedelic is mescaline, wherein the nelotanserin is administered to pretreat between about 15 minutes and about 150 minutes prior to the mescaline. In some embodiments, the serotonin receptor modulator is nelotanserin and the psychedelic is mescaline, wherein the nelotanserin is administered to pretreat at least 180 minutes prior to the mescaline. In some embodiments, the serotonin receptor modulator is nelotanserin and the psychedelic is mescaline, wherein the nelotanserin is administered to pretreat at least 210 minutes prior to the mescaline. In some embodiments, the serotonin receptor modulator is nelotanserin and the psychedelic is mescaline, wherein the nelotanserin is administered to pretreat at least 240 minutes prior to the mescaline. In some embodiments, the serotonin receptor modulator is nelotanserin and the psychedelic is mescaline, wherein the nelotanserin is administered to pretreat at least 270 minutes prior to the mescaline. In some embodiments, the serotonin receptor modulator is nelotanserin and the psychedelic is mescaline, wherein the nelotanserin is administered to pretreat at least 300 minutes prior to the mescaline. In some embodiments, the serotonin receptor modulator is nelotanserin and the psychedelic is mescaline, wherein the nelotanserin is administered to pretreat at least 330 minutes prior to the mescaline. In some embodiments, the serotonin receptor modulator is nelotanserin and the psychedelic is mescaline, wherein the nelotanserin is administered to pretreat at least 360 minutes prior to the mescaline. In some preferred embodiments, the serotonin receptor modulator is nelotanserin and the psychedelic is mescaline, wherein nelotanserin is administered to pretreat between about 60 minutes and about 180 minutes prior to the administration of mescaline.

In some embodiments, the serotonin receptor modulator is pruvanserin and the psychedelic is mescaline, wherein pruvanserin is administered to pretreat at least 15 minutes prior to the administration of mescaline. In some embodiments, the serotonin receptor modulator is pruvanserin and the psychedelic is mescaline, wherein the pruvanserin is administered to pretreat at least 30 minutes prior to the mescaline. In some embodiments, the serotonin receptor modulator is pruvanserin and the psychedelic is mescaline, wherein the pruvanserin is administered to pretreat at least 60 minutes prior to the mescaline. In some embodiments, the serotonin receptor modulator is pruvanserin and the psychedelic is mescaline, wherein the pruvanserin is administered to pretreat at least 90 minutes prior to the mescaline. In some embodiments, the serotonin receptor modulator is pruvanserin and the psychedelic is mescaline, wherein the pruvanserin is administered to pretreat at least 120 minutes prior to the mescaline. In some embodiments, the serotonin receptor modulator is pruvanserin and the psychedelic is mescaline, wherein the pruvanserin is administered to pretreat at least 150 minutes prior to the mescaline. In some embodiments, the serotonin receptor modulator is pruvanserin and the psychedelic is mescaline, wherein the pruvanserin is administered to pretreat between about 15 minutes and about 150 minutes prior to the mescaline. In some embodiments, the serotonin receptor modulator is pruvanserin and the psychedelic is mescaline, wherein the pruvanserin is administered to pretreat at least 180 minutes prior to the mescaline. In some embodiments, the serotonin receptor modulator is pruvanserin and the psychedelic is mescaline, wherein the pruvanserin is administered to pretreat at least 210 minutes prior to the mescaline. In some embodiments, the serotonin receptor modulator is pruvanserin and the psychedelic is mescaline, wherein the pruvanserin is administered to pretreat at least 240 minutes prior to the mescaline. In some embodiments, the serotonin receptor modulator is pruvanserin and the psychedelic is mescaline, wherein the pruvanserin is administered to pretreat at least 270 minutes prior to the mescaline. In some embodiments, the serotonin receptor modulator is pruvanserin and the psychedelic is mescaline, wherein the pruvanserin is administered to pretreat at least 300 minutes prior to the mescaline. In some embodiments, the serotonin receptor modulator is pruvanserin and the psychedelic is mescaline, wherein the pruvanserin is administered to pretreat at least 330 minutes prior to the mescaline. In some embodiments, the serotonin receptor modulator is pruvanserin and the psychedelic is mescaline, wherein the pruvanserin is administered to pretreat at least 360 minutes prior to the mescaline. In some preferred embodiments, the serotonin receptor modulator is pruvanserin and the psychedelic is mescaline, wherein pruvanserin is administered to pretreat between about 60 minutes and about 180 minutes prior to the administration of mescaline.

In yet another aspect, also provided herein are methods of treating a disease or disorder, wherein the methods comprise administering to the subject a therapeutically effective amount of 2C-B. In some embodiments, the methods further comprise administering to the subject a serotonin receptor modulator, wherein the serotonin receptor modulator is administered at most about 3 hours prior to the administration of 2C-B. In some embodiments, the disease or disorder is depression or a disease or disorder related to depression. In some embodiments, the disease or disorder is post-traumatic stress disorder. In some embodiments, the disease or disorder is fibromyalgia. In some embodiments, the disease or disorder is a stress-related disorder. In some embodiments, the stress-related disorder is acute stress disorder (ASD), an adjustment disorder, reactive attachment disorder (RAD), and/or disinhibited social engagement disorder (DSED). In some embodiments, the disease or disorder is a musculoskeletal pain disorder including fibromyalgia, muscle pain, joint stiffness, osteoarthritis, rheumatoid arthritis, and muscle cramps. In some embodiments, the disease or disorder is a disease of women's reproductive health including premenstrual dysphoric disorder (PMDD), premenstrual syndrome (PMS), post-partum depression, and menopause.

In yet another aspect, also provided herein are methods of treating fibromyalgia or a disease or disorder related to chronic widespread pain, fatigue or hypersensitivity, wherein the methods comprise administering to the subject a therapeutically effective amount of 2C-B. In some embodiments, the methods further comprise administering to the subject a serotonin receptor modulator, wherein the serotonin receptor modulator is administered at most about 3 hours prior to the administration of 2C-B.

In yet another aspect, also provided herein are methods of treating a disease or disorder, wherein the methods comprise administering to the subject a therapeutically effective amount of 1,3-Benzodioxolyl-N-methylbutanamine (MBDB). In some embodiments, the methods further comprise administering to the subject a serotonin receptor modulator, wherein the serotonin receptor modulator is administered at most about 3 hours prior to the administration of MBDB. In some embodiments, the disease or disorder is depression or a disease or disorder related to depression. In some embodiments, the disease or disorder is post-traumatic stress disorder. In some embodiments, the disease or disorder is fibromyalgia. In some embodiments, the disease or disorder is a stress-related disorder. In some embodiments, the stress-related disorder is acute stress disorder (ASD), an adjustment disorder, reactive attachment disorder (RAD), and/or disinhibited social engagement disorder (DSED). In some embodiments, the disease or disorder is a musculoskeletal pain disorder including fibromyalgia, muscle pain, joint stiffness, osteoarthritis, rheumatoid arthritis, and muscle cramps. In some embodiments, the disease or disorder is a disease of women's reproductive health including premenstrual dysphoric disorder (PMDD), premenstrual syndrome (PMS), post-partum depression, and menopause.

In yet another aspect, also provided herein are methods of treating fibromyalgia or a disease or disorder related to chronic widespread pain, fatigue or hypersensitivity, wherein the methods comprise administering to the subject a therapeutically effective amount of MBDB. In some embodiments, the methods further comprise administering to the subject a serotonin receptor modulator, wherein the serotonin receptor modulator is administered at most about 3 hours prior to the administration of MBDB.

In yet another aspect, also provided herein are methods of treating a disease or disorder, wherein the methods comprise administering to the subject a therapeutically effective amount of 3,4-methylenedioxy-N-methylcathinone (Methylone). In some embodiments, the methods further comprise administering to the subject a serotonin receptor modulator, wherein the serotonin receptor modulator is administered at most about 3 hours prior to the administration of 3,4-methylenedioxy-N-methylcathinone (Methylone). In some embodiments, the disease or disorder is depression or a disease or disorder related to depression. In some embodiments, the disease or disorder is post-traumatic stress disorder. In some embodiments, the disease or disorder is fibromyalgia. In some embodiments, the disease or disorder is a stress-related disorder. In some embodiments, the stress-related disorder is acute stress disorder (ASD), an adjustment disorder, reactive attachment disorder (RAD), and/or disinhibited social engagement disorder (DSED). In some embodiments, the disease or disorder is a musculoskeletal pain disorder including fibromyalgia, muscle pain, joint stiffness, osteoarthritis, rheumatoid arthritis, and muscle cramps. In some embodiments, the disease or disorder is a disease of women's reproductive health including premenstrual dysphoric disorder (PMDD), premenstrual syndrome (PMS), post-partum depression, and menopause.

In yet another aspect, also provided herein are methods of treating fibromyalgia or a disease or disorder related to chronic widespread pain, fatigue or hypersensitivity, wherein the methods comprise administering to the subject a therapeutically effective amount of 3,4-methylenedioxy-N-methylcathinone (Methylone). In some embodiments, the methods further comprise administering to the subject a serotonin receptor modulator, wherein the serotonin receptor modulator is administered at most about 3 hours prior to the administration of 3,4-methylenedioxy-N-methylcathinone (Methylone).

In yet another aspect, provided herein are uses of a psychedelic and a serotonin receptor modulator as disclosed herein in the preparation of a medicament for treating a disease or disorder (e.g., depression or a disease or disorder related to depression), wherein the serotonin receptor modulator is released at most about 3 hours prior to the release of the psychedelic.

In yet another aspect, provided herein are pharmaceutical composition comprising a psychedelic and a serotonin receptor modulator as disclosed herein for use in treating a disease or disorder (e.g., depression or a disease or disorder related to depression), wherein the serotonin receptor modulator is released at most about 3 hours prior to the release of the psychedelic.

In yet another aspect, provided herein are psychedelics for use in combination with a serotonin receptor modulator in a method of treating a disease or disorder (e.g., depression or a disease or disorder related to depression), wherein the serotonin receptor modulator is released at most about 3 hours prior to the release of the psychedelic.

In some embodiments, the subject is a mammal. In some embodiments, the subject is a mouse, rabbit, dog, pig, cattle, or human. In some embodiments, the subject is an infant, adult, or child. In some embodiments, the compositions described herein are administered orally, intravenously, subcutaneously, by inhalation, or by an injection. In some embodiments, the compositions described herein are administered orally. In some embodiments, the compositions described herein are administered orally via a pill, ampoule, vial, or tablet.

In some embodiments, the compositions described herein are formulated for immediate release, modified release, sustained release, extended release, controlled release and/or delayed release. In some embodiments, the compositions described herein are solid formulations for oral administration that may be formulated for immediate and/or modified release. In some embodiments, the compositions described herein are formulated to included “modified release” coatings encompassing coatings that delay release, sustain release, extend release, prevent release, minimize release, allow for pulsed release, allow for programmed release and/or otherwise prolong the release of a drug relative to formulations lacking such coatings which release a drug relatively quickly (i.e., “immediate release” compositions).

In various embodiments herein, a composition (e.g., pharmaceutical composition, dosage form, combination or formulation) provided herein is administered at any frequency. For example, in some embodiments, a single dose is provided. In other embodiments, the composition is or is formulated to be administered to an individual in need thereof at least once a day. In other embodiments, the composition is or is formulated to be administered to an individual in need thereof once a day. In other embodiments, the composition is or is formulated to be administered to an individual in need thereof at least twice a day. In various other embodiments, the composition is or is formulated for twice daily, once daily, twice weekly, thrice weekly, or the like administration.

For administration to a subject, the compositions as disclosed herein, in some embodiments, is provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers, in some embodiments, are formulated by conventional methods and can be administered to the subject at a suitable dose. Preferably, the compositions are sterile. These compositions, in some embodiments, contain adjuvants such as preservative, emulsifying agents and dispersing agents. Prevention of the action of microorganisms, in some embodiments, is ensured by the inclusion of various antibacterial and antifungal agents.

In some embodiments, the compositions are in any suitable form, (depending upon the desired method of administration). In some embodiments, the compositions are provided in unit dosage form, provided in a sealed container, or provided as part of a kit. In some embodiments, such a kit includes instructions for use.

In some embodiments, the compositions are adapted for administration by any appropriate route, including a parenteral (e.g., subcutaneous, intramuscular, intravenous, or inhalation) route. In some embodiments, the compositions are prepared by any method known in the art of pharmacy, for example by mixing the active ingredient with the carrier(s) or excipient(s) under sterile conditions.

In some embodiments, the methods described herein are for treating a disease or disorder that is a brain disease or disorder. In some embodiments, the methods described herein are for increasing at least one of translation, transcription or secretion of neurotrophic factors. In some embodiments, the compositions provided herein have, for example, anti-addictive properties, antidepressant properties, anxiolytic properties, or a combination thereof. In some embodiments, the brain disorder is a neuropsychiatric disease. In some embodiments, the methods described herein are for treating a disease or disorder that is a neuropsychiatric disease. In some embodiments, the neuropsychiatric disease is a mood or anxiety disorder. In some embodiments, brain disorders include, for example, migraine, cluster headache, post-traumatic stress disorder (PTSD), anxiety, depression, panic disorder, suicidality, schizophrenia, and addiction (e.g., substance abuse disorder). In some embodiments, brain disorders include, for example, migraines, addiction (e.g., substance use disorder for example alcohol abuse, opiate addition, or abuse), depression, and anxiety.

In some embodiments, the brain disease or disorder is a neurodegenerative disorder, Alzheimer's disease or Parkinson's disease. In some embodiments, the brain disease or disorder is psychological disorder, depression, addiction, anxiety, or a post-traumatic stress disorder. In some embodiments, the brain disorder is depression. In some embodiments, the brain disorder is addiction. In some embodiments, the brain disorder is treatment resistant depression, suicidal ideation, major depressive disorder, bipolar disorder, schizophrenia, stroke, traumatic brain injury or substance use disorder. In some embodiments, the brain disorder is treatment resistant depression, suicidal ideation, major depressive disorder, bipolar disorder, schizophrenia, or substance use disorder. In some embodiments, the brain disorder is stroke or traumatic brain injury. In some embodiments, the brain disorder is treatment resistant depression, suicidal ideation, major depressive disorder, bipolar disorder, or substance use disorder. In some embodiments, the brain disorder is schizophrenia. In some embodiments, the brain disorder is alcohol use disorder.

In some embodiments, the methods described herein are for treating a disease or disorder that is a neurological disease. For example, a compound provided herein can exhibit, anti-addictive properties, antidepressant properties, anxiolytic properties, or a combination thereof. In some embodiments, the neurological disease is a neuropsychiatric disease. In some embodiments, the neuropsychiatric disease is a mood or anxiety disorder. In some embodiments, the neurological disease is a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, hypoxic brain injury, Chronic traumatic encephalopathy (CTE), traumatic brain injury, dementia, and addiction (e.g., substance use disorder). In some embodiments, the neurological disease is a migraine or cluster headache. In some embodiments, the neurological disease is a neurodegenerative disorder, dementia, Alzheimer's disease, or Parkinson's disease. In some embodiments, the neurological disease is dementia. In some embodiments, the neurological disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease or neurological disease is post-traumatic stress disorder (PTSD), addiction (e.g., substance use disorder), schizophrenia, depression, or anxiety. In some embodiments, the neuropsychiatric disease or neurological disease is addiction (e.g., substance use disorder). In some embodiments, the neuropsychiatric disease or neurological disease is depression. In some embodiments, the neuropsychiatric disease or neurological disease is anxiety. In some embodiments, the neuropsychiatric disease or neurological disease is post-traumatic stress disorder (PTSD). In some embodiments, the neurological disease is stroke or traumatic brain injury. In some embodiments, the neuropsychiatric disease or neurological disease is schizophrenia.

In some embodiments, the methods described herein are for increasing neuronal plasticity and has, for example, anti-addictive properties, antidepressant properties, anxiolytic properties, or a combination thereof. In some embodiments, decreased neuronal plasticity is associated with a neuropsychiatric disease.

In additional embodiment, the disease or disorder treated by the methods provided herein is selected from the group consisting of substance-induced mood disorder, bipolar disorder, dysthymia, major depression, major depressive disorder, post-traumatic stress disorder, generalized anxiety disorder, obsessive-compulsive disorder, panic disorder, social anxiety disorder, phobias, dementia, Parkinson's Disease, Alzheimer's Disease, vascular dementia, Lewy body dementia, frontotemporal dementia, mixed dementia, mild cognitive impairment, Creutzfeldt-Jakob disease, Wernicke-Korsakoff Syndrome, stroke, schizophrenia, paranoid personality disorder, schizoid personality disorder, schizotypal personality disorder, antisocial personality disorder, borderline personality disorder, psychosis, disorders of social isolation, disorders of disorganized behavior, disorders of aggression, disorders of agitation, disorders of compulsive behavior, disorders of excitability, disorders of hostility, disorders of self-harm, disorders of lack of restraint, cognitive disorders, delusional disorders, amnesia, mood disorders, disorders of paranoia, disorders of hallucinations, disorders of apathy, disorders of fatigue, disorders of incoherent speech, disorders of impaired motor coordination, disorders of a lack of emotional response, disorders of mental concentration, chorea, negative schizophrenia symptoms including: lack of pleasure/trouble with speech/flattening affect/social withdrawal/lack of follow through with tasks/struggles with activities of daily living, hypoxic brain injury, traumatic brain injury, Chronic traumatic encephalopathy (CTE), drug addiction, alcohol abuse, substance abuse disorder, opiate addiction, cyclothymia, persistent depressive disorder, seasonal affective disorder, hyperactivity, defiant behavior, attention deficit hyperactivity disorders, anorexia nervosa, binge eating, bulimia, anxiety disorders, suicidal behavior, separation anxiety disorder, dissociative identity disorder, dissociative amnesia, depersonalization-derealization disorder, fugue state, disruptive mood dysregulation disorder, premenstrual dysphoric disorder, antenatal depression, postpartum depression, double depression, minor depressive disorder, atypical depression, psychotic depression, bipolar I disorder, bipolar II disorder, reactive attachment disorder, disinhibited social engagement disorder, acute stress disorder, adjustment disorder, complex post-traumatic stress disorder, prolonged grief disorder, social communication disorder, autism spectrum disorder, developmental coordination disorder, Tourette syndrome, Tic disorder, dyslexia, dyscalculia, insomnia, hypersomnia, idiopathic hypersomnia, Kleine-Levin syndrome, insufficient sleep syndrome, narcolepsy, restless leg syndrome, sleep apnea, night terrors, exploding head syndrome, narcissistic personality disorder, neuropathic pain, night eating syndrome, nocturia, nosophobia, alcohol use disorder, acrophobia, early-onset dementia, ailurophobia, algophobia, anosmia, anthropophobia, aphasia, arachnophobia, arithmophobia, Asperger Syndrome, astraphobia, ataxia, ataxophobia, atelophobia, ADHD—inattentive type in adults, bipolar disorder in children, bruxism, childhood schizophrenia, chronic pain, circadian rhythm sleep disorders, claustrophobia, cocaine addiction, concussion, conversion disorder in adults, conversion disorder in children and adolescents, delirium, delusional disorder, dependent personality disorder, derealization disorder, depression in Parkinson's Disease, developmental delay, diplopia, fibromyalgia, Gaucher Disease, Guillain-Barre Syndrome, haphephobia, hoarding disorder, iatrophobia, inhalant abuse, inherited metabolic disorders, jet lag, Joubert Syndrome, Klinefelter Syndrome, learning disabilities, leukophobia, locked-in-syndrome, mania, melanophobia, microphobia, migraine, migraine aura, nicotine headache, nicotine withdrawal, oppositional defiant disorder, ophidiophobia, ornithophobia, overactive bladder, phobophobia, Prader-Willi Syndrome, primary progressive aphasia, progressive supranuclear palsy, psychosomatic disorder, Rett Syndrome, Down Syndrome, Patau Syndrome, Edwards Syndrome, schizoaffective disorder, schizophreniform disorder, self-injury, serotonin syndrome, shift work sleep disorder, sleep anxiety, sleep paralysis, sleepwalking, speech impediment, epilepsy, tinnitus, transient global amnesia, transverse myelitis, Turner syndrome, traumatic brain injury, tuberous sclerosis complex, vocal cord paralysis, Williams Syndrome, and Zellweger syndrome.

In certain embodiments, the methods described herein are for treating a disease or disorder that is a musculoskeletal pain disorder including fibromyalgia, muscle pain, joint stiffness, osteoarthritis, rheumatoid arthritis, and muscle cramps. In certain embodiments, the methods described herein are for treating a disease or disorder that is a disease of women's reproductive health including premenstrual dysphoric disorder (PMDD), premenstrual syndrome (PMS), post-partum depression, and menopause.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Psilocybin and Ketanserin Studies

This Example demonstrates use of psychedelic compounds and 5HT2A receptor antagonists for therapies relating to depression.

### Materials and Methods

### Test Subjects

All procedures were approved by the University of Maryland Baltimore Animal Use and Care Committee and were conducted in full accordance with the National Institutes of Health Guide for the Care and Use of Laboratory Animals.

Two cohorts of male C57Bl/6J mice were bred in-house and used in the stress-induced anhedonia experimental protocols of this study. Separate cohorts of C57Bl/6J mice from Jacksonville Laboratories were used to test ketanserin's activity through in vivo electrophysiology and locomotion effects. All mice were 8 weeks-old at the start of the experiment, kept on a 12-hour light/dark cycle (lights on at 7 am), and provided food and water ad libitum. Animals were group housed prior to the experiment but single housed at the onset of the behavioral and stress protocols until the end of the study. Mice were assigned to balanced experimental and control groups based on hedonic behaviors assessed after stress.

### Chronic Multimodal Stress

Chronic multimodal stress (CMMS) was used to induce an anhedonic-like phenotype in the animals. The CMMS protocol consists of 4 hours/day of restraint stress, in which mice were immobilized in appropriately sized plastic restraint tubes and exposed to strobe lighting and white noise to minimize habituation, for 10-14 consecutive days. Stress was initiated in the morning hours, between 9-10 am, near the onset of the animals' light cycle. Following stress, rodents were returned to their home cages and singly housed.

### Hedonic Behavior

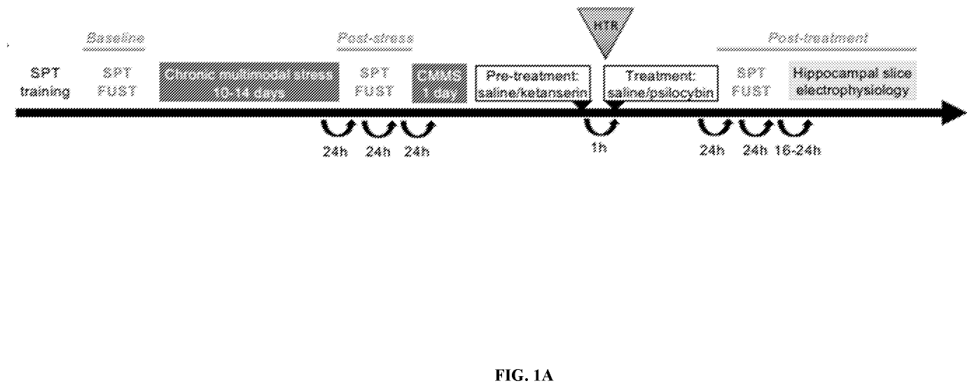

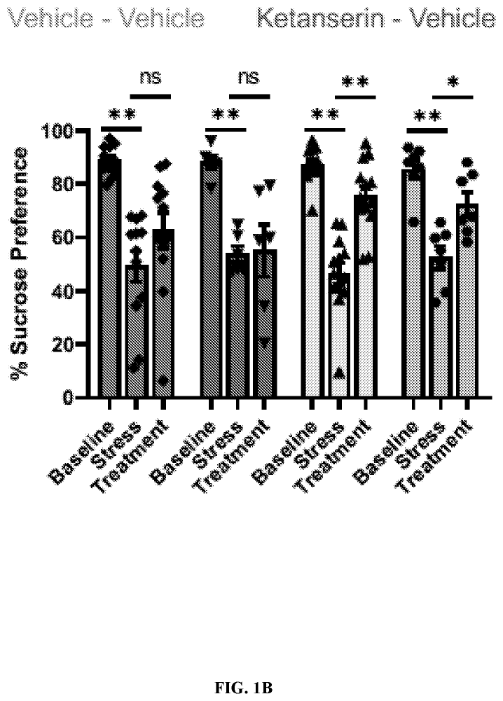

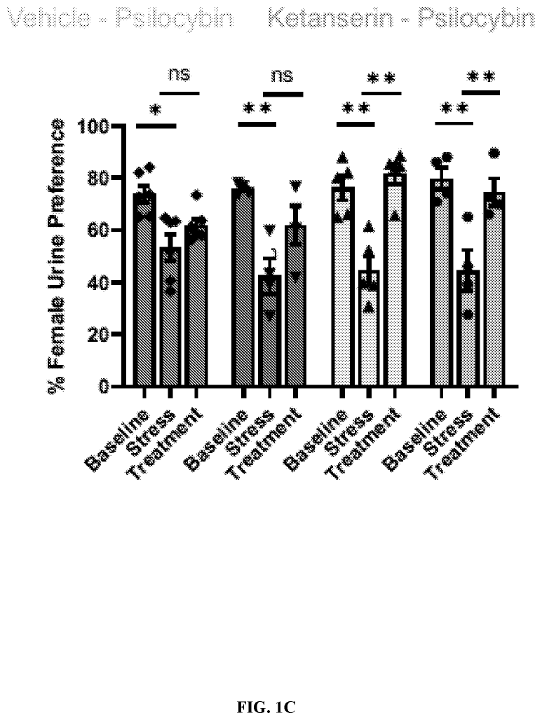

Hedonic state was assessed using the sucrose preference test (SPT) and female urine sniff test (FUST) prior to stress (baseline), after 10-14 days of CMMS, and 24 hours after drug injection (FIG. 1A). For the SPT, mice were trained to the presence of a 2% sucrose solution in their home cages prior to the baseline measurement. The baseline measurements began 1 day later. On each test day, one bottle containing tap water and another bottle containing a 1% sucrose solution were placed in the cages 1-2 hours prior to the onset of the animal's dark cycle. Mice were free to consume liquid from either bottle for 14-16 hours, after which bottles were weighed to measure consumption and replaced. The procedure was repeated for a second night with the position of the bottles reversed. Preference is expressed as a percentage and was calculated for each night as (the weight of 1% sucrose solution consumed/total liquid weight consumed)*100, and the preferences for the two nights were averaged.

For the FUST, mice were individually transferred to empty, freshly made cages and allowed to habituate for 15 minutes. A fresh single cotton swab was then affixed to the rim of the cage, such that the tip was within reach of the mouse. 1 hour later, the swab was removed and replaced with 2 swabs spaced apart at the same end of the cage, one soaked in freshly collected urine from male mice and the other with urine from female mice in estrous. Video recording was started and animals were given 3 minutes to interact with the swabs. Videos were later scored by a trained experimenter blinded to the position of male and female urine swabs. Time spent sniffing each swab was recorded and percent preference scored as (time spent sniffing the female urine swab/total time spent sniffing both swabs)*100. The position of the female urine swab was switched between timepoints to account for potential side preference.

As a priori criteria for inclusion in the study, mice had to have a preference for sucrose of >65% at baseline. Mice which displayed a decrease in sucrose preference to <70% following CMMS, representing >3 standard deviations less than the historical mean baseline sucrose preference for C57/B16J mice (89.2±6.4% (SD), n=107 animals), were considered stress susceptible. Of those mice, only those displaying a female urine preference of >65% at baseline and <70% following CMMS were included in the FUST arm of the study, accounting for the differences in the reported n's. Mice having a sucrose preference >70% after 14 days of CMMS were classified as resilient.

### Head Twitch Response (HTR)

HTR was quantified in a subset of mice exposed to CMMS and treated with vehicle-vehicle (n=7), ketanserin-vehicle (n=7), vehicle-psilocybin (n=7) and ketanserin-psilocybin (n=8). Immediately following the second injection of either vehicle or psilocybin, mice were placed in their home cage and video recording was initiated with a camera positioned directly above the cage. Three trained experimenters scored the first 0-15 minute of activity for HTR while blinded to treatment, noting the total number of and time stamp of each head twitch. Time stamps were compared across scorers and individual head twitches identified by at least ⅔ experimenters were counted.

### Locomotion

A separate cohort of 8-week old male mice were used to analyze the effects of psilocybin and ketanserin pretreatment on locomotion. As in the stress paradigm, mice were pretreated with ketanserin (2 mg/kg) or saline and returned to their home cage. Sixty minutes after pretreatment, mice were injected with a high dose of psilocybin (5 mg/kg) known to induce hypolocomotion. Following the psilocybin injection, animals were immediately placed in an open field arena and their behavior recorded. Locomotion was scored with TopScan Suite software (Clever Sys, Reston, Va).

### Forced Swim Test

A separate cohort of 9-week old male (n=20) and female (n=20) mice were used for the forced swim test (FST). Animals were group housed and injected with vehicle or psilocybin (1 mg/kg). The first FST session occurred 24 hours after injection. Briefly, each swim session consisted of a 6 minute swim in a plexiglass cylinder filled with 15 cm of water (23-25° C.). Each swim was recorded with a video camera. Immobility time was scored with ANY-maze (Stoelting, Wood Dale, IL) for the last 4 minutes of the 6 minute swim, with 2s of immobility set as threshold within the program before scoring began.

### In Vitro Electrophysiology

Standard methods were used to prepare 400 μM-thick hippocampal slices. Briefly, mice were euthanized via exposure to isoflurane followed by decapitation. Brains were excised and the hippocampus was quickly dissected from the brain and sectioned on a Leica VT1200 series vibratome in ice-cold artificial cerebrospinal fluid (ACSF) bubbled with 95% O2/5% CO2. The ACSF contained: 124 mM NaCl, 3 mM KCl, 1.25 mM NaH2PO4, 1.5 mM MgSO4, 2.5 mM CaCl2, 26 mM NaHCO₃, and 10 mM glucose. Slices were allowed to recover for a minimum of 60 minutes at room temperature in ACSF in a humidified interface chamber before recording.

Use of extracellular recording, rather than whole-cell recording, was chosen for quantification of AMPA:NMDA ratios because of the complications of stress-induced changes in dendritic structure and their electrotonic influence on recordings of distal TA-CAI synapses. For quantification of AMPA:NMDA ratios, ACSF was prepared as stated, but without MgSO4, to leave NMDA channels unblocked. Picrotoxin (100 μM) and CGP54626 (2 μM) were added to block GABAA and GABAB receptors, respectively. Slices were placed in a recording chamber and perfused with this ACSF (1 mL/min) for the duration of the experiment. Glass recording electrodes with resistance of 3-5MΩ were prepared and filled with recording ACSF. These electrodes were placed in stratum lacunosum moleculare (SLM) of area CA1. Concentric bipolar tungsten electrodes were positioned in SLM at least 500 μM from the recording electrode to stimulate temporoammonic afferents (TA). Field excitatory postsynaptic potentials (fEPSPs) were acquired using Clampex software (pCLAMP 10 Series, Molecular Devices), amplified (×1000, npi electronic), filtered (3 kHz), and digitized (10 kHz, Digidata 1440a, Molecular Devices). Slices were stimulated (100 us) at 0.1 Hz at five different intensities ranging from 0.01-1.0 mA, in order to collect a range of responses around a fiber volley (FV) of 0.1 mV. DNQX (50 μM) was then washed onto the slice for 15 minutes to block the AMPA component of the fEPSP and reveal the NMDA component. Five fEPSPs were again collected at the same stimulation intensities recorded prior to DNQX. The NMDAR antagonist D-APV (80 μM) was then washed onto the slice for 15 minutes to confirm that the fEPSP response remaining after DNQX was indeed NMDAR-mediated.

AMPA:NMDA ratios of the TA-CAI fEPSPs were quantified as described previously (A. J. Kallarackal et al., 2013 J. Neuroscience. 33, 15669-15764) in order to provide a measure of synaptic strength across slices from different mice. All traces at each intensity were first averaged and the amplitude of FVs quantified. The AMPA component of the fEPSP was quantified as the slope over 1.5 ms at the earliest part of the linear portion of the response for each stimulation intensity, typically 0.1-2.0 ms from its initiation. The NMDA component of the fEPSP slope was quantified over 4 ms at the earliest point of the post-DNQX response fully eliminated by APV. Both AMPA and NMDA slopes were normalized to their respective FVs. For quantification, pairs of responses at the same stimulation intensities were chosen in which the response in the presence of DNQX was closest to 0.1m V in amplitude. AMPA:NMDA ratios from each slice (1-6/mouse) were averaged to calculate each individual animal's mean AMPA:NMDA. As an independent measure of synaptic strength, AMPA:FV and NMDA:FV ratios was also computed, calculated from the same pair of responses used for AMPA:NMDA ratios. Analysis occurred while blinded to the treatment condition and values were confirmed by a second experimenter.

### In Vivo Electrophysiology

Anesthesia was induced using 3-4% isoflurane. Mice were placed in a stereotactic frame and anesthesia was maintained with continuous flow of 2-3% isoflurane. A Q4 silicon probe (Neuronexus Technologies, MI) was lowered into the pyramidal layer of the hippocampal CAI region (AP: −1.8, ML: −1.0, DV: −1.0). Local field potentials (LFPs) were amplified (headstage 20×amplification, Plexon Instruments), digitized (Digidata 1322A, Axon Instruments), and recorded with Clampex 10.3 (Molecular Devices) with a sampling rate of 3 kHz, and bandpass filtering between 1-300 Hz. Mice were injected with either saline or 2 mg/kg ketanserin (i.p.). 30 minutes after ketanserin injections, LFP recordings were initiated. Baseline LFPs were recorded for 30 minutes before mice were injected with psilocybin (10 mg/kg, i.p.). LFPs were then recorded for another 90 minutes. Electrode placement was histologically confirmed at the end of the experiment.

Binary files were loaded into MATLAB 2015a (Mathworks) using abfload script (Forrest Collman: fcollman/abfload, https://github.com/fcollman/abfload). Power spectra were analyzed using Chronux 2.12 spectral analysis script (chronux.org) and normalized to baseline using custom MATLAB script.

### Drug Treatment

Psilocybin was obtained from Cayman Chemical (Ann Arbor, MI) and diluted to 1 mg/mL in sterile 0.9% saline. Psilocybin was prepared within 1 week of administration and stored at −20° ° C. Ketanserin (+)-tartrate salt was purchased from MilliporeSigma (Burlington, MA) and also diluted to 1 mg/mL. Ketanserin was administered 60 minutes prior to injection with either vehicle control or psilocybin, consistent with previous studies of ketanserin's ability to block hallucinogenic behavioral responses in humans and rats.

Psilocybin injections were given at 1 mg/kg and ketanserin at 2 mg/kg, consistent with previous rodent studies, or equivalent volumes of saline. Each experimental animal received two injections to control for any effects of injection or handling.

For locomotion experiments or in vivo electrophysiology experiments, psilocybin was prepared at a 5 mg/mL concentration in sterile 0.9% saline and injected at 5 mg/kg or 10 mg/kg, respectively.

### Statistics

Statistical analysis consisted of Student's t-tests, one-, two-, and three-way ANOVAs using GraphPad Prism 8, with Tukey's correction for multiple comparisons performed in Prism 8 or Holm-Sidak multiple comparisons corrections performed manually in Excel (Microsoft) for comparisons of interest. Results from the two cohorts of animals were not statistically different and were therefore pooled. Statistical tests used are indicated within the figure legends. Where indicated, n=number of animals.

### Results

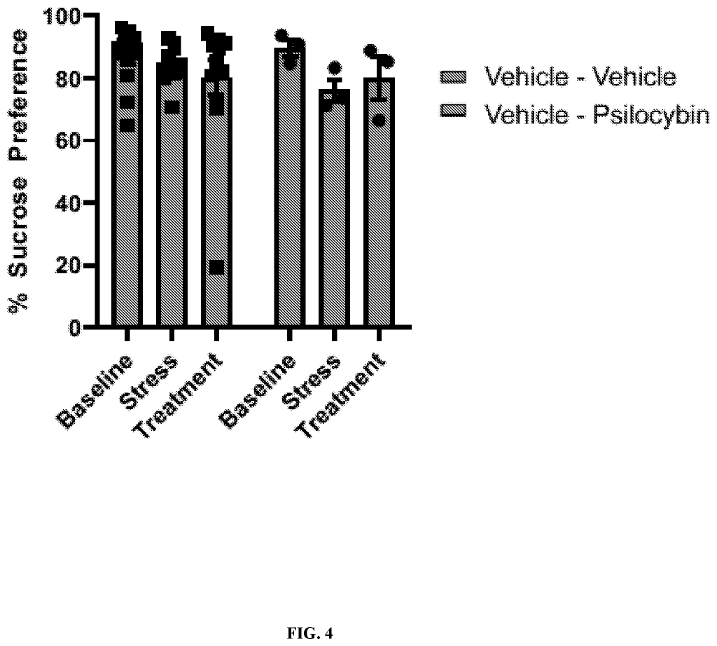

8 week-old male C57Bl/6J mice were exposed to a chronic multimodal stress paradigm (CMMS) and assayed hedonic state with two well characterized appetitive choice tasks involving different senses: a two-bottle sucrose preference test (SPT), comparing consumption of a 1% sucrose solution and water, and a female urine sniffing test (FUST), comparing interactions with swabs dipped in urine from male mice and female mice in estrous (FIG. 1A). Mice displayed strong preferences for the sucrose solution and for female urine at baseline and significant decreases in both sucrose and female urine preferences after 10-14 consecutive days of CMMS (FIGS. 1B-1C). Mice were then given a single intraperitoneal (i.p.) injection of psilocybin (1 mg/kg). Mice displayed a significant restoration of their preference for sucrose solution and female urine 24-48 hours after psilocybin injection, whereas mice given a saline vehicle injection retained low sucrose and female urine preferences. Stress-resilient mice that did not display loss of sucrose preference after CMMS did not display any significant change in their responses after psilocybin injection (FIG. 4). These data represent the first evidence of a rapid anti-anhedonic response to psilocybin in a stress-induced preclinical model of depression-relevant behaviors.

Whether activation of pro-hallucinatory 5-HT2Rs was necessary for the antidepressant-like response to psilocybin was determined. Pretreatment with the 5-HT2A/5-HT2C receptor antagonist ketanserin attenuates psilocybin-induced perceptual alterations in humans. In rodents, pretreatment with ketanserin decreases psilocybin-induced head twitching. Stress-susceptible mice were given an injection of ketanserin (2 mg/kg, i.p.), as shown to be effective in previous rat behavioral studies, followed 1 hour later by psilocybin (1 mg/kg, i.p.) or vehicle (0.9% saline). Psilocybin significantly increased sucrose and female urine preferences following stress in ketanserin-pretreated mice, whereas ketanserin pretreatment alone had no significant effect on either behavior (FIGS. 1B-1C).

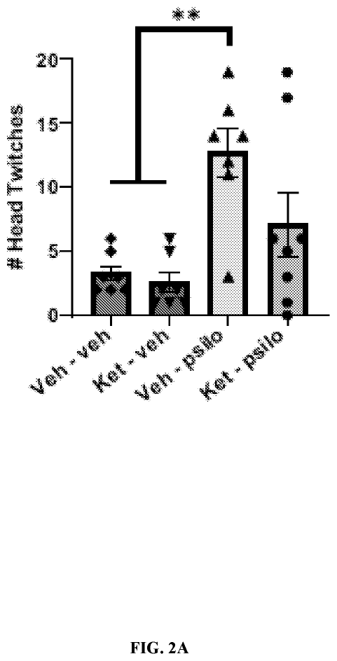

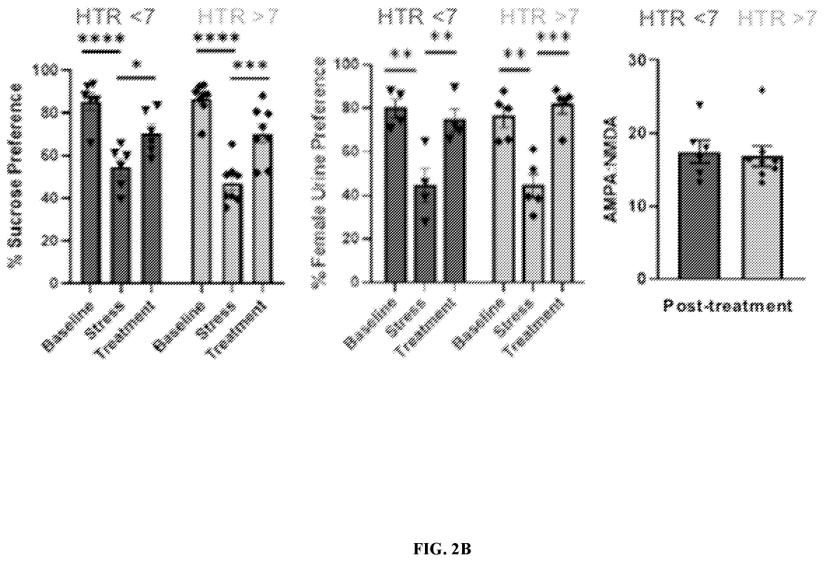

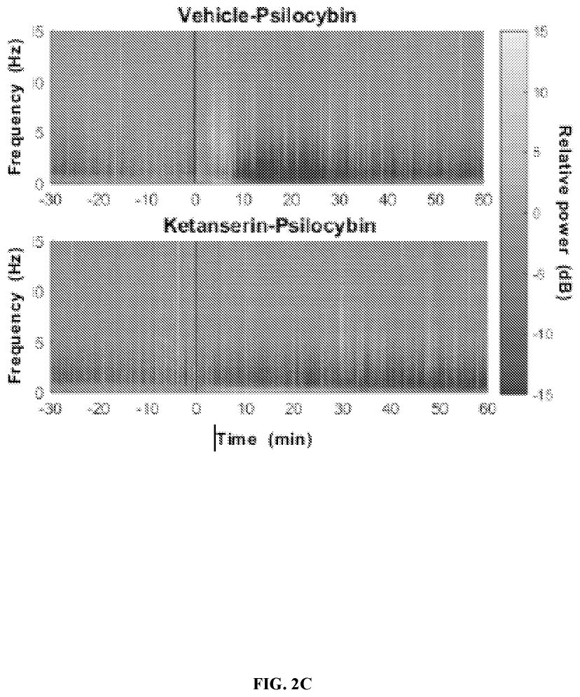

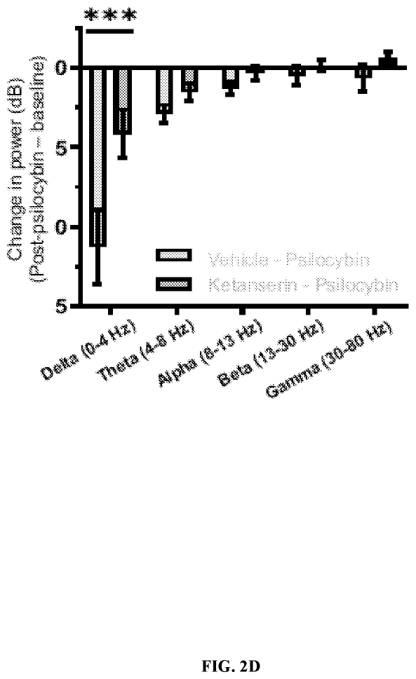

Vehicle pretreated mice receiving psilocybin demonstrated significantly more head twitching compared to mice receiving vehicle or ketanserin alone, serving as a behavioral indication of 5-HT2AR activation by psychedelic compounds. Increases in head twitching counts in mice given psilocybin alone were comparable to previous reports in mice (from ca. 1 twitch/3-4 minute to 1 twitch/minute). Head twitching counts were not significantly different in ketanserin pretreated mice receiving psilocybin compared to mice receiving vehicle alone, indicating that ketanserin had sufficiently blocked 5-HT2A receptors during psilocybin administration (FIG. 2A).

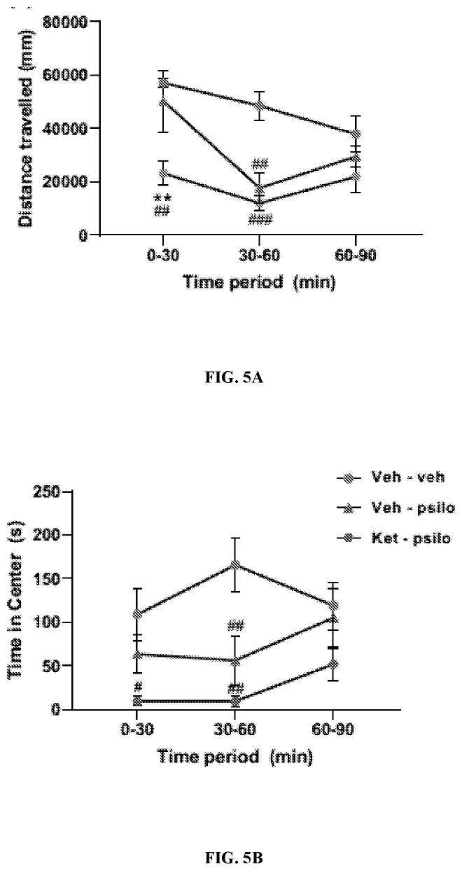

Psilocybin-induced increases in the number of HTRs require 5HT2AR activation, as shown above. To determine whether psilocybin's anti-anhedonic actions resulted from possible incomplete block of 5HT2ARs, post hoc separation of mice given psilocybin with or without ketanserin into a group was performed with a clear increase in head-twitches to >7/15 min, which is greater than any of the control mice and clear evidence of 5-HT2AR activation, and a group with little or no increase in head-twitches (<7/15 min, comparable to control mice) and thus little or no evidence of 5HT2AR activation. Significant improvements in both sucrose and female urine sniffing preferences were observed in mice having both few and many head twitches (FIG. 2B), suggesting that the restoration of reward behavior by psilocybin was independent of 5-HT2AR activation. Consistent with previously published reports, pretreatment with ketanserin induced hypo-locomotion, directly and/or by enhancing the effects of psilocybin, confirming its activity at the given dose (FIGS. 5A-5B).

In humans, psilocybin decreases low frequency EEG oscillations in cortical and limbic areas via activation of ketanserin-sensitive 5-HT2ARs. Similarly, another psychedelic compound, DOI, reduces low-frequency oscillatory local field potentials in the prefrontal cortex of anesthetized rats via 5-HT2A receptor activation. Local field potentials (LFPs) in hippocampal area CA1 in anesthetized mice in vivo was recorded and observed, demonstrating a high concentration of psilocybin (10 mg/kg, i.p.) decreased the power of oscillations in the delta frequency band (0-4 Hz) within 15 min, an effect that lasted for >60 minutes (FIGS. 2C-2D).

This decrease in LFP delta activity was used to determine whether ketanserin pre-injection protocol was effective in blocking 5HT2ARs while psilocybin was active. Ketanserin pretreatment greatly attenuated the decrease in LFP delta power in response to this high concentration of psilocybin, providing another strong positive control of its efficacy as a 5-HT2AR antagonist in the hippocampus under the experimental conditions. These results thus strengthen the conclusion that the anti-anhedonic actions of psilocybin in chronically stressed mice do not require 5-HT2AR activation.

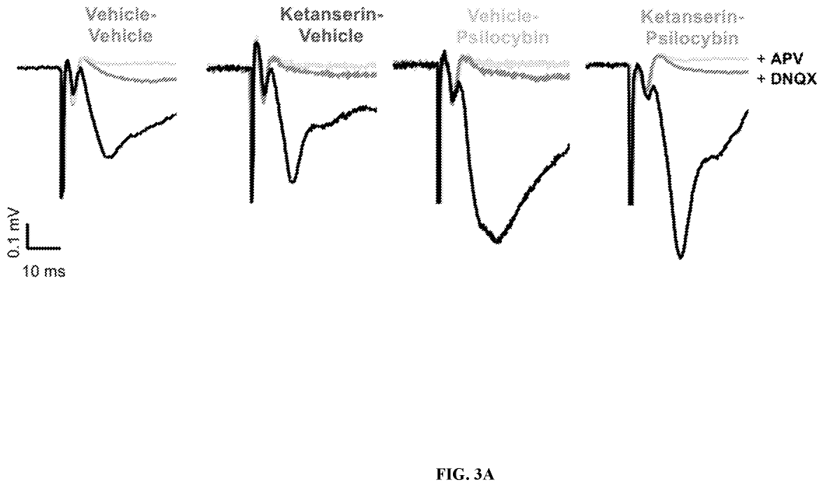

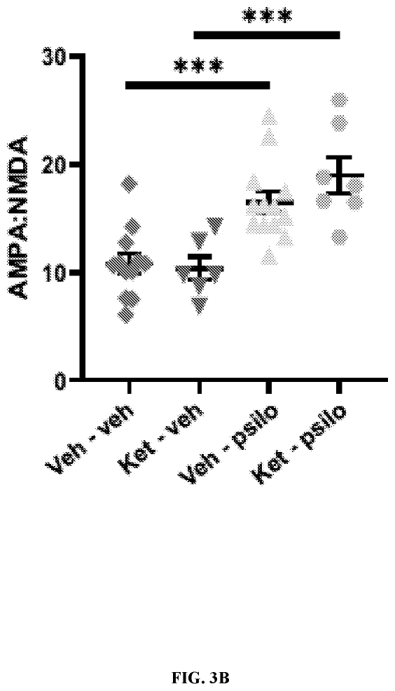

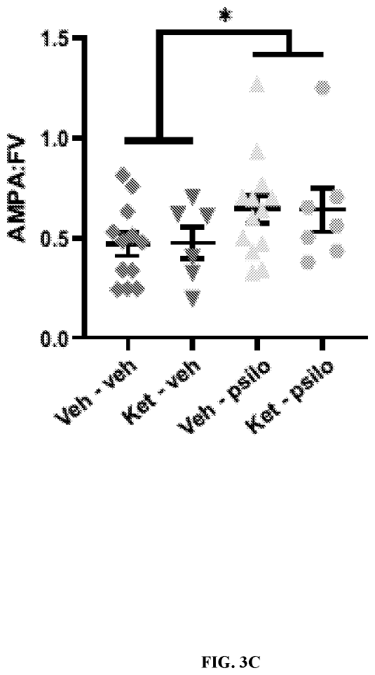

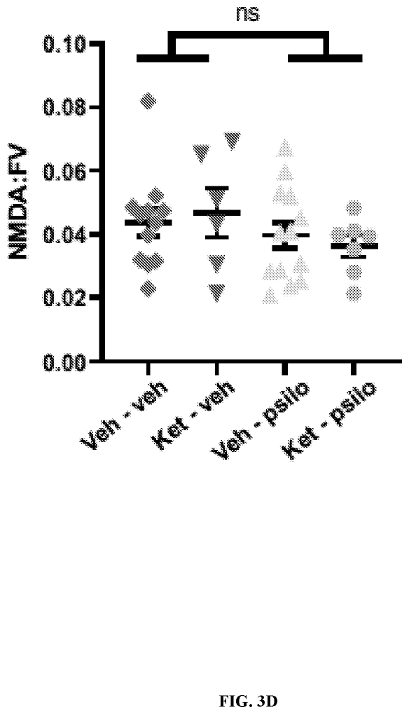

Whether restoration of hedonic state by psilocybin was accompanied by a potentiation of excitatory synaptic strength was determined, quantified as the ratio of the components of the field EPSP mediated by GluA (AMPA) and GluN (NMDA) receptors. After completion of the behavioral assays, hippocampal brain slices were taken from the animals for extracellular recordings. AMPA:NMDA ratios were measured at the archetypical stress-sensitive excitatory synapse formed by temporoammonic inputs to the distal dendrites of CA1 pyramidal cells (TA-CA1) (FIG. 3A).

AMPA:NMDA ratios in slices taken from psilocybin-injected CMMS-susceptible mice were significantly greater than those in slices taken from CMMS-susceptible animals injected with vehicle or ketanserin alone (FIG. 3B). Normalization of the individual components of the synaptic responses across slices to their fiber volley amplitudes revealed a greater amplitude of the AMPAR-mediated component of the response and no significant change in the NMDAR-mediated component in mice treated with psilocybin, consistent with the effects of chronic fluoxetine described previously (FIGS. 3C-3D). These results demonstrate that a single psilocybin administration in rodents promotes persistent synaptic strengthening in a depression-relevant brain region days after its elimination from the body (hours), much like the persistent effects of psilocybin on human brain functional connectivity.

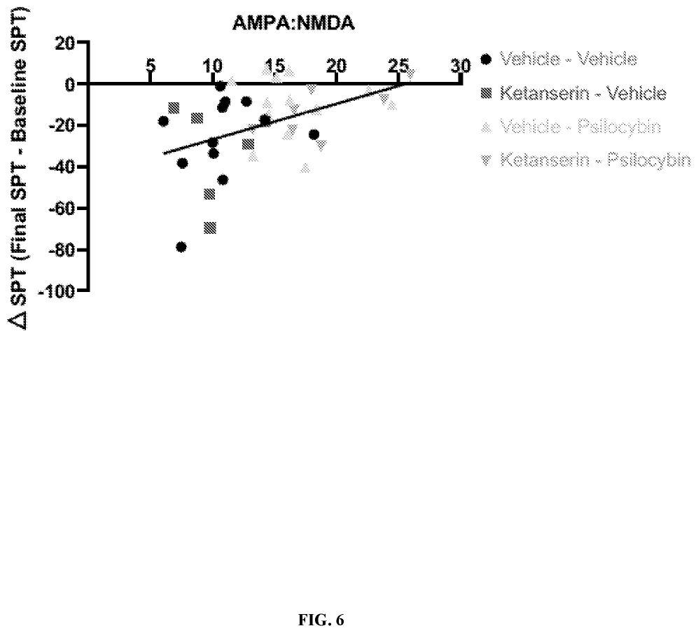

Consistent with the behavioral results, pretreatment with ketanserin did not impair the ability of psilocybin to restore AMPA:NMDA ratios (FIG. 3B). AMPA:NMDA ratios were significantly higher in slices taken from CMMS animals given psilocybin compared to vehicle, regardless of ketanserin pretreatment. As for the behavioral response to psilocybin, AMPA:NMDA ratios in slices from mice given psilocybin with and without ketanserin were elevated to the same extent in mice showing few and many head twitches (FIG. 2B). There was a significant positive correlation between AMPA:NMDA ratios in slices from a given mouse and its performance in the SPT (FIG. 6). Both the anti-anhedonic behavioral response and the hippocampal synaptic response to psilocybin in mice were concluded to be independent of 5-HT2R activation.

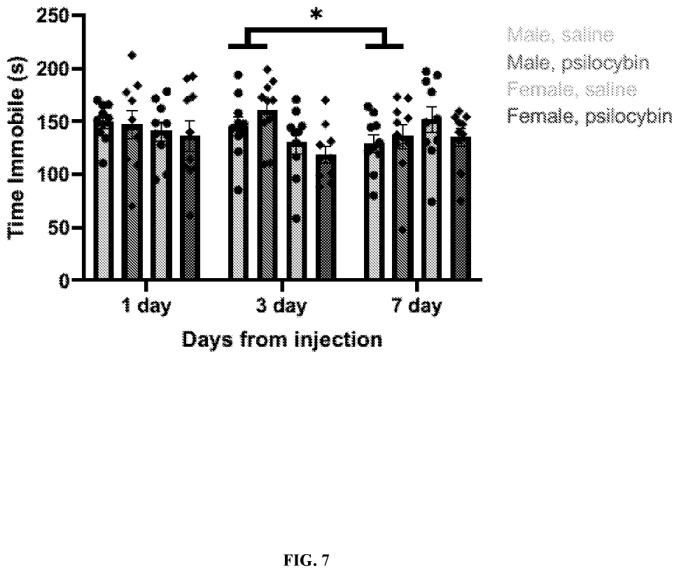

Delayed effects of psilocybin in the forced swim test have been reported previously in Wistar-Kyoto rats but not Flinders Sensitive or Resilient rats. Although female mice do not show a robust anhedonic response to chronic stress, they do respond to the fast-acting antidepressant ketamine in the forced swim test. The effects of psilocybin in the forced swim test in unstressed male or female C57Bl/6J mice were tested. No effect of psilocybin (1 mg/kg) on immobility time at one, three, or seven days post-injection was observed (FIG. 7).

### Conclusion

The results demonstrate that psilocybin exerts a rapid beneficial action in well-studied and well-validated models of chronic stress-induced deficits in depression-relevant hedonic behaviors. Although depression is a uniquely human disease, findings from animal experiments can provide insights into psilocybin's mechanisms of action that are challenging to obtain in humans, such as receptor pharmacology. Indeed, the results recapitulate the rapid antidepressant actions of psilocybin in humans and alterations in brain functional connectivity that outlast the presence of the drug, as reported previously. Although psilocybin has been reported to have delayed antidepressant-like effects in the forced swim test in some, but not all, strains of rats, this was not observed in mice.

Psychedelic compounds alter consciousness through activation of 5-HT2ARs. The prevailing view in developing psychedelic compounds for psychiatry is that the mind-altering effects of these compounds contribute to, or are responsible for, the therapeutic benefits. The data shows that pretreatment with ketanserin sufficiently attenuated the activation of 5-HT2ARs at the time psilocybin was administered.

Ketanserin was observed to block 5-HT2AR-dependent head twitching and 5-HT2AR-dependent deceases in low frequency oscillatory activity (FIGS. 2A-2D). Preclinical results suggest that 5-HT2ARs, and thus psychedelic responses, may not be required for an antidepressant response to psilocybin. Mice with little-or-no evidence of psilocybin-induced head twitches were observed, indicative of a lack of 5HT2AR activation, still exhibited a robust psilocybin-induced anti-anhedonic effect (FIG. 2B). These preclinical results therefore suggest that 5-HT2ARs, and thus psychedelic responses, may not be required for an antidepressant response to psilocybin.

### Example 2: LSD-Induced Neural Changes and Ketanserin Studies

This example demonstrates the effect of 5-HT2A receptor antagonism on psychedelic drug induced hallucinations and detection of LSD-induced neural changes in humans that are uncoupled from hallucinations.

### Materials and Methods

### Source of Data

The used dataset was acquired in the course of a registered clinical trial (ClinicalTrials.gov Identifier: NCT02451072) entitled: The Role of 5-HT2A Receptor in the Perception of Self and Personal Meaning in Healthy Volunteers. Participants, study design, neuroimaging data acquisition, preprocessing and global brain connectivity calculation are detailed in Preller et al. (eLife. 2018 Oct. 25; 7:e35082. Changes in global and thalamic brain connectivity in LSD-induced altered states of consciousness are attributable to the 5-HT2A receptor, PMID: 30355445). The relevant methodological sections pertinent to the current analysis are summarized below.

### Participants

24 subjects were included in the dataset (n=19 males; mean age=25.0 years, standard deviation=3.6 years, range=20-34 years). All included subjects were healthy, as confirmed by medical history, physical examination, blood analysis, electrocardiogra Mini-International Neuropsychiatric Interview (MINI-SCID) [Sheehan et al., 1998], the DSM-IV self-rating questionnaire for Axis-II personality disorders (SCIS-II) [Fydrich et al., 1997], and the Hopkins Symptom Checklist (SCL-90R) [Franke, 1995]. Subjects were required to abstain from prescription or illicit drugs, alcohol, smoking and caffeine.

### Study Design

The study was designed as a fully double-blind, randomized, within-subject cross-over study. During a session, subjects were pretreated with either a placebo or the 5-HT2A receptor antagonist Ketanserin 60 minutes before being treated with either a placebo or LSD. Thus, each subject participated in 3 sessions in which they underwent each of the following pretreatment · treatment conditions in a randomized, balanced order:
a) Placebo+placebo
b) Placebo+LSD
c) Ketanserin+LSD
   Two resting-state scans were performed, one 75 and the other 300 minutes after treatment. To assess participants' experience, a retrospective self-report questionnaire (5D-ASC, [Dittrich, 1998]) was administered 720 minutes after treatment, and a shorter version of the 5D-ASC 180, 250 and 360 minutes after treatment.

### Neuroimaging Data Acquisition

MRI data was acquired using a whole-brain gradient-echo planar imaging (EPI) sequence on a Philips Achieva 3.0T whole-body scanner. For each resting-scan, 240 volumes were acquired (total scan duration: 10 minutes). Additionally, high-resolution T1-weighted (via a 3D magnetization-prepared rapid gradient-echo sequence, MP-RAGE) and T2-weighted (via a turbo spin-echo sequence) anatomical images were acquired.

### Preprocessing

MRI data was preprocessed following the methods developed by the Human Connectome Project (Glasser et al., 2013, NeuroImage). For Tl-weighted and T2-weighted images, a bias-field correction was applied, the images were warped to the MNI-152 template, followed by brain-extraction, within-subjects registration, and individual cortical and subcortical anatomical segmentation (Reuter et al., 2012, NeuroImage). For functional MRI (BOLD) images, corrections for field inhomogeneity distortion, phase encoding direction distortions, susceptibility artifacts, and motion were applied. Subsequently, BOLD images were registered to the structural images, and non-brain tissue was removed via a brain-mask. Then, BOLD time series were high-pass filtered, and the ventricle, white matter and global (mean gray matter) signal were regressed out of the gray matter time series. Finally, frames were excluded if the sum of the displacements across all six rigid body movement correction parameters exceeded 0.5 mm or the normalized root mean square of intensity differences between a given frame and its preceding frame exceeded 1.6 times the median across scans.

### Parcellation

For some of the analyses, voxel-wise time series were parcellated using the Cole-Anticevic Brain-wide Network-Parcellation atlas (CAB-NP) (Ji et al., NeuroImage, 2019). Parcellated time-series were generated with Connectome Workbench.

### Functional Connectivity (FC) and Global Brain Connectivity (GBC) Calculation

Functional connectivity (FC) was calculated as the Pearson correlation coefficient between each pair of voxels' or each pair of parcels' BOLD time series, resulting in voxel×voxel or parcel×parcel FC matrix, respectively. Fisher-z transformed FC matrices, FC′z, were obtained by applying a Fisher-z transform individually to each element of a matrix:

FCzᵢⱼ=FisherZ(FCᵢⱼ)=artanh(FCᵢⱼ)

where i and j denote parcels or voxels. To avoid extreme values resulting from the Fisher z-transform, correlations coefficients above 0.999999 or below −0.999999 were treated as 0.999999 and −0.999999, respectively.

Global brain connectivity (GBC) was then calculated as the average across rows of the Fisher-z transformed FC matrices:

$${GBC}_{i} = {\sum }_{j} \frac{1}{N} {FCz}_{ij}$$

where, again, i and j denote parcels or voxels. The GBC metric provides an interpretable reduction of the full functional connectivity matrix whereby the map reflects the average connectivity of each voxel to all other voxels. This metric is highly desirable as a feature space reduction procedure without biasing the effects towards any one area.

The difference in FC and GBC across any two conditions, i.e.: ΔFC=FC(condition 2)−FC(condition 1); and ΔGBC=GBC(condition 2)−GBC(condition 1) were analyzed. Typically, these are matched for the scan time, i.e., early (75-min), late (300-min), or average (combined early and late).

### Visualization

Two types of visualizations are provided:

CIFTI brain maps: In the first visualization a surface-based representation and volume-based representation of the data were highlighted where the shade of grayscale reflects the ΔGBC value for a given contrast. The intensity of the value in the image reflects a Z statistic, where for instance a value of 1 can be interpreted in 1 standard deviation along a standard Z distribution indicating the difference between the relevant conditions (i.e. ΔGBC). Here lighter shades of grayscale denote higher GBC for the first condition relative to the second condition, whereas darker shades of grayscale denote lower GBC for the first condition relative to the second condition.

Chord plots: In the second visualization, a standard ‘chord’ plot was shown where each segment along the circle indicates a given left hemisphere cortical parcel from the CAB-NP parcellation (described above). The ‘chord’ connecting any two nodes reflects the difference across two conditions in functional connectivity (i.e. an edge) between any two nodes. That is, the value represents AFC between two relevant conditions. Light shades of gray denotes higher connectivity for the first condition relative to the second condition, whereas dark shades of gray denote lower connectivity for the first condition relative to the second condition. Connectivities that were very similar in both conditions were plotted in medium shade of gray.

### Statistical Analysis

ΔGBC contrasts were evaluated using a 1-sample t-test. Confidence intervals were calculated as

$$( \overline{x} - \frac{cs}{\sqrt{n}} , \overline{x} + \frac{cs}{\sqrt{n}} )$$

where x̅ is the mean of the observed contrasts, s their standard deviation, n the number of samples (subjects), and c is the 97.5th percentile of the t distribution with n−1 degrees of freedom. Raw p-values were adjusted for multiple comparisons using the Benjamini-Hochberg FDR correction.

### Results

Pretreatment with Ketanserin, Compared to Pretreatment with Placebo, Leads to Distinct Neural Changes at the Early Time Point (75 Minutes)

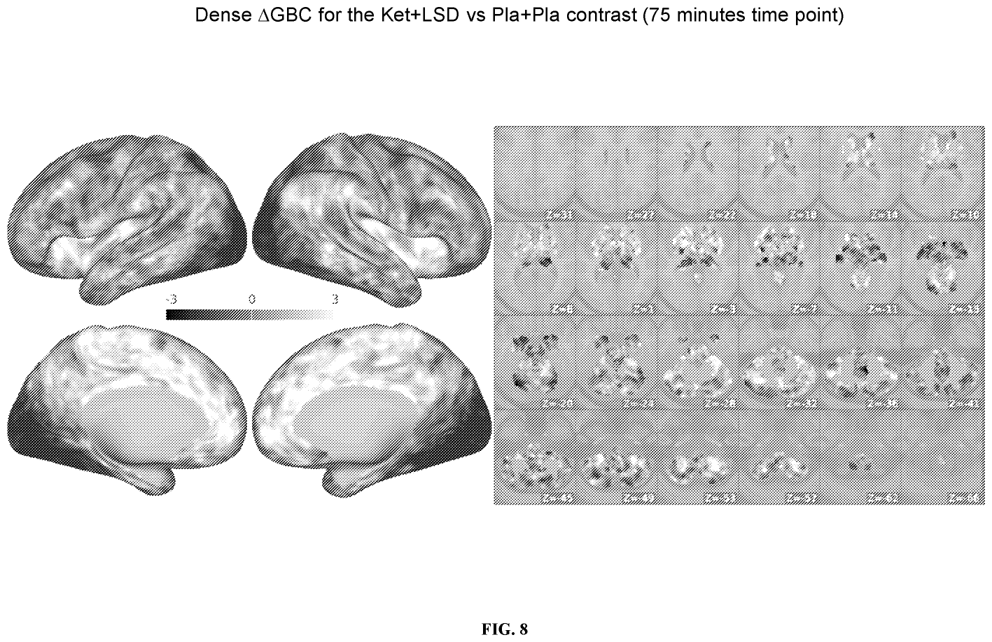

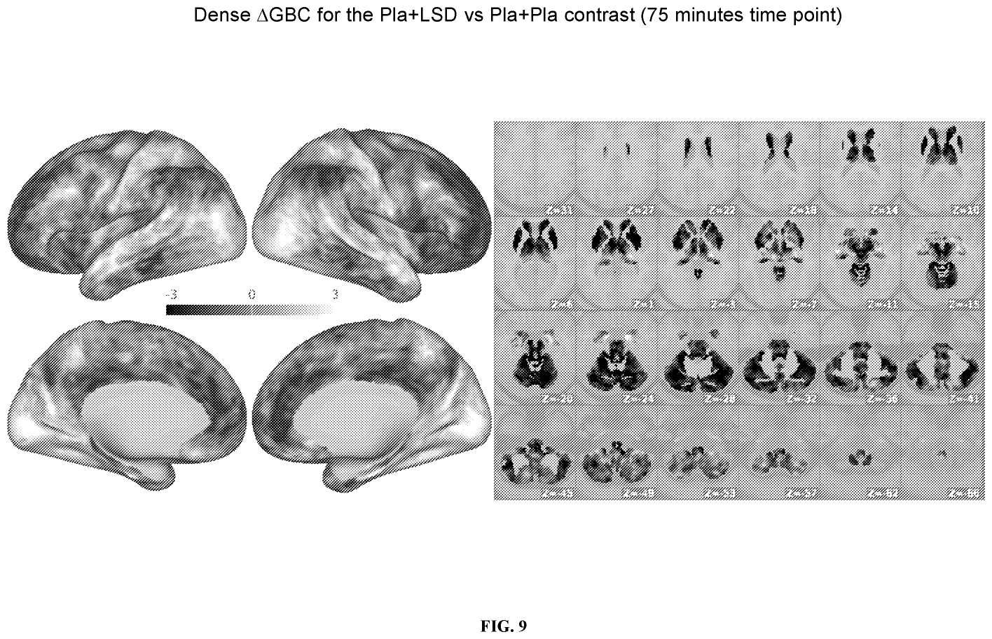

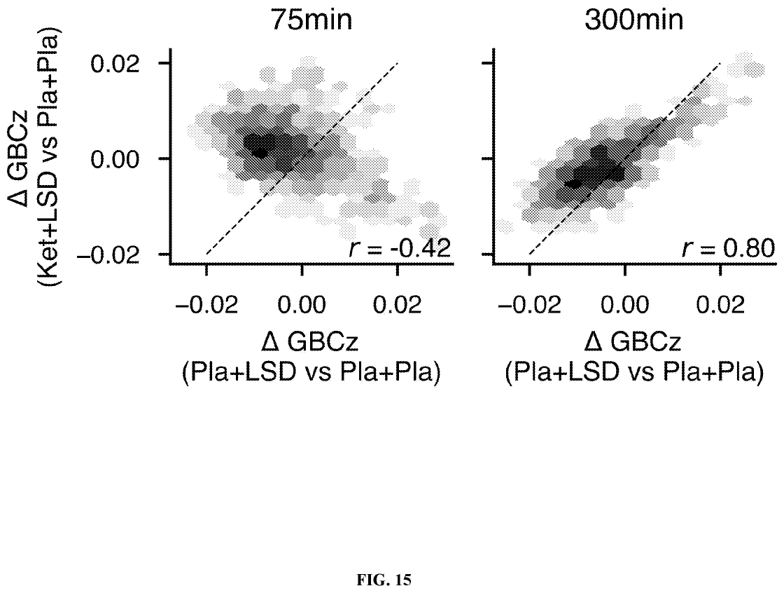

There was clear neural evidence that pretreatment with ketanserin, compared to pretreatment with placebo, led to differences at the whole-brain level at the early time point (75 minutes). This was apparent by visual inspection of dense GBC maps (FIG. 8 and FIG. 9). A more quantitative inspection of the two different pretreatment conditions at the early time point (75 minutes) is shown in FIG. 15 (left panel). While GBC changes between the two different pretreatment conditions have a net negative correlation (r=−0.42, see also Table 1).

TABLE 1
Correlations between GBC changes in parcels induced by Pla +
LSD and Ket + LSD (relative to Pla + Pla)

| Session | r | p |
|---|---|---|
| Early (75 minutes) | −0.42 | <10⁻³ |
| Late (300 minutes) | 0.80 | <10⁻³ |

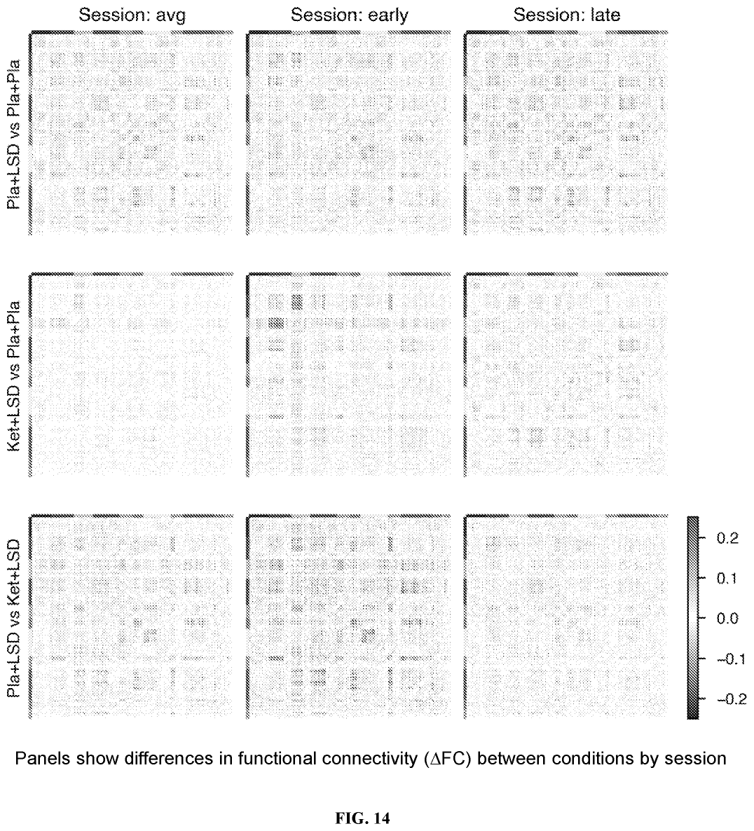

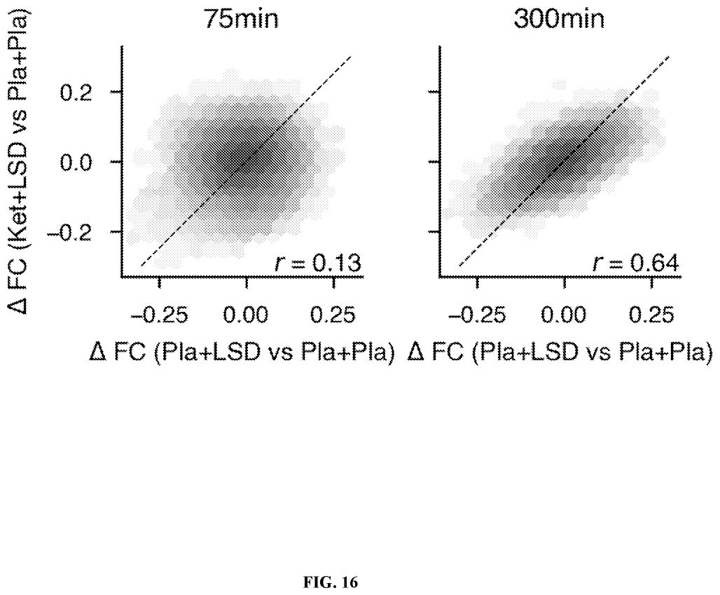

Next, pretreatment effects on FC values were investigated. FC contrasts between all condition pairs and for all time points are shown in FIG. 14. A closer, quantitative inspection of the connectivity contrasts between for the 718 parcels of the CAP-NP parcellation shows that FC changes between the two pretreatment conditions are only mildly correlated (r=0.13, see FIG. 16 left panel and Table 2). Note that, due to the large number of connections (˜ 260,000) this mild correlation is nonetheless strongly significant.

TABLE 2
Correlations between FC changes in parcels induced by Pla +
LSD and Ket + LSD (relative to Pla + Pla)

| Session | r | p |
|---|---|---|
| Early (75 minutes) | 0.13 | <10⁻³ |
| Late (300 minutes) | 0.64 | <10⁻³ |

Pretreatment with Ketanserin and Pretreatment with Placebo Lead to Similar Neural Changes at the Late Time Point

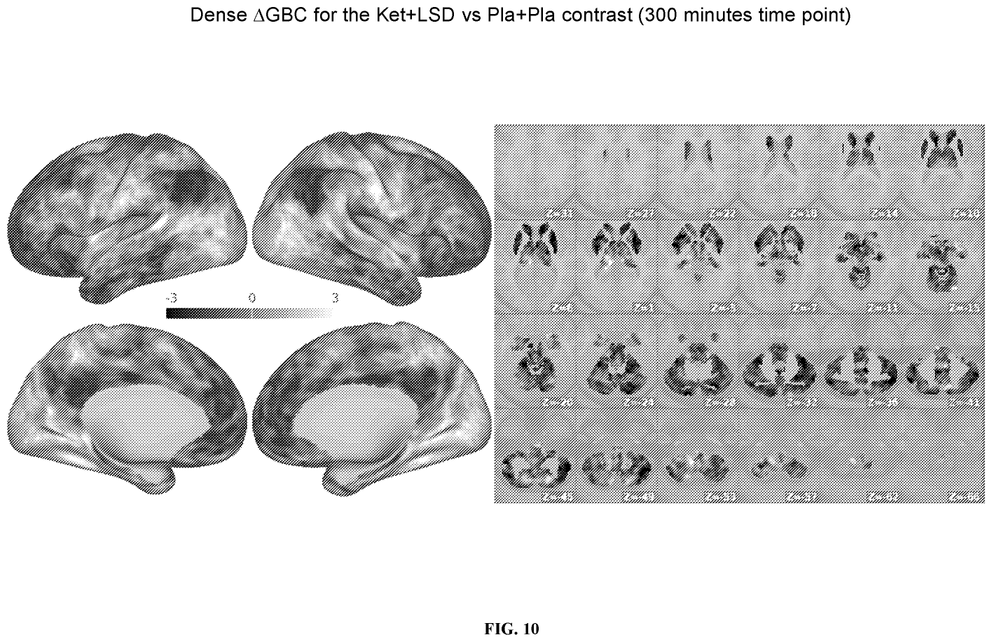

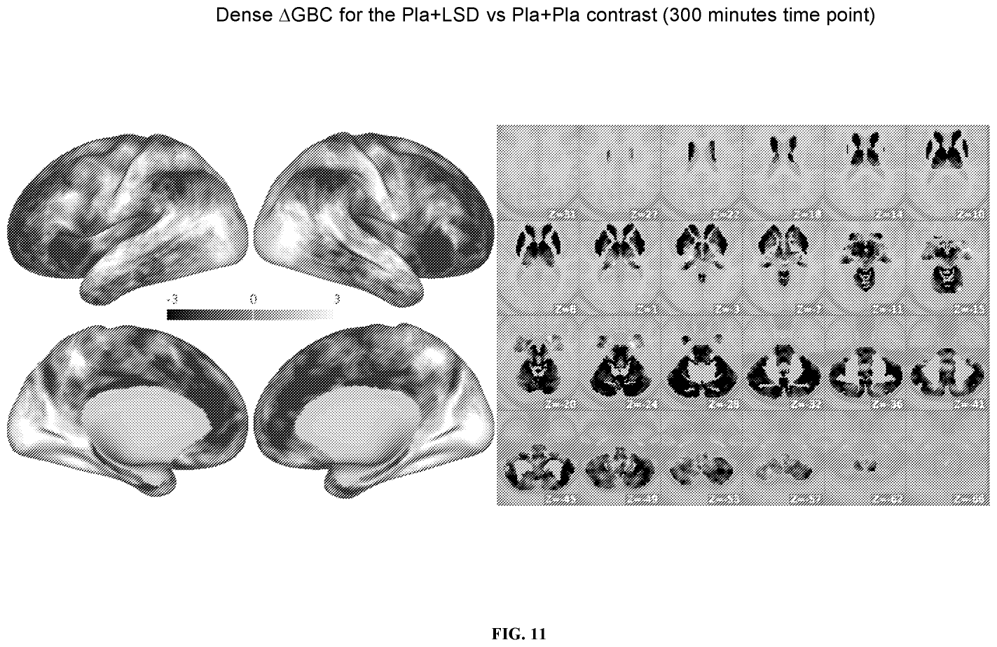

At the late time point (after 300 minutes), GBC changes in the two different pretreatment conditions are strongly positively correlated. This is immediately apparent by visual inspection (FIG. 10 and FIG. 11). Moreover, FIG. 15 (right panel) confirms this observation quantitatively. The correlation between the ΔGBC changes across parcels was 0.8 (see also Table 1).

This strong correlation was corroborated when analyzing FC contrasts. The correlation across connections between AFC values for the two different pretreatment conditions was found to be 0.64 (see FIG. 16 right panel and Table 2). A FC values were slightly stronger in the Pla+LSD as compared to the Ket+LSD condition.

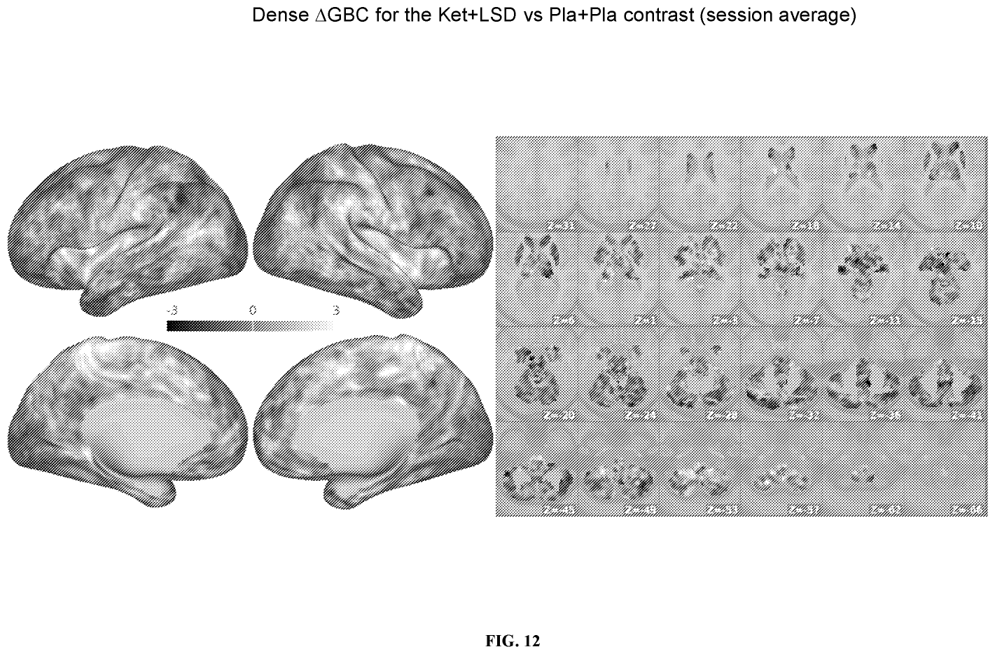

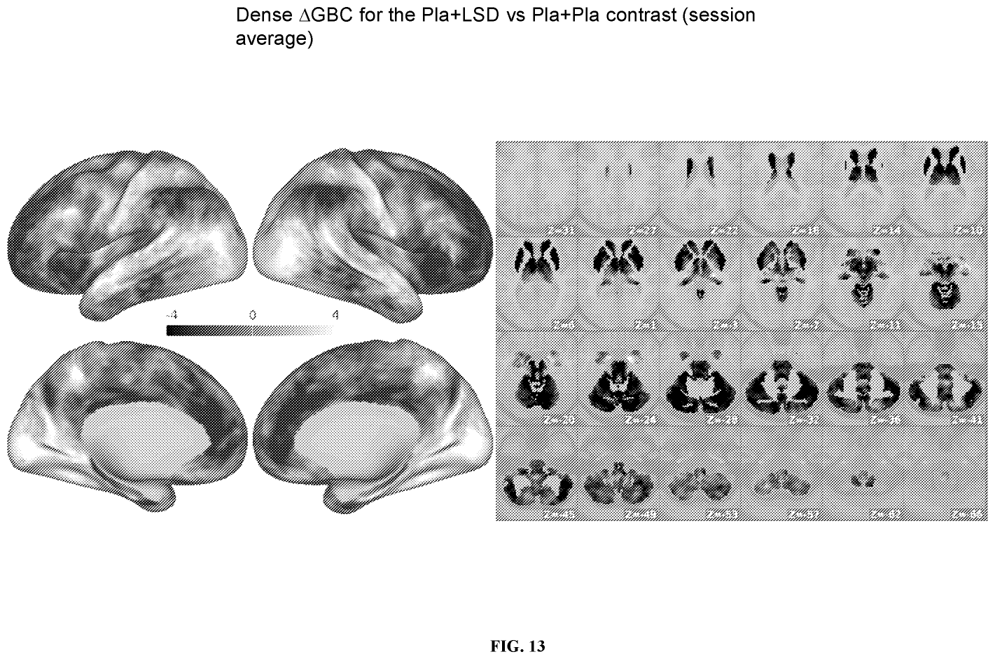

ΔGBC changes averaged across sessions (FIG. 12 and FIG. 13) show the combined measured overlap of early and late patterns of neural change.

Parcels that Might Underlie Effects

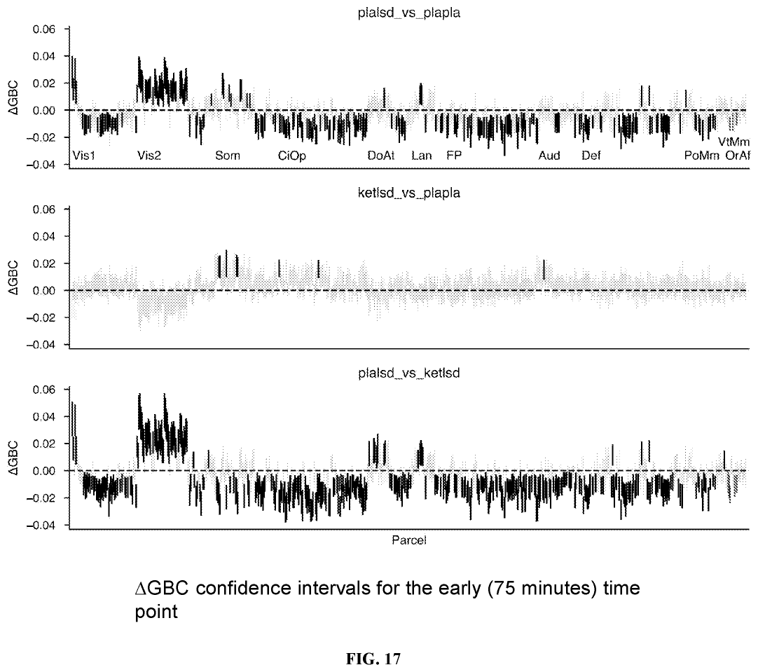

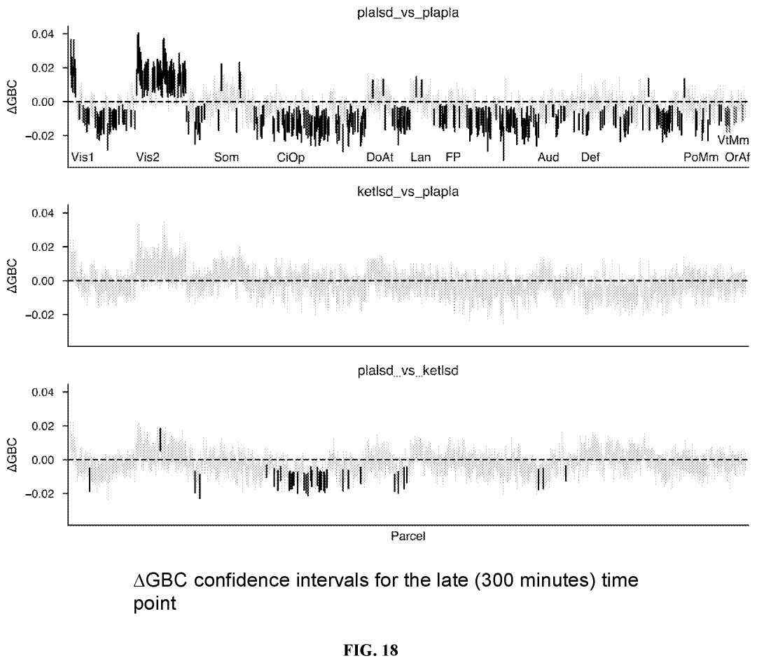

For which parcels a contrast's distribution across subjects is significant was tested. For each contrast, t-statistics, corresponding p-values, confidence intervals and FDR-BH corrected p-values were all calculated using 1-sample t-tests. The outcomes are provided in Table S1. Confidence intervals are visualized in FIG. 17 and FIG. 18 for the early and late time point, respectively. Desaturated (light) shades of grayscale were used in these figures when a parcel's contrast was not significant after FDR correction (i. e. when p_{corrected}≥0.05). Nonetheless, as shown above (FIG. 15), the overall pattern of contrasts across parcels resembled the Pla+LSD—vs—Pla+Pla pattern of contrasts, especially at the late time point.

### FC Network Matrix

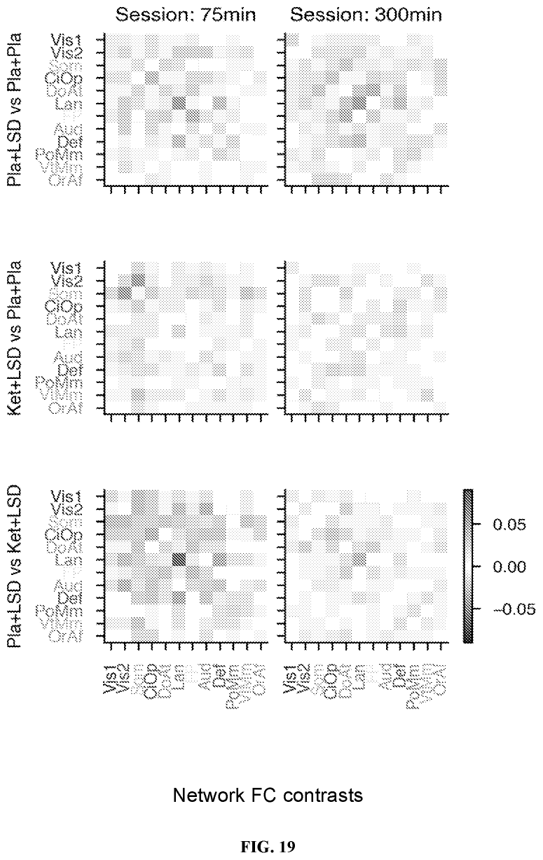

For networks defined in the CAP-NP atlas, pairwise comparison of FC was made for each network pair for the different test conditions at early, late and average session time points. For each network pair, all connection contrasts (i.e. AFC values) between these two networks were averaged resulting in a network×network FC contrast matrix (FIG. 19).

Pretreatment with Ketanserin Prevents Self-Reported Altered States of Consciousness in LDS-Treated Subjects

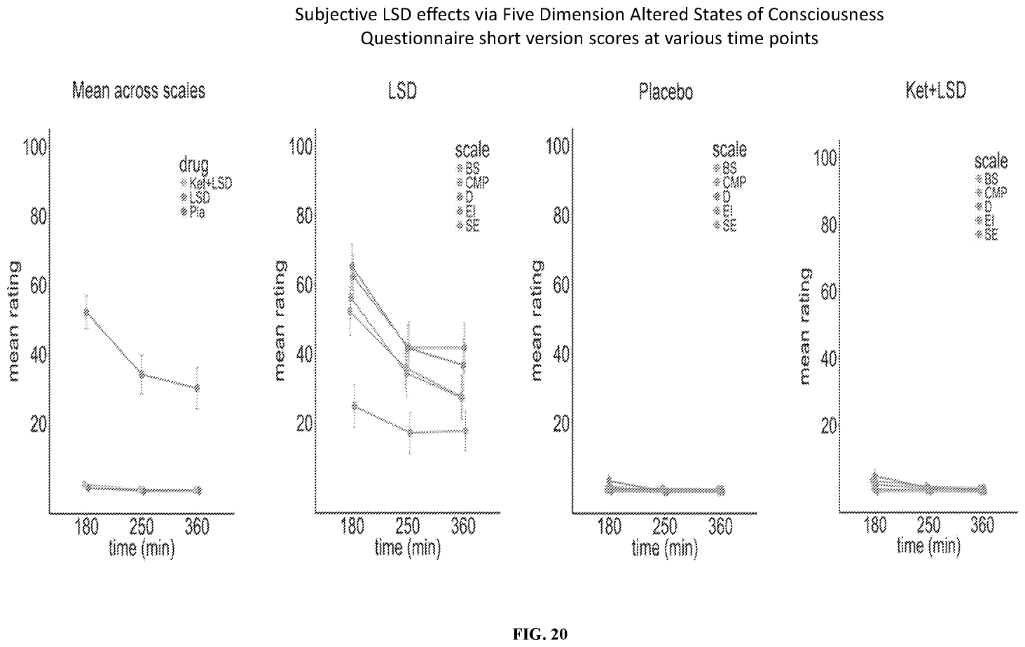

Test subjects treated with Pla+LSD exhibited substantial self-reported altered states of consciousness at the 180-minute, 250-minute and 360-minute time points (FIG. 20). Pretreatment with ketanserin prevented significant self-reported altered states of consciousness at all time points tested in the Ket+LSD test subject group (FIG. 20, right graph).

### Conclusions

Clear effects of pretreatment with ketanserin at the early time point in conjunction with LSD that were distinct from placebo (75 min after treatment administration) were observed. At this early time point, connections that exhibit a change under Ket+LSD may reflect additional therapeutic targets that were not blocked by Ketanserin, but were modulated by LSD.

A different picture was observed at the later time point (300 minutes after treatment administration). Here, both GBC and FC changes were strongly correlated when examining maps for Pla+LSD and Ket+LSD (across parcels and connections, respectively). This indicated that even in the absence of psychotomimetic effects of LSD at this later time point there were neural effects that strongly resemble the fully psychotomimetic state when only LSD is administered. Importantly, despite these similarities in neural effects, the two conditions differed behaviorally: when subjects received a placebo pre-treatment they reported psychotomimetic effects of LSD, but when they received ketanserin as pre-treatment they did not (Preller et al., eLife, 2018). Notably, the Pla+LSD group GBC maps did not significantly differ between 75 minutes and 300 minutes indicating that the LSD effects alone produced a robust and consistent effect across time. This contrasted with the Ket+LSD map which significantly changed from 75 minutes to 300 minutes. The finding that the LSD effects were comparable from 75 minutes to 300 minutes was consistent with the 5D-ASC scale data showing the psychotomimetic effects in the Pla+LSD group were still significantly elevated from placebo at the 360 minute timepoint (FIG. 20). This indicated that the neural effects observed via imaging of the Ket+LSD condition at 300 minutes reveal neural changes of the core effects of LSD.

However, while ketanserin pretreatment blocked all of the psychotomimetic effects of LSD at all timepoints and many of the immediate neural changes induced by LSD at 75 minutes, these LSD induced neural changes reached near full effect within 300 minutes in the presence of ketanserin and in the absence of psychotomimetic effects. This is the first evidence showing that while pretreatment with ketanserin fully blocked the behavioral effects of LSD (the “trip”), it did not block the neural changes induced by LSD that are thought to underlie its potential therapeutic effects.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A composition comprising:
a) a psychedelic;
b) a serotonin receptor modulator; and
c) an excipient,
wherein the serotonin receptor modulator is released at most about 3 hours prior to the release of the psychedelic.

2. The composition of claim 1, wherein the psychedelic is an agonist for a serotonin receptor.

3. The composition of claim 2, wherein the serotonin receptor is serotonin receptor 1B, serotonin receptor 4, serotonin receptor 6, or serotonin receptor 7.

4. The composition of claim 1, wherein the psychedelic is selected from psilocybin, psilocin, baeocystin, norbaeocystin, lisurgide, LSD, dimethyltryptamine, carboxamindotryptamine, ibogaine, tabernanthalog, 3,4-methylenedioxy-methamphetamine (MDMA), 1-acetyl LSD, O-acetyl psilocin, mescaline (3,4,5-trimethoxy phenethylamine), proscaline (2-(3,5-dimethoxy-4-propoxyphenyl)ethanamine), metaescaline (2-(3-ethoxy-4,5-dimethoxyphenyl)ethanamine), allylescaline (4-Allyloxy-3,5-dimethyloxy phenylethylamine), methallylescaline (4-Methallyloxy-3,5 dimethoxyphenethylamine), and asymbescaline (3,4-Diethoxy-5-methoxyphenethylamine), or a pharmaceutically acceptable salt, solvate, metabolite, deuterated analogue, derivative, prodrug, or combinations thereof.

5. The composition of claim 1, wherein the psychedelic is LSD or a pharmaceutically acceptable salt, solvate, metabolite, deuterated analogue, derivative, or prodrug thereof.

6. The composition of claim 5, wherein the LSD derivative is 1P-LSD, 1B-LSD, ETH-LAD, 1P-ETH-LAD, AL-LAD, LSZ, LSM-775, or 1-(4-Bromofuro[2,3-f][1]benzofuran-8-yl)propan-2-amine.

7. The composition of claim 1, wherein the psychedelic is mescaline or a pharmaceutically acceptable salt, solvate, metabolite, deuterated analogue, derivative, or prodrug thereof.

8. The composition of claim 7, wherein the mescaline derivative is mescaline-NBOMe, proscaline (2-(3,5-dimethoxy-4-propoxyphenyl)ethanamine), or metaescaline (2-(3-ethoxy-4,5-dimethoxyphenyl)ethanamine).

9. The composition of claim 1, wherein the psychedelic is a phenethylamine or a tryptamine, or a pharmaceutically acceptable salt, solvate, metabolite, deuterated analogue, derivative, or prodrug thereof.

10. The composition of claim 9, wherein the phenethylamine or the tryptaminedeuterated analogue is selected from the group consisting of 25I-NBOH, N-(2-Methoxybenzyl)-2-(3,4,5-trimethoxyphenyl)ethanamine, N-(2-hydroxybenzyl)-2,5-dimethoxy-4-iodo-phenethylamine, N-(2-hydroxybenzyl)-2,5-dimethoxy-4-chloro-phenethylamine, N-(2-hydroxybenzyl)-2,5-dimethoxy-4-bromo-phenethylamine, 4-Allyloxy-3,5-dimethyloxyphenylethylamine, N-(2-fluorobenzyl)-2,5-dimethoxy-4-iodo-phenethylamine, 2,5-dimethoxy-4-tert-butylthio-phenethylamine, 2,5-dimethoxy-4-propylthio-phenethylamine, 2,5-dimethoxy-4-propylphenethylamine, 2,5-dimethoxy-4-nitrophenethylamine, 2,5-dimethoxy-4-nitroamphetamine, 2,5-dimethoxy-4-methylphenethylamine, 2,5-dimethoxy-4-methylamphetamine, 2,5-dimethoxy-4-isopropylthio-phenethylamine, 2,5-dimethoxy-4-iodophenethylamine, 2,5-dimethoxy-4-iodoamphetamine, 2,5-dimethoxy-4-fluorophenethylamine, 2,5-dimethoxy-4-ethylthio-phenethylamine, 2,5-dimethoxy-4-ethylphenethylamine, 2,5-dimethoxy-4-cyclopropylmethylthio-phenethylamine, 2,5-dimethoxy-4-chlorophenethylamine, 2,5-dimethoxy-4-chloroamphetamine, 2,5-dimethoxy-4-bromophenethylamine, 2,5-dimethoxy-4-bromoamphetamine, 2,5-dimethoxy-4-bromo-ß-ketophenethylamine, 2,5-dimethoxy-4-(2-fluoroethylthio)-phenethylamine, 2-(4-propyl-2,5-dimethoxyphenyl)-N-[(2-methoxyphenyl)methyl]ethanamine, 2-(4-methyl-2,5-dimethoxyphenyl)-N-[(2-methoxyphenyl)methyl]ethanamine, 2-(4-iodo-2,5-dimethoxyphenyl)-N-[(2-methoxyphenyl)methyl]ethanamine, 2-(4-fluoro-2,5-dimethoxyphenyl)-N-[(2-methoxyphenyl)methyl]ethanamine, 2-(4-ethyl-2,5-dimethoxyphenyl)-N-[(2-methoxyphenyl)methyl]ethanamine, 2-(4-chloro-2,5-dimethoxyphenyl)-N-[(2-methoxyphenyl)methyl]ethanamine, 2-(4-bromo-2,5-dimethoxyphenyl)-N-[(2-methoxyphenyl)methyl]ethanamine, 4-AcO-MET, 4-AcO-MALT, 4-AcO-DALT, Aeruginascin, N,N,N-trimethyl-4-phosphoryloxytryptamine, 4-Hydroxy-N,N,N-trimethyltryptamine, 5-meo-DMT, Ibogaine, [3-(2-Dimethylaminoethyl)-1H-indol-4-yl] dihydrogen phosphate, 4-hydroxytryptamine, 4-hydroxy-N,N-dimethyl-tryptamine, [3-(2-methylaminoethyl)-1H-indol-4-yl] dihydrogen phosphate, 4-hydroxy-N-methyltryptamine, [3-(aminoethyl)-1H-indol-4-yl] dihydrogen phosphate, [3-(2-trimethylaminoethyl)-1H-indol-4-yl] dihydrogen phosphate, 4-hydroxy-N,N,N-trimethyltryptamine, 6-Allyl-N,N-diethyl-NL, N,N-Dibutyl-tryptamine, N,N-Diethyl-tryptamine, N,N-Diisopropyl-tryptamine, 5-Methyoxy-alpha-methyl-tryptamine, N,N-Dimethyl-tryptamine, 2,alpha-Dimethyl-tryptamine, alpha,N-Dimethyl-tryptamine, N,N-Dipropyl-tryptamine, N-Ethyl-N-isopropyl-tryptamine, alpha-Ethyl-tryptamine, 6,N,N-Triethyl-NL, 3,4-Dihydro-7-methoxy-1-methyl-C, 7-Methyoxy-1-methyl-C, N,N-Dibutyl-4-hydroxy-tryptamine, N,N-Diethyl-4-hydroxy-tryptamine, N,N-Diisopropyl-4-hydroxy-tryptamine, N,N-Dimethyl-4-hydroxy-tryptamine, N,N-Dimethyl-5-hydroxy-tryptamine, N,N-Dipropyl-4-hydroxy-tryptamine, N-Ethyl-4-hydroxy-N-methyl-tryptamine, 4-Hydroxy-N-isopropyl-N-methyl-tryptamine, 4-Hydroxy-N-methyl-N-propyl-tryptamine, 4-Hydroxy-N,N-tetram ethylene-tryptamine, N,N-Diethyl-L, N-Butyl-N-methyl-tryptamine, N,N-Diisopropyl-4,5-methylenedioxy-tryptamine, N,N-Diisopropyl-5,6-methylenedioxy-tryptamine, N,N-Dimethyl-4,5-methylenedioxy-tryptamine, N,N-Dimethyl-5,6-methylenedioxy-tryptamine, N-Isopropyl-N-methyl-5,6-methylenedioxy-tryptamine, N,N-Diethyl-2-methyl-tryptamine, 2, N, N-Trimethyl-tryptamine, N-Acetyl-5-methoxy-tryptamine, N,N-Diethyl-5-methoxy-tryptamine, N,N-Diisopropyl-5-methoxy-tryptamine, 5-Methoxy-N,N-dimethyl-tryptamine, N-Isopropyl-4-methoxy-N-methyl-tryptamine, N-Isopropyl-5-methoxy-N-methyl-tryptamine, 5,6-Dimethoxy-N-isopropyl-N-methyl-tryptamine, 5-Methoxy-N-methyl-tryptamine, 5-Methoxy-N,N-tetramethylene-tryptamine, 6-Methoxy-1-methyl-1,2,3,4-tetrahydroisoquinoline, 5-Methoxy-2,N,N-trimethyl-tryptamine, N,N-Dimethyl-5-methylthio-tryptamine, N-Isopropyl-N-methyl-tryptamine, alpha-Methyl-tryptamine, N-Ethyl-tryptamine, N-Methyl-tryptamine, 6-Propyl-N L, N,N-Tetramethylene-tryptamine, Tryptamine, 7-Methoxy-1-methyl-1, 2,3,4-tetrahydroisoquinoline, and alpha, N-Dimethyl-5-methoxy-tryptaminedeuterated analogue.

11. The composition of claim 1, wherein the psychedelic is psilocybin or a pharmaceutically acceptable salt, solvate, metabolite, deuterated analogue, derivative, or prodrug thereof.

12. The composition of claim 1, wherein the psychedelic is 1-acetyl LSD or a pharmaceutically acceptable salt, solvate, metabolite, deuterated analogue, derivative, or prodrug thereof.

13. The composition of claim 1, wherein the psychedelic is O-acetyl psilocin, or a pharmaceutically acceptable salt, solvate, metabolite, deuterated analogue, derivative, or prodrug thereof.

14. The composition of any one of claims 1-13, wherein the serotonin receptor modulator is a serotonin receptor antagonist.

15. The composition of any one of claims 1-13, wherein the serotonin receptor modulator is a serotonin receptor inverse agonist.

16. The composition of any one of claims 14-15, wherein the serotonin receptor is 5-HT2A receptor.

17. The composition of any one of claims 1-16, wherein the serotonin receptor modulator is selected from the group consisting of ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, AMDA, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, and RS-102221, or a pharmaceutically acceptable salt, solvate, metabolite, deuterated analogue, derivative, prodrug, or combinations thereof.

18. The composition of any one of claims 1-17, wherein the serotonin receptor modulator is ketanserin or a pharmaceutically acceptable salt, solvate, metabolite, deuterated analogue, derivative, or prodrug thereof.

19. The composition of any one of claims 1-17, wherein the serotonin receptor modulator is pimavanserin or a pharmaceutically acceptable salt, solvate, metabolite, deuterated analogue, derivative, or prodrug thereof.

20. The composition of any one of claims 1-19, wherein the serotonin receptor modulator is released at most about 2.5 hours prior to the release of the psychedelic.

21. The composition of any one of claims 1-19, wherein the serotonin receptor modulator is released at most about 2 hours prior to the release of the psychedelic.

22. The composition of any one of claims 1-19, wherein the serotonin receptor modulator is released at most about 1.5 hours prior to the release of the psychedelic.

23. The composition of any one of claims 1-19, wherein the serotonin receptor modulator is released at most about 1 hour prior to the release of the psychedelic.

24. The composition of any one of claims 1-19, wherein the serotonin receptor modulator is released at about 1 hour prior to the release of the psychedelic.

25. The composition of any one of claims 1-19, wherein the serotonin receptor modulator is released at least about 0.5 hours prior to the release of the psychedelic.

26. The composition of any one of claims 1-19, wherein the serotonin receptor modulator is released at least about 1 hour prior to the release of the psychedelic.

27. The composition of any one of claims 1-24, wherein the psychedelic is provided at a dose of about 100 milligrams (mg) to about 1 gram (g).

28. The composition of any one of claims 1-24, wherein the psychedelic is provided at a dose of about 100 mg to about 800 mg.

29. The composition of any one of claims 1-24, wherein the psychedelic is provided at a dose of about 100 mg to about 500 mg.

30. The composition of any one of claims 1-24, wherein the psychedelic is provided at a dose of about 10 mg to about 400 mg.

31. The composition of any one of claims 1-24, wherein the psychedelic is provided at a dose of about 20 mg to about 200 mg.

32. The composition of any one of claims 1-24, wherein the psychedelic is provided at a dose of about 10 mg to about 100 mg.

33. The composition of any one of claims 1-24, wherein the psychedelic is provided at a dose of about 10 micrograms (μg) to about 400 μg.

34. The composition of any one of claims 1-24, wherein the psychedelic is provided at a dose of about 20 μgμg to about 200 μg.

35. The composition of any one of claims 1-24, wherein the psychedelic is provided at a dose of about 10 μg μg to about 100 μg μg.

36. The composition of any one of claims 1-35, wherein the serotonin receptor modulator is provided at a dose of about 10 mg to about 350 mg.

37. The composition of any one of claims 1-35, wherein the serotonin receptor modulator is provided at a dose of about 20 mg to about 200 mg.

38. The composition of any one of claims 1-35, wherein the serotonin receptor modulator is provided at a dose of about 10 mg to about 100 mg.

39. The composition of any one of claims 1-38, wherein the psychedelic is provided at a dose of about 10 mg to about 100 mg and the serotonin receptor modulator is provided at a dose of about 10 mg to about 100 mg.

40. The composition of any one of claims 1-39, wherein the psychedelic is psilocybin or a pharmaceutically acceptable salt, solvate, metabolite, deuterated analogue, derivative, or prodrug thereof and the serotonin receptor modulator is ketanserin or a pharmaceutically acceptable salt thereof.

41. The composition of any one of claims 1-40, wherein the psychedelic is psilocybin or a pharmaceutically acceptable salt, solvate, metabolite, deuterated analogue, derivative, or prodrug thereof and provided at a dose of about 10 mg to about 100 mg; and the serotonin receptor modulator is ketanserin or a pharmaceutically acceptable salt thereof provided at a dose of about 10 mg to about 100 mg.

42. The composition of any one of claims 1-41, wherein the composition is in a form of a single pill, single ampoule, or single unit dosage form.

43. The composition of any one of claims 1-39, wherein the composition is in a single unit dosage form.

44. A method of treating a disease or disorder in a subject in need thereof, the method comprising administering to the subject a composition comprising:
a) a psychedelic;
b) a serotonin receptor modulator; and
c) an excipient,
wherein the serotonin receptor modulator is released at most about 3 hours prior to the release of the psychedelic.

45. The method of claim 44, wherein the disease or disorder is depression or a disease or disorder related to depression.

46. The method of claim 44, wherein the depression is major depressive disorder, persistent depressive disorder, bipolar disorder, treatment resistant depression (TRD), postpartum depression, premenstrual dysphoric disorder, or seasonal affective disorder.

47. The method of claim 44, wherein the disease or disorder related to depression is anxiety.

48. The method of any one of claims 44-47, wherein the psychedelic is an agonist for a serotonin receptor.

49. The method of claim 48, wherein the serotonin receptor is serotonin receptor 1B, serotonin receptor 4, serotonin receptor 6, or serotonin receptor 7.

50. The method of any one of claims 44-47, wherein the psychedelic is selected from psilocybin, psilocin, baeocystin, norbaeocystin, lisurgide, LSD, dimethyltryptamine, carboxamindotryptamine, ibogaine, tabernanthalog, 3,4-methylenedioxy-methamphetamine (MDMA), 1-acetyl LSD, O-acetyl psilocin, mescaline (3,4,5-trimethoxy phenethylamine), proscaline (2-(3,5-dimethoxy-4-propoxyphenyl)ethanamine), metaescaline (2-(3-ethoxy-4,5-dimethoxyphenyl)ethanamine), allylescaline (4-Allyloxy-3,5-dimethyloxy phenylethylamine), methallylescaline (4-Methallyloxy-3,5 dimethoxyphenethylamine), and asymbescaline (3,4-Diethoxy-5-methoxyphenethylamine), or a pharmaceutically acceptable salt, solvate, metabolite, deuterated analogue, derivative, prodrug, or combinations thereof.

51. The method of any one of claims 44-47, wherein the psychedelic is LSD or a pharmaceutically acceptable salt, solvate, metabolite, deuterated analogue, derivative, or prodrug thereof.

52. The method of claim 51, wherein the LSD derivative is 1P-LSD, 1B-LSD, ETH-LAD, 1P-ETH-LAD, AL-LAD, LSZ, LSM-775, or 1-(4-Bromofuro[2,3-f][1]benzofuran-8-yl)propan-2-amine.

53. The method of any one of claims 44-47, wherein the psychedelic is mescaline or a pharmaceutically acceptable salt, solvate, metabolite, deuterated analogue, derivative, or prodrug thereof.

54. The method of claim 53, wherein the mescaline derivative is mescaline-NBOMe, proscaline (2-(3,5-dimethoxy-4-propoxyphenyl)ethanamine), or metaescaline (2-(3-ethoxy-4,5-dimethoxyphenyl)ethanamine).

55. The method of any one of claims 44-47, wherein the psychedelic is a phenethylamine or a tryptamine, or a pharmaceutically acceptable salt, solvate, metabolite, deuterated analogue, derivative, or prodrug thereof.

56. The method of claim 55, wherein the phenethylamine or the tryptamineis selected from 25I-NBOH, N-(2-Methoxybenzyl)-2-(3,4,5-trimethoxyphenyl)ethanamine, N-(2-hydroxybenzyl)-2,5-dimethoxy-4-iodo-phenethylamine, N-(2-hydroxybenzyl)-2,5-dimethoxy-4-chloro-phenethylamine, N-(2-hydroxybenzyl)-2,5-dimethoxy-4-bromo-phenethylamine, 4-Allyloxy-3,5-dimethyloxyphenylethylamine, N-(2-fluorobenzyl)-2,5-dimethoxy-4-iodo-phenethylamine, 2,5-dimethoxy-4-tert-butylthio-phenethylamine, 2,5-dimethoxy-4-propylthio-phenethylamine, 2,5-dimethoxy-4-propylphenethylamine, 2,5-dimethoxy-4-nitrophenethylamine, 2,5-dimethoxy-4-nitroamphetamine, 2,5-dimethoxy-4-methylphenethylamine, 2,5-dimethoxy-4-methylamphetamine, 2,5-dimethoxy-4-isopropylthio-phenethylamine, 2,5-dimethoxy-4-iodophenethylamine, 2,5-dimethoxy-4-iodoamphetamine, 2,5-dimethoxy-4-fluorophenethylamine, 2,5-dimethoxy-4-ethylthio-phenethylamine, 2,5-dimethoxy-4-ethylphenethylamine, 2,5-dimethoxy-4-cyclopropylmethylthio-phenethylamine, 2,5-dimethoxy-4-chlorophenethylamine, 2,5-dimethoxy-4-chloroamphetamine, 2,5-dimethoxy-4-bromophenethylamine, 2,5-dimethoxy-4-bromoamphetamine, 2,5-dimethoxy-4-bromo-ß-ketophenethylamine, 2,5-dimethoxy-4-(2-fluoroethylthio)-phenethylamine, 2-(4-propyl-2,5-dimethoxyphenyl)-N-[(2-methoxyphenyl)methyl]ethanamine, 2-(4-methyl-2,5-dimethoxyphenyl)-N-[(2-methoxyphenyl)methyl]ethanamine, 2-(4-iodo-2,5-dimethoxyphenyl)-N-[(2-methoxyphenyl)methyl]ethanamine, 2-(4-fluoro-2,5-dimethoxyphenyl)-N-[(2-methoxyphenyl)methyl]ethanamine, 2-(4-ethyl-2,5-dimethoxyphenyl)-N-[(2-methoxyphenyl)methyl]ethanamine, 2-(4-chloro-2,5-dimethoxyphenyl)-N-[(2-methoxyphenyl)methyl]ethanamine, 2-(4-bromo-2,5-dimethoxyphenyl)-N-[(2-methoxyphenyl)methyl]ethanamine, 4-AcO-MET, 4-ACO-MALT, 4-AcO-DALT, Aeruginascin, N,N,N-trimethyl-4-phosphoryloxytryptamine, 4-Hydroxy-N,N,N-trimethyltryptamine, 5-meo-DMT, Ibogaine, [3-(2-Dimethylaminoethyl)-1H-indol-4-yl] dihydrogen phosphate, 4-hydroxytryptamine, 4-hydroxy-N,N-dimethyl-tryptamine, [3-(2-methylaminoethyl)-1H-indol-4-yl] dihydrogen phosphate, 4-hydroxy-N-methyltryptamine, [3-(aminoethyl)-1H-indol-4-yl] dihydrogen phosphate, [3-(2-trimethylaminoethyl)-1H-indol-4-yl] dihydrogen phosphate, 4-hydroxy-N,N,N-trimethyltryptamine, 6-Allyl-N,N-diethyl-NL, N,N-Dibutyl-tryptamine, N,N-Diethyl-tryptamine, N,N-Diisopropyl-tryptamine, 5-Methyoxy-alpha-methyl-tryptamine, N,N-Dimethyl-tryptamine, 2,alpha-Dimethyl-tryptamine, alpha,N-Dimethyl-tryptamine, N,N-Dipropyl-tryptamine, N-Ethyl-N-isopropyl-tryptamine, alpha-Ethyl-tryptamine, 6,N,N-Triethyl-NL, 3,4-Dihydro-7-methoxy-1-methyl-C, 7-Methyoxy-1-methyl-C, N,N-Dibutyl-4-hydroxy-tryptamine, N,N-Diethyl-4-hydroxy-tryptamine, N,N-Diisopropyl-4-hydroxy-tryptamine, N,N-Dimethyl-4-hydroxy-tryptamine, N,N-Dimethyl-5-hydroxy-tryptamine, N, N-Dipropyl-4-hydroxy-tryptamine, N-Ethyl-4-hydroxy-N-methyl-tryptamine, 4-Hydroxy-N-isopropyl-N-methyl-tryptamine, 4-Hydroxy-N-methyl-N-propyl-tryptamine, 4-Hydroxy-N,N-tetramethylene-tryptamine, N,N-Diethyl-L, N-Butyl-N-methyl-tryptamine, N,N-Diisopropyl-4,5-methylenedioxy-tryptamine, N,N-Diisopropyl-5,6-methylenedioxy-T, N,N-Dimethyl-4,5-methylenedioxy-tryptamine, N,N-Dimethyl-5,6-methylenedioxy-tryptamine, N-Isopropyl-N-methyl-5,6-methylenedioxy-tryptamine, N,N-Diethyl-2-methyl-tryptamine, 2,N,N-Trimethyl-tryptamine, N-Acetyl-5-methoxy-tryptamine, N,N-Diethyl-5-methoxy-tryptamine, N,N-Diisopropyl-5-methoxy-tryptamine, 5-Methoxy-N, N-dimethyl-tryptamine, N-Isopropyl-4-methoxy-N-methyl-tryptamine, N-Iso-propyl-5-methoxy-N-methyl-tryptamine, 5,6-Dimethoxy-N-isopropyl-N-methyl-tryptamine, 5-Methoxy-N-methyl-tryptamine, 5-Methoxy-N,N-tetramethylene-tryptamine, 6-Methoxy-1-methyl-1,2,3,4-tetrahydroisoquinoline, 5-Methoxy-2,N,N-trimethyl-tryptamine, N,N-Dimethyl-5-methylthio-tryptamine, N-Isopropyl-N-methyl-tryptamine, alpha-Methyl-tryptamine, N-Ethyl-tryptamine, N-Methyl-tryptamine, 6-Propyl-N L, N,N-Tetramethylene-tryptamine, Tryptamine, 7-Methoxy-1-methyl-1,2,3,4-tetrahydro-C, and alpha, N-Dimethyl-5-methoxy-T.

57. The method of any one of claims 44-47, wherein the psychedelic is psilocybin or a pharmaceutically acceptable salt, solvate, metabolite, deuterated analogue, derivative, or prodrug thereof.

58. The method of any one of claims 44-47, wherein the psychedelic is 1-acetyl LSD or a pharmaceutically acceptable salt, solvate, metabolite, deuterated analogue, derivative, or prodrug thereof.

59. The method of any one of claims 44-47, wherein the psychedelic is O-acetyl psilocin, or a pharmaceutically acceptable salt, solvate, metabolite, deuterated analogue, derivative, or prodrug thereof.

60. The method of any one of claims 44-59, wherein the serotonin receptor modulator is a serotonin receptor antagonist.

61. The method of any one of claims 44-59, wherein the serotonin receptor modulator is a serotonin receptor inverse agonist.

62. The method of any one of claims 60-61, wherein the serotonin receptor is 5-HT2A receptor.

63. The method of any one of claims 44-59, wherein the serotonin receptor modulator is selected from ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, AMDA, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, and RS-102221, or a pharmaceutically acceptable salt, solvate, metabolite, deuterated analogue, derivative, prodrug, or combinations thereof.

64. The method of any one of claims 44-59, wherein the serotonin receptor modulator is ketanserin or a pharmaceutically acceptable salt, solvate, metabolite, deuterated analogue, derivative, or prodrug thereof.

65. The method of any one of claims 44-59, wherein the serotonin receptor modulator is pimavanserin or a pharmaceutically acceptable salt, solvate, metabolite, deuterated analogue, derivative, or prodrug thereof.

66. The method of any one of claims 44-59, wherein the serotonin receptor modulator is eplivanserin (SR-46,349) or a pharmaceutically acceptable salt, solvate, metabolite, deuterated analogue, derivative, or prodrug thereof.

67. The method of any one of claims 44-66, the serotonin receptor modulator is released at most about 3 hours prior to the release of the psychedelic.

68. The method of any one of claims 44-66, the serotonin receptor modulator is released at most about 2.5 hours prior to the release of the psychedelic.

69. The method of any one of claims 44-66, wherein the serotonin receptor modulator is released at most about 2 hours prior to the release of the psychedelic.

70. The method of any one of claims 44-66, wherein the serotonin receptor modulator is released at most about 1.5 hours prior to the release of the psychedelic.

71. The method of any one of claims 44-66, wherein the serotonin receptor modulator is released at most about 1 hour prior to the release of the psychedelic.

72. The method of any one of claims 44-66, wherein the serotonin receptor modulator is released at about 1 hour prior to the release of the psychedelic.

73. The method of any one of claims 44-66, the serotonin receptor modulator is released at least about 0.5 hours prior to the release of the psychedelic.

74. The method of any one of claims 44-66, the serotonin receptor modulator is released at least about 1 hour prior to the release of the psychedelic.

75. The method of any one of claims 44-74, wherein the psychedelic is provided at a dose of about 100 milligrams (mg) to about 1 gram (g).

76. The method of any one of claims 44-74, wherein the psychedelic is provided at a dose of about 100 mg to about 800 mg.

77. The method of any one of claims 44-74, wherein the psychedelic is provided at a dose of about 100 mg to about 500 mg.

78. The method of any one of claims 44-74, wherein the psychedelic is provided at a dose of about 10 mg to about 400 mg.

79. The method of any one of claims 44-74, wherein the psychedelic is provided at a dose of about 20 mg to about 200 mg.

80. The method of any one of claims 44-74, wherein the psychedelic is provided at a dose of about 10 mg to about 100 mg.

81. The method of any one of claims 44-74, wherein the psychedelic is provided at a dose of about 10 micrograms (ug) to about 400 μg.

82. The method of any one of claims 44-74, wherein the psychedelic is provided at a dose of about 20 μg to about 200 μg.

83. The method of any one of claims 44-74, wherein the psychedelic is provided at a dose of about 10 μg to about 100 μg.

84. The method of any one of claims 44-83, wherein the serotonin receptor modulator is provided at a dose of about 10 mg to about 350 mg.

85. The method of any one of claims 44-83, wherein the serotonin receptor modulator is provided at a dose of about 20 mg to about 200 mg.

86. The method of any one of claims 44-83, wherein the serotonin receptor modulator is provided at a dose of about 10 mg to about 100 mg.

87. The method of any one of claims 44-86, wherein the psychedelic is provided at a dose of about 10 mg to about 100 mg and the serotonin receptor modulator is provided at a dose of about 10 mg to about 100 mg.

88. The method of any one of claims 44-86, wherein the psychedelic is psilocybin or a pharmaceutically acceptable salt, solvate, metabolite, deuterated analogue, derivative, or prodrug thereof and the serotonin receptor modulator is ketanserin or a pharmaceutically acceptable salt thereof.

89. The method of any one of claims 44-86, wherein the psychedelic is psilocybin or a pharmaceutically acceptable salt, solvate, metabolite, deuterated analogue, derivative, or prodrug thereof and provided at a dose of about 10 mg to about 100 mg; and the serotonin receptor modulator is ketanserin or a pharmaceutically acceptable salt thereof provided at a dose of about 10 mg to about 100 mg

90. The method of any one of claims 44-89, wherein the composition is in a form of a single pill, single ampoule, or single unit dosage form.

91. The method of any one of claims 44-89, wherein the composition is in a single unit dosage form.

92. A method of treating a disease or disorder in a subject in need thereof, the method comprising administering to the subject a psychedelic and a serotonin receptor modulator, wherein the serotonin receptor is administered prior to the administration of the psychedelic, and wherein the serotonin receptor modulator is administered at most about 3 hours prior to the administration of the psychedelic.

93. A method of treating a disease or disorder in a subject in need thereof, the method comprising (i) administering to the subject a serotonin receptor modulator to pretreat the subject, and (ii) administering to the subject a psychedelic, and wherein the serotonin receptor modulator is administered at most about 3 hours prior to the administration of the psychedelic.

94. A method of reducing the adverse effects of a psychedelic in the treatment of a disease or disorder in a subject in need thereof, the method comprising (i) administering to the subject a serotonin receptor modulator to pretreat the subject, and (ii) administering to the subject a psychedelic, and wherein the serotonin receptor modulator is administered at most about 3 hours prior to the administration of the psychedelic.

95. The method of any one of claims 92-94, wherein the disease or disorder is depression or a disease or disorder related to depression.
